(19) United States
(12) Patent Application Publication
(10) Pub. No.: US 2026/0235949 A1
(43) Pub. Date: Aug. 13, 2026

(54) RESIST COMPOSITION, RESIST PATTERN FORMATION METHOD, COMPOUND, AND POLYMER COMPOUND

(71) Applicant: TOKYO OHKA KOGYO CO., LTD., Kawasaki-shi (JP)

(72) Inventors: Koshi ONISHI, Kawasaki-shi (JP); Takashi NAGAMINE, Kawasaki-shi (JP)

(21) Appl. No.: 19/157,993

(22) PCT Filed: Feb. 16, 2024
(86) PCT No.: PCT/JP2024/005476

§ 371 Date: Aug. 19, 2025

(30) Foreign Application Priority Data
Feb. 22, 2023 (JP) 2023-026053

(51) Int. Cl. G03F 7/004

(52) U.S. Cl. CPC G03F 7/0045

(57) ABSTRACT

A resist composition which generates an acid upon light exposure and whose solubility in a developing solution is changed by an action of an acid. The resist composition includes a resin component whose solubility in a developing solution is changed by an action of an acid, in which the resin component has a constitutional unit represented by Formula (a0-1) in which A0 is an oxygen atom or a sulfur atom; Y01 is an (n01+1)-valent linking group; L01 is a single bond or a divalent linking group; R01 is a hydrogen atom or a group represented by Formula (r01-1) or (r01-2); V01 and V02 are a single bond or a methylene group; Mm+ is an m-valent cation; Y02 is an (n02+1)-valent linking group; L02 is a single bond or a divalent linking group; Y03 is a single bond or a divalent linking group; and R03 is a hydrocarbon group which may have a substituent

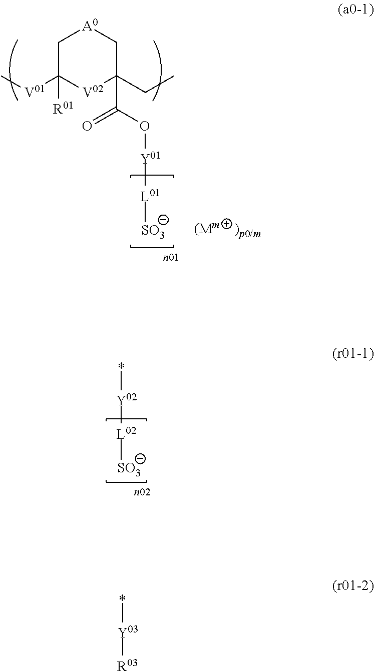

### TECHNICAL FIELD

The present invention relates to a resist composition, a resist pattern formation method, a compound, and a polymer compound.

Priority is claimed on Japanese Patent Application No. 2023-026053, filed on Feb. 22, 2023, the content of which is incorporated herein by reference.

### BACKGROUND ART

In recent years, in the manufacture of semiconductor elements and liquid crystal display elements, advances in lithography technologies have led to rapid progress in the field of pattern fining. These pattern fining techniques typically involve shortening the wavelength (increasing the energy) of the exposure light source.

Resist materials are required to have lithography characteristics such as sensitivity to these exposure light sources and resolution that enables reproduction of patterns with minute dimensions.

As a resist material that satisfies these requirements, a chemically amplified resist composition containing a base material component whose solubility in a developing solution is changed by an action of an acid and an acid generator component that generates an acid upon light exposure has been used in the related art.

In addition, as the acid generator component, a resin in which a constitutional unit containing an acid-generating group that generates an acid upon light exposure is introduced has been suggested (for example. Patent Document 1). Such a resin has both a function as an acid generator and a function as a base material component.

### CITATION LIST

### Patent Document

[Patent Document 1] Japanese Unexamined Patent Application, First Publication No. 2014-197168

### SUMMARY OF INVENTION

### Technical Problem

With further advances in lithography technologies, and rapid progress in the field of pattern fining, for example, the aim is to form a fine pattern with a size of several tens of nanometers in lithography by EUV and EB. As the resist pattern dimensions decrease, each of the lithography characteristics such as sensitivity, roughness, and resolution is required to be improved without trade-off.

However, these lithography characteristics are in a trade-off relationship, and it is difficult to satisfy all of these characteristics.

The present invention has been made in view of the above circumstances, and an object thereof is to provide a resist composition having good sensitivity, roughness, and resolution, a resist pattern formation method using the resist composition, a polymer compound that can be used for producing the resist composition, and a compound that can be used for synthesizing the polymer compound.

### Solution to Problem

In order to solve the above-described problems, the present invention has adopted the following configurations.

That is, according to a first aspect of the present invention, there is provided a resist composition which generates an acid upon light exposure and whose solubility in a developing solution is changed by an action of an acid, the resist composition including: a resin component (A1) whose solubility in a developing solution is changed by the action of the acid, in which the resin component (A1) has a constitutional unit (a0) represented by General Formula (a0-1).

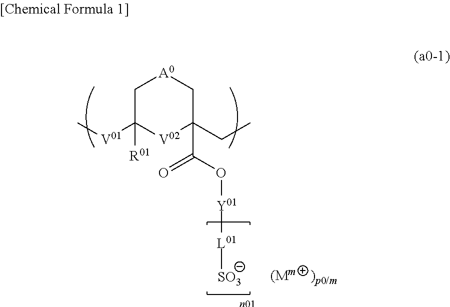

[In the formula, A⁰ represents an oxygen atom or a sulfur atom. Y⁰¹ represents an (n01+1)-valent linking group. L⁰¹ represents a single bond or a divalent linking group. R⁰¹ represents a hydrogen atom or a group represented by General Formula (r01-1) or (r01-2). V⁰¹ and V⁰² each represents a single bond or a methylene group, where V⁰² is a methylene group in a case where V⁰¹ is a single bond, and V⁰² is a single bond in a case where V⁰¹ is a methylene group. n01 represents an integer of 1 or greater as long as the valence is allowed. m represents an integer of 1 or greater, and M^{m+} represents an m-valent cation. p0=n01 in a case where R⁰¹ is a hydrogen atom or a group represented by General Formula (r01-2), and p0=n01+n02 in a case where R⁰¹ is a group represented by General Formula (r01-1).]

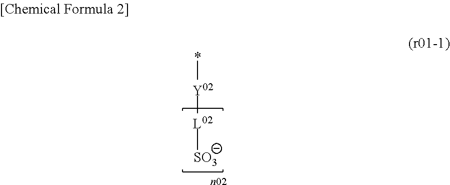

[in the formula, Y⁰² represents an (n02+1)-valent linking group. L⁰² represents a single bond or a divalent linking group. n02 represents an integer of 1 or greater as long as the valence is allowed.]

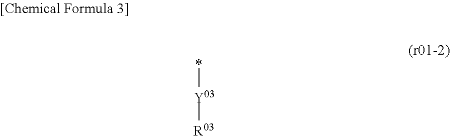

[In the formula, Y⁰³ represents a single bond or a divalent linking group. R⁰³ represents a hydrocarbon group which may have a substituent.]

According to a second aspect of the present invention, there is provided a resist pattern formation method, including: a step of forming a resist film on a support using the resist composition according to the first aspect; a step of exposing the resist film to light; and a step of developing the resist film exposed to light to form a resist pattern.

According to a third aspect of the present invention, there is provided a compound which is represented by General Formula (m0-1).

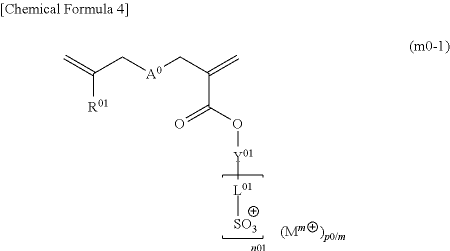

[In the formula. A⁰ represents an oxygen atom or a sulfur atom. Y⁰¹ represents an (n01+1)-valent linking group. L⁰¹ represents a single bond or a divalent linking group. R⁰¹ represents a hydrogen atom or a group represented by General Formula (r01-1) or (r01-2). n01 represents an integer of 1 or greater as long as the valence is allowed. m represents an integer of 1 or greater, and M^{m+} represents an m-valent cation. p0=n01 in a case where R⁰¹ is a hydrogen atom or a group represented by General Formula (r01-2), and p0=n01+n02 in a case where R⁰¹ is a group represented by General Formula (r01-1).]

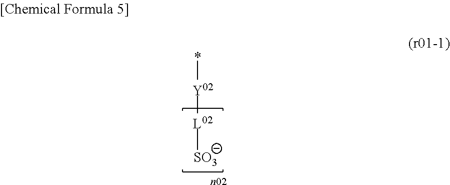

[In the formula, Y⁰² represents an (n02+1)-valent linking group. L⁰² represents a single bond or a divalent linking group. n02 represents an integer of 1 or greater as long as the valence is allowed.]

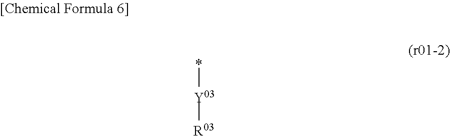

[In the formula. Y⁰³ represents a single bond or a divalent linking group. R⁰³ represents a hydrocarbon group which may have a substituent.]

According to a fourth aspect of the present invention, there is provided a polymer compound which has a constitutional unit (a0) represented by General Formula (a0-1).

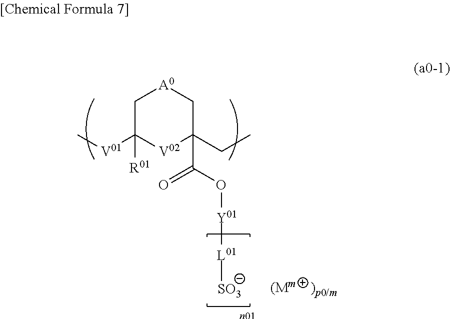

[In the formula, A⁰ represents an oxygen atom or a sulfur atom. Y⁰¹ represents an (n01+1)-valent linking group. L⁰¹ represents a single bond or a divalent linking group. R⁰¹ represents a hydrogen atom or a group represented by General Formula (r01-1) or (r01-2). V⁰¹ and V⁰² each represents a single bond or a methylene group, where V⁰² is a methylene group in a case where V⁰¹ is a single bond, and V⁰² is a single bond in a case where V⁰¹ is a methylene group. n01 represents an integer of 1 or greater as long as the valence is allowed. m represents an integer of 1 or greater, and M^{m+} represents an m-valent cation. p0=n01 in a case where R⁰¹ is a hydrogen atom or a group represented by General Formula (r01-2), and p0=n01+n02 in a case where R⁰¹ is a group represented by General Formula (r01-1).]

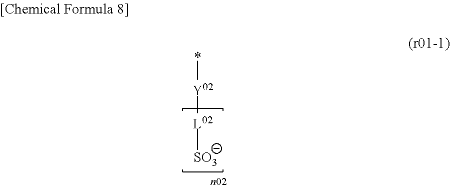

[In the formula, Y⁰² represents an (n02+1)-valent linking group. L⁰² represents a single bond or a divalent linking group. n02 represents an integer of 1 or greater as long as the valence is allowed.]

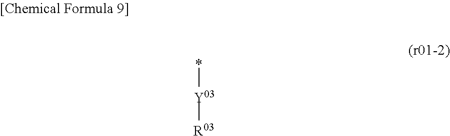

[In the formula, Y⁰³ represents a single bond or a divalent linking group. R⁰³ represents a hydrocarbon group which may have a substituent.]

### Advantageous Effects of Invention

According to the present invention, it is possible to provide a resist composition having good sensitivity, roughness, and resolution, a resist pattern formation method using the resist composition, a polymer compound that can be used for producing the resist composition, and a compound that can be used for synthesizing the polymer compound.

### DESCRIPTION OF EMBODIMENTS

In the present specification and the scope of the present claims, the term “aliphatic” is a relative concept used with respect to “aromatic” and defines a group, a compound, or the like that has no aromaticity.

The term “alkyl group” includes a linear, branched, or cyclic monovalent saturated hydrocarbon group unless otherwise specified. The same applies to the alkyl group in an alkoxy group.

The term “alkylene group” includes a linear, branched, or cyclic divalent saturated hydrocarbon group unless otherwise specified.

Examples of “halogen atom” include a fluorine atom, a chlorine atom, a bromine atom, and an iodine atom.

The term “constitutional unit” indicates a monomer unit constituting a polymer compound (a resin, a polymer, or a copolymer).

The expression “may have a substituent” includes both a case where a hydrogen atom (—H) is substituted with a monovalent group and a case where a methylene (—CH₂—) group is substituted with a divalent group.

The term “light exposure” is a general concept for irradiation with radiation.

The term “acid-decomposable group” indicates a group having acid decomposability in which at least part of a bond in the structure of the acid-decomposable group can be cleaved by the action of an acid.

Examples of the acid-decomposable group whose polarity is increased by the action of an acid include groups which are decomposed by the action of an acid to generate a polar group.

Examples of the polar group include a carboxy group, a hydroxyl group, an amino group, and a sulfo group (—SO₃H).

More specific examples of the acid-decomposable group include a group in which the above-described polar group has been protected by an acid-dissociable group (such as a group in which a hydrogen atom of the OH-containing polar group has been protected by an acid-dissociable group).

Here, the term “acid-dissociable group” indicates both a group (i) having an acid dissociation property in which a bond between the acid-dissociable group and an atom adjacent to the acid-dissociable group can be cleaved by the action of an acid and a group (ii) in which some bonds are cleaved by the action of an acid, a decarboxylation reaction occurs, and thus the bond between the acid-dissociable group and the atom adjacent to the acid-dissociable group can be cleaved.

It is necessary that the acid-dissociable group that constitutes the acid-decomposable group be a group which exhibits a lower polarity than that of the polar group generated by the dissociation of the acid-dissociable group. Thus, in a case where the acid-dissociable group is dissociated by the action of an acid, a polar group exhibiting a higher polarity than that of the acid-dissociable group is generated so that the polarity is increased. As a result, the polarity of an entire component (A1) is increased. Due to the increase in the polarity, the solubility in a developing solution is relatively changed such that the solubility is increased in a case where the developing solution is an alkali developing solution and the solubility is decreased in a case where the developing solution is an organic developing solution.

The term “base material component” denotes an organic compound having a film-forming ability. Organic compounds used as the base material component are classified into non-polymers and polymers. As the non-polymers, typically non-polymers having a molecular weight of 500 or greater and less than 4000 (hereinafter, referred to as “low-molecular-weight compounds”) are used. Hereinafter, the term “resin”, “polymer compound”, or “polymer” indicates a polymer having a molecular weight of 1000 or greater. As the molecular weight of the polymer, the weight-average molecular weight in terms of polystyrene according to gel permeation chromatography (GPC) is used.

The expression “constitutional unit to be derived” denotes a constitutional unit formed by cleavage of a multiple bond between carbon atoms, for example, an ethylenic double bond.

In “acrylic acid ester”, the hydrogen atom bonded to the carbon atom at the α-position may be substituted with a substituent. The substituent (R^{αx}) that substitutes the hydrogen atom bonded to the carbon atom at the α-position is an atom other than the hydrogen atom or a group. Further, the acrylic acid ester includes itaconic acid diester in which the substituent (R^{αx}) has been substituted with a substituent having an ester bond and α-hydroxyacryl ester in which the substituent (R^{αx}) has been substituted with a hydroxyalkyl group or a group obtained by modifying a hydroxyl group thereof. Further, the carbon atom at the α-position of acrylic acid ester indicates the carbon atom to which the carbonyl group of acrylic acid is bonded, unless otherwise specified.

Hereinafter, acrylic acid ester in which the hydrogen atom bonded to the carbon atom at the α-position has been substituted with a substituent is also referred to as α-substituted acrylic acid ester.

The concept “derivative” includes those obtained by substituting a hydrogen atom at the α-position of a target compound with another substituent such as an alkyl group or a halogenated alkyl group, and derivatives thereof. Examples of the derivatives thereof include those obtained by substituting a hydrogen atom of a hydroxyl group of a target compound, in which the hydrogen atom at the α-position may be substituted with a substituent, with an organic group; and those obtained by bonding a substituent other than a hydroxyl group to a target compound in which the hydrogen atom at the α-position may be substituted with a substituent. Further, the α-position denotes the first carbon atom adjacent to a functional group unless otherwise specified.

Examples of the substituent that substitutes the hydrogen atom at the α-position of hydroxystyrene include the same substituents as those for R^{αx}.

In the present specification and the scope of the present claims, asymmetric carbons may be present and enantiomers or diastereomers may be present depending on the structures of the chemical formulae. In this case, these isomers are represented by one chemical formula. These isomers may be used alone or in the form of a mixture.

### (Resist Composition)

The resist composition according to the present embodiment is a resist composition which generates an acid upon light exposure and whose solubility in a developing solution is changed by the action of the acid.

The resist composition contains a base material component (A) (hereinafter, also referred to as “component (A)”) whose solubility in a developing solution is changed by the action of the acid. In the resist composition according to the present embodiment, the component (A) generates an acid upon light exposure. It is preferable that the component (A1) described below in the resist composition according to the present embodiment be a resin which generates an acid upon light exposure and whose solubility in a developing solution is changed by an action of the acid.

In a case where a resist film is formed of the resist composition according to the present embodiment and the resist film is selectively exposed, since an acid is generated from the component (A) in an exposed portion of the resist film and the solubility of the component (A) in a developing solution is changed by the action of the acid while the solubility of the component (A) in a developing solution is not changed in an unexposed portion of the resist film, a difference in solubility in the developing solution occurs between the exposed portion and the unexposed portion. Therefore, in a case where the resist film is developed, the exposed portion of the resist film is dissolved and removed to form a positive-tone resist pattern in a case where the resist composition is of a positive-tone, whereas the unexposed portion of the resist film is dissolved and removed to form a negative-tone resist pattern in a case where the resist composition is of a negative tone.

The resist composition according to the present embodiment may be a positive-tone resist composition or a negative-tone resist composition. Further, in the formation of a resist pattern, the resist composition according to the present embodiment may be applied to an alkali developing process using an alkali developing solution in the developing treatment, or a solvent developing process using a developing solution containing an organic solvent (organic developing solution) in the developing treatment.

### <Base Material Component (A)>

In the resist composition according to the present embodiment, it is preferable that the component (A) have a resin component (A1) whose solubility in a developing solution is changed by the action of an acid (hereinafter, also referred to as “component (A1)”). Since the polarity of the base material component before and after light exposure is changed using the component (A1), a satisfactory development contrast can be obtained not only in an alkali developing process but also in a solvent developing process.

As the component (A), another polymer compound and/or a low-molecular-weight compound may be used in combination with the component (A1).

In the resist composition according to the present embodiment, the component (A) may be used alone or in combination of two or more kinds thereof.

### In Regard to Component (A1)

The component (A1) is a resin component whose solubility in a developing solution is changed by the action of an acid.

The component (A1) has a constitutional unit (a0) represented by General Formula (a0-1).

As the component (A1), those having a constitutional unit (a1) containing an acid-decomposable group whose polarity is increased by the action of an acid are preferable.

Further, the component (A1) may have other constitutional units as necessary in addition to the constitutional unit (a1).

<<Constitutional Unit (a0)>>

The constitutional unit (a0) is a constitutional unit represented by General Formula (a0-1).

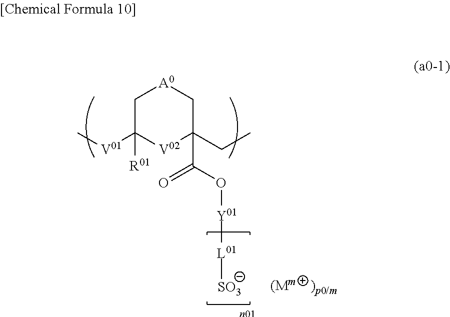

[In the formula, A⁰ represents an oxygen atom or a sulfur atom. Y⁰¹ represents an (n01+1)-valent linking group. L⁰¹ represents a single bond or a divalent linking group. R⁰¹ represents a hydrogen atom or a group represented by General Formula (r01-1) or (r01-2). V⁰¹ and V⁰² each represents a single bond or a methylene group, where V⁰² is a methylene group in a case where V⁰¹ is a single bond, and V⁰² is a single bond in a case where V⁰¹ is a methylene group. n01 represents an integer of 1 or greater as long as the valence is allowed. m represents an integer of 1 or greater, and M^{m+} represents an n-valent cation. p0=n01 in a case where R⁰¹ is a hydrogen atom or a group represented by General Formula (r0-2), and p0=n01+n02 in a case where R⁰¹ is a group represented by General Formula (r01-1).]

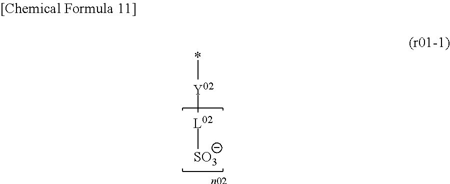

[In the formula, Y⁰² represents an (n02+1)-valent linking group. L⁰² represents a single bond or a divalent linking group. n02 represents an integer of 1 or greater as long as the valence is allowed.]

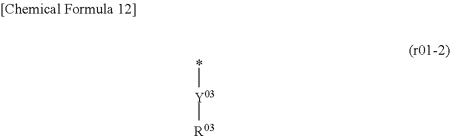

[In the formula, Y⁰³ represents a single bond or a divalent linking group. R⁰³ represents a hydrocarbon group which may have a substituent.]

In Formula (a0-1), A⁰ is preferably an oxygen atom.

In Formula (a0-1), examples of the (n01+1)-valent linking group as Y⁰¹ include an (n01+1)-valent hydrocarbon group which may have a substituent, and an (n01+1)-valent linking group having a heteroatom.

(n01+1)-Valent Hydrocarbon Group which May have a Substituent:

In a case where Y⁰¹ represents an (n01+1)-valent hydrocarbon group which may have a substituent, the hydrocarbon group may be an aliphatic hydrocarbon group or an aromatic hydrocarbon group.

### Aliphatic Hydrocarbon Group as Y⁰¹

The aliphatic hydrocarbon group indicates a hydrocarbon group that has no aromaticity. The aliphatic hydrocarbon group may be saturated or unsaturated. In general, it is preferable that the aliphatic hydrocarbon group be saturated.

Examples of the aliphatic hydrocarbon group include a linear or branched aliphatic hydrocarbon group and an aliphatic hydrocarbon group having a ring in the structure thereof.

### Linear or Branched Aliphatic Hydrocarbon Group

The linear aliphatic hydrocarbon group preferably has 1 to 10 carbon atoms, more preferably has 1 to 6 carbon atoms, still more preferably has 1 to 4 carbon atoms, and particularly preferably has 1 to 3 carbon atoms.

As the linear aliphatic hydrocarbon group, a group in which (n01−1) hydrogen atoms have been removed from a linear alkylene group is preferable. Examples of the linear alkylene group include a methylene group [—CH₂—], an ethylene group [—(CH₂)₂—], a trimethylene group [—(CH₂)₃—], and a tetramethylene group [—(CH₂)₄—], and a pentamethylene group [—(CH₂)₅—].

The branched aliphatic hydrocarbon group preferably has 2 to 10 carbon atoms, more preferably has 3 to 6 carbon atoms, still more preferably has 3 or 4 carbon atoms, and particularly preferably has 3 carbon atoms.

As the branched aliphatic hydrocarbon group, a group in which (n01−1) hydrogen atoms have been removed from a branched alkylene group is preferable. Examples of the branched alkylene group include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—, alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—, alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

The linear or branched aliphatic hydrocarbon group may or may not have a substituent. Examples of the substituent include a fluorine atom, a fluorinated alkyl group having 1 to 5 carbon atoms which has been substituted with a fluorine atom, and a carbonyl group.

### Aliphatic Hydrocarbon Group Having Ring in Structure Thereof

Examples of the aliphatic hydrocarbon group having a ring in the structure thereof include a cyclic aliphatic hydrocarbon group which may have a substituent having a heteroatom in the ring structure thereof (a group in which (n+1) hydrogen atoms have been removed from an aliphatic hydrocarbon ring), a group in which the cyclic aliphatic hydrocarbon group is bonded to the terminal of a linear or branched aliphatic hydrocarbon group, and a group in which the cyclic aliphatic hydrocarbon group is interposed in the middle of a linear or branched aliphatic hydrocarbon group. Examples of the linear or branched aliphatic hydrocarbon group include the same groups as described above.

The cyclic aliphatic hydrocarbon group preferably has 3 to 20 carbon atoms and more preferably has 3 to 12 carbon atoms.

The cyclic aliphatic hydrocarbon group may be a polycyclic group or a monocyclic group. As the monocyclic alicyclic hydrocarbon group, a group in which (n+1) hydrogen atoms have been removed from a monocycloalkane is preferable. The monocycloalkane preferably has 3 to 6 carbon atoms, and specific examples thereof include cyclopentane and cyclohexane. As the polycyclic alicyclic hydrocarbon group, a group in which (n+1) hydrogen atoms have been removed from a polycycloalkane is preferable. The polycycloalkane preferably has 7 to 12 carbon atoms, and specific examples thereof include adamantane, norbornane, isobornane, tricyclo[5.2.1.0^{2,6}]decane, and tetracyclododecane.

The cyclic aliphatic hydrocarbon group may or may not have a substituent. Examples of the substituent include an alkyl group, an alkoxy group, a halogen atom, a halogenated alkyl group, a hydroxyl group, and a carbonyl group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is still more preferable.

As the halogen atom as a substituent, a fluorine atom or an iodine atom is preferable.

Examples of the halogenated alkyl group as the substituent include groups in which some or all hydrogen atoms in the above-described alkyl groups have been substituted with the above-described halogen atoms.

In the cyclic aliphatic hydrocarbon group, some carbon atoms constituting the ring structure thereof may be substituted with a substituent having a heteroatom. As the substituent having a heteroatom, —O—, —C(═O)—O—, —S—, —S(═O)₂—, or —S(═O)₂—O— is preferable.

### Aromatic Hydrocarbon Group as Y⁰¹

The aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring preferably has 5 to 30 carbon atoms, more preferably has 5 to 20 carbon atoms, still more preferably has 6 to 15 carbon atoms, and particularly preferably has 6 to 12 carbon atoms. The number of carbon atoms does not include the number of carbon atoms in the substituent.

Specific examples of the aromatic ring include aromatic hydrocarbon rings such as benzene, naphthalene, anthracene, and phenanthrene; and aromatic heterocyclic rings in which some carbon atoms constituting the above-described aromatic hydrocarbon rings have been substituted with heteroatoms. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group include a group in which (n+1) hydrogen atoms have been removed from the above-described aromatic hydrocarbon ring or aromatic heterocyclic ring (an arylene group or a heteroarylene group), a group in which (n+1) hydrogen atoms have been removed from an aromatic compound having two or more aromatic rings (for example, biphenyl or fluorene), and a group in which one hydrogen atom of a group (an aryl group or a heteroaryl group) obtained by removing one hydrogen atom from the above-described aromatic hydrocarbon ring or aromatic heterocyclic ring has been substituted with an alkylene group (for example, a group obtained by further removing one hydrogen atom from an aryl group in an arylalkyl group such as a benzyl group, a phenethyl group, a 1-naphthylmethyl group, a 2-naphthylmethyl group, a 1-naphthylethyl group, or a 2-naphthylethyl group). The number of carbon atoms in the alkylene group bonded to the aryl group or the heteroaryl group is preferably in a range of 1 to 4, more preferably 1 or 2, and particularly preferably 1.

In the aromatic hydrocarbon group, the hydrogen atom in the aromatic hydrocarbon group may be substituted with a substituent. For example, the hydrogen atom bonded to the aromatic ring in the aromatic hydrocarbon group may be substituted with a substituent. Examples of substituents include an alkyl group, an alkoxy group, a halogen atom, a halogenated alkyl group, and a hydroxyl group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group, the halogen atom, and the halogenated alkyl group as the substituents, the groups described as the substituents that substitute a hydrogen atom in the cyclic aliphatic hydrocarbon group are exemplary examples. As the halogen atom, a fluorine atom or an iodine atom is preferable, and an iodine atom is more preferable.

(n01+1)-Valent Linking Group Having Heteroatom:

In a case where Y⁰¹ represents an (n01+1) linking group having a hetero atom, preferred examples of the linking group include an (n01+1)-valent linking group including —O—, —C(═O)—O—, —O—C(═O)—, —C(═O)—, —O—C(═O)—O—, —C(═O)—NH—, —NH—, —NH—C(═NH)— (H may be substituted with a substituent such as an alkyl group, an acyl group, or the like), —S—, —S(═O)₂, —S(═O)₂—O—, and a group represented by General Formula: —Y²¹—O—Y²²—, —Y²¹—O—, —Y²¹—C(═O)—O—, —C(═O)—O—Y²¹—, —[Y²¹—C(═O)—O]ₘ—Y²², —Y²¹—O—C(═O)—Y²²—, or —Y²¹—S(═O)₂—O—Y²²—[in the formulae, Y²¹ and Y²² each independently represents a divalent hydrocarbon group which may have a substituent, 0 represents an oxygen atom, and m″ represents an integer of 1 to 3].

It is preferable that Y⁰¹ represent the (n01+1)-valent hydrocarbon group which may have a substituent. In a case where n0 is 2 or greater, Y⁰¹ preferably represents an (n01+1)-valent branched aliphatic hydrocarbon group which may have a substituent, an (n01+1)-valent cyclic aliphatic hydrocarbon group which may have a substituent, or an (n01+1)-valent aromatic hydrocarbon group which may have a substituent, and preferably represents an (n01+1)-valent aromatic hydrocarbon group which may have a substituent.

In a case where n01 is 1, Y⁰¹ preferably represents a divalent hydrocarbon group which may have a substituent, and more preferably a linear or branched alkylene group which may have a substituent, a divalent cyclic aliphatic hydrocarbon group which may have a substituent, or a divalent aromatic hydrocarbon group which may have a substituent.

Specific examples of the linear or branched alkylene group include the same groups as described above. Specific examples of the divalent cyclic aliphatic hydrocarbon group include a group in which two hydrogen atoms have been removed from a monocycloalkane or a polycycloalkane. Examples of the monocycloalkane and the polycycloalkane include the same groups as described above.

Examples of the divalent aromatic hydrocarbon group include a group in which two hydrogen atoms have been removed from an aromatic hydrocarbon ring or an aromatic heterocyclic ring. Examples of the aromatic hydrocarbon ring or the aromatic heterocyclic ring include the same groups as described above.

n01 preferably represents an integer of 1 to 5, more preferably an integer of 1 to 4, still more preferably an integer of 1 to 3, particularly preferably 1 or 2, and most preferably 1.

In Formula (a0-1), the divalent linking group as L⁰¹ is not particularly limited, and examples thereof include a divalent hydrocarbon group which may have a substituent, a divalent linking group having a heteroatom. Examples of the divalent linking group as L⁰¹ include the same as those in a case where the (n01+1)-valent linking group as Y⁰¹ is a divalent linking group.

The divalent linking group as L⁰¹ is preferably a group represented by General Formula (l01-1).

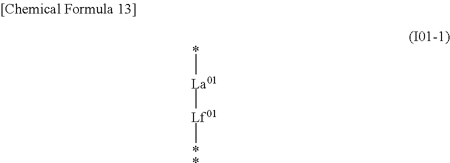

[In the formula, La⁰¹ represents a divalent linking group having an oxygen atom or a single bond. Lf⁰¹ represents a hydrocarbon group having a fluorine atom or a single bond. * represents a bonding site with respect to Y⁰¹ in General Formula (a0-1). ** represents a bonding site with respect to the sulfur atom (S) in General Formula (a0-1).]

The divalent linking group having an oxygen atom as La⁰¹ in Formula (l01-1) may have an atom other than oxygen. Examples of the atom other than oxygen, which may be included as La⁰¹, include a carbon atom, a nitrogen atom, and a sulfur atom.

Examples of the divalent linking group having an oxygen atom as La⁰¹ include a linking group represented by —O—, —C(═O)—O—, —O—C(═O)—, —C(═O)—, —O—C(═O)—O—, —C(═O)—NH—, —NH—C(═O)—, —S(═O)₂—O—, or —O—S(═O)₂—; and a group which is a combination of one or more of the linking groups and a hydrocarbon group which may have a substituent. Examples of the group which is the combination include a group represented by -Va-Ra-(Vb-Rb)ₘ—. In the formula, Va and Vb each independently represents a linking group selected from the group consisting of —O—, —C(═O)—O—, —O—C(═O)—, —C(═O)—, —O—C(═O)—O—, —C(═O)—NH—, —NH—C(═O)—, —S(═O)₂—O—, and —O—S(═O)₂—; Ra represents a hydrocarbon group which may have a substituent, or a single bond; and Rb represents a hydrocarbon group which may have a substituent, and n represents an integer of 0 to 2. m preferably represents 0 or 1, and more preferably 0. In a case where nm represent 1 or 2, Ra is not a single bond.

The hydrocarbon group in Ra and Rb preferably has 1 to 15 carbon atoms, more preferably has 1 to 10 carbon atoms, and still more preferably has 1 to 8 carbon atoms. The hydrocarbon group may be an aliphatic hydrocarbon group or an aromatic hydrocarbon group, but is preferably an aliphatic hydrocarbon group. The aliphatic hydrocarbon group may be saturated or unsaturated, but is preferably saturated.

The aliphatic hydrocarbon group which may be contained as La⁰¹ may be linear or branched, or may have a ring structure.

The linear aliphatic hydrocarbon group is preferably a linear alkylene group, preferably has 1 to 6 carbon atoms, more preferably has 1 to 4 carbon atoms, still more preferably has 1 to 3 carbon atoms, and particularly preferably a methylene group or an ethylene group.

The branched aliphatic hydrocarbon group is preferably a branched alkylene group, and preferably has 2 to 6 carbon atoms, more preferably 2 to 4 carbon atoms, and still more preferably 2 or 3 carbon atoms.

The aliphatic hydrocarbon group having a ring structure may be a cyclic aliphatic hydrocarbon group, a group in which a cyclic aliphatic hydrocarbon group is bonded to a terminal of a linear or branched aliphatic hydrocarbon group, or a group in which a cyclic aliphatic hydrocarbon group is interposed in the middle of a linear or branched aliphatic hydrocarbon group. The cyclic aliphatic hydrocarbon group preferably has 3 to 12 carbon atoms, more preferably has 3 to 10 carbon atoms, and still more preferably has 1 to 8 carbon atoms.

The cyclic aliphatic hydrocarbon group is preferably a group in which two hydrogen atoms have been removed from a monocycloalkane or a polycycloalkane. Specific examples of the monocycloalkane and the polycycloalkane include the same groups as those of Y⁰¹.

In the cyclic aliphatic hydrocarbon group, a part of carbon atoms in the ring skeleton may be substituted with a substituent having a heteroatom. As the substituent having a heteroatom, —O—, —C(═O)—O—, —S—, —S(═O)₂—, or —S(═O)₂—O— is preferable.

The hydrocarbon group as Lf⁰¹ in Formula (l01-1) may be an aliphatic hydrocarbon group or an aromatic hydrocarbon group, but is preferably an aliphatic hydrocarbon group. The aliphatic hydrocarbon group may be saturated or unsaturated, but is preferably saturated.

Lf⁰¹ is preferably a linear or branched fluorinated alkylene group or a fluorinated arylene group.

In a case where Lf⁰¹ is a linear fluorinated alkylene group, the linear fluorinated alkylene group preferably has 1 to 10 carbon atoms, more preferably has 1 to 6 carbon atoms, still more preferably has 1 to 4 carbon atoms, even more preferably has 1 to 3 carbon atoms, and particularly preferably has 1 or 2 carbon atoms.

In a case where Lf⁰¹ is a linear or branched fluorinated alkylene group, the fluorinated alkylene group preferably has at least one fluorine atom or fluorinated alkyl group bonded to a carbon atom adjacent to the sulfur atom (S) in Formula (a0-1), more preferably a fluorine atom or a trifluoromethyl group bonded to a carbon atom adjacent to the sulfur atom, and still more preferably a fluorine atom bonded to a carbon atom adjacent to the sulfur atom.

In a case where Lf⁰¹ is a branched fluorinated alkylene group, the branched fluorinated alkylene group preferably has 2 to 10 carbon atoms, more preferably has 2 to 6 carbon atoms, still more preferably has 2 to 4 carbon atoms, and particularly preferably has 2 or 3 carbon atoms.

In a case where Lf⁰¹ is a fluorinated arylene group, the branched fluorinated arylene group preferably has 6 to 10 carbon atoms, and more preferably has 6 carbon atoms. The fluorinated arylene group is preferably a perfluoroarylene group, more preferably a perfluorophenylene group or a perfluoronaphthylene group, and still more preferably a pertluorophenylene group.

In Formula (a0-1), R⁰¹ represents a hydrogen atom or a group represented by Formula (r01-1) or (r01-2).

Group Represented by General Formula (r01-1):

The (n02+1)-valent linking group as Y⁰² in Formula (r01-1) is preferably a group represented by —C(═O)—O—Yx⁰²-. Yx⁰² represents an (n02+1)-valent linking group, and is a group bonded to L⁰² in Formula (r01-1). Examples of the (n02+1)-valent linking group as Yx⁰² include the same groups as the (n01+1)-valent linking group as Y⁰¹.

Examples of the divalent linking group in L⁰² as Formula (r01-1) include the same groups as the divalent linking group as L⁰¹. The divalent linking group as L⁰² is preferably a group represented by General Formula (l01-2).

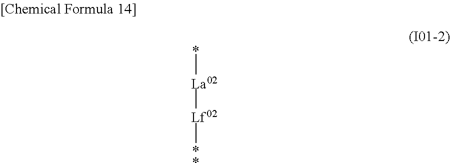

In the formula, La⁰² represents a divalent linking group having an oxygen atom or a single bond. Lf⁰² represents a hydrocarbon group having a fluorine atom or a single bond. * represents a bonding site with respect to Y⁰² in Formula (r01-1). ** represents a bonding site with respect to the sulfur atom (S) in Formula (r01-1).]

Examples of La⁰² in Formula (l01-2) include the same ones as those of La⁰¹ in Formula (l01-1).

Examples of Lf⁰² in Formula (l01-2) include the same ones as those of Lf⁰¹ in Formula (l01-1).

n02 in Formula (r01-1) is preferably an integer of 1 to 5, more preferably an integer of 1 to 4, still more preferably an integer of 1 to 3, particularly preferably 1 or 2, and most preferably 1.

Group Represented by General Formula (r01-2):

Examples of the divalent linking group as Y⁰³ in Formula (r0-2) include a hydrocarbon group which may have a substituent and a divalent linking group having a heteroatom. Examples of the divalent linking group as Y⁰³ include the same as those in a case where the (n01+1)-valent linking group as Y⁰¹ is a divalent linking group.

As the divalent linking group as Y⁰³, a divalent linking group having a heteroatom is preferable. Examples of the divalent linking group having a heteroatom include the same groups as those of the divalent linking group having an oxygen atom as La⁰¹ in Formula (l01-1). As the divalent linking group as Y⁰³, a group which is a combination of —C(═O)—O— or —C(═O)—O— and a linear or branched alkylene group is preferable.

The hydrocarbon group as R⁰³ of Formula (r01-2) may be an aliphatic hydrocarbon group or an aromatic hydrocarbon group.

The aliphatic hydrocarbon group may be saturated or unsaturated, but is preferably saturated. Examples of the aliphatic hydrocarbon group as R⁰³ include a linear or branched aliphatic hydrocarbon group and a cyclic aliphatic hydrocarbon group.

The linear or branched aliphatic hydrocarbon group is preferably a linear or branched alkyl group.

The linear alkyl group preferably has 1 to 5 carbon atoms, more preferably 1 to 4 carbon atoms, and still more preferably 1 or 2 carbon atoms.

The branched alkyl group preferably has 3 to 10 carbon atoms and more preferably has 3 to 5 carbon atoms. Specific examples thereof include an isopropyl group, an isobutyl group, a tert-butyl group, an isopentyl group, a neopentyl group a 1,1-diethylpropyl group, and a 2,2-dimethylbutyl group.

The linear or branched aliphatic hydrocarbon group may have a substituent or may not have a substituent. Examples of the substituent include a fluorine atom, a fluorinated alkyl group having 1 to 5 carbon atoms which has been substituted with a fluorine atom, and a carbonyl group.

The cyclic aliphatic hydrocarbon group may be a monocyclic group or a polycyclic group.

As the aliphatic hydrocarbon group which is a monocyclic group, a group in which one hydrogen atom has been removed from a monocycloalkane is preferable. The monocycloalkane preferably has 3 to 6 carbon atoms, and specific examples thereof include cyclopentane and cyclohexane.

As the aliphatic hydrocarbon group which is a polycyclic group, a group in which one hydrogen atom has been removed from a polycycloalkane is preferable. The polycycloalkane preferably has 7 to 12 carbon atoms, and specific examples thereof include adamantane, norbornane, isobornane, tricyclo[5.2.1.0^{2,6}]decane, and tetracyclododecane.

The aromatic hydrocarbon group may be monocyclic or polycyclic. The aromatic ring contained in the aromatic hydrocarbon group preferably has 5 to 30 carbon atoms, more preferably 5 to 20 carbon atoms, still more preferably 6 to 15 carbon atoms, and particularly preferably 6 to 12 carbon atoms.

Specific examples of the aromatic ring include an aromatic hydrocarbon ring such as benzene, naphthalene, anthracene, and phenanthrene; and an aromatic heterocyclic ring in which some carbon atoms constituting the aromatic hydrocarbon ring have been substituted with heteroatoms. Examples of the heteroatom in the aromatic heterocyclic rings include an oxygen atom, a sulfur atom, and a nitrogen atom. Specific examples of the aromatic heterocyclic ring include a pyridine ring and a thiophene ring.

The cyclic aliphatic hydrocarbon group and the aromatic hydrocarbon group may or may not have a substituent. Examples of the substituent include an alkyl group, an alkoxy group, a halogen atom, a halogenated alkyl group, a hydroxyl group, and a carbonyl group. Examples of these substituents include the same substituents as the substituents as Y⁰¹.

R⁰³ is preferably a linear or branched alkyl group, more preferably a linear or branched alkyl group having 1 to 5 carbon atoms, still more preferably a linear alkyl group having 1 to 3 carbon atoms, and particularly preferably a methyl group or an ethyl group.

In Formula (a0-1), in a case where R⁰¹ is a group represented by Formula (r01-1) or (r01-2), it is preferable that V⁰¹ be a single bond and V⁰² be a methylene group.

In Formula (a0-1), in a case where R⁰¹ is a hydrogen atom. V⁰¹ is a methylene group, and V⁰² is preferably a single bond.

Examples of the constitutional unit (a0) include a constitutional unit represented by General Formula (a01-1), (a02-1), or (a03-1).

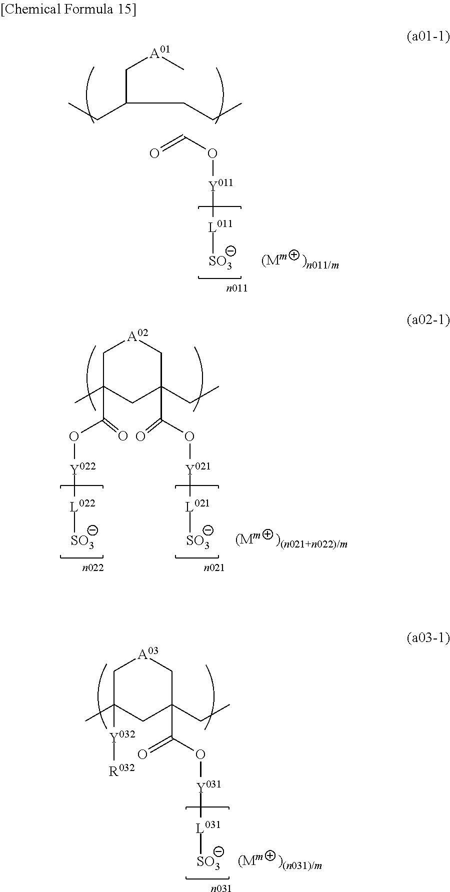

[In the formula, A⁰¹ to A⁰³ each independently represents an oxygen atom or a sulfur atom. Y⁰¹¹ represents an (n011+1)-valent linking group. Y⁰²¹ represents an (n021+1)-valent linking group. Y⁰²² represents an (n022+1)-valent linking group. Y⁰³¹ represents an (n031+1)-valent linking group. L⁰¹¹, L⁰²¹, L⁰²², L⁰³¹, and Y⁰³² each independently represents a single bond or a divalent linking group. R⁰³² represents a hydrocarbon group which may have a substituent. n011, n021, n022, and n031 each independently represents an integer of 1 or greater as long as the valence is allowed, m represents an integer of 1 or greater, and M^{m+} represents an m-valent cation.]

A⁰¹ to A⁰³ in Formulae (a01-1) to (a03-1) are each preferably an oxygen atom.

Examples of Y⁰¹¹, Y⁰²¹, Y⁰²², and Y⁰³¹ in Formulae (a01-1) to (a03-1) include the same ones as Y⁰¹ in Formula (a0-1).

Examples of L⁰¹¹, L⁰²¹, L⁰²², and L⁰³¹ in Formulae (a01-1) to (a03-1) include the same ones as L⁰¹ in Formula (a0-1).

Examples of n011, n021, n022, and n031 in Formulae (a01-1) to (a03-1) include the same ones as n01 in Formula (a0-1). n011, n021, n022, and n031 are preferably an integer of 1 to 5, more preferably an integer of 1 to 4, still more preferably an integer of 1 to 3, particularly preferably 1 or 2, and most preferably 1.

In Formula (a03-1), examples of Y⁰³² include the same ones as those of Y⁰³ in Formula (r01-2).

In Formula (a03-1), examples of R⁰³² include the same ones as those of R⁰³ in Formula (r01-2).

As the constitutional unit (a0), a constitutional unit represented by General Formula (a01-1-1), (a02-1-1), or (a03-1-1) is preferable.

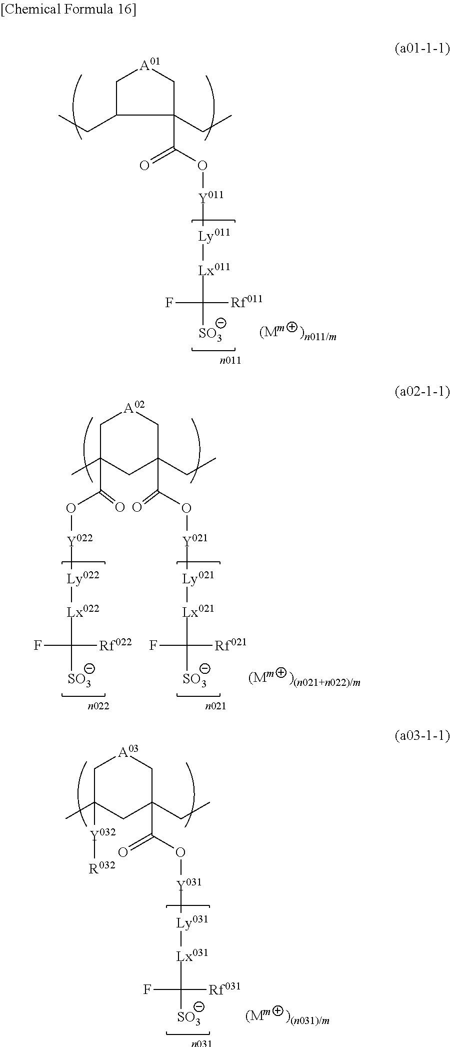

[In the formula, A⁰¹ to A⁰³, Y⁰¹¹, Y⁰²¹, Y⁰²², Y⁰³¹, Y⁰³², R⁰³², n011, n021, n022, and n031 are each the same as those in Formulae (a01-1) to (a03-1). Ly⁰¹¹, Ly⁰²¹, Ly⁰²², and Ly⁰³¹ each independently represents a single bond or a divalent linking group. Lx⁰¹¹, Lx⁰²¹, Lx⁰²², and Lx⁰³¹ each independently represents a single bond, an alkylene group, or a fluorinated alkylene group. Rf⁰¹¹, Rf⁰²¹, Rf⁰²², and Rf⁰³¹ each independently represents a fluorinated alkyl group or a fluorine atom, in represents an integer of 1 or greater, and M^{m+} represents an m-valent cation.

In Formulae (a01-1-1), (a02-1-1), and (a03-1-1), examples of the divalent linking group as Ly⁰¹¹, Ly⁰²¹, Ly⁰²², and Ly⁰¹³ include the same groups as those for La⁰¹ in Formula (l01-1). The divalent linking group is preferably a linking group represented by —O—, —C(═O)—O—, or —O—C(═O)—; or a group which is a combination of one or more of the linking groups and a linear or branched alkylene group.

In Formulae (a01-1-1). (a02-1-1), and (a03-1-1), examples of the alkylene group in Lx⁰¹¹, Lx⁰²¹, Lx⁰²², and Lx⁰³¹ include a linear or branched alkylene group. The linear alkylene group preferably has 1 to 4 carbon atoms, more preferably has 1 to 3 carbon atoms, and still more preferably 1 or 2 carbon atoms. The branched alkylene group preferably has 2 to 4 carbon atoms, and more preferably has 2 or 3 carbon atoms.

Examples of the fluorinated alkylene group as Lx⁰¹¹, Lx⁰²¹, Lx⁰²², and Lx⁰³ include an alkylene group in which some or all of hydrogen atoms have been substituted with fluorine atoms.

Lx⁰¹¹, Lx⁰²¹, Lx⁰²², and Lx⁰³¹ are preferably a single bond, a linear or branched alkylene group having 1 to 4 carbon atoms, or a linear or branched fluorinated alkylene group having 1 to 4 carbon atoms, and more preferably a single bond, a methylene group, or —CH(CF₃)—.

In Formulae (a01-1-1), (a02-1-1), and (a03-1-1), examples of the fluorinated alkyl group in Rf⁰¹¹, Rf⁰²¹, Rf⁰²², and Rf⁰³¹ include a linear or branched fluorinated alkyl group. The linear fluorinated alkyl group preferably has 1 to 5 carbon atoms, more preferably has 1 to 3 carbon atoms, and still more preferably has 1 or 2 carbon atoms. The branched fluorinated alkyl group preferably has 3 to 5 carbon atoms, and more preferably has 3 or 4 carbon atoms. The fluorinated alkyl group is preferably a perfluoroalkyl group and more preferably a trifluoromethyl group.

Specific examples of the constitutional unit (a0) are shown below, but the present invention is not limited thereto. In the following formula, m represents an integer of 1 or greater, and M^{m+} represents an m-valent cation.

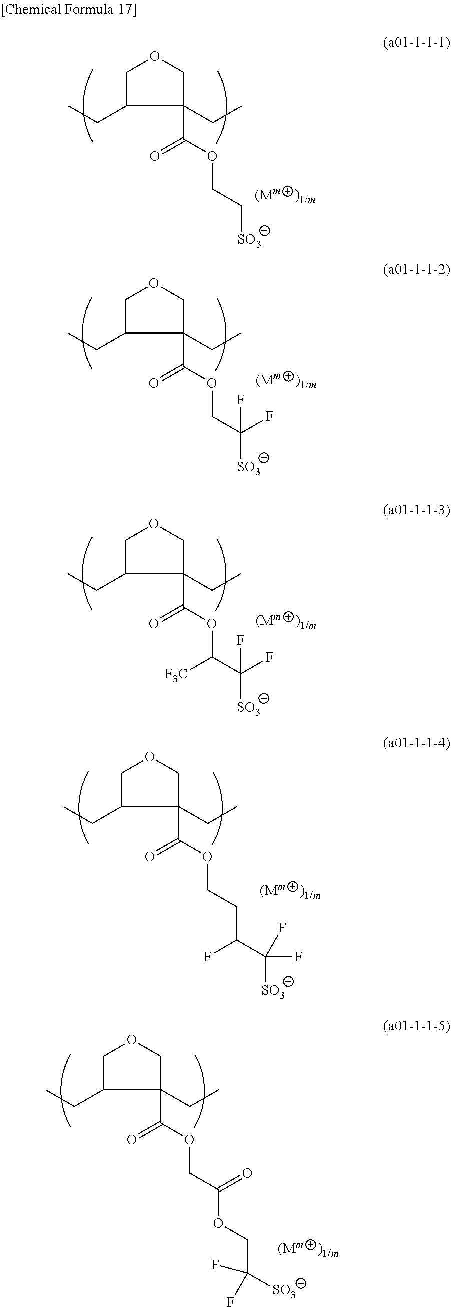

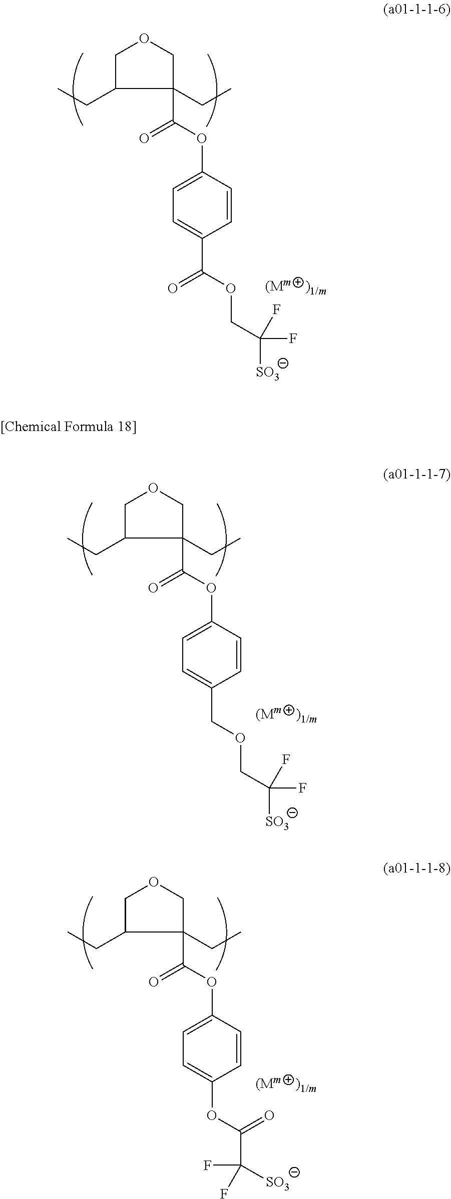

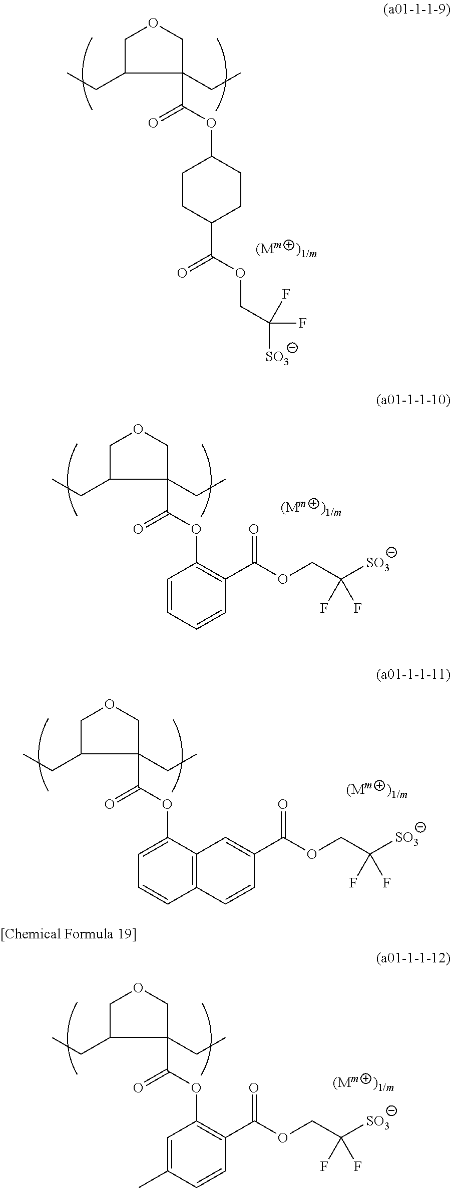

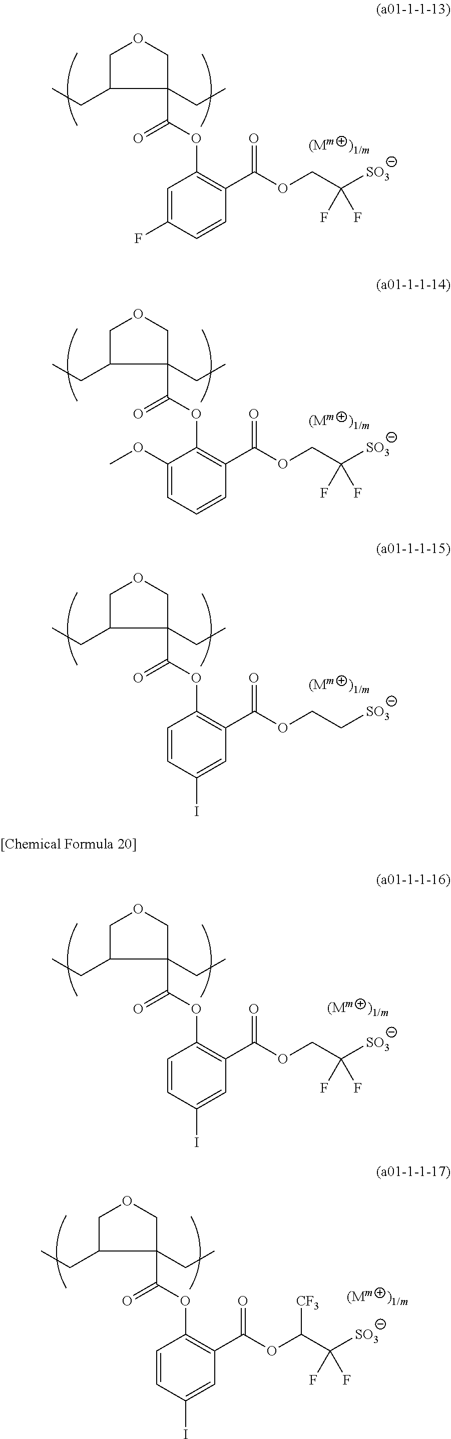

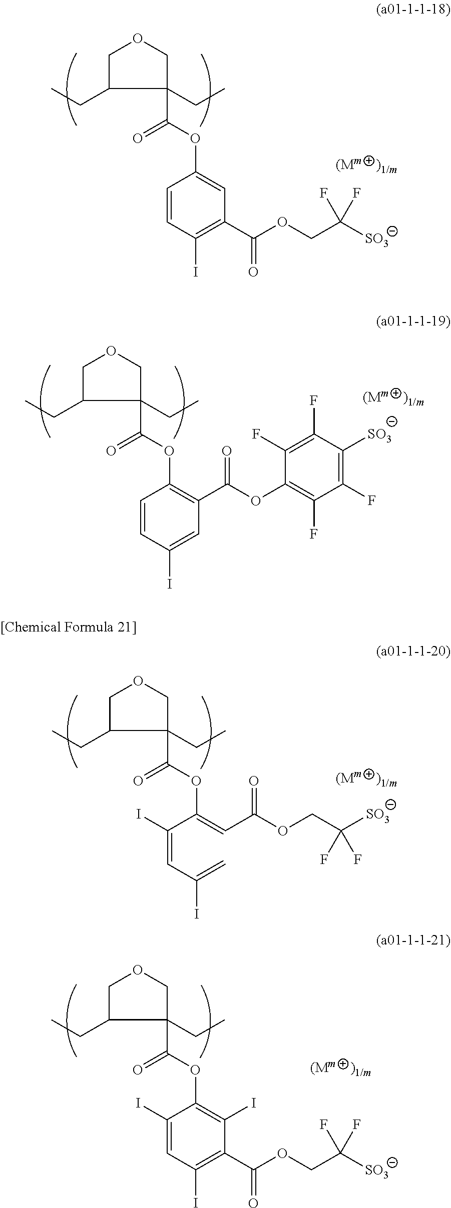

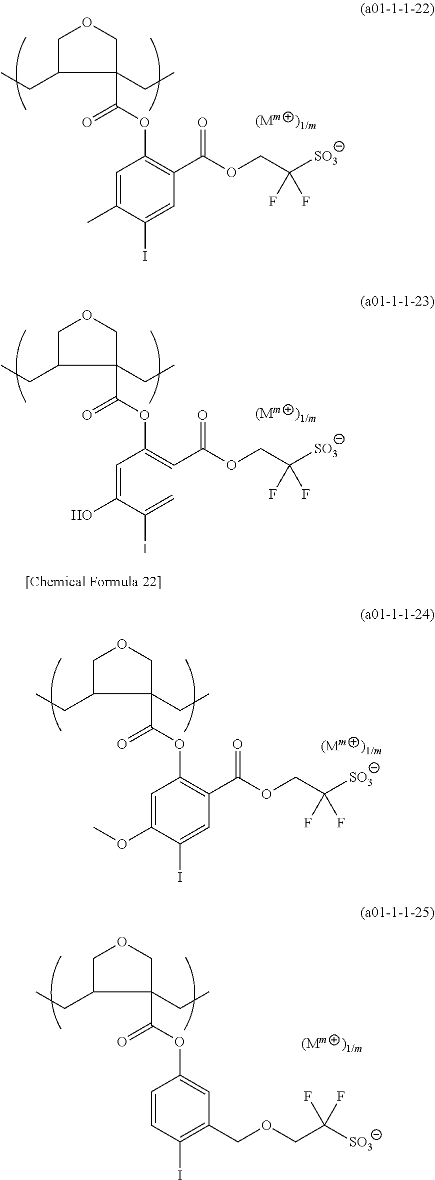

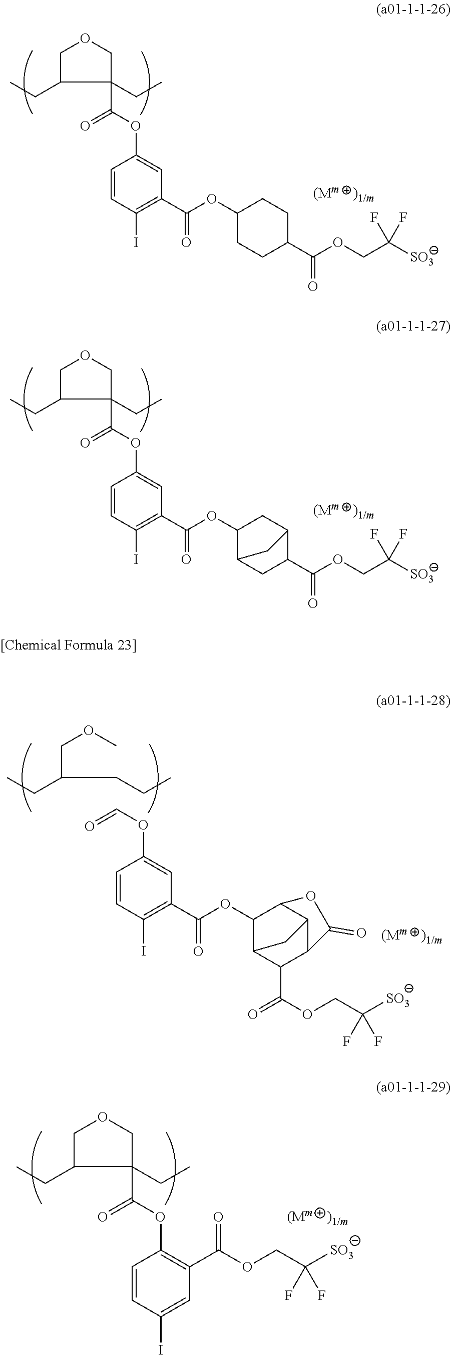

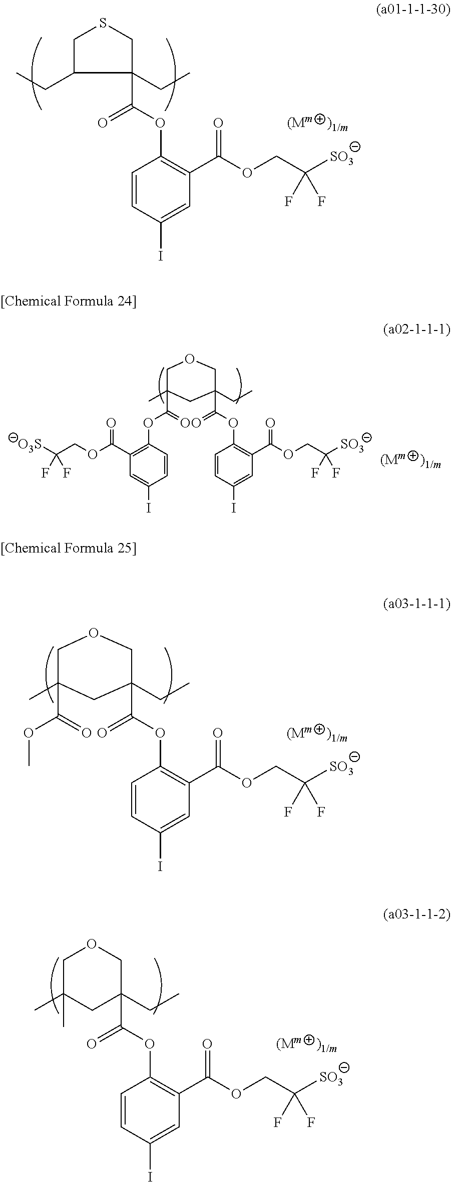

### [Cation Moiety]

In the formulae shown above, M^{m+} represents an m-valent cation. A sulfonium cation or an iodonium cation is preferable as the cation. m represents an integer of 1 or greater.

Examples of the cation moiety include organic cations each represented by General Formulae (ca-1) to (ca-3).

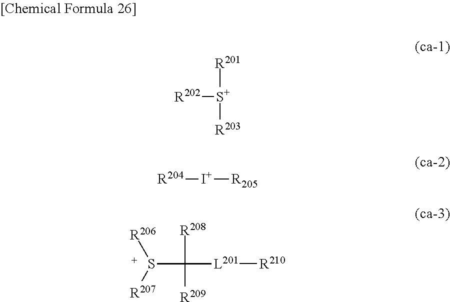

[in the formula, R²⁰¹ to R²⁰⁷ each independently represents an aryl group which may have a substituent, an alkyl group which may have a substituent, or an alkenyl group which may have a substituent. R²⁰¹ to R²⁰³, and R²⁰⁶ and R²¹⁷ may be bonded to each other to form a ring with the sulfur atoms in the formulae. R²⁰⁸ and R²⁰⁹ each independently represents a hydrogen atom or an alkyl group having 1 to 5 carbon atoms. R²¹⁰ represents an aryl group which may have a substituent, an alkyl group which may have a substituent, an alkenyl group which may have a substituent, or a —SO₂-containing cyclic group which may have a substituent. L²⁰¹ represents —C(═O)— or —C(═O)—O—]

In General Formulae (ca-1) to (ca-3), examples of the aryl group as R²⁰¹ to R²⁰⁷ include an unsubstituted aryl group having 6 to 20 carbon atoms. Among these, a phenyl group or a naphthyl group is preferable.

As the alkyl group as R²⁰¹ to R²⁰⁷, a chain-like or cyclic alkyl group having 1 to 30 carbon atoms is preferable.

It is preferable that the alkenyl group as R²⁰¹ to R²⁰⁷ have 2 to 10 carbon atoms. Examples of the substituent that R²⁰¹ to R²⁰⁷ and R²¹⁰ may have include an alkyl group, a halogen atom, a halogenated alkyl group, a carbonyl group, a cyano group, an amino group, an aryl group, and groups each represented by General Formulae (ca-r-1) to (ca-r-7).

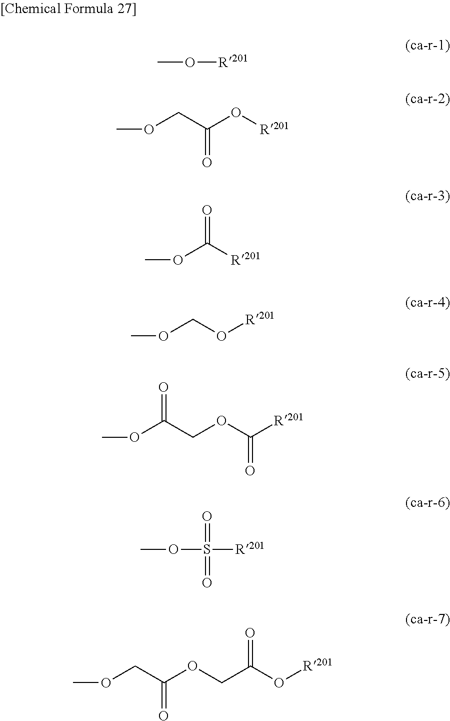

[In the formulae, R′²⁰¹'s each independently represents a hydrogen atom, a cyclic group which may have a substituent, a chain-like alkyl group which may have a substituent, or a chain-like alkenyl group which may have a substituent.]

### Cyclic Group Which May Have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group that has no aromaticity. Further, the aliphatic hydrocarbon group may be saturated or unsaturated. In general, it is preferable that the aliphatic hydrocarbon group be saturated.

The aromatic hydrocarbon group as R′²⁰¹ is a hydrocarbon group having an aromatic ring. The aromatic hydrocarbon group preferably has 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring contained in the aromatic hydrocarbon group as R′²⁰¹ include benzene, fluorene, naphthalene, anthracene, phenanthrene, biphenyl, or an aromatic heterocyclic ring in which some carbon atoms constituting any of these aromatic rings have been substituted with hetero atoms. Examples of the heteroatom in the aromatic heterocyclic rings include an oxygen atom, a sulfur atom, and a nitrogen atom.

Specific examples of the aromatic hydrocarbon group as R′²⁰¹ include a group in which one hydrogen atom has been removed from the aromatic ring (an aryl group such as a phenyl group or a naphthyl group), and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group). The alkylene group (alkyl chain in the arylalkyl group) preferably has 1 to 4 carbon atoms, more preferably 1 or 2 carbon atoms, and particularly preferably 1 carbon atom.

Examples of the cyclic aliphatic hydrocarbon group as R′²⁰¹ include an aliphatic hydrocarbon group having a ring in the structure thereof.

Examples of the aliphatic hydrocarbon group having a ring in the structure thereof include an alicyclic hydrocarbon group (group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group preferably has 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane preferably has 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring polycyclic skeleton such as adamantane, norbornane, isobornane, tricyclo[5.2.1.0^{2,6}]decane, or tetracyclododecane; and a polycycloalkane having a condensed ring polycyclic skeleton such as a cyclic group having a steroid skeleton are preferable as the polycycloalkane.

Among these examples, as the cyclic aliphatic hydrocarbon group as R′²⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is particularly preferable, and an adamantyl group is most preferable.

The linear or branched aliphatic hydrocarbon group which may be bonded to the alicyclic hydrocarbon group preferably has 1 to 10 carbon atoms, more preferably 1 to 6 carbon atoms, still more preferably 1 to 4 carbon atoms, and particularly preferably 1 to 3 carbon atoms.

As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

Further, the cyclic hydrocarbon group as R′²⁰¹ may have a hetero atom such as a heterocyclic ring. Specific examples thereof include lactone-containing cyclic groups each represented by General Formulae (a2-r-1) to (a2-r-7), —SO₂-containing cyclic groups each represented by General Formulae (b5-r-1) to (b5-r-4), and other heterocyclic groups each represented by Chemical Formulae (r-hr-1) to (r-hr-16).

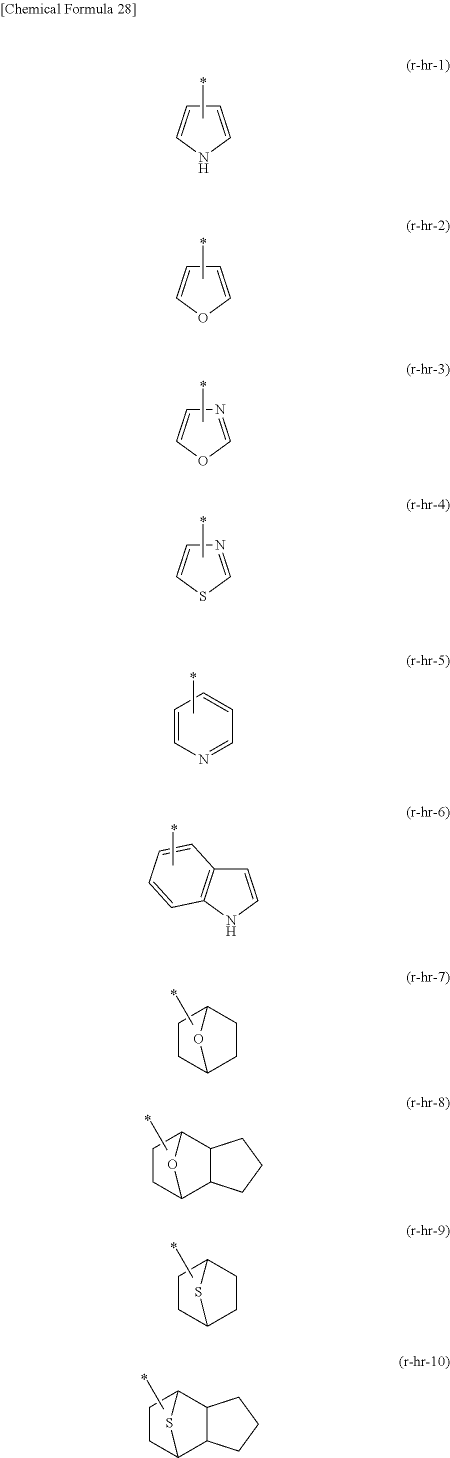

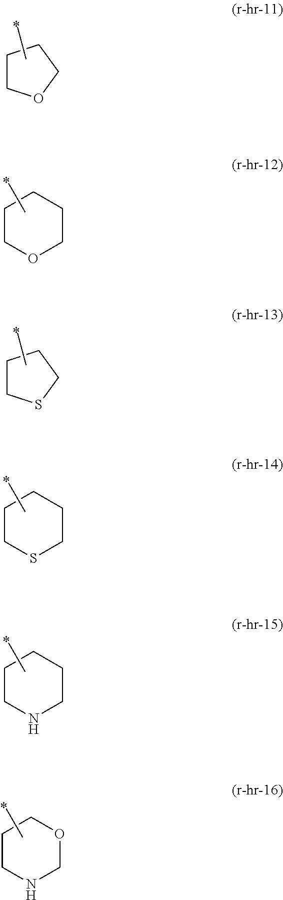

Examples of the substituent for the cyclic group as R′²⁰¹ include an alkyl group, an alkoxy group, a halogen atom, a halogenated alkyl group, a hydroxyl group, a carbonyl group, and a nitro group.

As the alkyl group as the substituent, an alkyl group having 1 to 5 carbon atoms is preferable, and a methyl group, an ethyl group, a propyl group, an n-butyl group, or a tert-butyl group is most preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

As the halogen atom as a substituent, a fluorine atom is preferable.

An example of the above-described halogenated alkyl group as the substituent includes a group in which some or all hydrogen atoms in an alkyl group having 1 to 5 carbon atoms such as a methyl group, an ethyl group, a propyl group, an n-butyl group, or a tert-butyl group have been substituted with the above-described halogen atoms.

The carbonyl group as the substituent is a group that substitutes a methylene group (—CH₂—) constituting the cyclic hydrocarbon group.

Chain-Like Alkyl Group which May have Substituent:

The chain-like alkyl group as R′²⁰¹ may be linear or branched.

The linear alkyl group preferably has 1 to 20 carbon atoms, more preferably 1 to 15 carbon atoms, and most preferably 1 to 10 carbon atoms.

The branched alkyl group preferably has 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-Like Alkenyl Group which May have Substituent:

The chain-like alkenyl group as R′²⁰¹ may be linear or branched, and the number of carbon atoms thereof is preferably 2 to 10, more preferably 2 to 5, still more preferably 2 to 4, and particularly preferably 3. Examples of the linear alkenyl group include a vinyl group, a propenyl group (allyl group), and a butenyl group. Examples of the branched alkenyl group include a 1-methylvinyl group, a 2-methylvinyl group, a 1-methylpropenyl group, and a 2-methylpropenyl group.

Among the examples, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R′²⁰¹ include an alkoxy group, a halogen atom, a halogenated alkyl group, a hydroxyl group, a carbonyl group, a nitro group, an amino group, and a cyclic group as R′²⁰¹.

Examples of the cyclic group which may have a substituent, the chain-like alkyl group which may have a substituent, and the chain-like alkenyl group which may have a substituent as R′²⁰¹ include the same groups as those for the acid-dissociable group represented by Formula (a1-r-2) which are exemplary examples of the cyclic group which may have a substituent and the chain-like alkyl group which may have a substituent, in addition to those described above.

Among the examples, R′²⁰¹ preferably represents a cyclic group which may have a substituent and more preferably a cyclic hydrocarbon group which may have a substituent. More specific preferred examples thereof include a phenyl group, a naphthyl group, a group in which one or more hydrogen atoms have been removed from a polycycloalkane, a lactone-containing cyclic group represented by any of General Formulae (a2-r-1) to (a2-r-7), and a —SO₂-containing cyclic group represented by any of General Formulae (b5-r-1) to (b5-r-4).

In General Formulae (ca-1) to (ca-3), in a case where R²⁰¹ to R²⁰³ and R²⁰⁶ and R²⁰⁷ are bonded to each other to form a ring with a sulfur atom in the formula, these groups may be bonded to each other via a hetero atom such as a sulfur atom, an oxygen atom, or a nitrogen atom, or a functional group such as a carbonyl group, —SO—, —SO₂—, —SO₃—, —COO—, —CONH— or —N(R_{N})— (R_{N} represents an alkyl group having 1 to 5 carbon atoms). As a ring to be formed, a ring containing the sulfur atom in the formula in the ring skeleton thereof is preferably a 3- to 10-membered ring and particularly preferably a 5- to 7-membered ring containing the sulfur atom. Specific examples of the ring to be formed include a thiophene ring, a thiazole ring, a benzothiophene ring, a dibenzothiophene ring, a 9H-thioxanthene ring, a thioxanthone ring, a thianthrene ring, a phenoxathiin ring, a tetrahydrothiophenium ring, and a tetrahydrothiopyranium ring.

R²⁰⁸ and R²⁰⁹ each independently represents a hydrogen atom or an alkyl group having 1 to 5 carbon atoms and preferably a hydrogen atom or an alkyl group having 1 to 3 carbon atoms. In a case where R²⁰⁸ and R²⁰⁹ represent an alkyl group, R²⁰⁸ and R²⁰⁹ may be bonded to each other to form a ring.

R²¹⁰ represents an aryl group which may have a substituent, an alkyl group which may have a substituent, an alkenyl group which may have a substituent, or a —SO₂-containing cyclic group which may have a substituent.

Examples of the aryl group as R²¹⁰ include an unsubstituted aryl group having 6 to 20 carbon atoms. Among these, a phenyl group or a naphthyl group is preferable.

As the alkyl group as R²¹⁰, a chain-like or cyclic alkyl group having 1 to 30 carbon atoms is preferable.

It is preferable that the alkenyl group as R²¹⁰ have 2 to 10 carbon atoms.

The —SO₂-containing cyclic group as R²¹⁰ is not particularly limited, and any —SO₂-containing cyclic group can be used. Specific examples thereof include groups each represented by General Formulae (b5-r-1) to (b5-r-4), where “—SO₂-containing polycyclic group” is preferable, and a group represented by General Formula (b5-r-1) is more preferable.

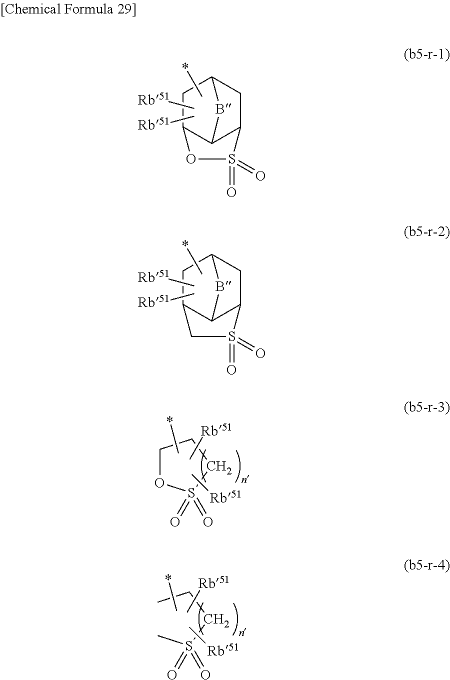

[In the formulae, Rb′⁵¹'s each independently represents a hydrogen atom, an alkyl group, an alkoxy group, a halogen atom, a halogenated alkyl group, a hydroxyl group, —COOR″, —OC(═O)R″, a hydroxyalkyl group, or a cyano group; R″ represents a hydrogen atom, an alkyl group, a lactone-containing cyclic group, or a —SO₂-containing cyclic group; B″ represents an alkylene group having 1 to 5 carbon atoms which may have an oxygen atom or a sulfur atom, an oxygen atom, or a sulfur atom, and n′ represents an integer of 0 to 2. * represents a bonding site.]

In General Formulae (b5-r-1) and (b5-r-2), B″ represents an alkylene group having 1 to 5 carbon atoms which may have an oxygen atom or a sulfur atom, an oxygen atom, or a sulfur atom.

B″ preferably represents an alkylene group having 1 to 5 carbon atoms or —O—, more preferably an alkylene group having 1 to 5 carbon atoms, and still more preferably a methylene group.

In General Formulae (b5-r-1) to (b5-r-4), Rb′⁵¹'s each independently represents a hydrogen atom, an alkyl group, an alkoxy group, a halogen atom, a halogenated alkyl group, a hydroxyl group, —COOR″, —OC(═O)R″, a hydroxyalkyl group, or a cyano group. Among these, it is preferable that Rb′⁵¹'s each independently represent a hydrogen atom or a cyano group.

Specific examples of the group-s each represented by General Formulae (b5-r-1) to (b5-r-4) are shown below. In the formulae shown below, “Ac” represents an acetyl group.

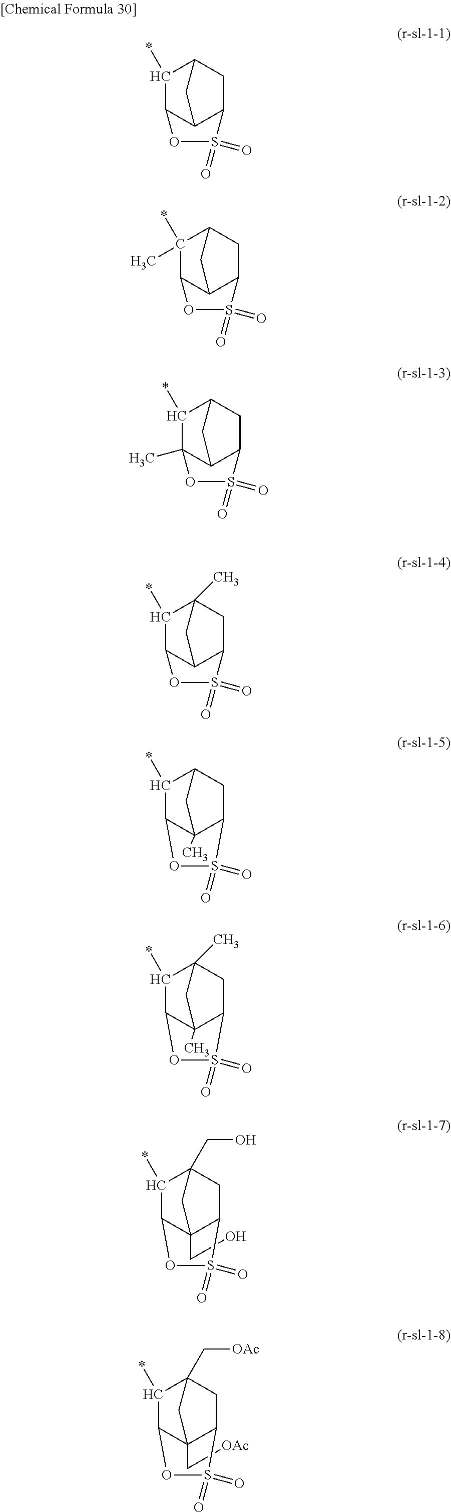

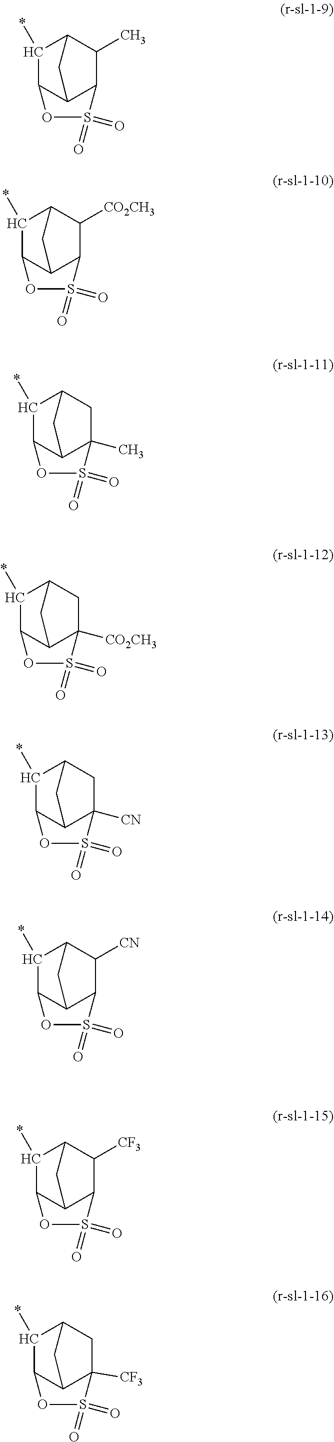

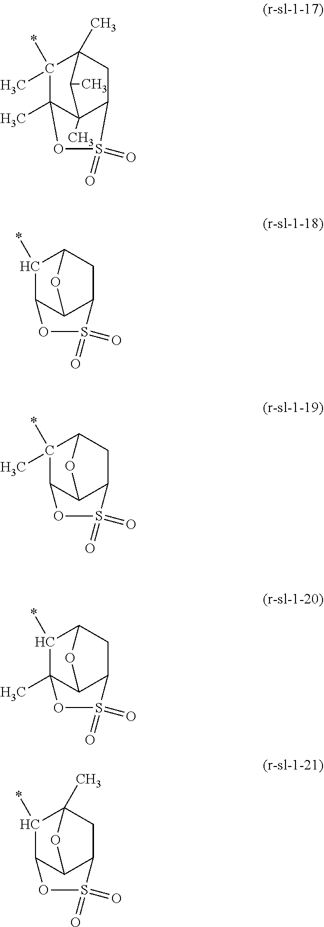

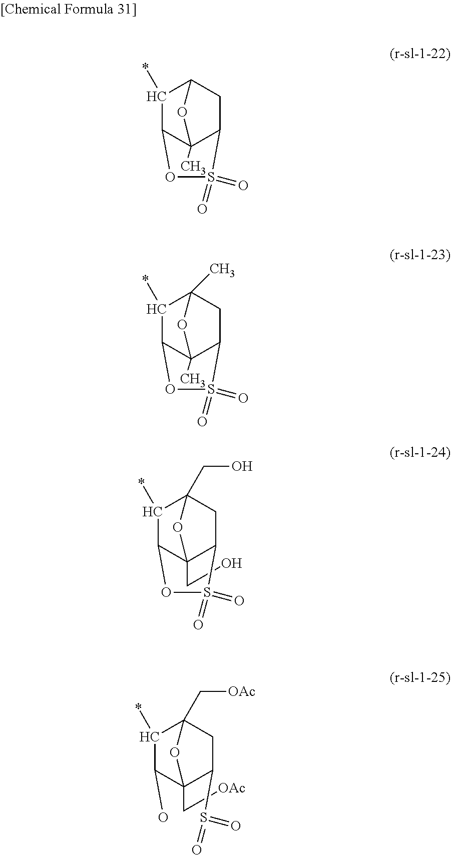

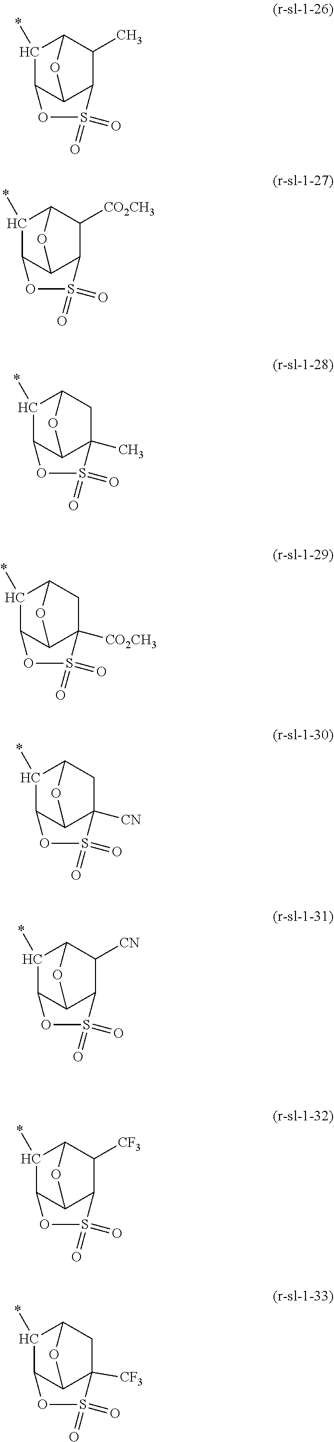

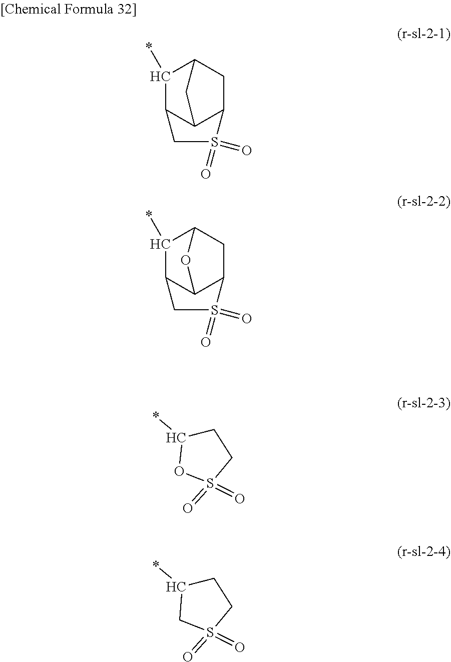

Specific examples of the cation represented by Formula (ca-1) are shown below.

Specific examples of the suitable cation represented by Formula (ca-1) include cations each represented by the following chemical formulae.

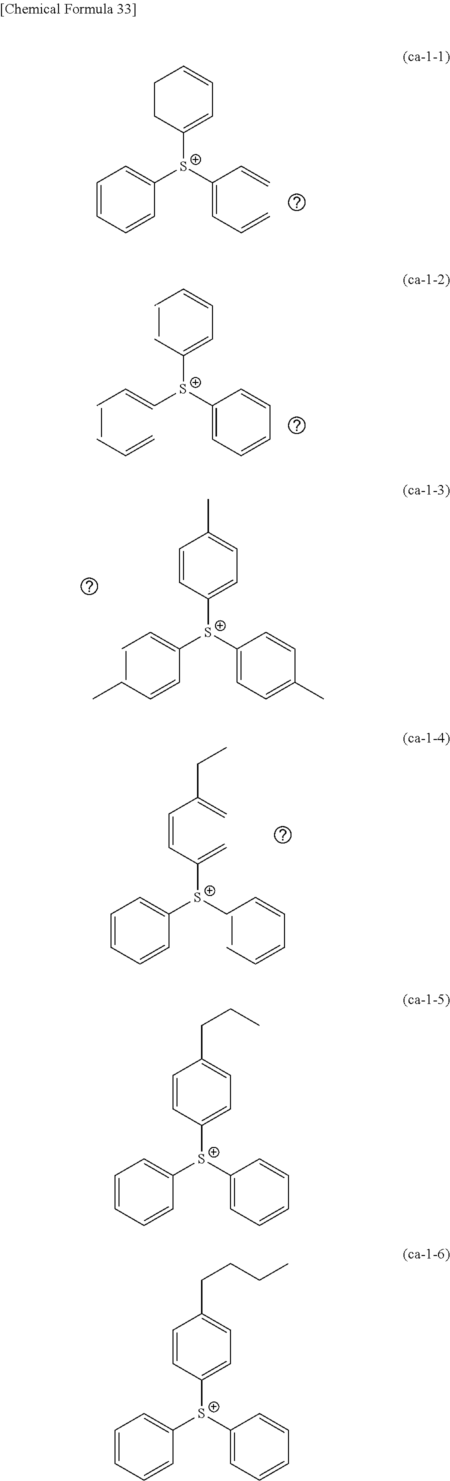

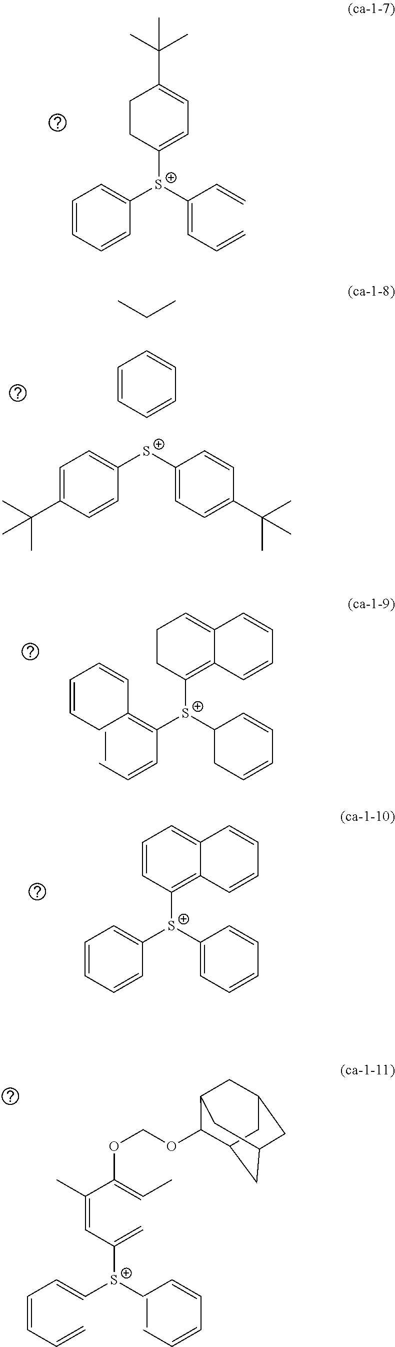

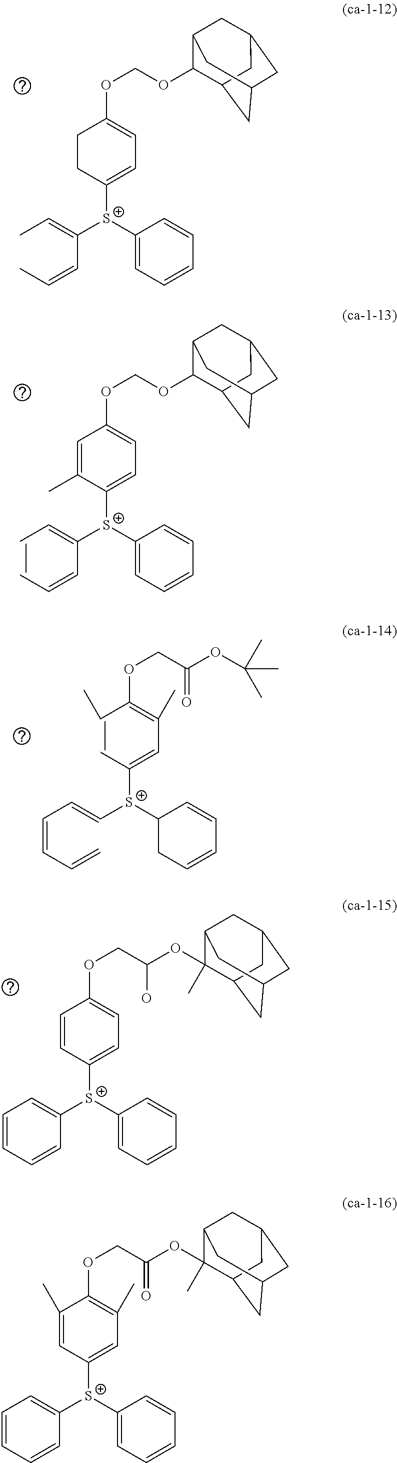

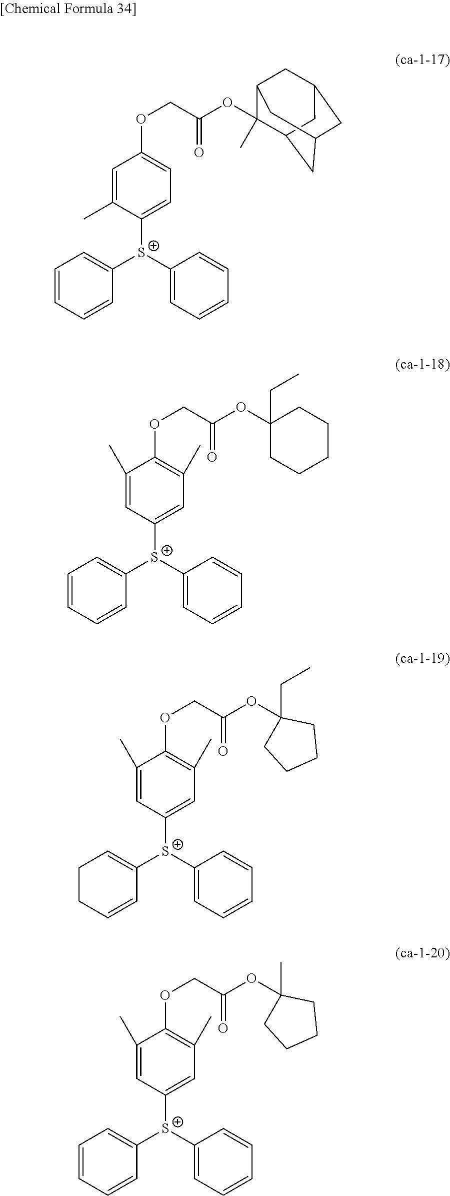 [SMILES: Cc1cc(OCC(=O)OC2(C)C3CC4CC(C3)CC2C4)ccc1[S+](c1ccccc1)c1ccccc1 ; CCC1(OC(=O)COc2c(C)cc([S+](c3ccccc3)c3ccccc3)cc2C)CCCCC1 ; CCC1(OC(=O)COc2c(C)cc([S+](C3=CCCC=C3)c3ccccc3)cc2C)CCCC1 ; Cc1cc([S+](c2ccccc2)c2ccccc2)cc(C)c1OCC(=O)OC1(C)CCCC1]

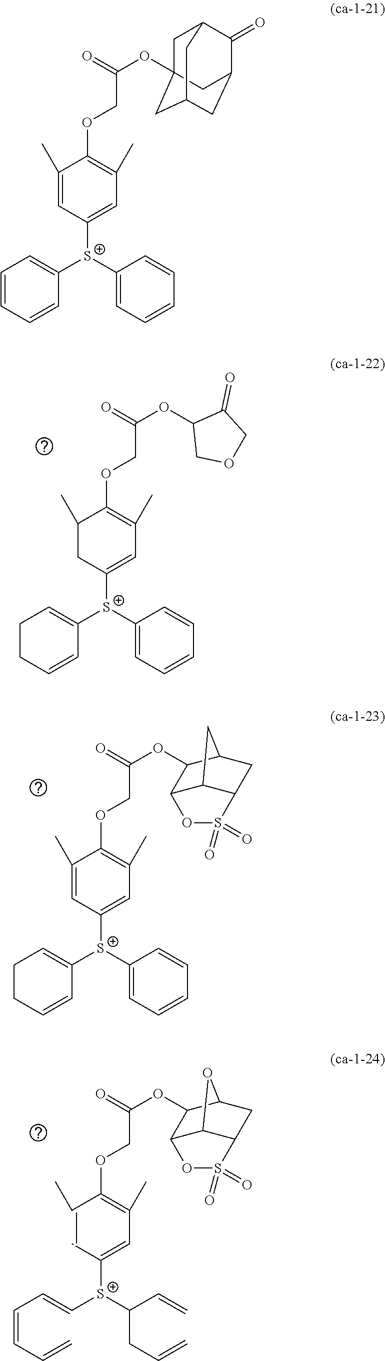

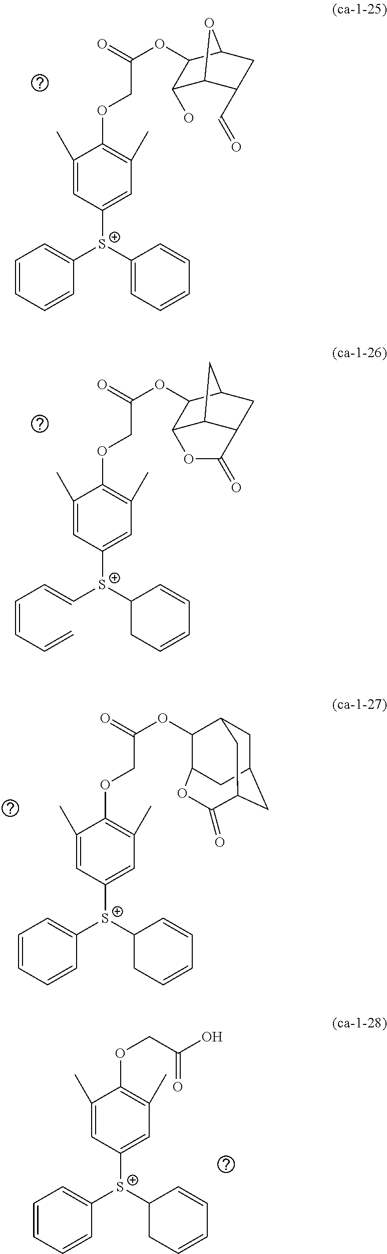 [SMILES: Cc1cc([S+](c2ccccc2)c2ccccc2)cc(C)c1OCC(=O)OC1C2CC(C=O)C(O2)C1O ; C=C/C=C\C=C\[S+](c1cc(C)c(OCC(=O)OC2C3CC4C(=O)OC2C4C3)c(C)c1)C1C=CC=CC1 ; Cc1cc([S+](c2ccccc2)C2C=CC=CC2)cc(C)c1OCC(=O)OC1C2CC3CC(C2)C(=O)OC1C3 ; Cc1cc([S+](c2ccccc2)C2C=CC=CC2)cc(C)c1OCC(=O)O]

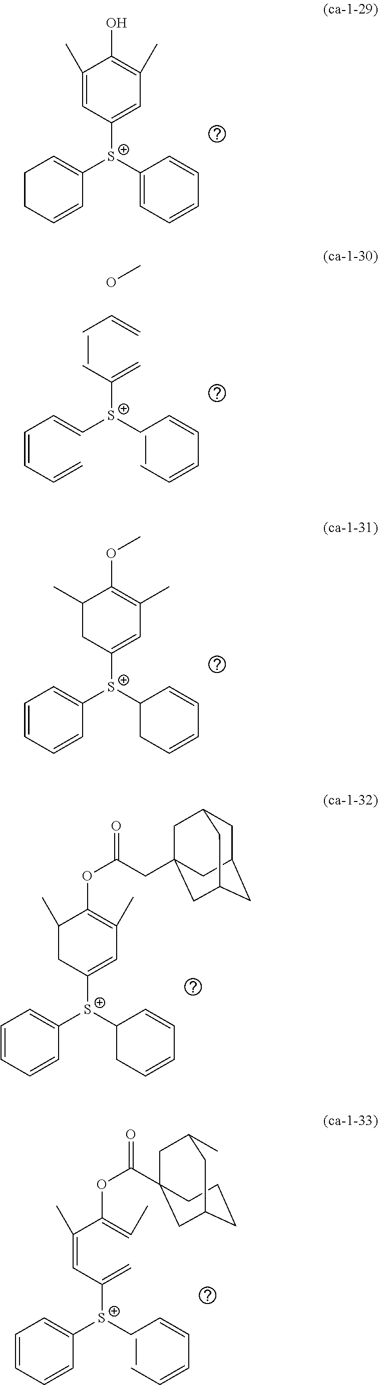

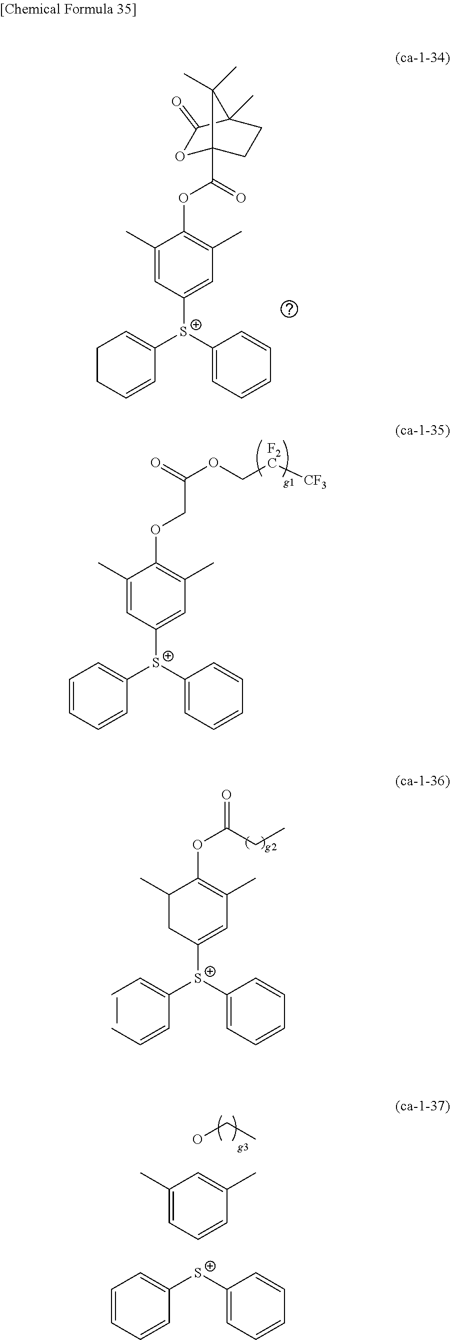

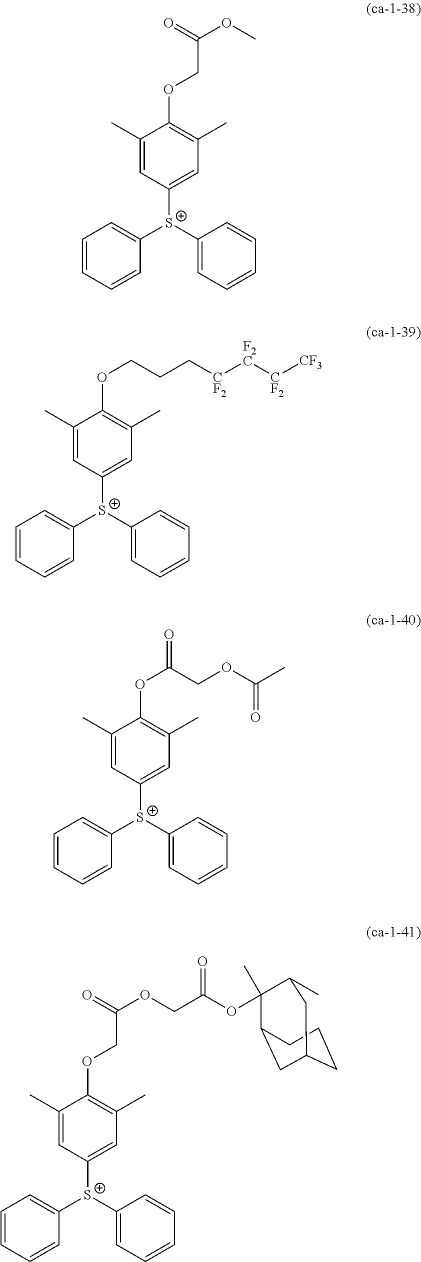 [SMILES: COC(=O)COc1c(C)cc([S+](c2ccccc2)c2ccccc2)cc1C ; Cc1cc([S+](c2ccccc2)c2ccccc2)cc(C)c1OCCCC(F)(F)C(F)(F)C(F)(F)C(F)(F)F ; CC(=O)OCC(=O)Oc1c(C)cc([S+](c2ccccc2)c2ccccc2)cc1C ; Cc1cc([S+](c2ccccc2)c2ccccc2)cc(C)c1OCC(=O)OCC(=O)OC1(C)C(C)CC2CCCC1C2]

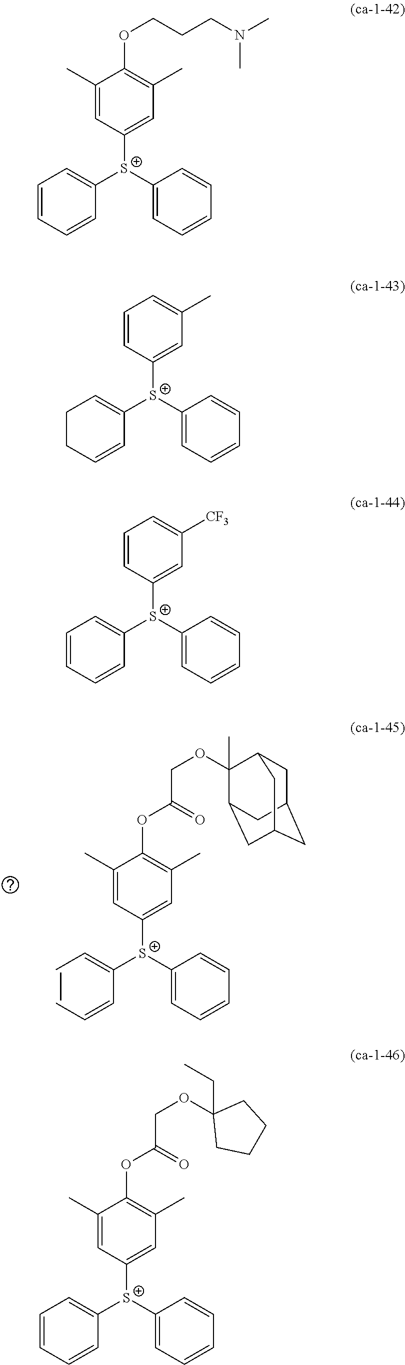

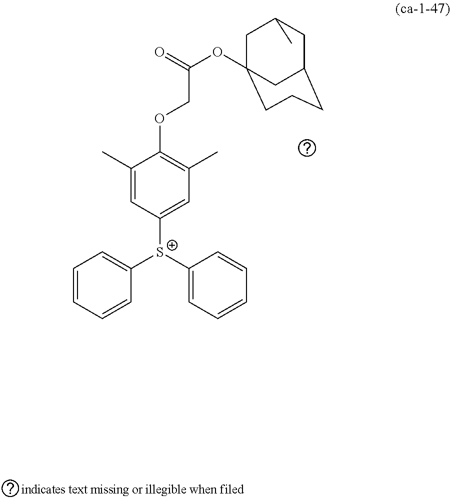 [SMILES: Cc1cc([S+](c2ccccc2)c2ccccc2)cc(C)c1OCC(=O)OC12CCCC(CC(C)C1)C2]

[In the formulae, g1, g2, and g3 represent a repeating number, g1 represents an integer of 1 to 5, g2 represents an integer of 0 to 20, and g3 represents an integer of 0 to 20.]

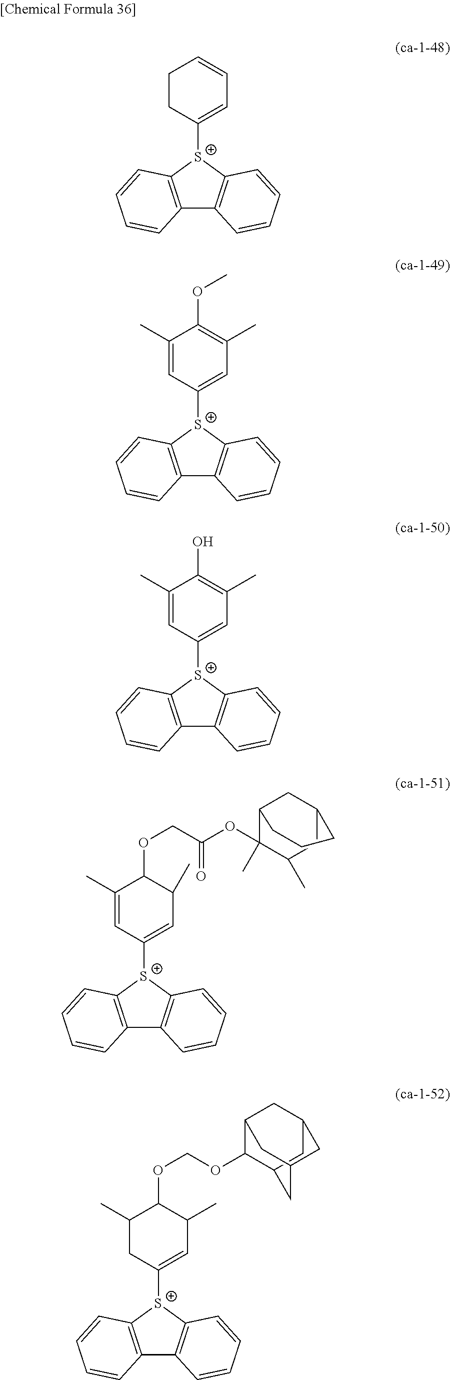 [SMILES: C1=CCCC([s+]2c3ccccc3c3ccccc32)=C1 ; COc1c(C)cc(-[s+]2c3ccccc3c3ccccc32)cc1C ; Cc1cc(-[s+]2c3ccccc3c3ccccc32)cc(C)c1O ; CC1=CC([s+]2c3ccccc3c3ccccc32)=CC(C)C1OCC(=O)OC1(C)C(C)CC2CCCC1C2 ; CC1C=C([s+]2c3ccccc3c3ccccc32)CC(C)C1OCOC1C2CC3CC(C2)CC1C3]

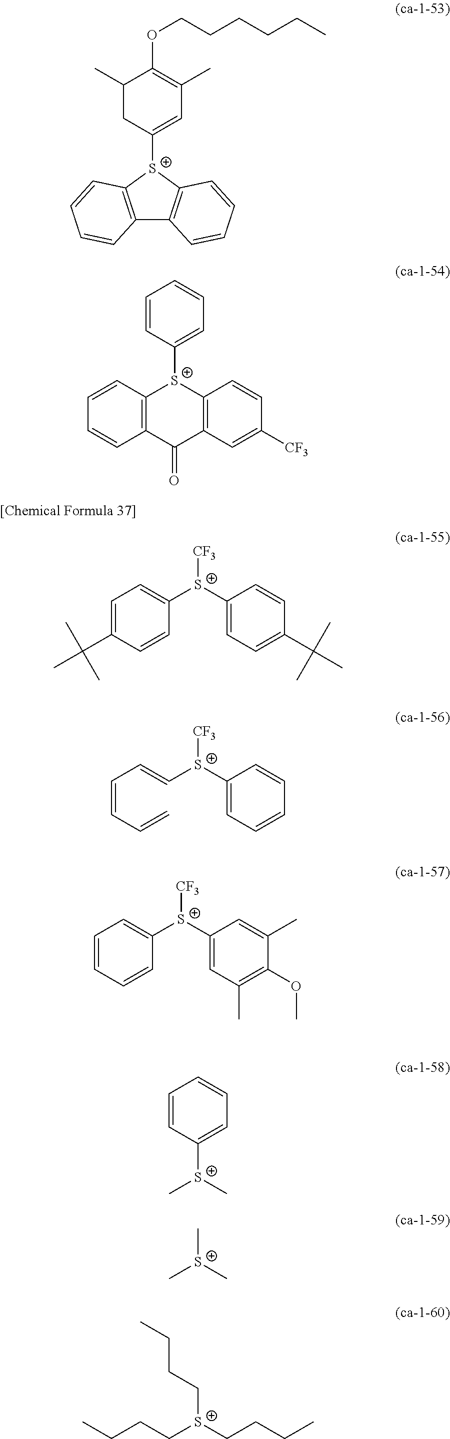

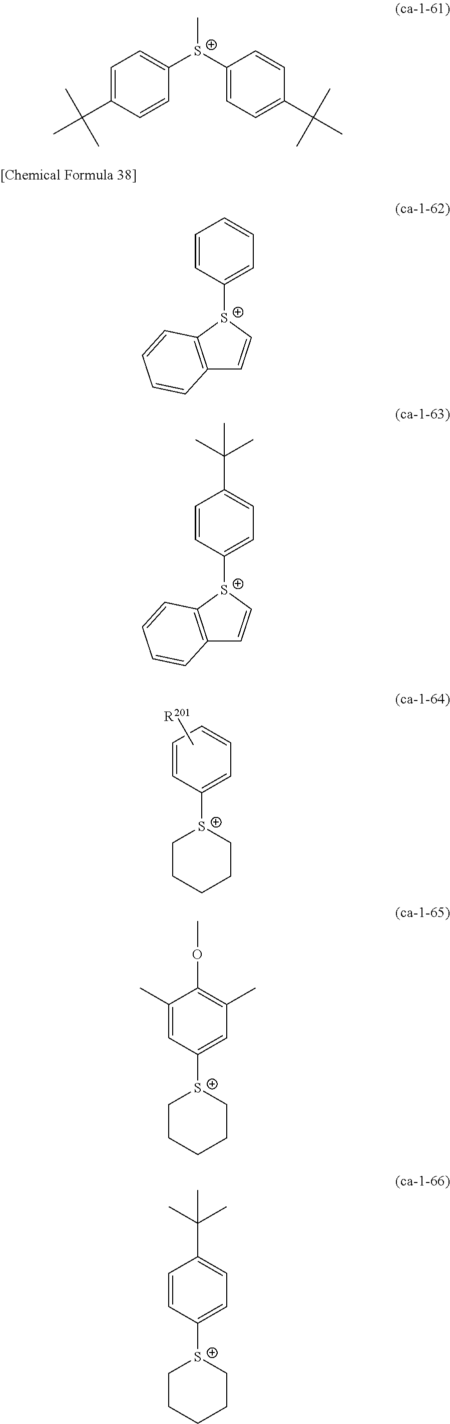

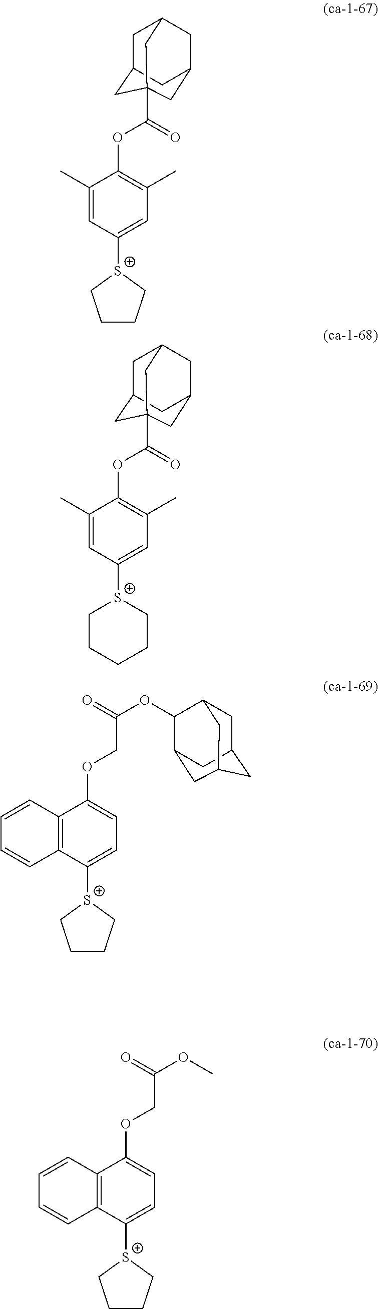 [SMILES: Cc1cc([S+]2CCCC2)cc(C)c1OC(=O)C12CC3CC(CC(C3)C1)C2 ; Cc1cc([S+]2CCCCC2)cc(C)c1OC(=O)C12CC3CC(CC(C3)C1)C2 ; O=C(COc1ccc([S+]2CCCC2)c2ccccc12)OC1C2CC3CC(C2)CC1C3 ; COC(=O)COc1ccc([S+]2CCCC2)c2ccccc12]

[In the formulae, R″²⁰¹ represents a hydrogen atom or a substituent, and examples of the substituent include the same groups as those for the substituents which may be included in R²⁰¹ to R²⁰⁷ and R²¹⁰ to R²¹².]

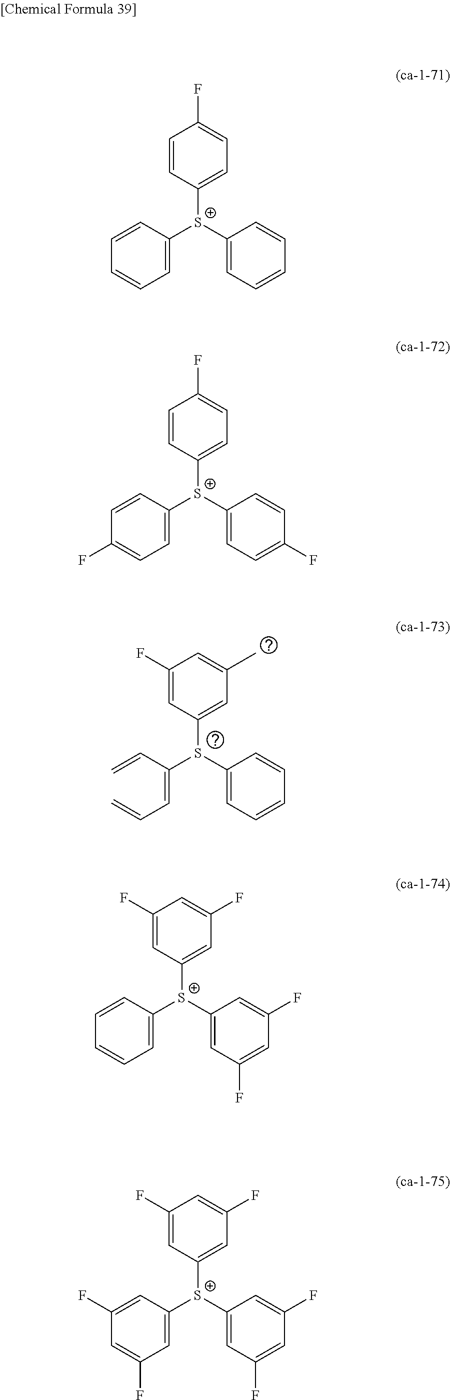

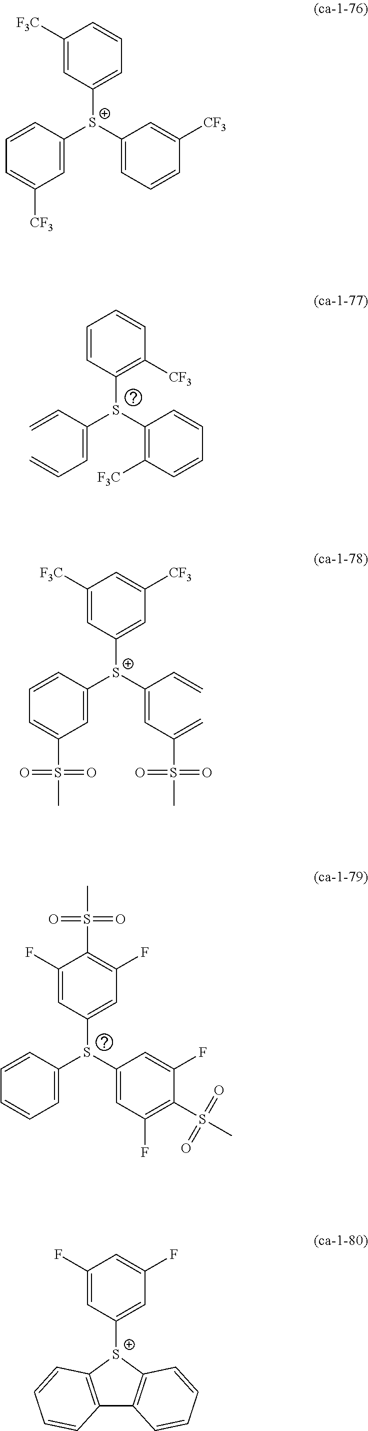

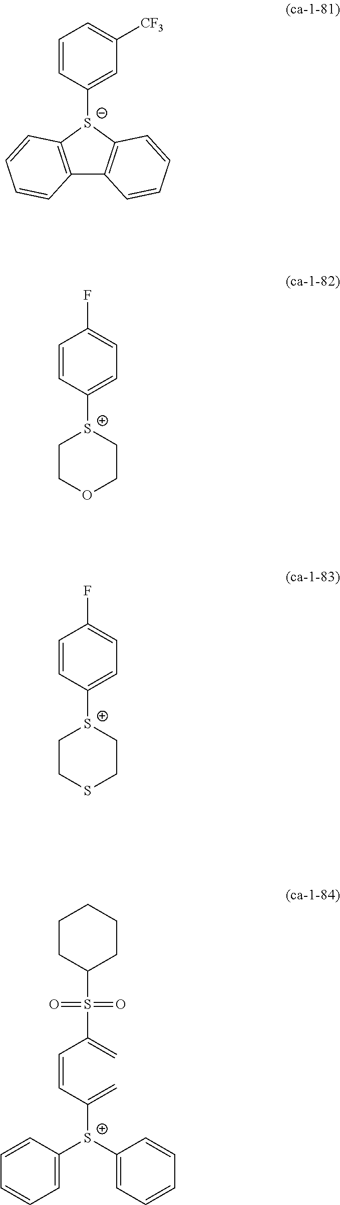 [SMILES: FC(F)(F)c1cccc([S-]2c3ccccc3-c3ccccc32)c1 ; Fc1ccc([S+]2CCOCC2)cc1 ; Fc1ccc([S+]2CCSCC2)cc1 ; C=C(/C=C\C(=C)S(=O)(=O)C1CCCCC1)[S+](c1ccccc1)c1ccccc1]

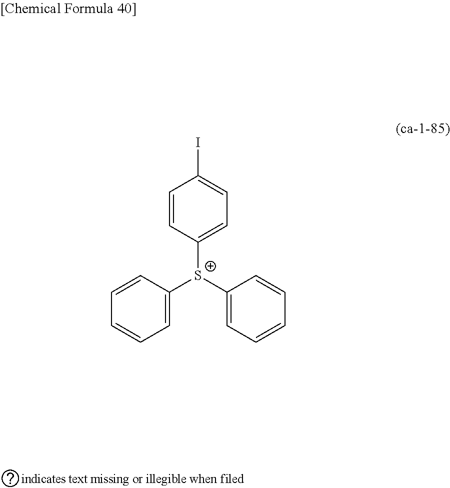 [SMILES: Ic1ccc([S+](c2ccccc2)c2ccccc2)cc1]

Specific examples of suitable cations represented by Formula (ca-2) include a diphenyliodoniumn cation and a bis(4-tert-butylphenyl)iodonium cation.

Specific examples of suitable cations represented by Formula (ca-3) include cations each represented by Formulae (ca-3-1) to (ca-3-6).

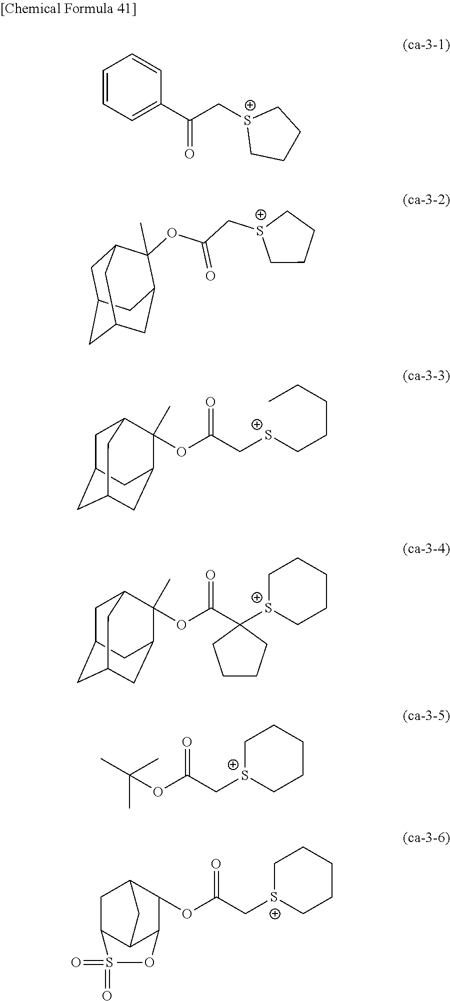

As the cation moiety, a sulfonium cation is preferable, a cation represented by any of Formulae (ca-1) to (ca-3) is more preferable, a cation represented by Formula (ca-1) is still more preferable, and a cation represented by any of Formulae (ca-1-1) to (ca-1-84) is particularly preferable.

In particular, from the viewpoint of achieving high sensitivity, the suitable cation represented by Formula (ca-1) preferably has, as a substituent, an electron-withdrawing group such as a fluorine atom, a fluorinated alkyl group, or a sulfonyl group and is, for example, particularly preferably a cation selected from the group consisting of cations each represented by Chemical Formulae (ca-1-44) and (ca-1-71) to (ca-1-84).

Specific examples of the constitutional unit (a0) are shown below, but the examples are not limited thereto.

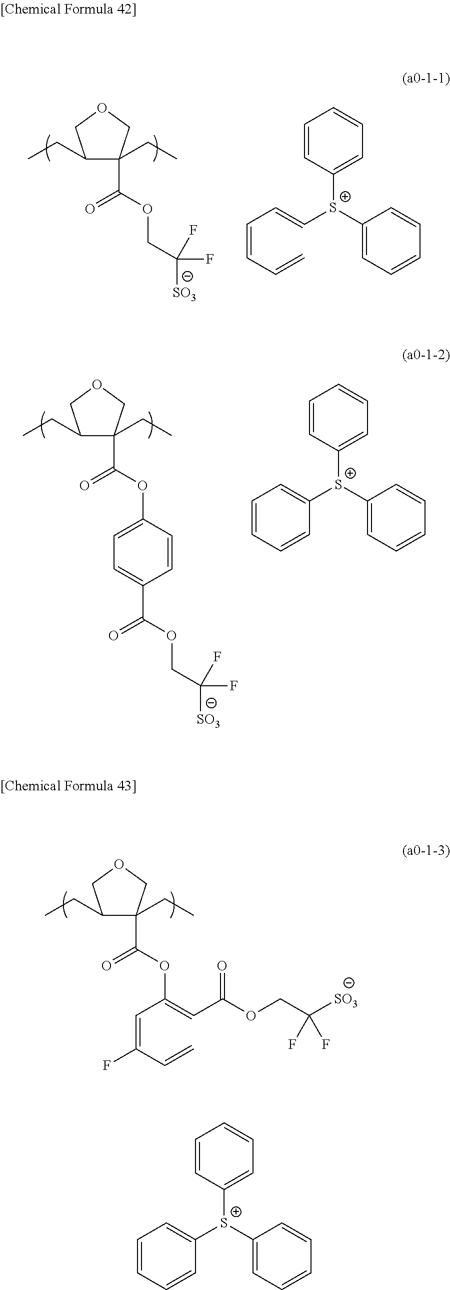

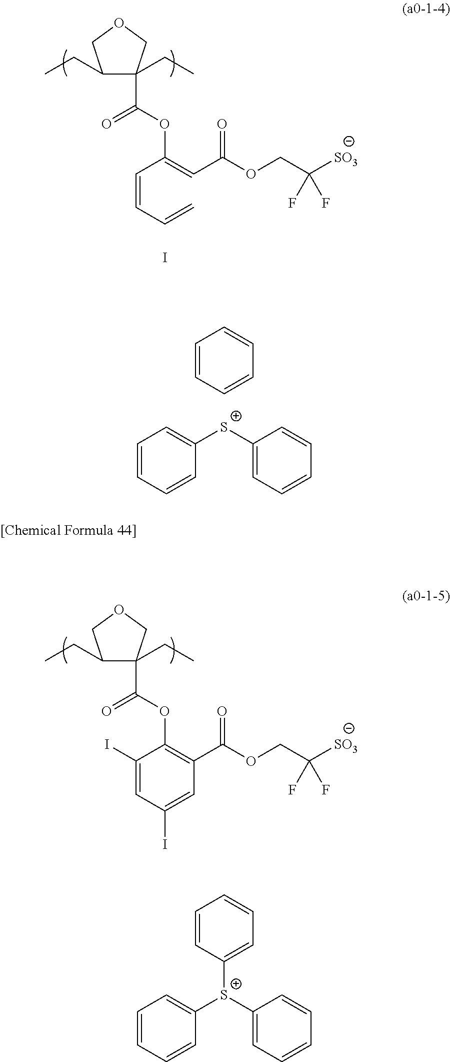

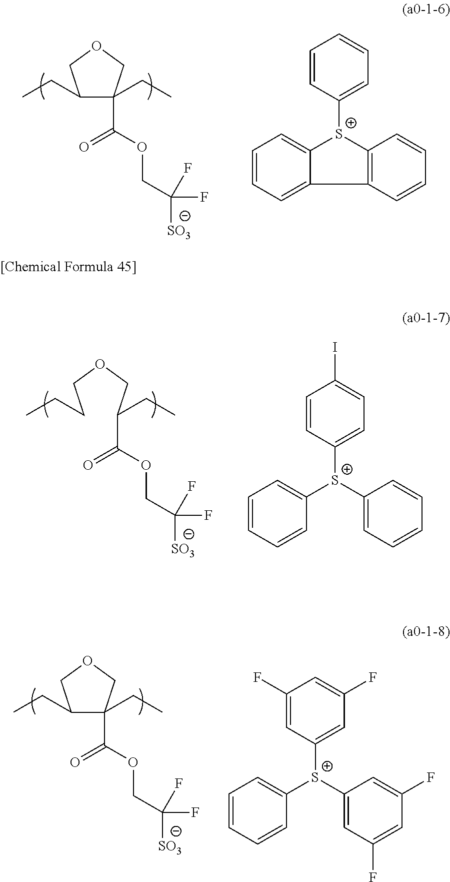

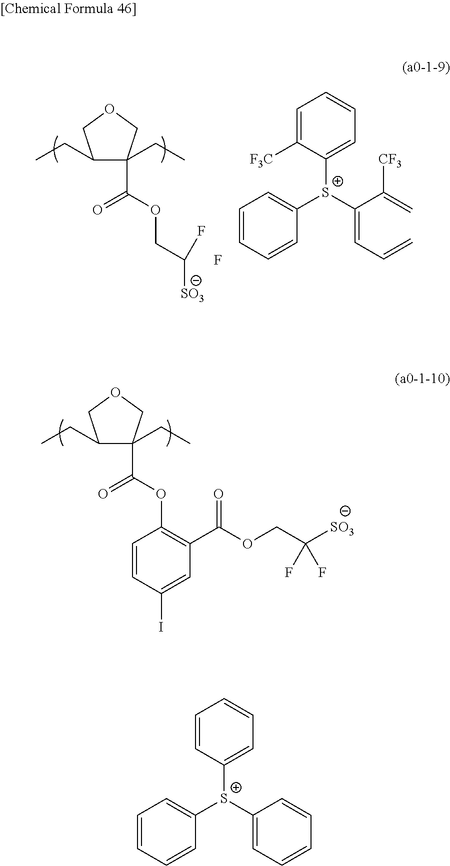

The constitutional unit (a0) contained in the component (A1) may be one kind or may be two or more kinds.

The proportion of the constitutional unit (a0) in the component (A1) is preferably in a range of 1% to 50% by mole, more preferably in a range of 2% to 35% by mole, still more preferably in a range of 5% to 30% by mole, and particularly preferably in a range of 10% to 25% by mole with respect to the total amount (100% by mole) of all constitutional units constituting the component (A1).

In a case where the proportion of the constitutional unit (a0) is equal to or greater than the lower limits of the above-described preferable ranges, roughness and resolution are further improved while maintaining good sensitivity. In a case where the proportion of the constitutional unit (a0) is equal to or less than the upper limits of the above-described preferable ranges, the constitutional unit (a0) and other constitutional units can be balanced, and various lithography characteristics are enhanced.

### <<Other Constitutional Units>>

The component (A1) may have other constitutional units in addition to the constitutional unit (a0) described above.

Examples of the other constitutional units include a constitutional unit (a1) containing an acid-decomposable group whose polarity is increased by the action of an acid; a constitutional unit (a10) represented by General Formula (a10-1); a constitutional unit (a2) containing a lactone-containing cyclic group; and a constitutional unit (a8) derived from a compound represented by General Formula (a8-1).

### Constitutional Unit (a1):

The constitutional unit (a1) is a constitutional unit that contains an acid-decomposable group whose polarity is increased due to the action of an acid.

Examples of the acid-dissociable group are the same as those which have been suggested as the acid-dissociable groups of the base resin for a chemically amplified resist composition.

Specific examples of the suggested acid-dissociable group of the base resin for a chemically amplified resist composition include an “acetal type acid-dissociable group”, “tertiary alkyl ester type acid-dissociable group”, “tertiary alkyloxycarbonyl acid-dissociable group”, and “secondary alkyloxycarbonyl acid-dissociable group” described below.

### Acetal Type Acid-Dissociable Group:

Examples of the acid-dissociable group that protects a carboxy group or a hydroxyl group in the polar groups include an acid-dissociable group represented by General Formula (a1-r-1) (hereinafter, also referred to as “acetal type acid-dissociable group”).

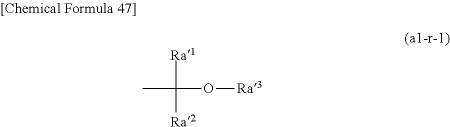

[In the formula, Ra′¹ and Ra′² represent a hydrogen atom or an alkyl group. Ra′³ represents a hydrocarbon group, and Ra′³ may be bonded to any of Ra′¹ and Ra′² to form a ring.]

In Formula (a1-r-1), it is preferable that at least one of Ra′¹ or Ra′² represent a hydrogen atom and more preferable that both Ra′¹ and Ra′² represent a hydrogen atom.

In a case where Ra′¹ or Ra′² represents an alkyl group, examples of the alkyl group include the same alkyl groups as those for the substituent which may be bonded to the carbon atom at the α-position in the description of the α-substituted acrylic acid ester. Among these, an alkyl group having 1 to 5 carbon atoms is preferable. Specific preferred examples thereof include linear or branched alkyl groups. More specific examples thereof include a methyl group, an ethyl group, a propyl group, an isopropyl group, an n-butyl group, an isobutyl group, a tert-butyl group, a pentyl group, an isopentyl group, and a neopentyl group. Among these, a methyl group or an ethyl group is more preferable, and a methyl group is particularly preferable.

In Formula (a1-r-1), examples of the hydrocarbon group as Ra′³ include a linear or branched alkyl group and a cyclic hydrocarbon group.

The linear alkyl group preferably has 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group preferably has 3 to 10 carbon atoms and more preferably 3 to 5 carbon atoms. Specific examples thereof include an isopropyl group, an isobutyl group, a tert-butyl group, an isopentyl group, a neopentyl group a 1,1-diethylpropyl group, and a 2,2-dimethylbutyl group. Among these, an isopropyl group is preferable.

In a case where Ra′³ represents a cyclic hydrocarbon group, the hydrocarbon group may be an aliphatic hydrocarbon group or an aromatic hydrocarbon group and may be a polycyclic group or a monocyclic group.

As the aliphatic hydrocarbon group which is a monocyclic group, a group in which one hydrogen atom has been removed from a monocycloalkane is preferable. The monocycloalkane preferably has 3 to 6 carbon atoms, and specific examples thereof include cyclopentane and cyclohexane.

As the aliphatic hydrocarbon group which is a polycyclic group, a group in which one hydrogen atom has been removed from a polycycloalkane is preferable. As the polycycloalkane, a group having 7 to 12 carbon atoms is preferable, and specific examples thereof include adamantane, norbornane, isobornane, tricyclo[5.2.1.0^{2,6}]decane, and tetracyclododecane.

In a case where the cyclic hydrocarbon group as Ra′³ is an aromatic hydrocarbon group, the aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring preferably has 5 to 30 carbon atoms, more preferably 5 to 20 carbon atoms, still more preferably 6 to 15 carbon atoms, and particularly preferably 6 to 12 carbon atoms.

Specific examples of the aromatic ring include aromatic hydrocarbon rings such as benzene, naphthalene, anthracene, and phenanthrene; and aromatic heterocyclic rings in which some carbon atoms constituting the above-described aromatic hydrocarbon rings have been substituted with heteroatoms. Examples of the heteroatom in the aromatic heterocyclic ring include an oxygen atom, a sulfur atom, and a nitrogen atom. Specific examples of the aromatic heterocyclic ring include a pyridine ring and a thiophene ring.

Specific examples of the aromatic hydrocarbon group as Ra′³ include a group in which one hydrogen atom has been removed from the above-described aromatic hydrocarbon ring or aromatic heterocyclic ring (such as an aryl group or a heteroaryl group); a group in which one hydrogen atom has been removed from an aromatic compound having two or more aromatic rings (such as biphenyl or fluorene); and a group in which one hydrogen atom of the above-described aromatic hydrocarbon ring or aromatic heterocyclic ring has been substituted with an alkylene group (for example, an arylalkyl group such as a benzyl group, a phenethyl group, a 1-naphthylmethyl group, a 2-naphthylmethyl group, a 1-naphthylethyl group, or a 2-naphthylethyl group). The number of carbon atoms in the alkylene group bonded to the aromatic hydrocarbon ring or aromatic heterocyclic ring is preferably in a range of 1 to 4, more preferably 1 or 2, and particularly preferably 1.

The cyclic hydrocarbon group as Ra′³ may include a substituent. Examples of the substituent include —R^{P1}, —R^{P2}—O—R^{P1}, —R^{P2}—CO—R^{P1}, —R^{P2}—CO—OR^{P1}, —R^{P1}—O—CO—R^{P1}, —R^{P2}—OH, —R^{P2}—CN, and —R^{P2}—COOH (hereinafter, these substituents will also be collectively referred to as “Rax5”).

Here, R^{P1} represents a chain-like monovalent saturated hydrocarbon group having 1 to 10 carbon atoms, a monovalent aliphatic cyclic saturated hydrocarbon group having 3 to 20 carbon atoms, or a monovalent aromatic hydrocarbon group having 6 to 30 carbon atoms. Further, R^{P2} represents a single bond, a chain-like divalent saturated hydrocarbon group having 1 to 10 carbon atoms, a divalent aliphatic cyclic saturated hydrocarbon group having 3 to 20 carbon atoms, or a divalent aromatic hydrocarbon group having 6 to 30 carbon atoms. Here, some or all hydrogen atoms in the chain-like saturated hydrocarbon group, the aliphatic cyclic saturated hydrocarbon group, and the aromatic hydrocarbon group as R^{P1} and R^{P2} may be substituted with fluorine atoms. The aliphatic cyclic hydrocarbon group may have one or more of one kind of substituents or one or more of each of plural kinds of the substituents.

Examples of the chain-like monovalent saturated hydrocarbon group having 1 to 10 carbon atoms include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2]octanyl group, a tricyclo[5.2.1.02,6]decanyl group, a tricyclo[3.3.1.13,7]decanyl group, a tetracyclo[6.2.1.13,6.02,7]dodecanyl group, or an adamantyl group.

Examples of the monovalent aromatic hydrocarbon group having 6 to 30 carbon atoms include a group formed by removing one hydrogen atom from an aromatic hydrocarbon ring such as benzene, biphenyl, fluorene, naphthalene, anthracene, or phenanthrene.

In a case where Ra′³ is bonded to any of Ra′¹ and Ra′² to form a ring, the cyclic group is preferably a 4- to 7-membered ring and more preferably a 4- to 6-membered ring. Specific examples of the cyclic group include a tetrahydropyranyl group and a tetrahydrofuranyl group.

### Tertiary Alkyl Ester Type Acid-Dissociable Group:

Examples of the acid-dissociable group that protects a carboxy group among the polar groups include an acid-dissociable group represented by General Formula (a1-r-2).

Among examples of the acid-dissociable group represented by Formula (a1-r-2), hereinafter, a group formed of an alkyl group will also be referred to as “tertiary alkyl ester type acid-dissociable group” for convenience.

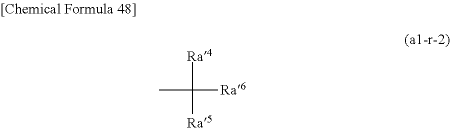

[In the formula, Ra′⁴ to Ra′⁶ each represents a hydrocarbon group, and Ra′⁵ and Ra′⁶ may be bonded to each other to form a ring.]

Examples of the hydrocarbon group as Ra′⁴ include a linear or branched alkyl group, a chain-like or cyclic alkenyl group, and a cyclic hydrocarbon group.

Examples of the linear or branched alkyl group and the cyclic hydrocarbon group (an aliphatic hydrocarbon group which is a monocyclic group, an aliphatic hydrocarbon group which is a polycyclic group, or an aromatic hydrocarbon group) as Ra′⁴ include the same groups as those for Ra′³.

As the chain-like or cyclic alkenyl group as Ra′⁴, an alkenyl group having 2 to 10 carbon atoms is preferable.

Examples of the hydrocarbon group as Ra′⁵ or Ra′⁶ include the same groups as those for Ra′³.

In a case where Ra′⁵ and Ra′⁶ are bonded to each other to form a ring, suitable examples thereof include a group represented by General Formula (a1-r2-1), a group represented by General Formula (a1-r2-2), and a group represented by General Formula (a1-r2-3).

Meanwhile, in a case where Ra′⁴ to Ra′⁶ represent an independent hydrocarbon group without being bonded to each other, suitable examples thereof include a group represented by General Formula (a1-r2-4).

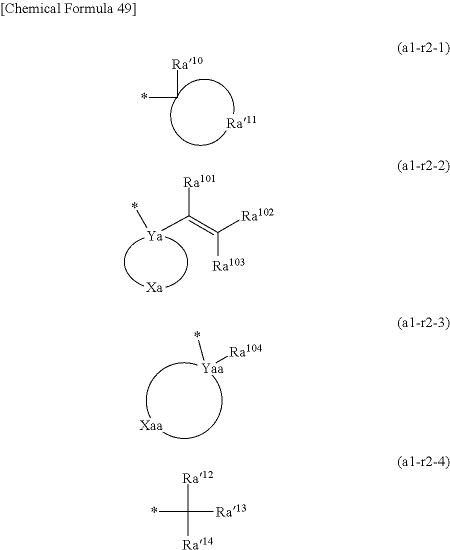

[In Formula (a1-r2-1), Ra′¹⁰ represents a linear or branched alkyl group having 1 to 12 carbon atoms, in which a part thereof may be substituted with a halogen atom or a heteroatom-containing group. Ra′¹¹ represents a group that forms an aliphatic cyclic group with the carbon atom to which Ra′¹⁰ has been bonded. In Formula (a1-r2-2), Ya represents a carbon atom. Xa represents a group that forms a cyclic hydrocarbon group with Ya. Some or all hydrogen atoms in this cyclic hydrocarbon group may be substituted. Ra¹⁰¹ to Ra¹⁰³ each independently represents a hydrogen atom, a chain-like monovalent saturated hydrocarbon group having 1 to 10 carbon atoms, or a monovalent aliphatic cyclic saturated hydrocarbon group having 3 to 20 carbon atoms. Some or all hydrogen atoms in the chain-like saturated hydrocarbon group and the aliphatic cyclic saturated hydrocarbon group may be substituted. Two or more of Ra¹⁰¹ to Ra¹⁰³ may be bonded to each other to form a cyclic structure. In Formula (a1-r2-3). Yaa represents a carbon atom. Xaa represents a group that forms an aliphatic cyclic group with Yaa. Ra¹⁰⁴ represents an aromatic hydrocarbon group which may have a substituent. In Formula (a1-r2-4), Ra′¹² and Ra′¹³ each independently represents a chain-like monovalent saturated hydrocarbon group having 1 to 10 carbon atoms. Some or all hydrogen atoms contained in the chain-like saturated hydrocarbon group may be substituted. Ra′¹⁴ represents a hydrocarbon group which may have a substituent. * represents a bonding site (the same applies hereinafter).]

In Formula (a1-r2-1), Ra′¹⁰ represents a linear or branched alkyl group having 1 to 12 carbon atoms, in which a part thereof may be substituted with a halogen atom or a heteroatom-containing group.

The linear alkyl group as Ra′¹⁰ has 1 to 12 carbon atoms, preferably 1 to 10 carbon atoms, and particularly preferably 1 to 5 carbon atoms.

Examples of the branched alkyl group as Ra′¹⁰ include the same groups as those for Ra′³ described above.

The alkyl group in Ra′¹⁰ may be partially substituted with a halogen atom or a heteroatom-containing group. For example, some hydrogen atoms constituting the alkyl group may be substituted with a halogen atom or a heteroatom-containing group. Further, some carbon atoms (methylene group or the like) constituting the alkyl group may be substituted with a heteroatom-containing group.

Examples of the heteroatoms here include an oxygen atom, a nitrogen atom, and a sulfur atom. Examples of the heteroatom-containing group include (—O—), —C(═O)—O—, —O—C(═O)—, —C(═O)—, —O—C(═O)—O—, —C(═O)—NH—, —NH—, —S—, —S(═O)₂—, and —S(═O)₂—O—.

In Formula (a1-r2-1), preferred examples of Ra′¹¹ (an aliphatic cyclic group that is formed together with a carbon atom to which Ra′¹⁰ is bonded) include the groups described as the aliphatic hydrocarbon group (alicyclic hydrocarbon group) which is a monocyclic group or a polycyclic group as Ra′³ in Formula (a1-r-1). Among these, it is preferably a monocyclic alicyclic hydrocarbon group, and specifically, it is more preferably a cyclopentyl group or a cyclohexyl group.

In Formula (a1-r2-2), examples of the cyclic hydrocarbon group that is formed by Xa together with Ya include a group in which one or more hydrogen atoms have been further removed from the cyclic monovalent hydrocarbon group (aliphatic hydrocarbon group) as Ra′³ in Formula (a1-r-1).

The cyclic hydrocarbon group that is formed by Xa together with Ya may have a substituent. Examples of the substituent include those which are the same as the substituents which may be included in the cyclic hydrocarbon group as Ra′³.

In Formula (a1-r2-2), examples of the chain-like monovalent saturated hydrocarbon group having 1 to 10 carbon atoms as Ra¹⁰¹ to Ra¹⁰³ include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms as Ra¹⁰¹ to Ra¹⁰³ include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2]octanyl group, a tricyclo[5.2.1.0^{2,6}] decanyl group, a tricyclo[3.3.1.1^{3,7}] decanyl group, a tetracyclo[6.2.1.1^{3,6}0.^{2,7}] dodecanyl group, or an adamantyl group.

From the viewpoint of ease of synthesis, Ra¹⁰¹ to Ra¹⁰³ preferably represent a hydrogen atom or a chain-like monovalent saturated hydrocarbon group having 1 to 10 carbon atoms, more preferably a hydrogen atom, a methyl group, or an ethyl group, and particularly preferably a hydrogen atom.

Examples of the substituent included in the chain-like saturated hydrocarbon group or the aliphatic cyclic saturated hydrocarbon group represented by Ra¹⁰¹ to Ra¹⁰³ are the same groups as those for Ra′⁵.

Examples of the group having a carbon-carbon double bond generated by two or more of Ra¹⁰¹ to Ra¹⁰³ being bonded to each other to form a cyclic structure include a cyclopentenyl group, a cyclohexenyl group, a methylcyclopentenyl group, a methylcyclohexenyl group, a cyclopentylidenethenyl group, and a cyclohexylidenethenyl group. Among these, from the viewpoint of ease of synthesis, a cyclopentenyl group, a cyclohexenyl group, or a cyclopentylidenethenyl group is preferable.

In Formula (a1-r2-3), as the aliphatic cyclic group that is formed by Xaa together with Yaa, the group exemplified as the aliphatic hydrocarbon group which is a monocyclic group or a polycyclic group as Ra′³ in Formula (a1-r-1) is preferable.

In Formula (a1-r2-3), examples of the aromatic hydrocarbon group as Ra¹⁰⁴ include a group in which one or more hydrogen atoms have been removed from an aromatic hydrocarbon ring having 5 to 30 carbon atoms. Among the examples, Ra¹⁰⁴ preferably represents a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from benzene or naphthalene, and most preferably a group in which one or more hydrogen atoms have been removed from benzene.

Examples of the substituent that Ra¹⁰⁴ in Formula (a1-r2-3) may have include a methyl group, an ethyl group, a propyl group, a hydroxy group, a carboxy group, a halogen atom, an alkoxy group (such as a methoxy group, an ethoxy group, a propoxy group, or a butoxy group), and an alkyloxycarbonyl group.

In Formula (a1-r2-4), Ra′¹² and Ra′¹³ each independently represents a chain-like monovalent saturated hydrocarbon group having 1 to 10 carbon atoms. Examples of the chain-like monovalent saturated hydrocarbon group having 1 to 10 carbon atoms as Ra′¹² and Ra′¹³ include those exemplified as the chain-like monovalent saturated hydrocarbon group having 1 to 10 carbon atoms as Ra¹⁰¹ to Ra¹⁰³. Some or all hydrogen atoms contained in the chain-like saturated hydrocarbon group may be substituted.

Ra′¹² and Ra′¹³ preferably represent an alkyl group having 1 to 5 carbon atoms, more preferably an alkyl group having 1 to 5 carbon atoms, still more preferably a methyl group or an ethyl group, and particularly preferably a methyl group.

In a case where the chain-like saturated hydrocarbon group represented by Ra′¹² and Ra′¹³ is substituted, examples of the substituent include the same groups as those for Ra^{x5} described above.

In Formula (a1-r2-4), Ra′¹⁴ represents a hydrocarbon group which may have a substituent. Examples of the hydrocarbon group as Ra′¹⁴ include a linear or branched alkyl group and a cyclic hydrocarbon group.

The linear alkyl group as Ra′¹⁴ preferably has 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group as Ra′¹⁴ preferably has 3 to 10 carbon atoms and more preferably 3 to 5 carbon atoms. Specific examples thereof include an isopropyl group, an isobutyl group, a tert-butyl group, an isopentyl group, a neopentyl group a 1,1-diethylpropyl group, and a 2,2-dimethylbutyl group. Among these, an isopropyl group is preferable.

In a case where Ra′¹⁴ represents a cyclic hydrocarbon group, the hydrocarbon group may be an aliphatic hydrocarbon group or an aromatic hydrocarbon group and may be a polycyclic group or a monocyclic group.

As the aliphatic hydrocarbon group which is a monocyclic group, a group in which one hydrogen atom has been removed from a monocycloalkane is preferable. The monocycloalkane preferably has 3 to 6 carbon atoms, and specific examples thereof include cyclopentane and cyclohexane.

As the aliphatic hydrocarbon group which is a polycyclic group, a group in which one hydrogen atom has been removed from a polycycloalkane is preferable. As the polycycloalkane, a group having 7 to 12 carbon atoms is preferable, and specific examples thereof include adamantane, norbornane, isobornane, tricyclo[5.2.1.0^{2,6}]decane, and tetracyclododecane.

Examples of the aromatic hydrocarbon group as Ra′¹⁴ include the same groups as those for the aromatic hydrocarbon group as Ra¹⁰⁴. Among these, Ra′¹⁴ preferably represents a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from naphthalene or anthracene, and most preferably a group in which one or more hydrogen atoms have been removed from naphthalene.

Examples of the substituent that Ra′¹⁴ may have include the same groups as those for the substituent that Ra¹⁰⁴ may have.

In a case where Ra′¹⁴ in Formula (a1-r2-4) represents a naphthyl group, the position bonded to the tertiary carbon atom in Formula (a1-r2-4) may be any of the 1-position or the 2-position of the naphthyl group.

In a case where Ra′¹⁴ in Formula (a1-r2-4) represents an anthryl group, the position bonded to the tertiary carbon atom in Formula (a1-r2-4) may be any of the 1-position, the 2-position, or the 9-position of the anthryl group.

Specific examples of the group represented by Formula (a1-r2-1) are shown below.

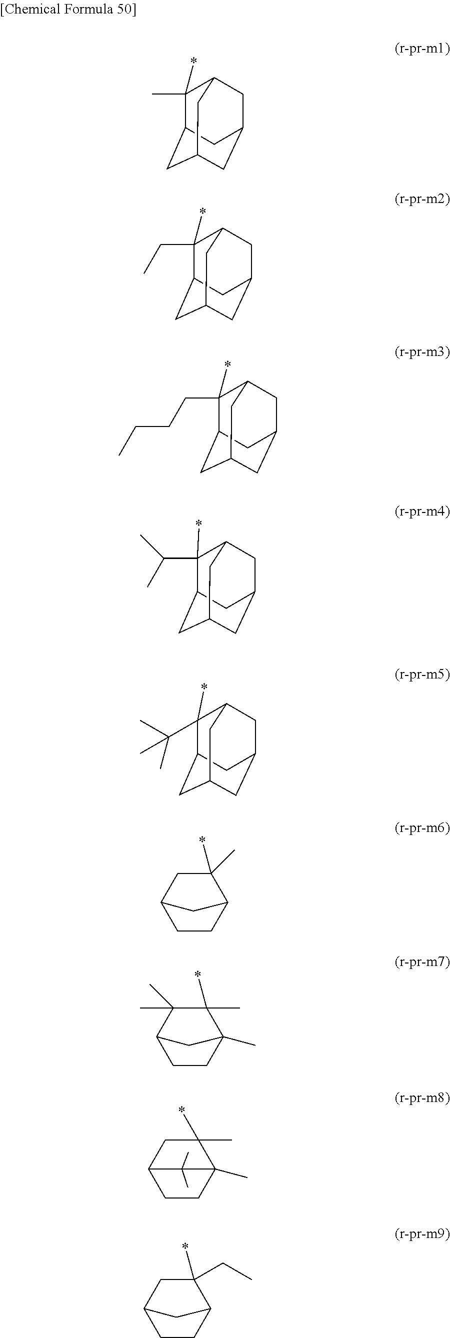

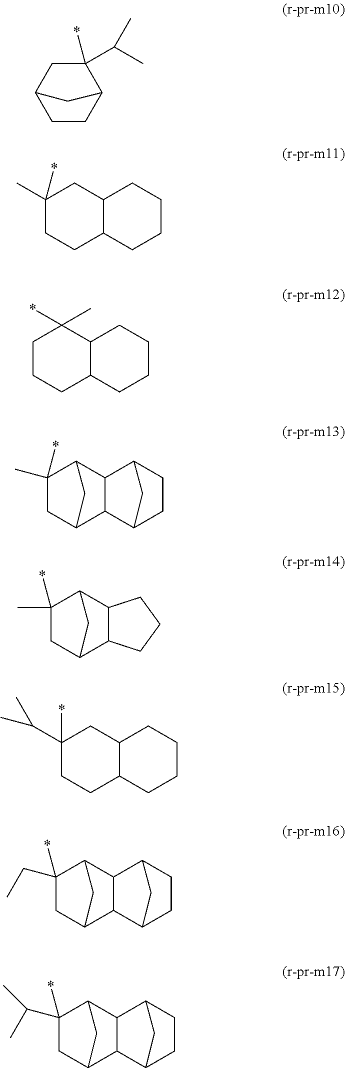

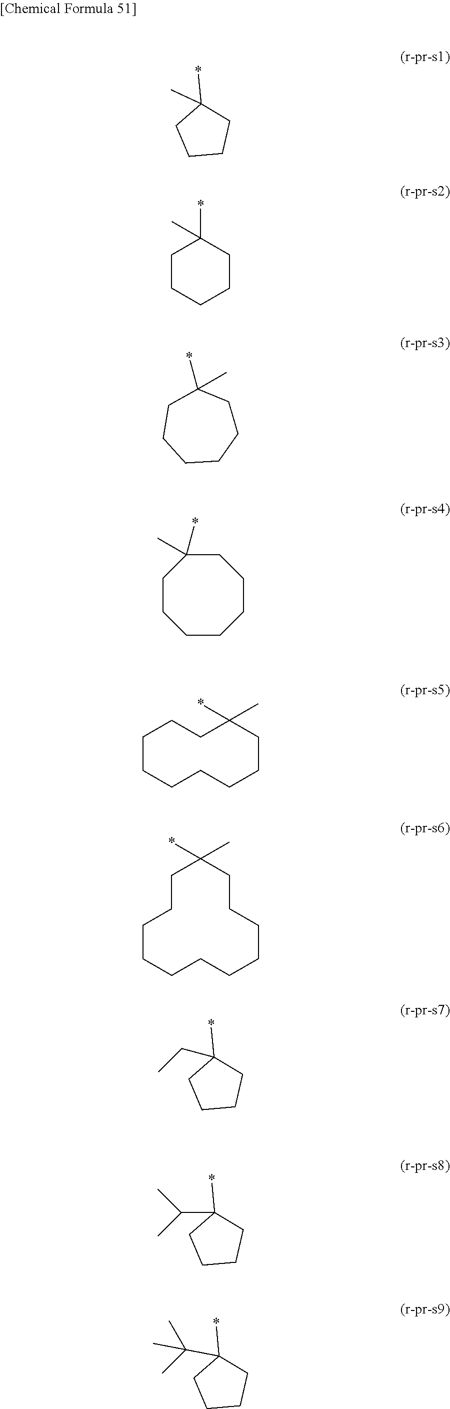

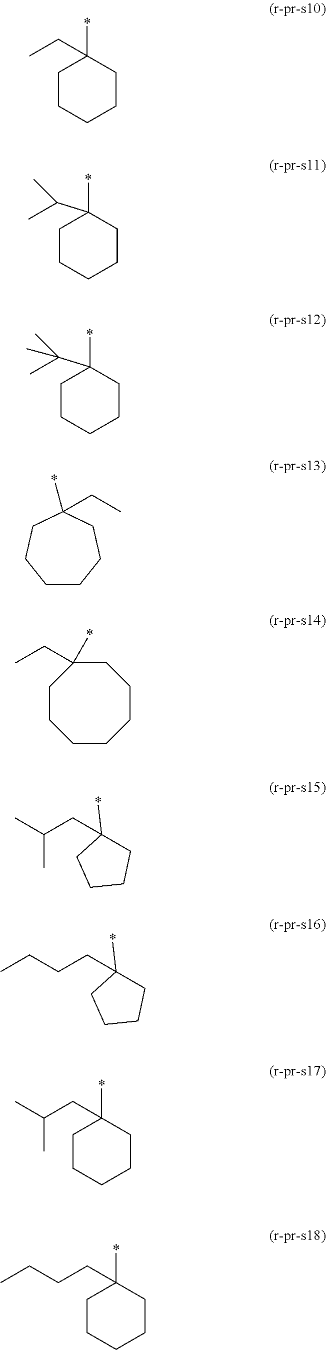

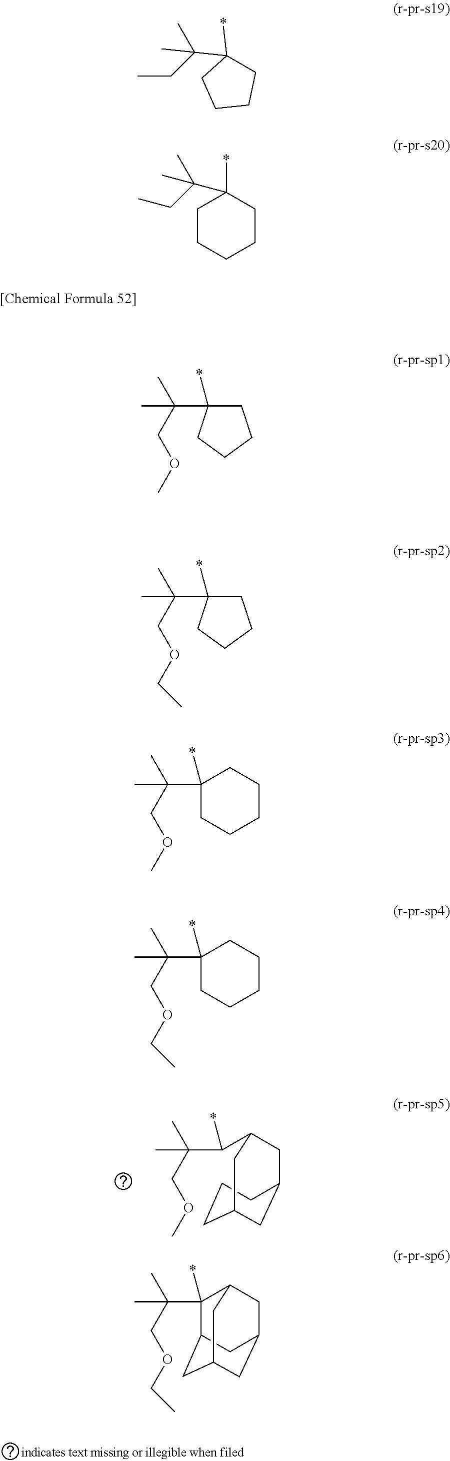

Specific examples of the group represented by Formula (a1-r2-2) are shown below.

[chemical structure]

Specific examples of the group represented by Formula (a1-r2-3) are shown below.

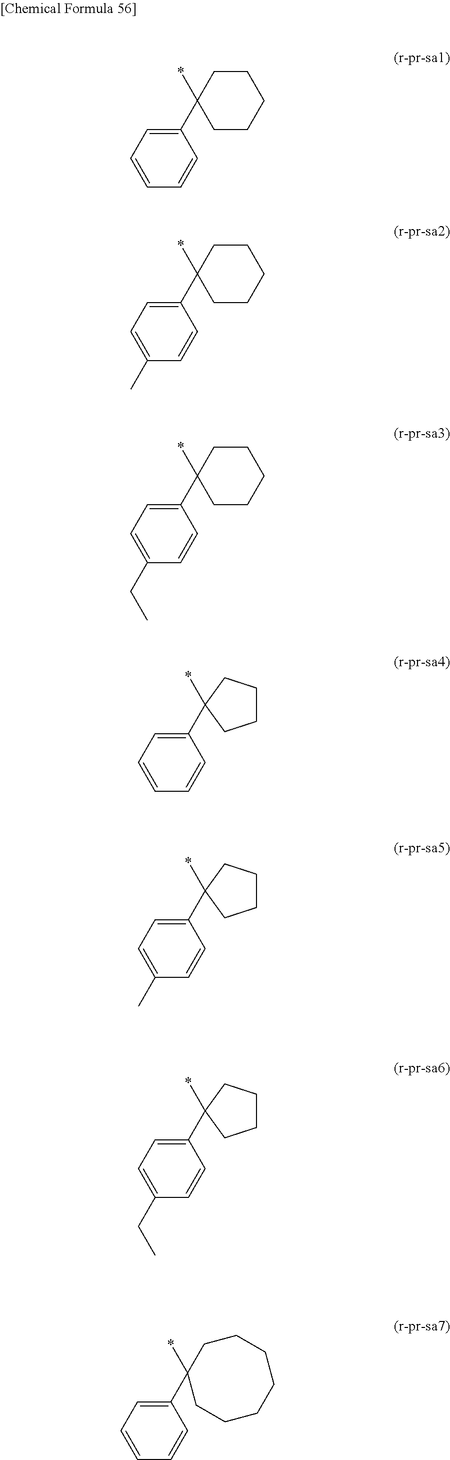

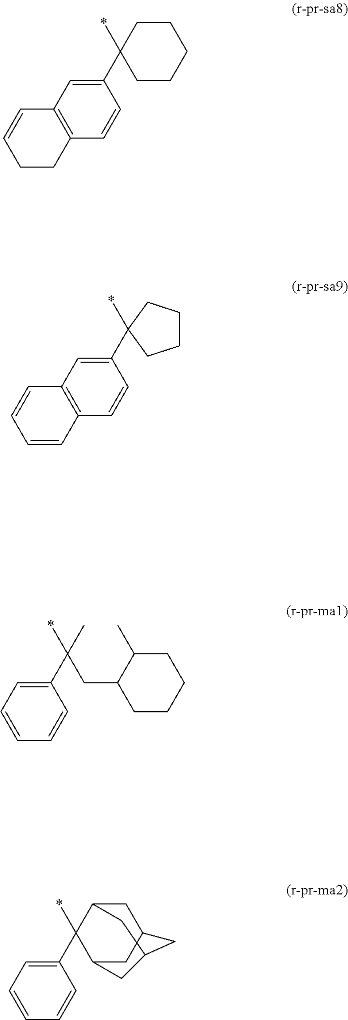 [SMILES: *C1(c2ccc3c(c2)C=CCC3)CCCCC1 ; *C1(c2ccc3ccccc3c2)CCCC1 ; *C(C)(CC1CCCCC1C)c1ccccc1 ; *C1(c2ccccc2)C2CC3CC(C2)CC1C3]

Specific examples of the group represented by Formula (a1-r2-4) are shown below.

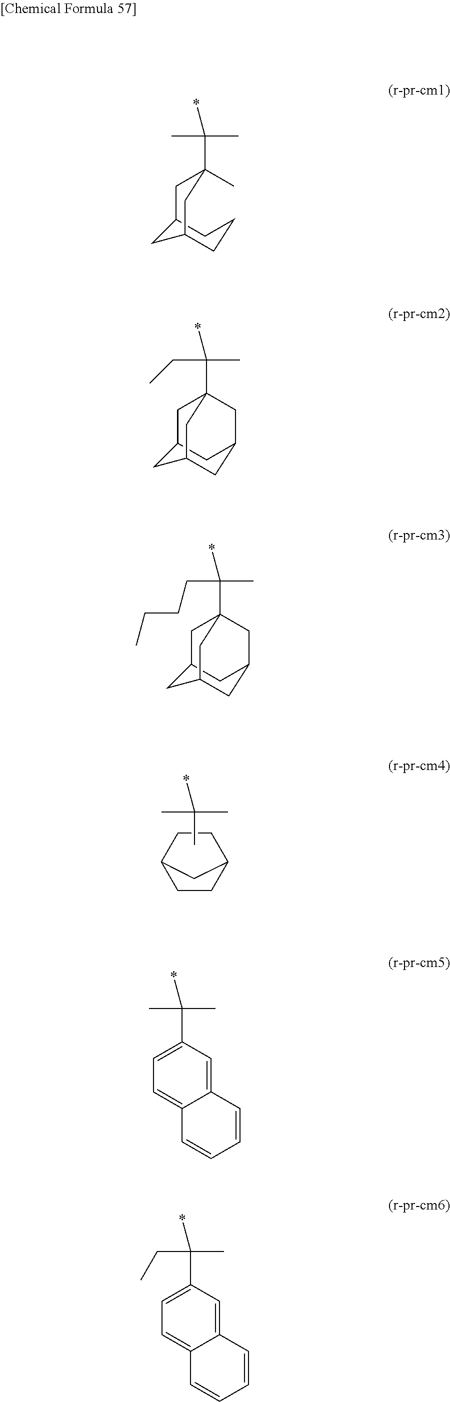

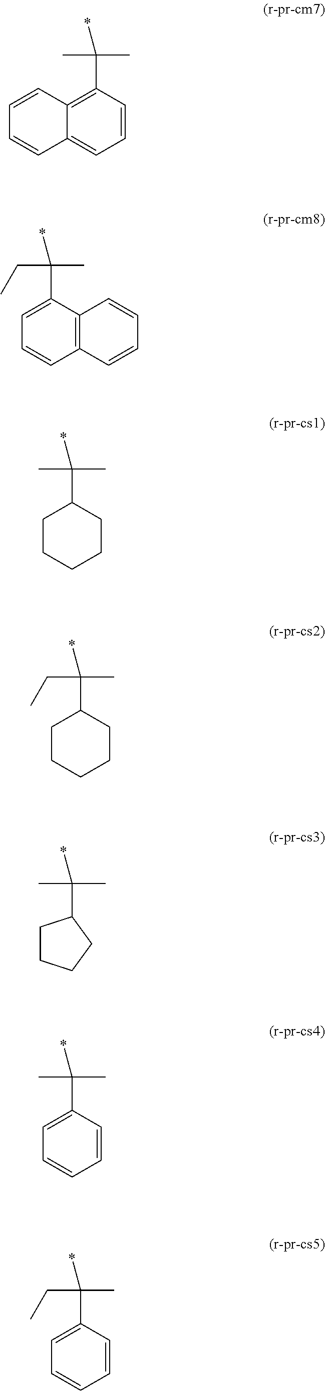

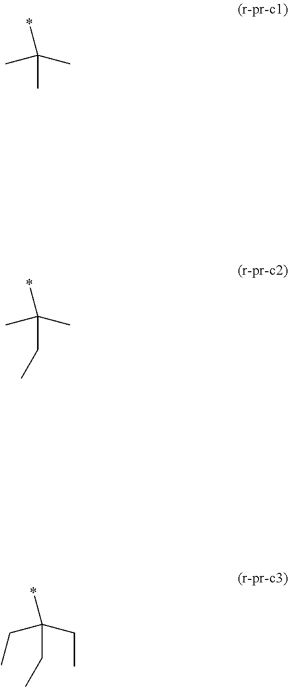

### Tertiary Alkyloxycaronyl Acid-Dissociable Group:

Examples of the acid-dissociable group that protects a hydroxyl group among the polar groups include an acid-dissociable group (hereinafter, also referred to as “tertiary alkyloxycarbonyl acid-dissociable group” for convenience) represented by General Formula (a1-r-3).

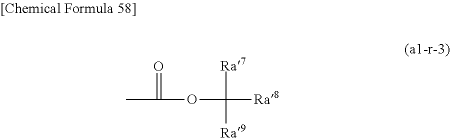

[In the formula, Ra′⁷ to Ra′⁹ each represents an alkyl group.]

In Formula (a1-r-3), Ra′⁷ to Ra′⁹ each preferably represents an alkyl group having 1 to 5 carbon atoms and more preferably an alkyl group having 1 to 3 carbon atoms.

Further, the total number of carbon atoms in each alkyl group is preferably in a range of 3 to 7, more preferably in a range of 3 to 5, and most preferably 3 or 4.

### Secondary Alkyl Ester Type Acid-Dissociable Group:

Examples of the acid-dissociable group that protects a carboxy group among the polar groups include an acid-dissociable group represented by General Formula (a1-r-4).

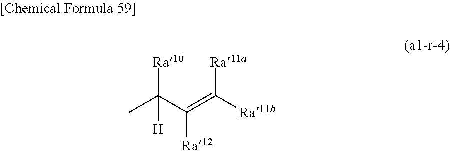

[In the formula. Ra′¹ represents a hydrocarbon group. Ra′¹¹ and Ra′^{11b} each independently represents a hydrogen atom, a halogen atom, or an alkyl group. Ra′¹² represents a hydrogen atom or a hydrocarbon group. Ra′¹⁰ and Ra′^{11a} or Ra′^{11b} may be bonded to each other to form a ring. Ra′^{11a} or Ra′^{11b} and Ra′¹² may be bonded to each other to form a ring.]

Examples of the hydrocarbon group as Ra′¹⁰ or Ra′¹² in the formula include the same groups as those for Ra′³.

Examples of the alkyl group as Ra′^{11a} and Ra′^{11b} in the formula include the same groups as those for the alkyl group as Ra′¹.

In the formula, the hydrocarbon group as Ra′¹⁰ or Ra′¹² and the alkyl group as Ra′^{11a} and Ra′^{11b} may have a substituent. Examples of the substituent include Ra^{x5} described above.

Ra′¹⁰ and Ra′^{11a} or Ra′^{11b} may be bonded to each other to form a ring. The ring may be a polycyclic ring or a monocyclic ring, and may be an alicyclic ring or an aromatic ring.

The alicyclic ring and the aromatic ring may have a heteroatom.

Among the examples described above, as the ring formed by Ra′¹⁰ and Ra′^{11 a} or Ra′^{11b} being bonded to each other, monocycloalkene, a ring in which some carbon atoms of monocycloalkene have been substituted with heteroatoms (such as an oxygen atom and a sulfur atom), or monocycloalkadiene is preferable, cycloalkene having 3 to 6 carbon atoms is preferable, and cyclopentene or cyclohexene is preferable.

The ring formed by Ra′¹⁰ and Ra′^{11a} or Ra′^{11b} being bonded to each other may be a condensed ring. Specific examples of the condensed ring include indane.

The ring formed by Ra′¹⁰ and Ra′^{11a} or Ra′^{11b} being bonded to each other may have a substituent. Examples of the substituent include Ra^{x5} described above.

Ra′^{11a} or Ra′^{11b} and Ra′¹² may be bonded to each other to form a ring, and examples of the ring include the same rings as those formed by Ra′¹⁰ and Ra′^{11a} or Ra′^{11b} being bonded to each other.

Specific examples of the group represented by Formula (a1-r-4) are shown below.

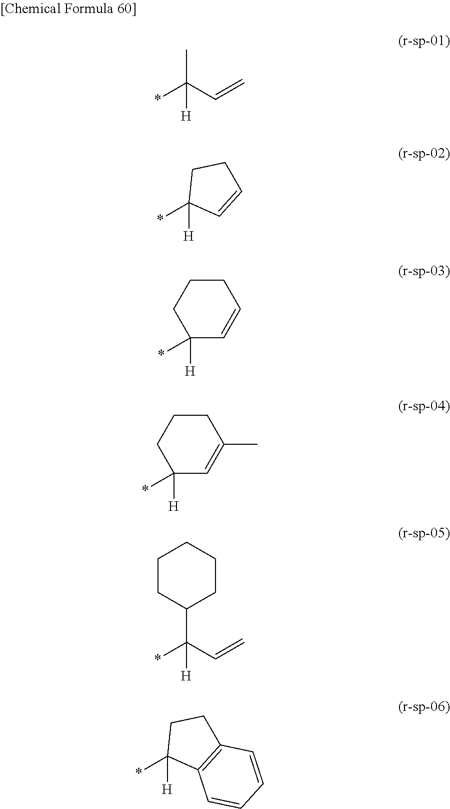

Examples of the constitutional unit (a1) include a constitutional unit derived from acrylic acid ester in which the hydrogen atom bonded to the carbon atom at the α-position may be substituted with a substituent; a constitutional unit derived from acrylamide; a constitutional unit in which at least some hydrogen atoms in a hydroxyl group of a constitutional unit derived from hydroxystyrene or a hydroxystyrene derivative are protected by a substituent containing the acid-decomposable group; and a constitutional unit in which at least some hydrogen atoms in —C(═O)—OH of a constitutional unit derived from vinylbenzoic acid or a vinylbenzoic acid derivative are protected by a substituent containing the acid-decomposable group.

Among the examples, as the constitutional unit (a1), a constitutional unit derived from acrylic acid ester in which the hydrogen atom bonded to the carbon atom at the α-position may be substituted with a substituent is preferable.

Specific preferred examples of such a constitutional unit (a1) include a constitutional unit represented by General Formula (a1-1), (a1-2), or (a1-3).

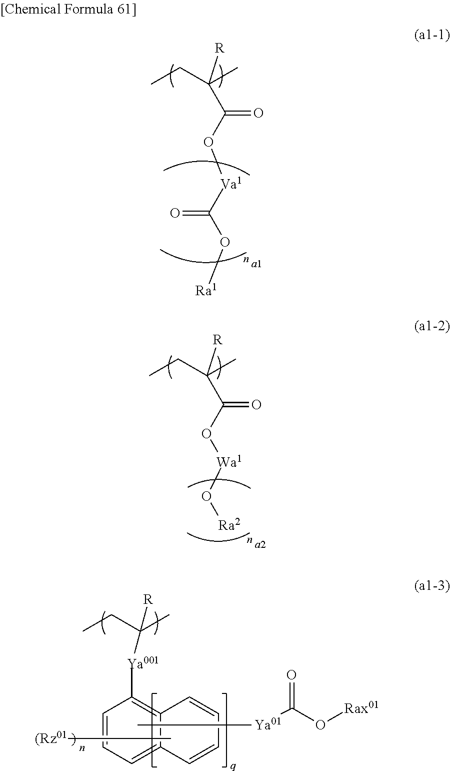

[In the formula, R represents a hydrogen atom, an alkyl group having 1 to 5 carbon atoms, or a halogenated alkyl group having 1 to 5 carbon atoms. Va¹ represents a divalent hydrocarbon group which may have an ether bond. nₐ₁ represents an integer of 0 to 2. Ra¹ represents an acid-dissociable group represented by General Formula (a1-r-1), (a1-r-2), or (a1-r-4). Wa¹ represents an (nₐ₂+1)-valent hydrocarbon group. nₐ₂ represents an integer of 1 to 3. Ra² represents an acid-dissociable group represented by General Formula (a1-r-1) or (a1-r-3). Ya⁰⁰¹ represents a single bond or a divalent linking group. Ya⁰¹ represents a single bond or a divalent linking group. Rax⁰¹ represents an acid-dissociable group represented by General Formula (a1-r-1), (a1-r-2), or (a1-r-4). Rz⁰¹ represents an alkyl group, a halogen atom, a halogenated alkyl group, a hydroxy group, or an alkoxy group. q represents an integer of 0 to 3. n represents an integer of 0 or greater. However, n≤q×2+4 is satisfied.]

In Formulae (a1-1) to (a1-3), as the alkyl group having 1 to 5 carbon atoms as R, a linear or branched alkyl group having 1 to 5 carbon atoms is preferable, and specific examples thereof include a methyl group, an ethyl group, a propyl group, an isopropyl group, an n-butyl group, an isobutyl group, a tert-butyl group, a pentyl group, an isopentyl group, and a neopentyl group. The halogenated alkyl group having 1 to 5 carbon atoms is a group in which some or all hydrogen atoms in the alkyl group having 1 to 5 carbon atoms have been substituted with halogen atoms. As the halogen atom, a fluorine atom is particularly preferable.

R preferably represents a hydrogen atom, an alkyl group having 1 to 5 carbon atoms, or a fluorinated alkyl group having 1 to 5 carbon atoms, and most preferably a hydrogen atom or a methyl group from the viewpoint of the industrial availability.

In Formula (a1-1), the divalent hydrocarbon group as Va¹ may be an aliphatic hydrocarbon group or an aromatic hydrocarbon group.

The aliphatic hydrocarbon group as the divalent hydrocarbon group represented by Va¹ may be saturated or unsaturated. In general, it is preferable that the aliphatic hydrocarbon group be saturated.

More specific examples of the aliphatic hydrocarbon group include a linear or branched aliphatic hydrocarbon group and an aliphatic hydrocarbon group having a ring in the structure thereof.

The linear aliphatic hydrocarbon group preferably has 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group preferably has 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

Examples of the aliphatic hydrocarbon group having a ring in the structure thereof include an alicyclic hydrocarbon group (a group in which two hydrogen atoms have been removed from an aliphatic hydrocarbon ring), a group in which the alicyclic hydrocarbon group is bonded to the terminal of the linear or branched aliphatic hydrocarbon group, and a group in which the alicyclic hydrocarbon group is interposed in the middle of the linear or branched aliphatic hydrocarbon group. Examples of the linear or branched aliphatic hydrocarbon group include the same groups as those for the linear aliphatic hydrocarbon group or the branched aliphatic hydrocarbon group.

The alicyclic hydrocarbon group preferably has 3 to 20 carbon atoms and more preferably 3 to 12 carbon atoms.

The alicyclic hydrocarbon group may be monocyclic or polycyclic. As the monocyclic alicyclic hydrocarbon group, a group in which two hydrogen atoms have been removed from a monocycloalkane is preferable. The monocycloalkane preferably has 3 to 6 carbon atoms, and specific examples thereof include cyclopentane and cyclohexane. As the polycyclic alicyclic hydrocarbon group, a group in which two hydrogen atoms have been removed from a polycycloalkane is preferable. The number of carbon atoms in the polycycloalkane is preferably in a range of 7 to 12, and specific examples thereof include adamantane, norbornane, isobornane, tricyclo[5.2.1.0^{2,6}]decane, and tetracyclododecane.

The aromatic hydrocarbon group as the divalent hydrocarbon group represented by Va¹ is a hydrocarbon group having an aromatic ring.

The aromatic hydrocarbon group preferably has 3 to 30 carbon atoms, more preferably 5 to 30 carbon atoms, still more preferably 5 to 20 carbon atoms, particularly preferably 6 to 15 carbon atoms, and most preferably 6 to 12 carbon atoms. Here, the number of carbon atoms in a substituent is not included in the number of carbon atoms.

Specific examples of the aromatic ring contained in the aromatic hydrocarbon group include aromatic hydrocarbon rings such as benzene, biphenyl, fluorene, naphthalene, anthracene, and phenanthrene; and aromatic heterocyclic rings in which some carbon atoms constituting the above-described aromatic hydrocarbon rings have been substituted with heteroatoms. Examples of the heteroatom in the aromatic heterocyclic rings include an oxygen atom, a sulfur atom, and a nitrogen atom.

Specific examples of the aromatic hydrocarbon group include a group in which two hydrogen atoms have been removed from the above-described aromatic hydrocarbon ring (an arylene group); and a group in which one hydrogen atom of a group (an aryl group) formed by removing one hydrogen atom from the aromatic hydrocarbon ring has been substituted with an alkylene group (for example, a group formed by further removing one more hydrogen atoms from an aryl group in an arylalkyl group such as a benzyl group, a phenethyl group, a 1-naphthylmethyl group, a 2-naphthylmethyl group, a 1-naphthylethyl group, or a 2-naphthylethyl group). The number of carbon atoms in the alkylene group (alkyl chain in the arylalkyl group) is preferably in a range of 1 to 4, more preferably 1 or 2 carbon atoms, and particularly preferably 1.

In Formula (a1-1), Ra¹ preferably represents an acid-dissociable group represented by General Formula (a1-r-2) or (a1-r-4) and, among these, more preferably a group represented by General Formula (a1-r2-1) or an acid-dissociable group represented by General Formula (a1-r-4).

In Formula (a1-2), the (nₐ₂+1)-valent hydrocarbon group as Wa¹ may be an aliphatic hydrocarbon group or an aromatic hydrocarbon group. The aliphatic hydrocarbon group indicates a hydrocarbon group that has no aromaticity and may be saturated or unsaturated. In general, it is preferable that the aliphatic hydrocarbon group be saturated. Examples of the aliphatic hydrocarbon group include a linear or branched aliphatic hydrocarbon group, an aliphatic hydrocarbon group having a ring in the structure thereof, and a group obtained by combining the linear or branched aliphatic hydrocarbon group and the aliphatic hydrocarbon group having a ring in the structure thereof.

The valency of nₐ₂+1 is preferably divalent to tetravalent and more preferably divalent or trivalent.

In Formula (a1-2), it is preferable that Ra² represent an acid-dissociable group represented by General Formula (a1-r-1).

In Formula (a1-3), the divalent linking group as Ya⁰⁰¹ is not particularly limited, and suitable examples thereof include a divalent hydrocarbon group which may have a substituent and a divalent linking group having a heteroatom.

Among these, it is preferable that Ya⁰⁰¹ represent an ester bond [—C(═O)—O— or —O—C(═O)—], an ether bond (—O—), a linear or branched alkylene group, an aromatic hydrocarbon group, a combination thereof, or a single bond. The alkylene group preferably has 1 to 10 carbon atoms, more preferably 1 to 6 carbon atoms, still more preferably 1 to 4 carbon atoms, and particularly preferably 1 to 3 carbon atoms.

Among these, Ya⁰⁰¹ more preferably represents a combination of an ester bond [—C(═O)—O— or —O—C(═O)—] and a linear alkylene group, or a single bond and still more preferably a single bond.

In Formula (a1-3), the divalent linking group as Ya⁰¹ is not particularly limited, and suitable examples thereof include a divalent hydrocarbon group which may have a substituent, and a divalent linking group having a heteroatom.

Among these, it is preferable that Ya⁰¹ represent an ester bond [—C(═O)—O— or —O—C(═O)—], an ether bond (—O—), a linear or branched alkylene group, an aromatic hydrocarbon group, a combination thereof, or a single bond. Among these, Ya⁰¹ more preferably represents a combination of an ester bond [—C(═O)—O— or —O—C(═O)—] and a linear alkylene group, or a single bond and still more preferably a single bond.

In Formula (a1-3), Rax⁰¹ preferably represents an acid-dissociable group represented by General Formula (a1-r-2) or (a1-r-4) and, among these, more preferably an acid-dissociable group represented by General Formula (a1-r-2) and still more preferably a group represented by General Formula (a1-r2-1).

In Formula (a1-3), the alkyl group, the halogenated alkyl group, and the alkoxy group as Rz⁰¹ preferably have 1 to 10 carbon atoms, more preferably 1 to 5 carbon atoms, still more preferably 1 to 3 carbon atoms, and particularly preferably 1 or 2 carbon atoms. The alkyl group, the halogenated alkyl group, and the alkoxy group may be linear or branched.

As the halogen atom represented by Rz⁰¹, an iodine atom is preferable. As the halogen atom of the halogenated alkyl group represented by Rz⁰¹, a fluorine atom, an iodine atom, or a bromine atom is preferable, and a fluorine atom is more preferable.

Rz⁰¹ preferably represents an alkoxy group or a hydroxy group and more preferably a hydroxy group.

In Formula (a1-3), q represents an integer of 0 to 3. A benzene structure is formed in a case where q represents 0, a naphthalene structure is formed in a case where q represents 1, an anthracene structure is formed in a case where q represents 2, and a tetracene structure is formed in a case where q represents 3.

In Formula (a1-3), n represents an integer of 0 or greater, preferably 0 to 5, more preferably 0 to 3, and still more preferably 1 or 2. In a case where n represents an integer of 2 or greater, two or more Rz⁰¹'s may be the same as or different from each other.

In Formula (a1-3), n≤q×2+4 is satisfied. For example, in a case where q represents 1 and thus a naphthalene structure is formed, all six hydrogen atoms of the naphthalene may be substituted with hydroxy groups. In addition, the substitution positions of Ya⁰⁰¹, the -Ya⁰¹-C(═O)—O—Ra⁰¹ group, and the hydroxy group in the naphthalene are not particularly limited.

Specific examples of the constitutional unit (a1) are shown below. In the formulae shown below, R^{α} represents a hydrogen atom, a methyl group, or a trifluoromethyl group.

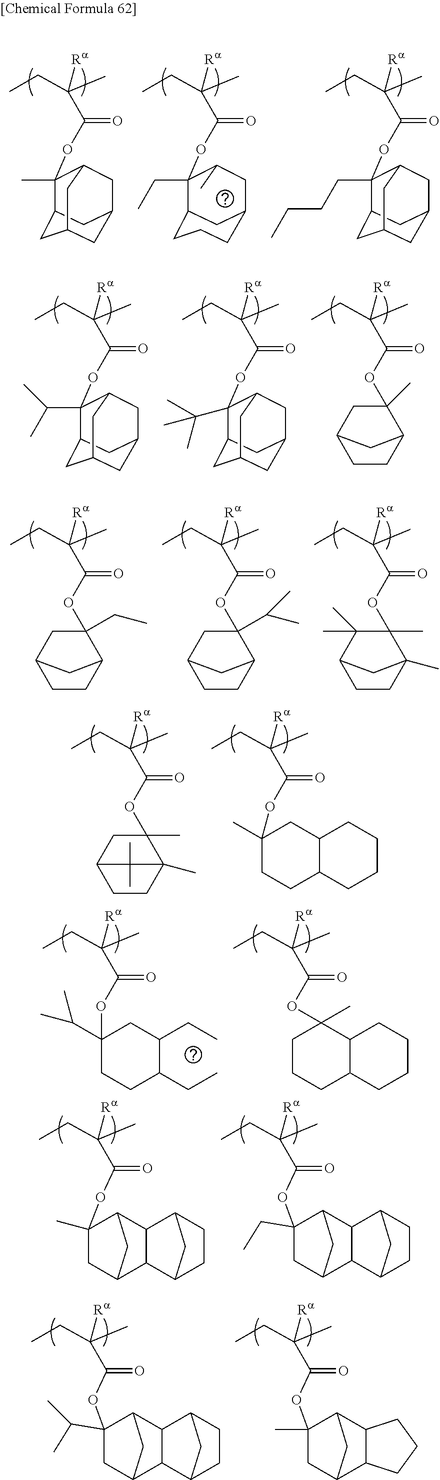

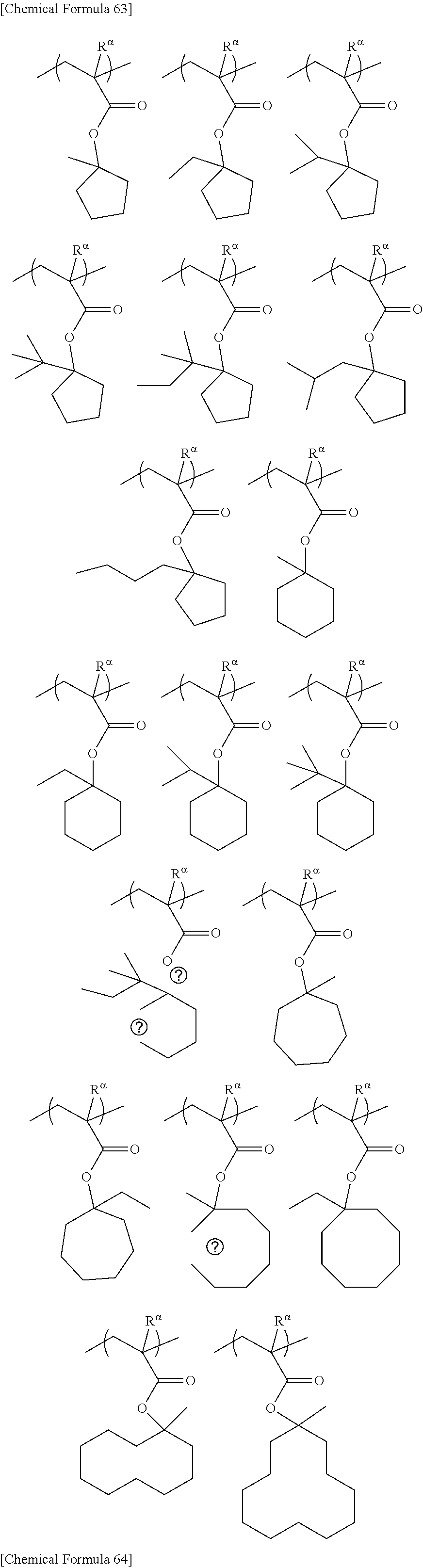

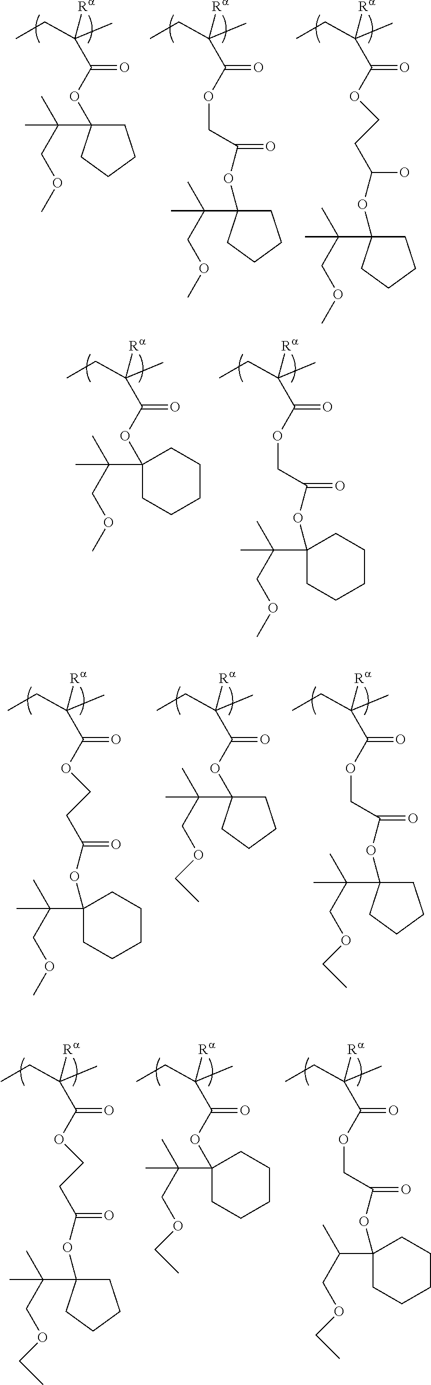

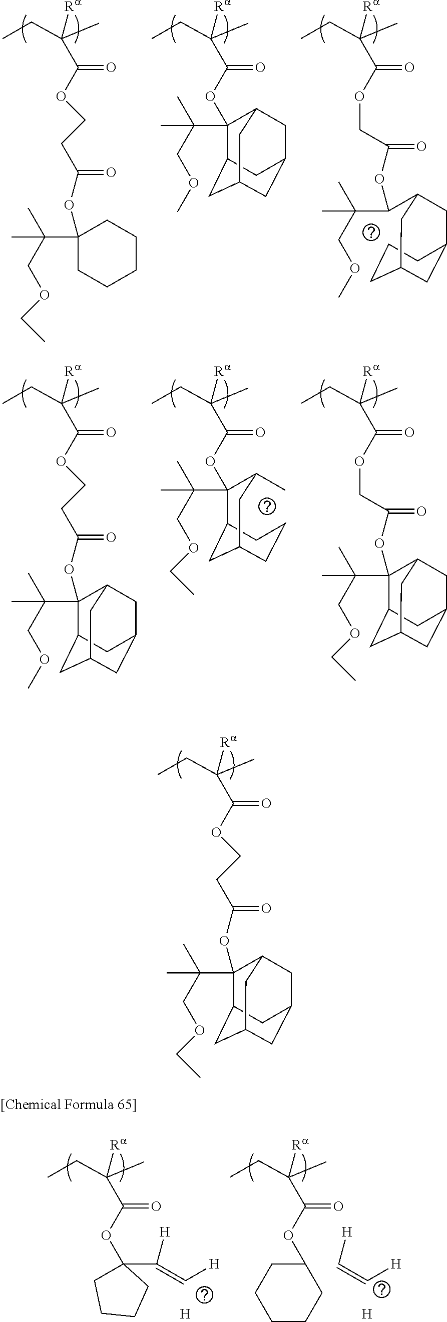

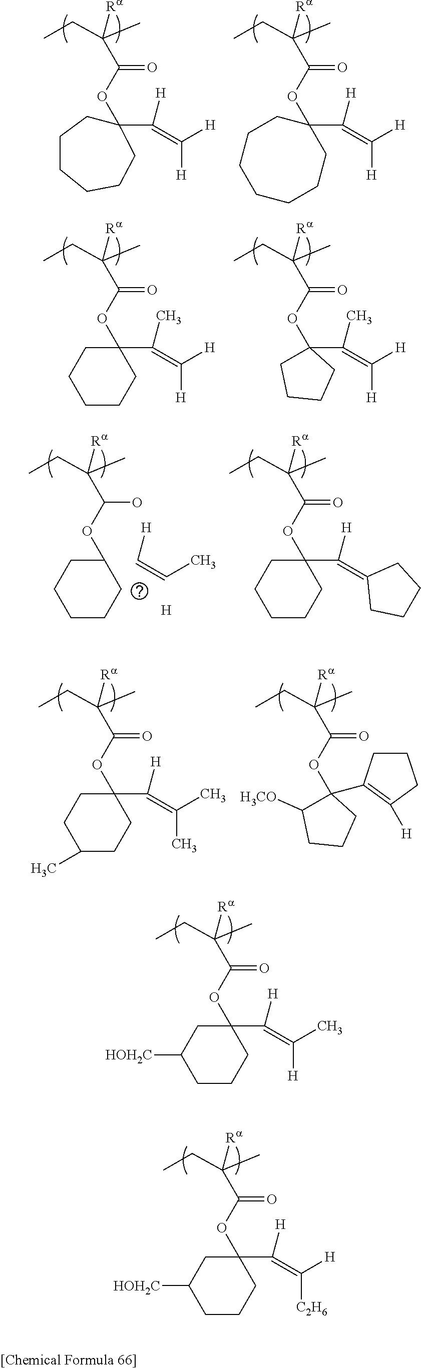

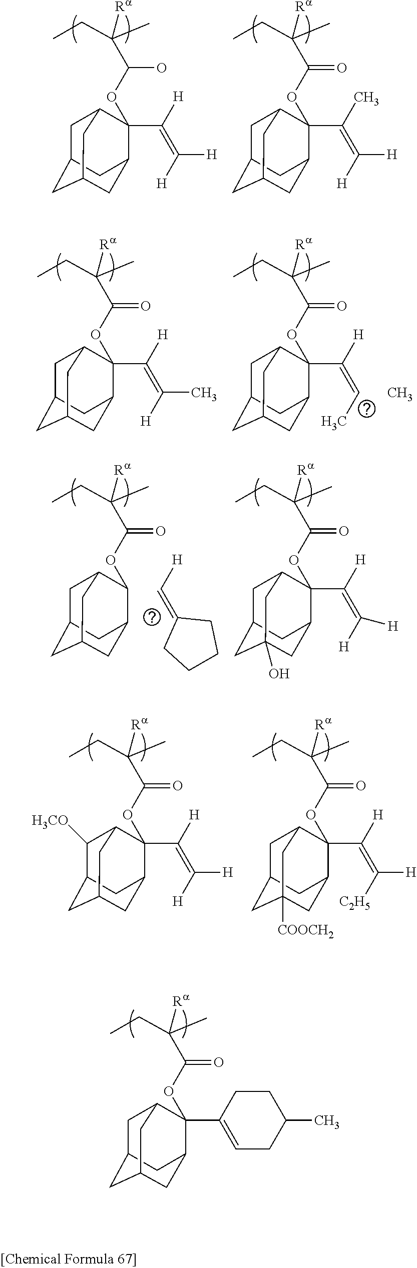

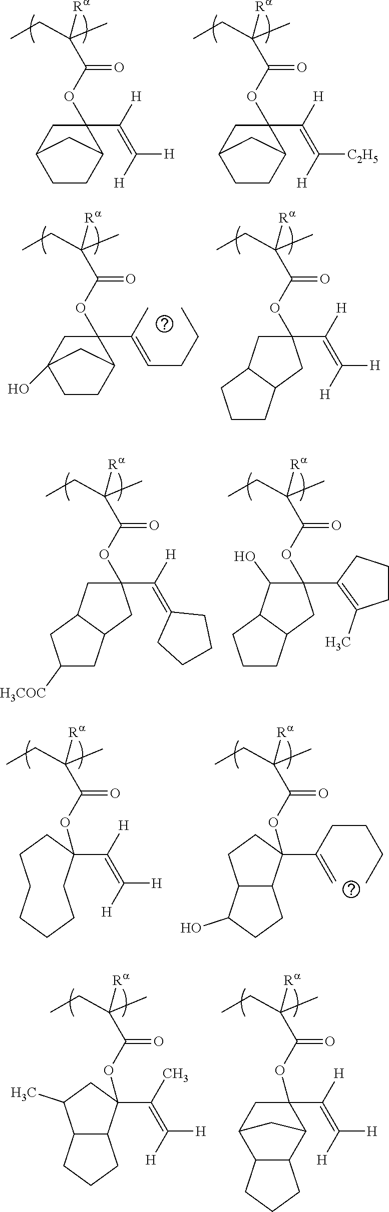

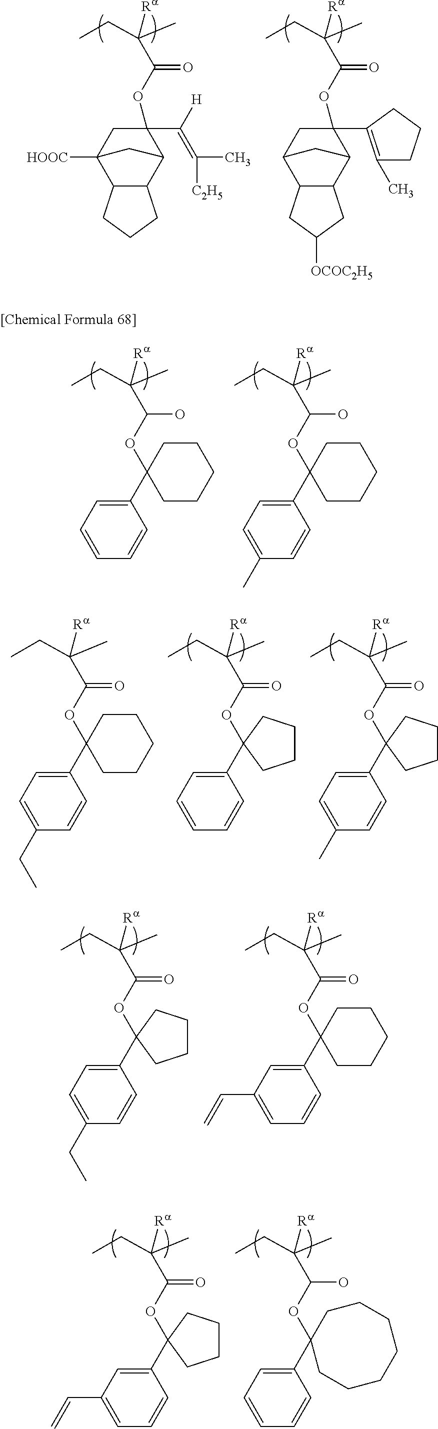

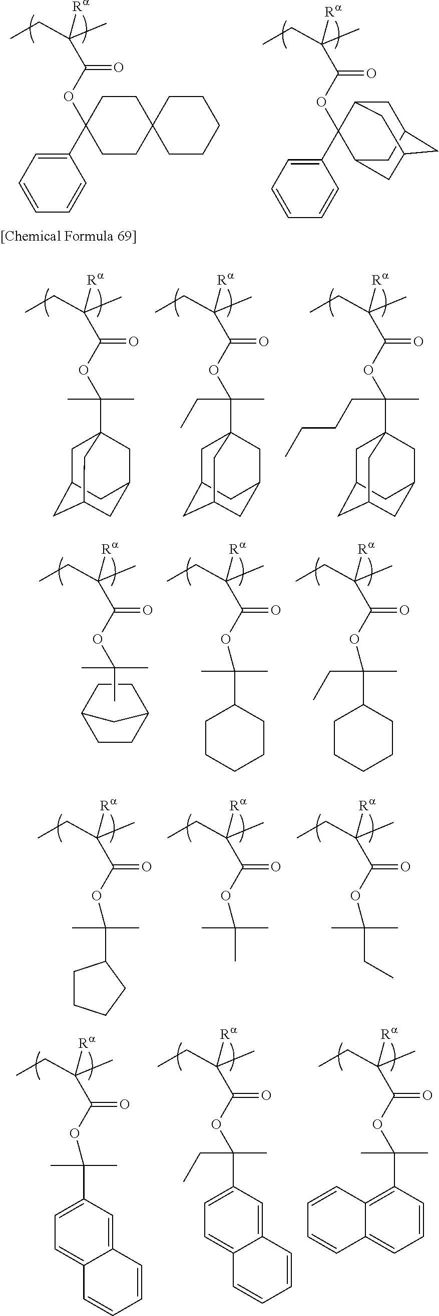

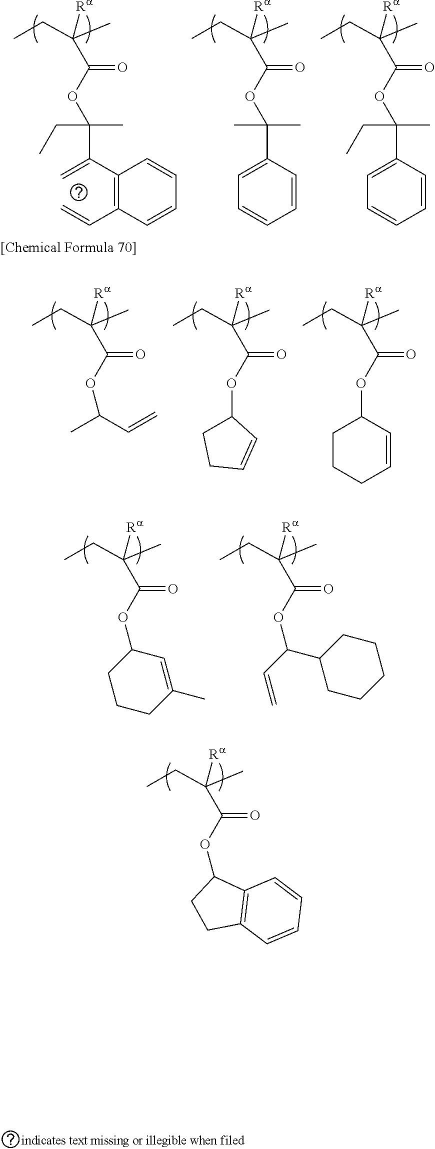

In the formulae shown below, R^{α} represents a hydrogen atom, a methyl group, or a trifluoromethyl group. Rz represents a hydrogen atom, an alkyl group, a halogen atom, a halogenated alkyl group, a hydroxy group, or an alkoxy group.

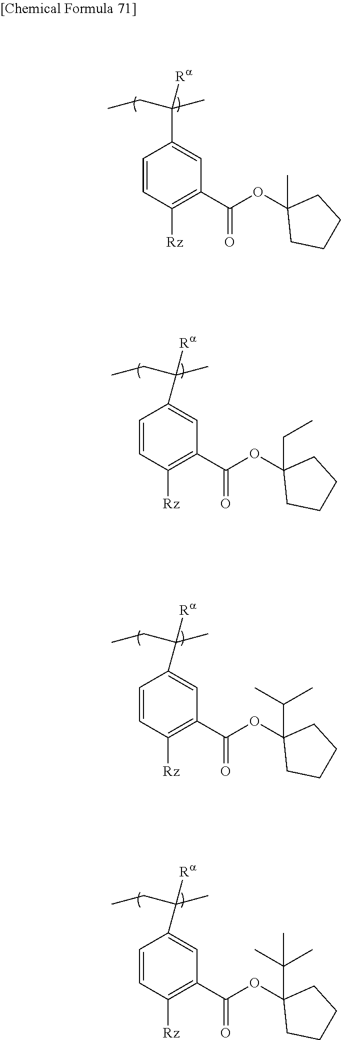

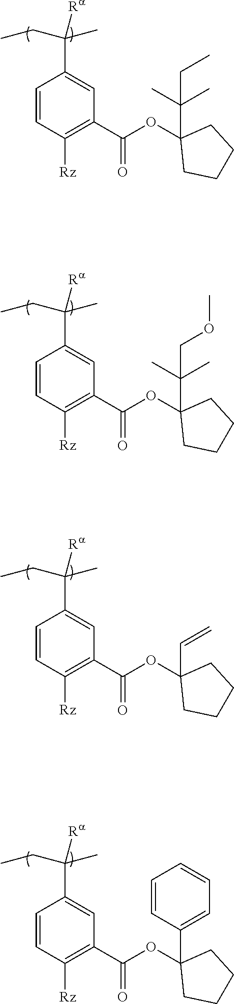

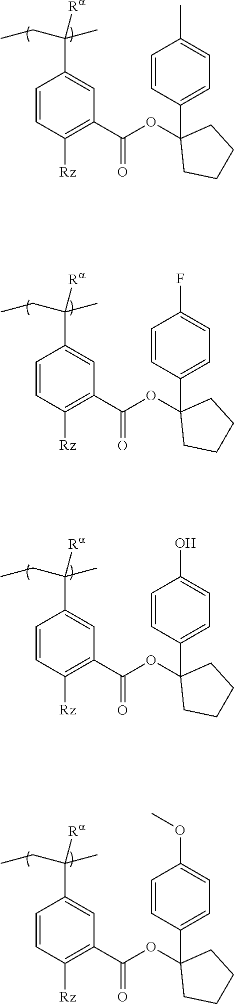

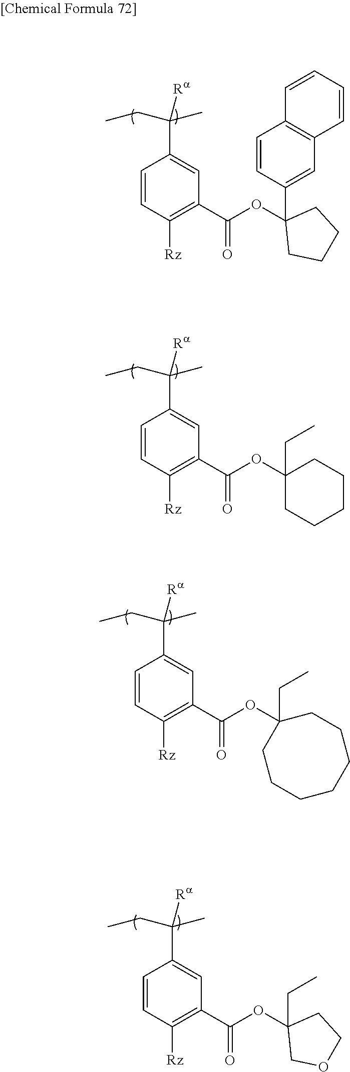

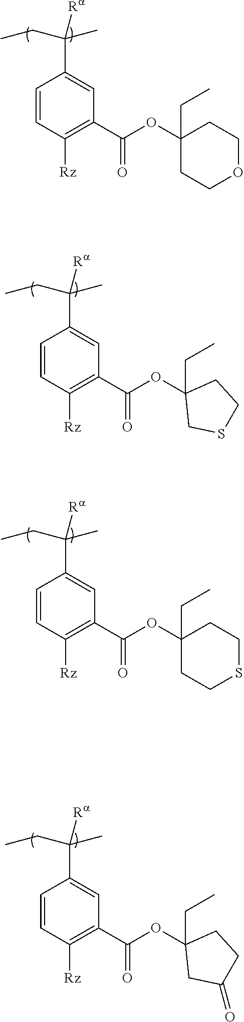

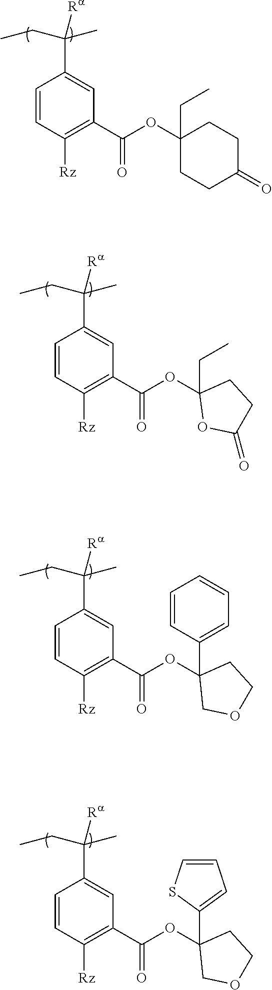

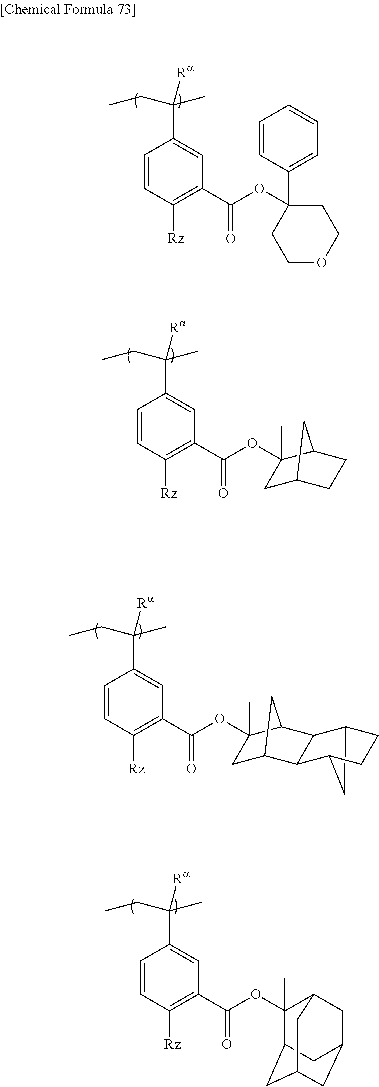

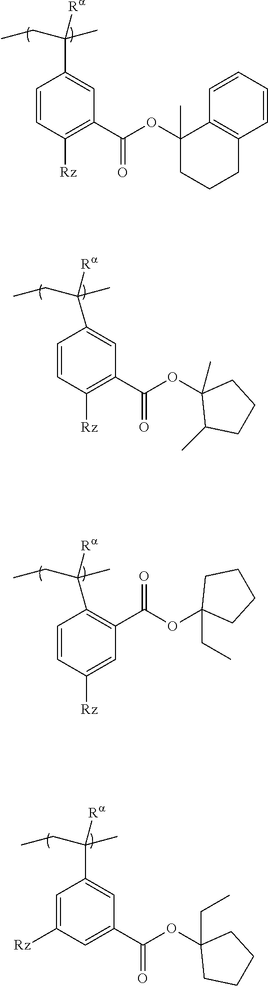

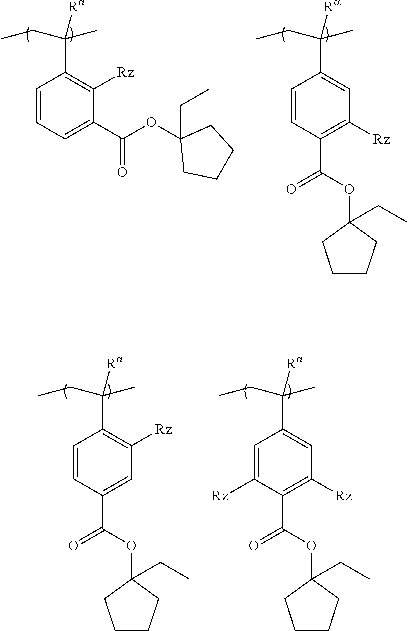

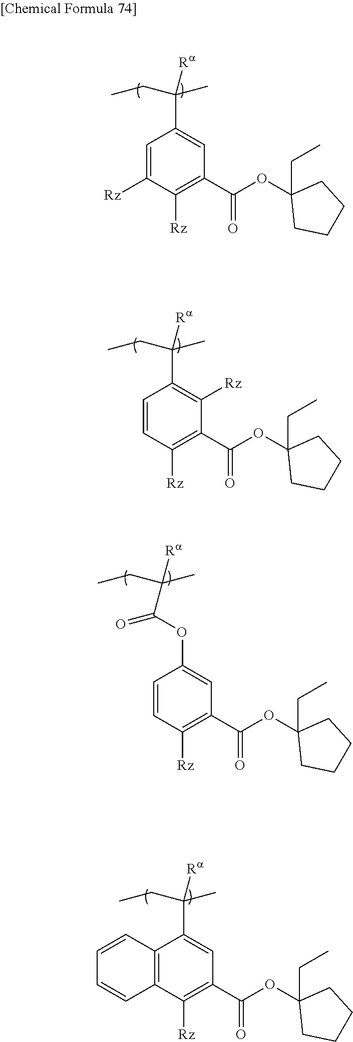

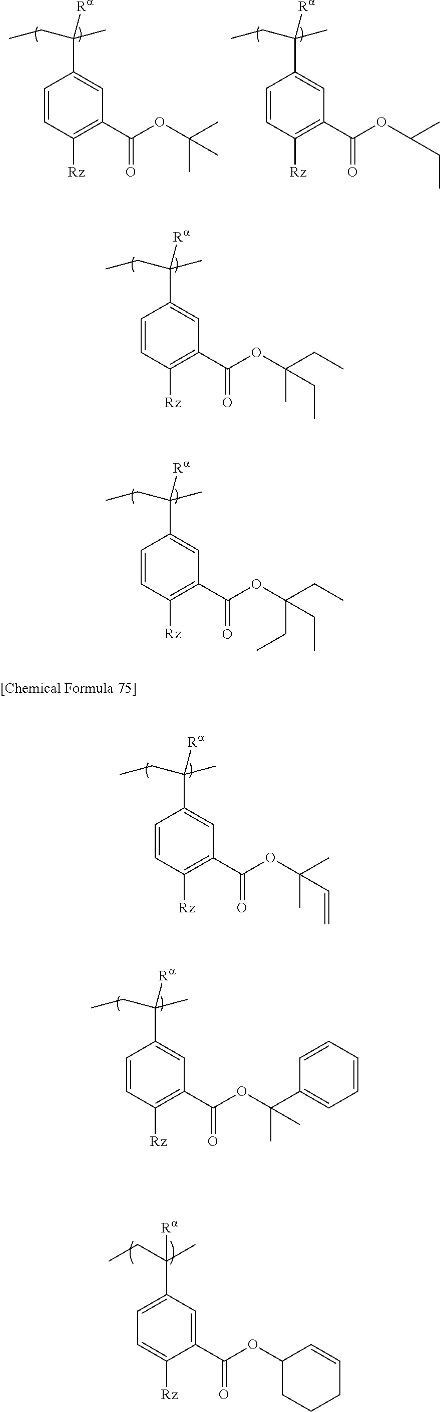

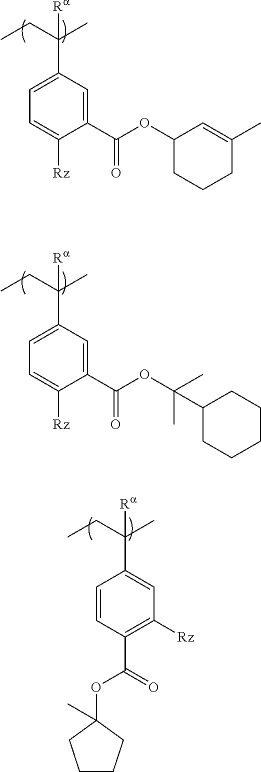

The constitutional unit (a1) included in the component (A1) may be used alone or two or more kinds thereof.

From the viewpoint of further easily enhancing the lithography characteristics (the sensitivity, the shape, and the like) using electron beams or EUV, a constitutional unit represented by Formula (a1-1) or a constitutional unit represented by Formula (a1-3) is more preferable as the constitutional unit (a1).

Among these, from the viewpoint of suitably increasing the reactivity for EB or EUV, the acid-dissociable groups (Ra¹, Rax⁰¹) are each preferably an acid-dissociable group represented by General Formula (a1-r2-1), (a1-r2-3), (a1-r2-4), or (a1-r-4). Among these, it is particularly preferable that the acid-dissociable groups be selected from the cyclic groups.

Alternatively, the constitutional unit (a1) may include a constitutional unit represented by General Formula (a1-1-1).

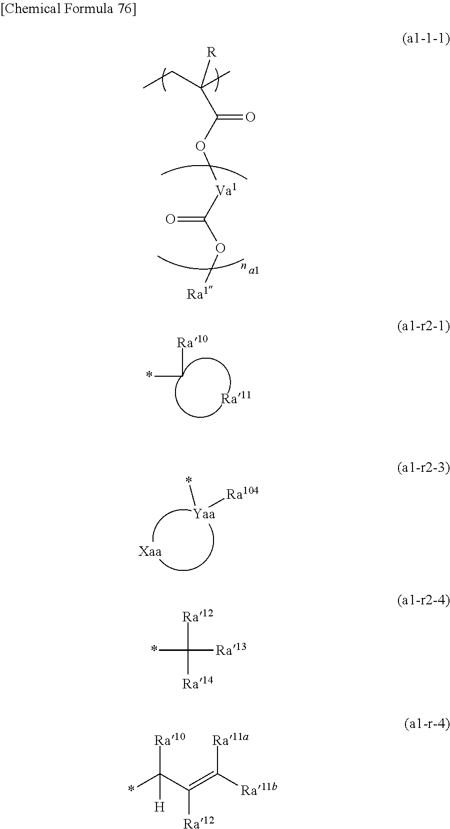

[In the formulae, Ra¹″ represents an acid-dissociable group represented by General Formula (a1-r2-1), (a1-r2-3), (a1-r2-4), or (a1-r-4). * represents a bonding site.]

In Formula (a1-1-1), R, Va¹, and nₐ₁ each has the same definition as that for R, Va¹, and nₐ₁ in Formula (a1-1).

The description of the acid-dissociable group represented by General Formula (a1-r2-1), (a1-r2-3), (a1-r2-4), or (a1-r-4) is the same as described above. Among these, it is preferable to select those in which the acid-dissociable group is a cyclic group because the reactivity is enhanced for EB or EUV, which is suitable.

The proportion of the constitutional unit (a1) in the component (A1) is preferably in a range of 5% to 80% by mole, more preferably in a range of 10% to 75% by mole, still more preferably in a range of 30% to 70% by mole, and particularly preferably in a range of 40% to 70% by mole with respect to the total amount (100% by mole) of all constitutional units constituting the component (A1).

In a case where the proportion of the constitutional unit (a1) is set to be equal to or greater than the lower limits of the above-described preferable ranges, lithography characteristics such as sensitivity, resolution, and roughness amelioration are improved. Further, in a case where the proportion of the constitutional unit (a1) is less than or equal to the upper limits of the above-described preferable ranges, the constitutional unit (a1) and other constitutional units can be balanced, and various lithography characteristics are enhanced.

Constitutional Unit (a10):

The constitutional unit (a10) is a constitutional unit represented by General Formula (a10-1).

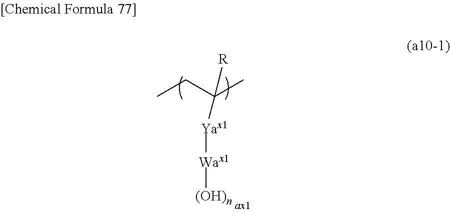

[In the formula, R represents a hydrogen atom, an alkyl group having 1 to 5 carbon atoms, or a halogenated alkyl group having 1 to 5 carbon atoms. Ya^{x1} represents a single bond or a divalent linking group. Wa^{x1} represents an aromatic hydrocarbon group which may have a substituent. nₐₓ₁ represents an integer of 1 or greater.]

In Formula (a10-1), R has the same definition as that for R in General Formula (a1-1). R preferably represents a hydrogen atom, an alkyl group having 1 to 5 carbon atoms, or a fluorinated alkyl group having 1 to 5 carbon atoms and particularly preferably a hydrogen atom or a methyl group from the viewpoint of the industrial availability.

In Formula (a10-1), Ya^{x1} represents a single bond or a divalent linking group.

In the chemical formula, the divalent linking group as Ya^{x1} is not particularly limited, and suitable examples thereof include a divalent hydrocarbon group which may have a substituent and a divalent linking group having a hetero atom.

Divalent Hydrocarbon Group which May have Substituent:

The divalent hydrocarbon group which may have a substituent may be an aliphatic hydrocarbon group or an aromatic hydrocarbon group.

### Aliphatic Hydrocarbon Group

The aliphatic hydrocarbon group indicates a hydrocarbon group that has no aromaticity. The aliphatic hydrocarbon group may be saturated or unsaturated. In general, it is preferable that the aliphatic hydrocarbon group be saturated.

Examples of the aliphatic hydrocarbon group include a linear or branched aliphatic hydrocarbon group and an aliphatic hydrocarbon group having a ring in the structure thereof.

### Linear or Branched Aliphatic Hydrocarbon Group

The linear aliphatic hydrocarbon group preferably has 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group preferably has 2 to 10 carbon atoms, more preferably has 3 to 6 carbon atoms, still more preferably has 3 or 4 carbon atoms, and most preferably has 3 carbon atoms.

As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

The linear or branched aliphatic hydrocarbon group may or may not have a substituent. Examples of the substituent include a fluorine atom, a fluorinated alkyl group having 1 to 5 carbon atoms which has been substituted with a fluorine atom, and a carbonyl group.

### Aliphatic Hydrocarbon Group Having Ring in Structure Thereof

Examples of the aliphatic hydrocarbon group having a ring in the structure thereof include a cyclic aliphatic hydrocarbon group which may have a substituent having a heteroatom in the ring structure thereof (a group in which two hydrogen atoms have been removed from an aliphatic hydrocarbon ring), a group in which the cyclic aliphatic hydrocarbon group is bonded to the terminal of a linear or branched aliphatic hydrocarbon group, and a group in which the cyclic aliphatic hydrocarbon group is interposed in the middle of a linear or branched aliphatic hydrocarbon group. Examples of the linear or branched aliphatic hydrocarbon group include the same groups as described above.

The cyclic aliphatic hydrocarbon group preferably has 3 to 20 carbon atoms and more preferably has 3 to 12 carbon atoms.

The cyclic aliphatic hydrocarbon group may be a polycyclic group or a monocyclic group. As the monocyclic alicyclic hydrocarbon group, a group in which two hydrogen atoms have been removed from a monocycloalkane is preferable. The monocycloalkane preferably has 3 to 6 carbon atoms, and specific examples thereof include cyclopentane and cyclohexane. As the polycyclic alicyclic hydrocarbon group, a group in which two hydrogen atoms have been removed from a polycycloalkane is preferable. The number of carbon atoms in the polycycloalkane is preferably 7 to 12, and specific examples thereof include adamantane, norbornane, isobomane, tricyclo[5.2.1.0^{2,6}]decane, and tetracyclododecane.

The cyclic aliphatic hydrocarbon group may or may not have a substituent. Examples of the substituent include an alkyl group, an alkoxy group, a halogen atom, a halogenated alkyl group, a hydroxyl group, and a carbonyl group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is still more preferable.

As the halogen atom as the substituent, a fluorine atom is preferable.

Examples of the halogenated alkyl group as the substituent include groups in which some or all hydrogen atoms in the above-described alkyl groups have been substituted with the above-described halogen atoms.

In the cyclic aliphatic hydrocarbon group, some carbon atoms constituting the ring structure thereof may be substituted with a substituent having a heteroatom. As the substituent having a heteroatom, —O—, —C(═O)—O—, —S—, —S(═O)₂—, or —S(═O)₂—O— is preferable.

### Aromatic Hydrocarbon Group

The aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring preferably has 5 to 30 carbon atoms, more preferably 5 to 20 carbon atoms, still more preferably 6 to 15 carbon atoms, and particularly preferably 6 to 12 carbon atoms. Here, the number of carbon atoms in a substituent is not included in the number of carbon atoms.

Specific examples of the aromatic ring include aromatic hydrocarbon rings such as benzene, naphthalene, anthracene, and phenanthrene; and aromatic heterocyclic rings in which some carbon atoms constituting the above-described aromatic hydrocarbon rings have been substituted with heteroatoms. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group include a group in which two hydrogen atoms have been removed from the above-described aromatic hydrocarbon ring or aromatic heterocyclic ring (an arylene group or a heteroarylene group), a group in which two hydrogen atoms have been removed from an aromatic compound having two or more aromatic rings (for example, biphenyl or fluorene), and a group in which one hydrogen atom of a group (an aryl group or a heteroaryl group) obtained by removing one hydrogen atom from the above-described aromatic hydrocarbon ring or aromatic heterocyclic ring has been substituted with an alkylene group (for example, a group obtained by further removing one hydrogen atom from an aryl group in an arylalkyl group such as a benzyl group, a phenethyl group, a 1-naphthylmethyl group, a 2-naphthylmethyl group, a 1-naphthylethyl group, or a 2-naphthylethyl group). The number of carbon atoms in the alkylene group bonded to the aryl group or the heteroaryl group is preferably in a range of 1 to 4, more preferably 1 or 2, and particularly preferably 1.

In the aromatic hydrocarbon group, the hydrogen atom in the aromatic hydrocarbon group may be substituted with a substituent. For example, the hydrogen atom bonded to the aromatic ring in the aromatic hydrocarbon group may be substituted with a substituent. Examples of substituents include an alkyl group, an alkoxy group, a halogen atom, a halogenated alkyl group, and a hydroxyl group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group, the halogen atom, and the halogenated alkyl group as the substituents, the groups described as the substituents that substitute a hydrogen atom in the cyclic aliphatic hydrocarbon group are exemplary examples.

### Divalent Linking Group Having Heteroatom:

Examples of the divalent linking group having a heteroatom include —O—, —C(═O)—O—, —O—C(═O)—, —C(═O)—, —O—C(═O)—O—, —C(═O)—NH—, —NH—, —NH—C(═NH)— (H may be substituted with a substituent such as an alkyl group or an acyl group). —S—, —S(═O)₂—, —S(═O)₂—O—, and a group represented by General Formula —Y²¹—O—Y²²—, —Y²¹—O—, —Y²¹—C(═O)—O—, —C(═O)—O—Y²¹—, —[Y²¹—C(═O)—O]_{m″}—Y²²—, —Y²¹—O—C(═O)—Y²²—, or —Y²¹—S(═O)₂—O—Y²²— [in the formulae, Y²¹ and Y²² each independently represents a divalent hydrocarbon group which may have a substituent. O represents an oxygen atom, and m″ represents an integer of 1 to 3].

In a case where the above-described divalent linking group having a heteroatom is —C(═O)—NH—, —C(═O)—NH—C(═O)—, —NH—, or —NH—C(═NH)—, H may be substituted with a substituent such as an alkyl group and an acyl group. The substituent (alkyl group, acyl group, and the like) preferably has 1 to 10 carbon atoms, more preferably has 1 to 8 carbon atoms, and particularly preferably has 1 to 5 carbon atoms.

In General Formula —Y²¹—O—Y²²—, —Y²¹—O—, —Y²¹—C(═O)—O—, —C(═O)—O—Y²¹—, —[Y²¹—C(═O)—O]_{m″}—Y²²—, —Y²¹—O—C(═O)—Y²²—, or —Y²¹—S(═O)₂—O—Y²²—, Y²¹ and Y²² each independently represents a divalent hydrocarbon group which may have a substituent. Examples of the divalent hydrocarbon group include the same groups as described above.

Y²¹ preferably represents a linear aliphatic hydrocarbon group, more preferably a linear alkylene group, still more preferably a linear alkylene group having 1 to 5 carbon atoms, and particularly preferably a methylene group or an ethylene group.

Y²² preferably represents a linear or branched aliphatic hydrocarbon group and more preferably a methylene group, an ethylene group, or an alkylmethylene group. The alkyl group in the alkylmethylene group is preferably a linear alkyl group having 1 to 5 carbon atoms, more preferably a linear alkyl group having 1 to 3 carbon atoms, and most preferably a methyl group.

In the group represented by Formula —[Y²¹—C(═O)—O]_{m″}—Y²²—, m″ represents an integer of 1 to 3, preferably 1 or 2, and more preferably 1. That is, a group represented by Formula —Y²¹—C(═O)—O—Y²²— is particularly preferable as the group represented by Formula —[Y²¹—C(═O)—O]_{m″}—Y²²—. Among these, a group represented by Formula —(CH₂)_{a′}—C(═O)—O—(CH₂)_{b′}— is preferable. In the formula, a′ represents an integer of 1 to 10, preferably an integer of 1 to 8, more preferably an integer of 1 to 5, still more preferably 1 or 2, and most preferably 1. b′ represents an integer of 1 to 10, preferably an integer of 1 to 8, more preferably an integer of 1 to 5, still more preferably 1 or 2, and most preferably 1.

Ya^{x1} preferably represents a single bond, an ester bond [—C(═O)—O— or —O—C(═O)—], an ether bond (—O—), a linear or branched alkylene group, or a combination thereof and more preferably a single bond or an ester bond [—C(═O)—O— or —O—C(═O)—].

In Formula (a10-1), Wa^{x1} represents an aromatic hydrocarbon group which may have a substituent.

Examples of the aromatic hydrocarbon group as Wa^{x1} include a group in which (nₐₓ₁+1) hydrogen atoms have been removed from an aromatic ring which may have a substituent. The aromatic ring is not particularly limited as long as the aromatic ring is a cyclic conjugated system having (4n+2) π electrons. The aromatic ring preferably has 5 to 30 carbon atoms, more preferably 5 to 20 carbon atoms, still more preferably 6 to 15 carbon atoms, and particularly preferably 6 to 12 carbon atoms. Specific examples of the aromatic ring include aromatic hydrocarbon rings such as benzene, naphthalene, anthracene, and phenanthrene; and an aromatic heterocyclic ring obtained by substituting some carbon atoms constituting the above-described aromatic hydrocarbon ring with a heteroatom. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Further, examples of the aromatic hydrocarbon group as Wa^{x1} also include a group in which (nₐₓ₁+1) hydrogen atoms have been removed from an aromatic compound having an aromatic ring (for example, biphenyl or fluorene) which may have two or more substituents.

Among the above, Wa^{x1} preferably represents a group in which (nₐₓ₁+1) hydrogen atoms have been removed from benzene, naphthalene, anthracene, or biphenyl, more preferably a group in which (nₐₓ₁+1) hydrogen atoms have been removed from benzene or naphthalene, and still more preferably a group in which (nₐₓ₁+1) hydrogen atoms have been removed from benzene.

The aromatic hydrocarbon group as Wa^{x1} may or may not have a substituent. Examples of substituents include an alkyl group, an alkoxy group, a halogen atom, and a halogenated alkyl group. Examples of the alkyl group, the alkoxy group, the halogen atom, and the halogenated alkyl group as the substituent include those described as the substituent of the cyclic aliphatic hydrocarbon group as Ya^{x1}. The substituent is preferably a linear or branched alkyl group having 1 to 5 carbon atoms, more preferably a linear or branched alkyl group having 1 to 3 carbon atoms, still more preferably an ethyl group or a methyl group, and particularly preferably a methyl group. It is preferable that the aromatic hydrocarbon group as Wa^{x1} have no substituent.

In Formula (a10-1), nₐₓ₁ represents an integer of 1 or greater, preferably an integer of 1 to 10, more preferably an integer of 1 to 5, still more preferably 1, 2, or 3, and particularly preferably 1 or 2.

Specific examples of the constitutional unit (a10) represented by Formula (a10-1) are described below.

In the formulae shown below, R^{α} represents a hydrogen atom, a methyl group, or a trifluoromethyl group.

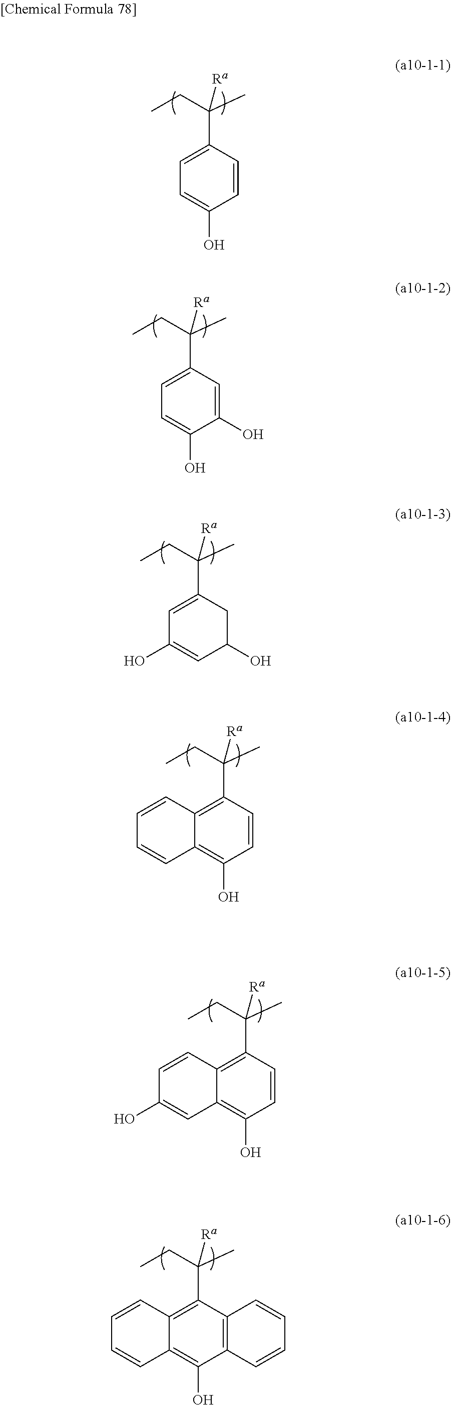

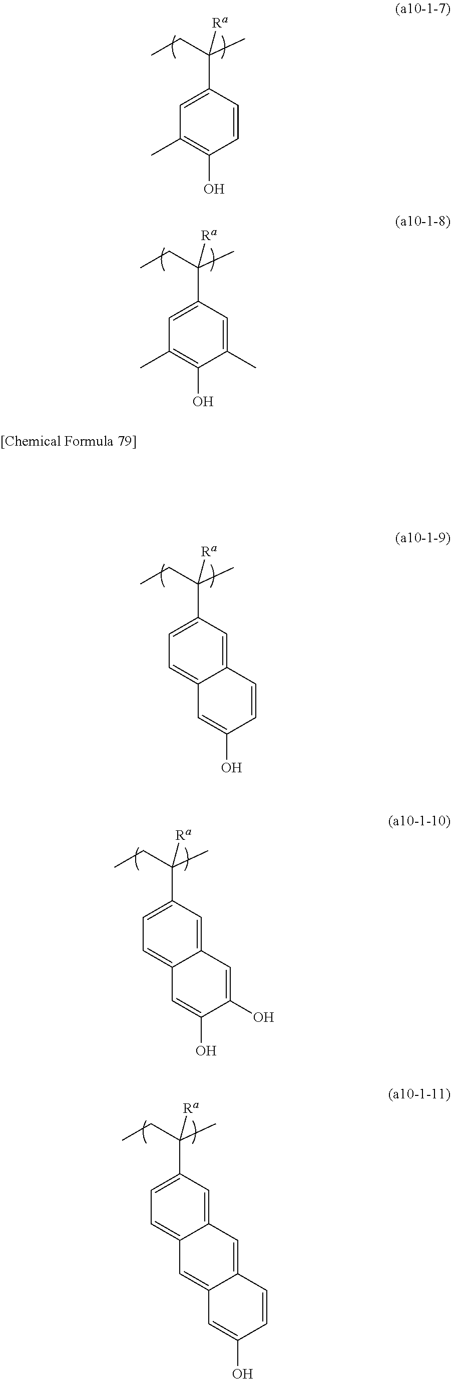

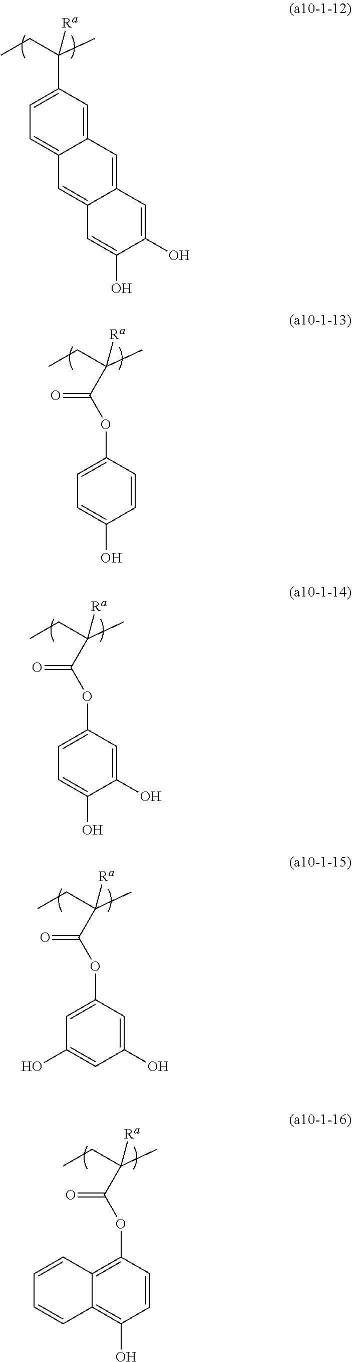

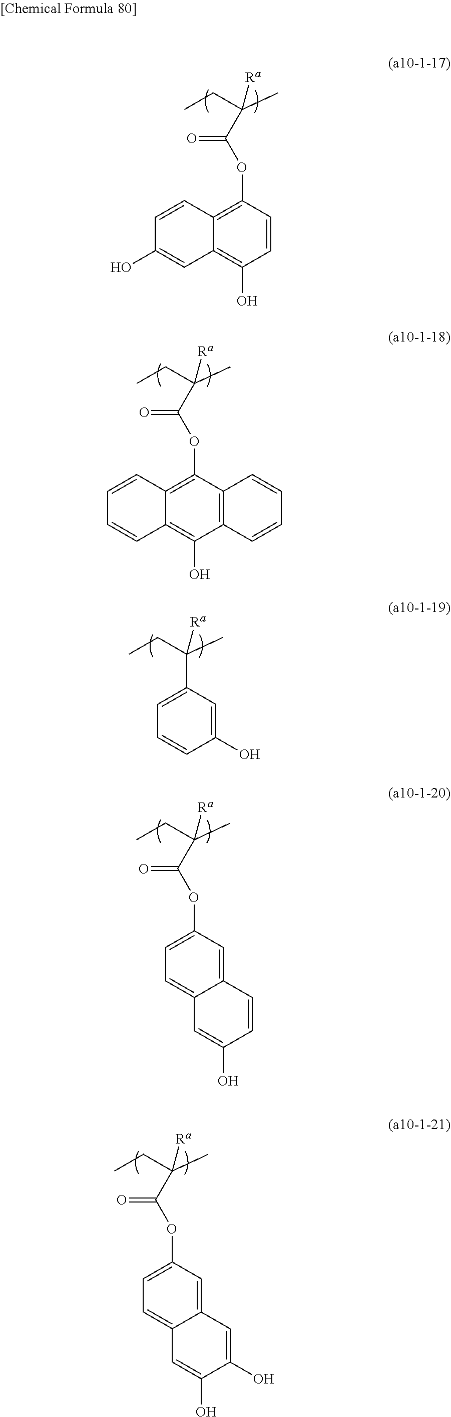

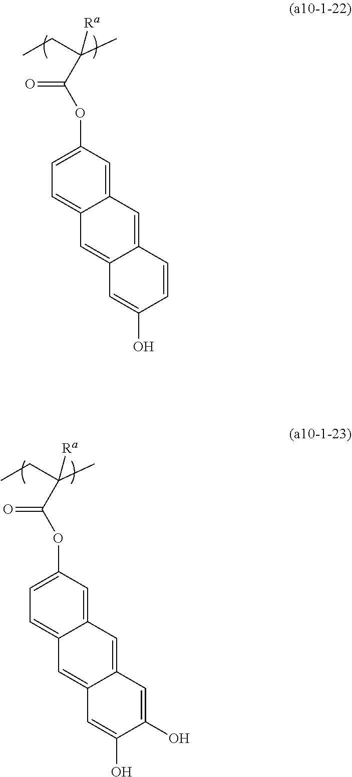

The constitutional unit (a10) included in the component (A1) may be used alone or two or more kinds thereof. The component (A1) may or may not have the constitutional unit (a10), but it is preferable that the component (A1) have the constitutional unit (a10).

In a case where the component (A1) has the constitutional unit (a10), the proportion of the constitutional unit (a10) in the component (A1) is preferably in a range of 20% to 80% by mole, more preferably in a range of 25% to 70% by mole, still more preferably in a range of 30% to 60% by mole, and particularly preferably in a range of 30% to 50% by mole with respect to the total amount (100% by mole) of all constitutional units constituting the component (A1).

In a case where the proportion of the constitutional unit (a10) is set to be greater than or equal to the above-described lower limits, the sensitivity is likely to be enhanced. Meanwhile, in a case where the proportion thereof is set to be less than or equal to the upper limits, the constitutional unit (a10) and other constitutional units are likely to be balanced.

### Constitutional Unit (a2):

The component (A1) may have a constitutional unit (a2) containing a lactone-containing cyclic group (here, constitutional units corresponding to the constitutional unit (a1) are excluded).

In a case where the component (A1) is used to form a resist film, the lactone-containing cyclic group of the constitutional unit (a2) is effective for increasing the adhesiveness of the resist film to the substrate. Further, in a case where the component (A1) contains the constitutional unit (a2), the lithography characteristics and the like are enhanced, for example, due to the effects of appropriately adjusting the acid diffusion length, increasing the adhesiveness of the resist film to the substrate, appropriately adjusting the solubility during the development, and the like.

The term “lactone-containing cyclic group” indicates a cyclic group that has a ring (lactone ring) containing —O—C(═O)— in the ring skeleton. In a case where the lactone ring is counted as the first ring and the group contains only the lactone ring, the group is referred to as a monocyclic group. Further, in a case where the group has other ring structures, the group is referred to as a polycyclic group regardless of the structures. The lactone-containing cyclic group may be a monocyclic group or a polycyclic group.

The lactone-containing cyclic group in the constitutional unit (a2) is not particularly limited, and an optional constitutional unit can be used. Specific examples thereof include groups each represented by General Formulae (a2-r-1) to (a2-r-7).

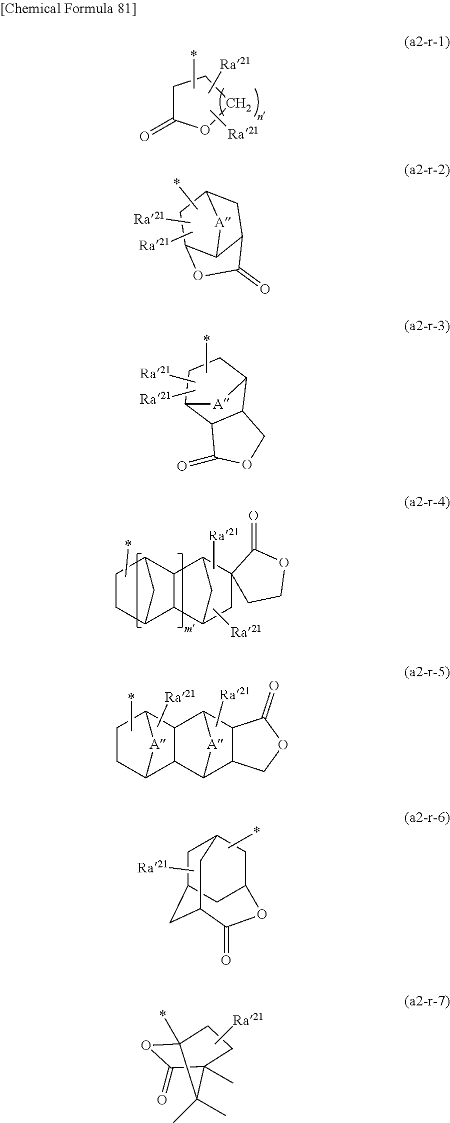

[In the formulae, Ra′²¹'s each independently represents a hydrogen atom, an alkyl group, an alkoxy group, a halogen atom, a halogenated alkyl group, a hydroxyl group, —COOR″, —OC(═O)R″, a hydroxyalkyl group, or a cyano group, R″ represents a hydrogen atom, an alkyl group, or a lactone-containing cyclic group. A″ represents an alkylene group having 1 to 5 carbon atoms which may have an oxygen atom (—O—) or a sulfur atom (—S—), an oxygen atom, or a sulfur atom, n′ represents an integer of 0 to 2, and m′ represents 0 or 1. * represents a bonding site (the same applies hereinafter).]

In General Formulae (a2-r-1) to (a2-r-7), it is preferable that the alkyl group as Ra′²¹ be an alkyl group having 1 to 6 carbon atoms. Further, it is preferable that the alkyl group be linear or branched. Specific examples thereof include a methyl group, an ethyl group, a propyl group, an isopropyl group, an n-butyl group, an isobutyl group, a tert-butyl group, a pentyl group, an isopentyl group, a neopentyl group, and a hexyl group. Among these, a methyl group or ethyl group is preferable, and a methyl group is particularly preferable.

It is preferable that the alkoxy group as Ra′²¹ be an alkoxy group having 1 to 6 carbon atoms. Further, it is preferable that the alkoxy group be linear or branched. Specific examples of the alkoxy groups include a group formed by linking the above-described alkyl group described as the alkyl group represented by Ra′²¹ to an oxygen atom (—O—).

As the halogen atom as Ra′²¹, a fluorine atom is preferable.

Examples of the halogenated alkyl group as Ra′²¹ include groups in which some or all hydrogen atoms in the alkyl group as Ra′²¹ have been substituted with the halogen atoms. As the halogenated alkyl group, a fluorinated alkyl group is preferable, and a perfluoroalkyl group is particularly preferable.

In —COOR″ as Ra′²¹ and —OC(═O)R″ as Ra″, any R″ represents a hydrogen atom, an alkyl group, or a lactone-containing cyclic group.

The alkyl group as R″ may be linear, branched, or cyclic and preferably has 1 to 15 carbon atoms.

In a case where R″ represents a linear or branched alkyl group, an alkyl group having 1 to 10 carbon atoms is preferable, an alkyl group having 1 to 5 carbon atoms is more preferable, and a methyl group or an ethyl group is particularly preferable.

In a case where R″ represents a cyclic alkyl group, the number of carbon atoms thereof is preferably in a range of 3 to 15, more preferably in a range of 4 to 12, and most preferably in a range of 5 to 10. Specific examples thereof include groups in which one or more hydrogen atoms have been removed from a monocycloalkane, which may or may not be substituted with a fluorine atom or a fluorinated alkyl group; and groups in which one or more hydrogen atoms have been removed from a polycycloalkane such as bicycloalkane, tricycloalkane, or tetracycloalkane. More specific examples thereof include a group in which one or more hydrogen atoms have been removed from a monocycloalkane such as cyclopentane or cyclohexane; and a group in which one or more hydrogen atoms have been removed from a polycycloalkane such as adamantane, norbornane, isobornane, tricyclo[5.2.1.0^{2,6}]decane, or tetracyclododecane.

Examples of the lactone-containing cyclic group as R″ include the same groups as those for the groups each represented by General Formulae (a2-r-1) to (a2-r-7).

As the hydroxyalkyl group as Ra′²¹, a hydroxyalkyl group having 1 to 6 carbon atoms is preferable, and specific examples thereof include a group in which at least one hydrogen atom in the alkyl group as Ra′²¹ has been substituted with a hydroxyl group.

Among the above, it is preferable that Ra′²¹'s each independently represent a hydrogen atom or a cyano group.

In General Formulae (a2-r-2), (a2-r-3) and (a2-r-5), as the alkylene group having 1 to 5 carbon atoms as A″, a linear or branched alkylene group is preferable, and examples thereof include a methylene group, an ethylene group, an n-propylene group, and an isopropylene group. In a case where the alkylene group has an oxygen atom or a sulfur atom, specific examples thereof include groups in which —O— or —S— is interposed in the terminal of the alkylene group or between the carbon atoms of the alkylene group. Further, examples thereof include —O—CH₂—, —CH₂—O—CH₂—, —S—CH₂—, and —CH₂—S—CH₂—. A″ preferably represents an alkylene group having 1 to 5 carbon atoms or —O—, more preferably an alkylene group having 1 to 5 carbon atoms, and most preferably a methylene group.

Specific examples of the groups each represented by General Formulae (a2-r-1) to (a2-r-7) are shown below.

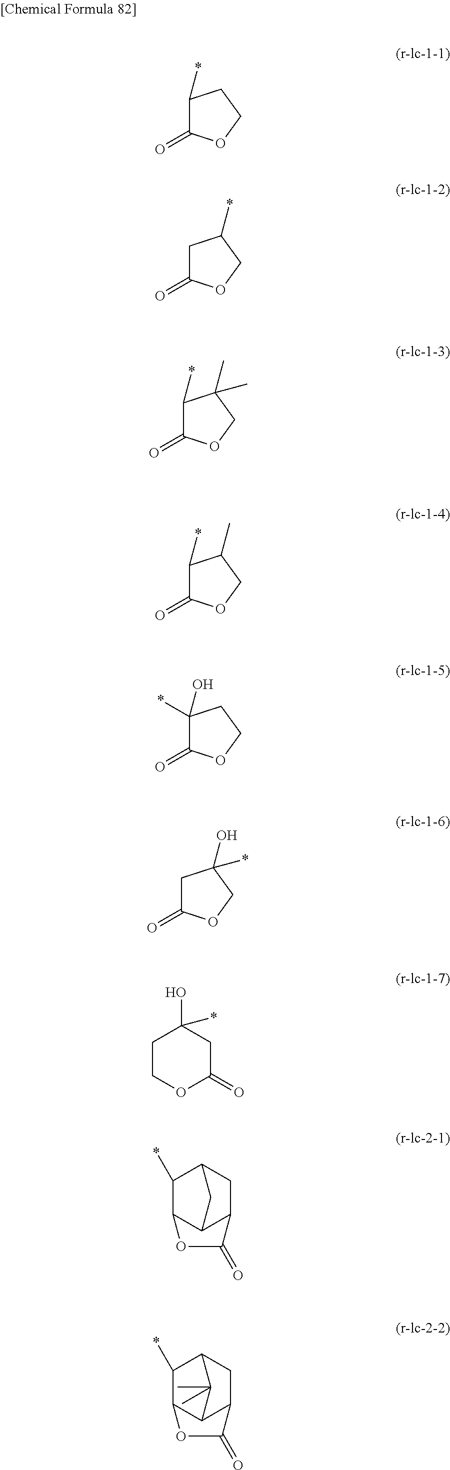

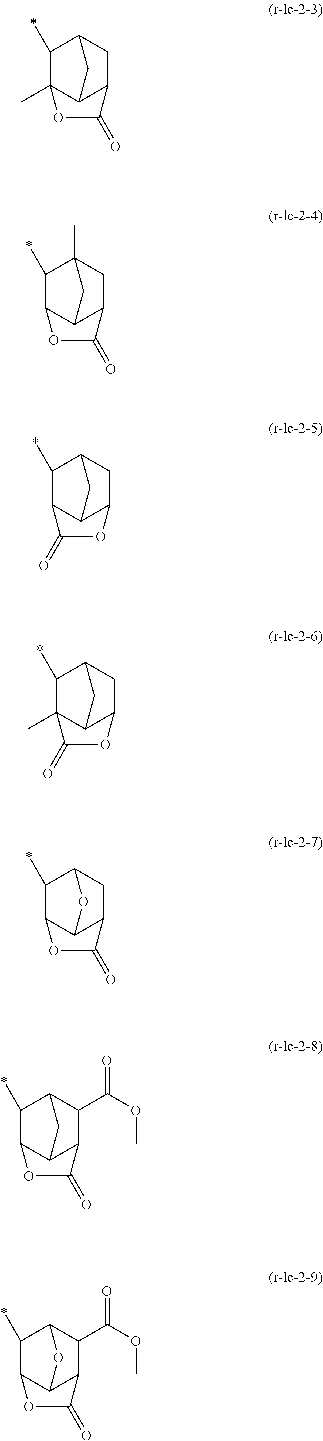

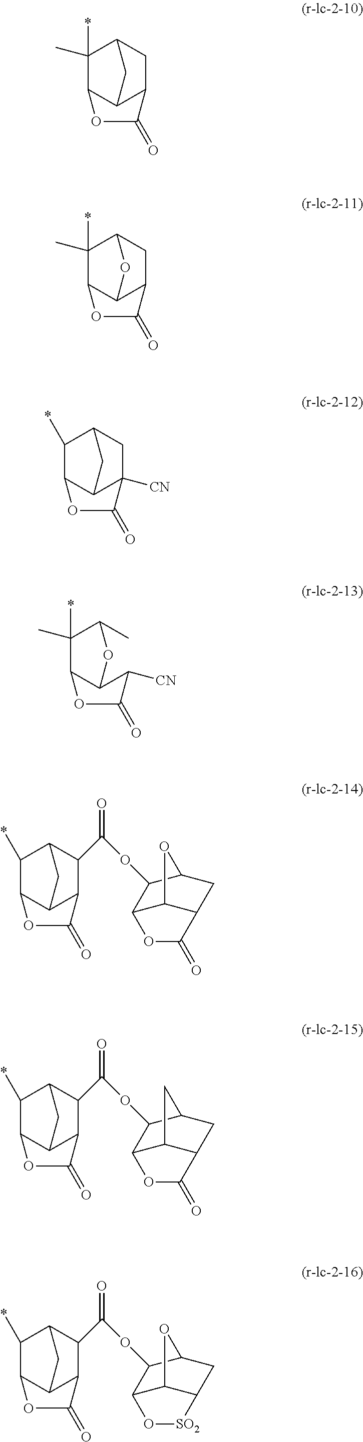

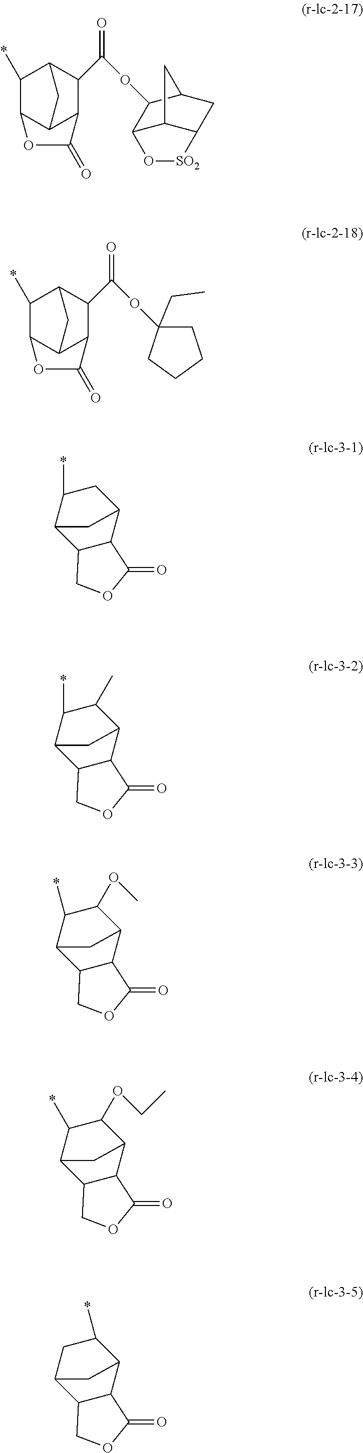

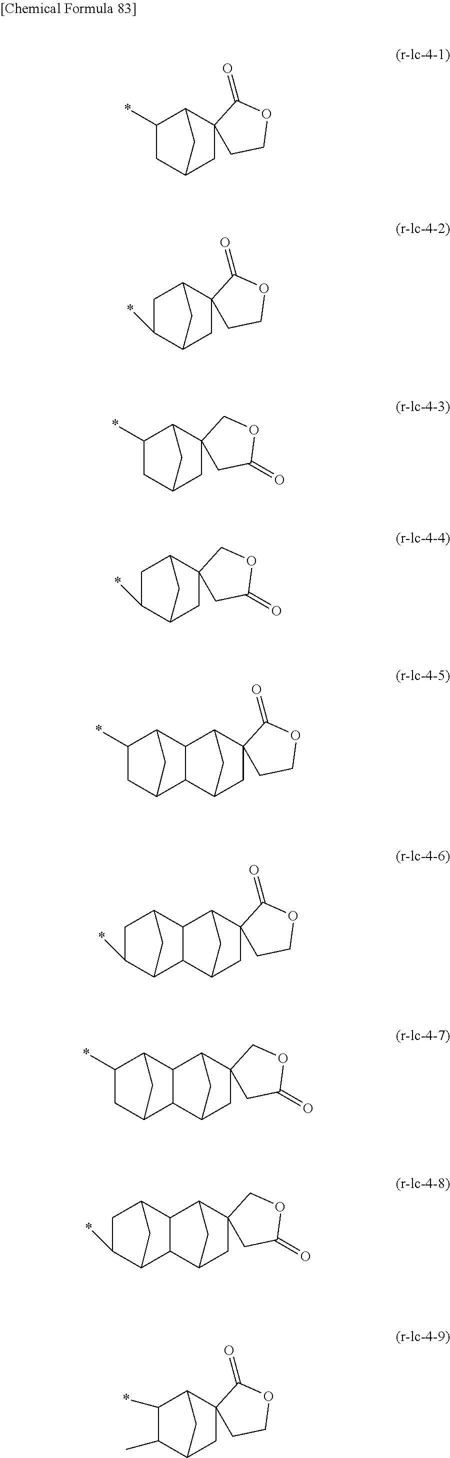

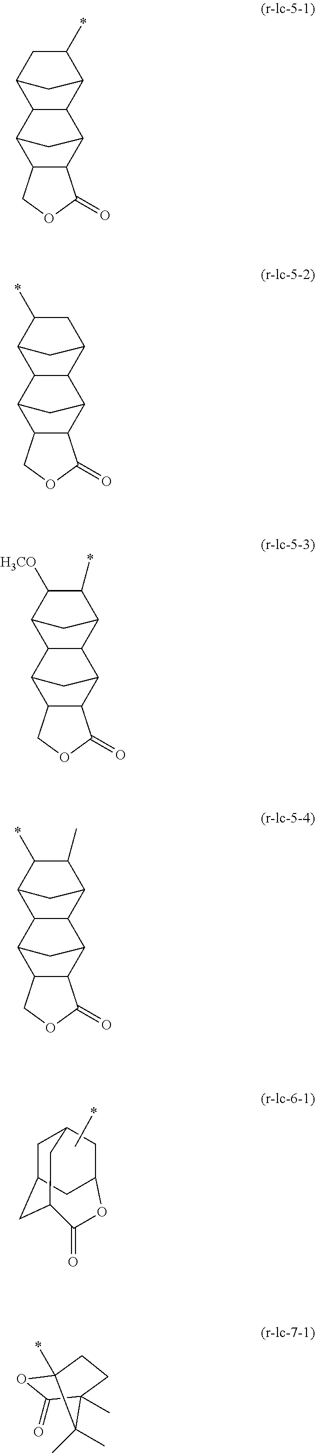

As the constitutional unit (a2), a constitutional unit derived from acrylic acid ester in which the hydrogen atom bonded to the carbon atom at the α-position may be substituted with a substituent is preferable.

It is preferable that such a constitutional unit (a2) be a constitutional unit represented by General Formula (a2-1).

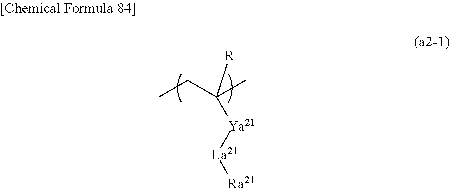

[In the formula. R represents a hydrogen atom, an alkyl group having 1 to 5 carbon atoms, or a halogenated alkyl group having 1 to 5 carbon atoms. Ya²¹ represents a single bond or a divalent linking group. La²¹ represents —O—, —COO—, —CON(R′)—, —OCO—, —CONHCO—, or —CONHCS—, and R′ represents a hydrogen atom or a methyl group. In a case where La²¹ represents —O—, Ya²¹ does not represent —CO—. Ra²¹ represents a lactone-containing cyclic group.]

In Formula (a2-1), R has the same definition as described above. R preferably represents a hydrogen atom, an alkyl group having 1 to 5 carbon atoms, or a fluorinated alkyl group having 1 to 5 carbon atoms and particularly preferably a hydrogen atom or a methyl group from the viewpoint of the industrial availability.

In Formula (a2-1), the divalent linking group as Ya²¹ is not particularly limited, and suitable examples thereof include a divalent hydrocarbon group which may have a substituent and a divalent linking group having a hetero atom. Examples of the divalent linking group as Ya²¹ include the same groups as those for the divalent linking group as Ya^{x1} in General Formula (a10-1).

It is preferable that Ya²¹ represent a single bond, an ester bond [—C(═O)—O—], an ether bond (—O—), a linear or branched alkylene group, or a combination thereof.

In Formula (a2-1), it is preferable that Ya²¹ represent a single bond and La²¹ represent —COO— or —OCO—.

In Formula (a2-1), Ra²¹ represents a lactone-containing cyclic group.

Suitable examples of the lactone-containing cyclic group as Ra²¹ include groups each represented by General Formulae (a2-r-1) to (a2-r-7).

The constitutional unit (a2) included in the component (A1) may be used alone or two or more kinds thereof. The component (A1) may or may not have the constitutional unit (a2).

In a case where the component (A1) has the constitutional unit (a2), the proportion of the constitutional unit (a2) is preferably in a range of 1% to 20% by mole, more preferably in a range of 1% to 15% by mole, and still more preferably in a range of 1% to 10% by mole with respect to the total amount (100% by mole) of all constitutional units constituting the component (A1).

In a case where the proportion of the constitutional unit (a2) is set to be greater than or equal to the above-described preferable lower limits, the effect to be obtained by allowing the component (A1) to have the constitutional unit (a2) is sufficiently obtained by the above-described effects. Further, in a case where the proportion thereof is set to be less than or equal to the upper limits of the above-described preferable ranges, the constitutional unit (a2) and other constitutional units can be balanced, and various lithography characteristics are enhanced.

### Constitutional Unit (a8):

The constitutional unit (a8) is a constitutional unit derived from a compound represented by General Formula (a8-1).

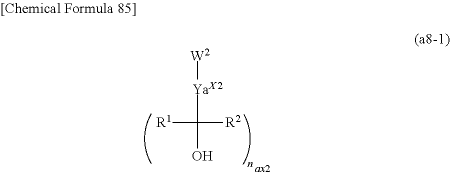

[In the formula, W² represents a polymerizable group-containing group. Ya^{x2} represents a single bond or an (nₐₓ₂+)-valent linking group. Ya^{x2} and W² may form a condensed ring. R¹ represents a fluorinated alkyl group having 1 to 12 carbon atoms. R² represents an organic group having 1 to 12 carbon atoms, which may have a fluorine atom, or a hydrogen atom. R² and Ya^{x2} may be bonded to each other to form a ring structure. nₐₓ₂ represents an integer of 1 to 3.]

The term “polymerizable group” in the polymerizable group-containing group as W² is a group that enables a compound having the polymerizable group to be polymerized by radical polymerization or the like, and refers to a group containing multiple bonds between carbon atoms, such as an ethylenic double bond.

The polymerizable group-containing group may be a group formed of only a polymerizable group or a group formed of a polymerizable group and a group other than the polymerizable group. Examples of the group other than the polymerizable group include a divalent hydrocarbon group which may have a substituent and a divalent linking group having a heteroatom.

Suitable examples of the polymerizable group-containing group include a group represented by a chemical formula: C(R^{X11})(R^{X12})═C(R^{X13})-Ya^{x0}-.

In the chemical formula, R^{X11}, R^{X12}, and R^{X13} each represents a hydrogen atom, an alkyl group having 1 to 5 carbon atoms, or a halogenated alkyl group having 1 to 5 carbon atoms, and Ya^{x0} represents a single bond or a divalent linking group.

Examples of the condensed ring formed by Ya^{x2} and W² include a condensed ring formed by a polymerizable group of the W² moiety and by Ya^{x2} and a condensed ring formed by a group other than the polymerizable group of the W² moiety and by Ya^{x2}.

The condensed ring formed by Ya^{x2} and W² may have a substituent.

Specific examples of the constitutional unit (a8) are shown below.

In the following formulae, R^{α} represents a hydrogen atom, a methyl group, or a trifluoromethyl group.

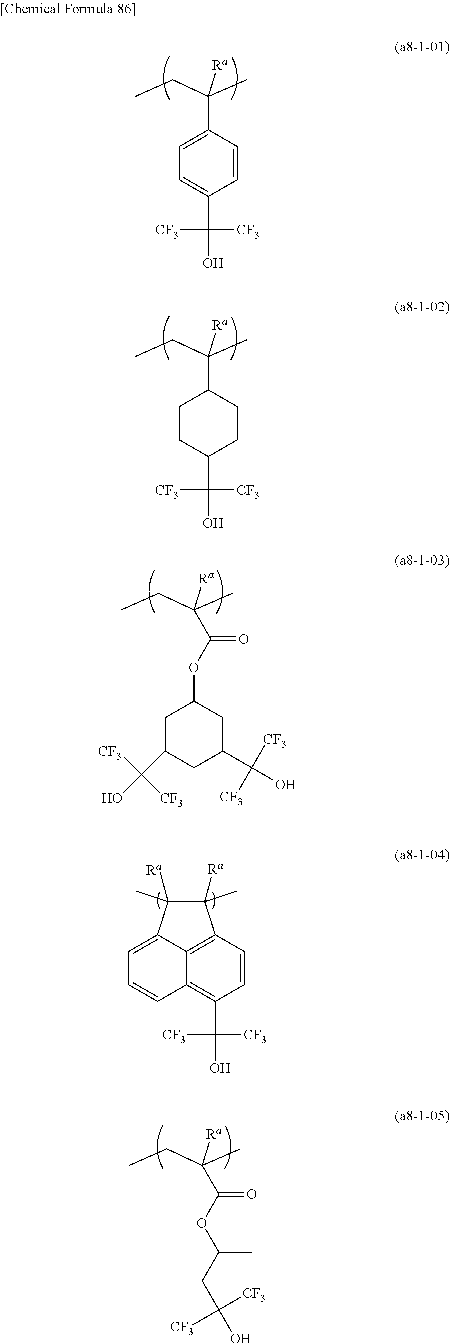

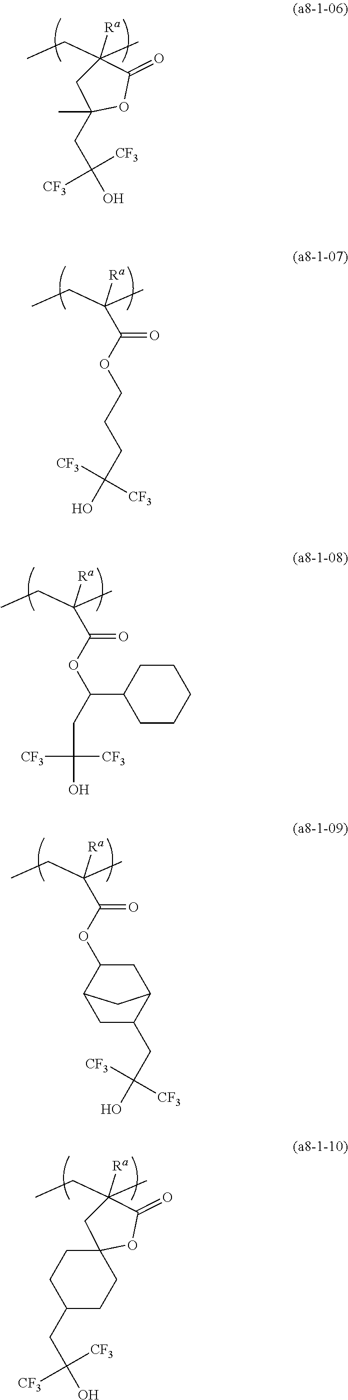

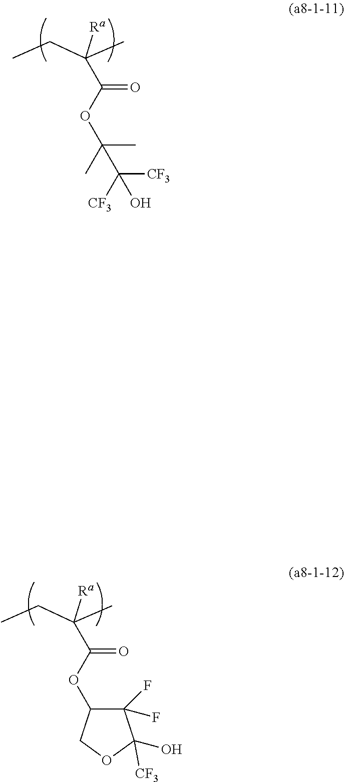

Among the examples, the constitutional unit (a8) is preferably at least one selected from the group consisting of constitutional units each represented by Chemical Formulae (a8-1-01) to (a8-1-04), (a8-1-06), (a8-1-08), (a8-1-09), and (a8-1-10) and more preferably at least one selected from the group consisting of constitutional units each represented by Chemical Formulae (a8-1-01) to (a8-1-04) and (a8-1-09).

The constitutional unit (a8) contained in the component (A1) may be used alone or two or more kinds thereof. The component (A1) may or may not have the constitutional unit (a8).

The proportion of the constitutional unit (a8) in the component (A1) is preferably in a range of 0% to 50% by mole and more preferably in a range of 0% to 30% by mole with respect to the total amount (100% by mole) of all constitutional units constituting the component (A1).

The component (A1) contained in the resist composition may be used alone or in combination of two or more kinds thereof.

Examples of the component (A1) include a polymer compound having a repeating structure of the constitutional unit (a0), the constitutional unit (a1), and the constitutional unit (a10); and a polymer compound having a repeating structure of the constitutional unit (a0), the constitutional unit (a1), the constitutional unit (a10), and the constitutional unit (a2).

The proportion of the constitutional unit (a0) in the polymer compound having a repeating structure of the constitutional unit (a0), the constitutional unit (a1), and the constitutional unit (a10) is preferably in a range of 2% to 30% by mole, more preferably in a range of 5% to 25% by mole, and still more preferably in a range of 10% to 15% by mole with respect to the total amount (100% by mole) of all constitutional units constituting the polymer compound.

The proportion of the constitutional unit (a1) in the polymer compound is preferably in a range of 20% to 80% by mole, more preferably in a range of 30% to 70% by mole, and still more preferably in a range of 40% to 60% by mole with respect to the total amount (100% by mole) of all constitutional units constituting the polymer compound.

Further, the proportion of the constitutional unit (a10) in the polymer compound is preferably in a range of 10% to 80% by mole, more preferably in a range of 15% to 70% by mole, still more preferably in a range of 20% to 60% by mole, and particularly preferably in a range of 20% to 50% by mole with respect to the total amount (100% by mole) of all constitutional units constituting the polymer compound.

The proportion of the constitutional unit (a0) in the polymer compound having a repeating structure of the constitutional unit (a0), the constitutional unit (a1), the constitutional unit (a10), and the constitutional unit (a2) is preferably in a range of 2% to 30% by mole, more preferably in a range of 5% to 25% by mole, and still more preferably in a range of 10% to 15% by mole with respect to the total amount (100% by mole) of all constitutional units constituting the polymer compound.

The proportion of the constitutional unit (a1) in the polymer compound is preferably in a range of 20% to 80% by mole, more preferably in a range of 30% to 70% by mole, and still more preferably in a range of 40% to 60% by mole with respect to the total amount 100% by mole) of all constitutional units constituting the polymer compound.

The proportion of the constitutional unit (a10) in the polymer compound is preferably in a range of 5% to 60% by mole, more preferably in a range of 10% to 50% by mole, and still more preferably in a range of 20% to 40% by mole with respect to the total amount (100% by mole) of all constitutional units constituting the polymer compound.

The proportion of the constitutional unit (a2) in the polymer compound is preferably greater than 0% by mole and 20% by mole or less, more preferably in a range of 5% to 20% by mole, and still more preferably in a range of 5% to 15% by mole with respect to the total amount (100% by mole) of all constitutional units constituting the polymer compound.

Such a component (A1) can be produced by dissolving a monomer, from which each constitutional unit is derived, in a polymerization solvent and adding a radical polymerization initiator such as azobisisobutylonitrile (AIBN) or dimethyl azobisisobutyrate (for example, V-601) to the solution so that the polymerization is carried out.

Alternatively, such a component (A1) can be produced by dissolving, in a polymerization solvent, a monomer from which the constitutional unit (a1) is derived and a monomer from which an optional constitutional unit (for example, the constitutional unit (a1), the constitutional unit (a10) or the like) is derived, adding a radical polymerization initiator as described above thereto to carry out polymerization, and carrying out a deprotection reaction.

Further, a —C(CF₃)₂—OH group may be introduced to the terminal during the polymerization using a combination of chain transfer agents such as HS—CH₂—CH₂—CH₂—C(CF₃)₂—OH. As described above, a copolymer into which a hydroxyalkyl group, formed by substitution of some hydrogen atoms in the alkyl group with fluorine atoms, has been introduced is effective for reducing development defects and reducing line edge roughness (LER: uneven irregularities of a line side wall).

The weight-average molecular weight (Mw) (in terms of polystyrene according to gel permeation chromatography (GPC)) of the component (A1) is not particularly limited, but is preferably in a range of 1000 to 50000, more preferably in a range of 5000 to 40000, and still more preferably in a range of 5000 to 30000.

In a case where the Mw of the component (A1) is less than or equal to the upper limits of the above-described preferable ranges, the resist composition exhibits a satisfactory solubility in a resist solvent for a resist enough to be used as a resist. On the contrary, in a case where the Mw of the component (A1) is greater than or equal to the lower limits of the above-described preferable ranges, the dry etching resistance and the cross-sectional shape of the resist pattern are satisfactory.

Further, the dispersity (Mw/Mn) of the component (A1) is not particularly limited, but is preferably in a range of 1.0 to 4.0, more preferably in a range of 1.0 to 3.0, and particularly preferably in a range of 1.0 to 2.0. Further, Mn represents the number average molecular weight.

### In Regard to Component (A2)

In the resist composition according to the present embodiment, a base material component (hereinafter, also referred to as “component (A2)”) which does not correspond to the component (A1) and whose solubility in a developing solution is changed due to the action of an acid may be used in combination as the component (A).

The component (A2) is not particularly limited and may be optionally selected from a plurality of components of the related art which have been known as base material components for a chemically amplified resist composition and used.

As the component (A2), a polymer compound or a low-molecular-weight compound may be used alone or in combination of two or more kinds thereof.

The proportion of the component (A1) in the component (A) is preferably 25% by mass or greater, more preferably 50% by mass or greater, and still more preferably 75% by mass or greater, and may be 100% by mass with respect to the total mass of the component (A). In a case where the proportion thereof is 25% by mass or greater, a resist pattern having excellent various lithography characteristics such as high sensitivity, high resolution, and improved roughness is likely to be formed.

In the resist composition according to the present embodiment, the content of the component (A) may be adjusted according to the thickness and the like of the resist film intended to be formed.

### <Other Components>

The resist composition according to the present embodiment may further contain other components in addition to the component (A) described above. Examples of the other components include a component (B), a component (D), a component (E), a component (F), and a component (S), which are described below.

<<Acid generator component (B)>>

The resist composition according to the present embodiment may further contain an acid generator component (B) which generates an acid upon light exposure.

The component (B) is not particularly limited, and those which have been suggested so far as an acid generator for a chemically amplified resist composition in the related art can be used.

Examples of the acid generator include various acid generators, for example, onium salt-based acid generators such as iodonium salts and sulfonium salts; oxime sulfonate-based acid generators; diazomethane-based acid generators such as bisalkyl or bisaryl sulfonyl diazomethanes and poly(bis-sulfonyl)diazomethanes; nitrobenzyl sulfonate-based acid generators, iminosulfonate-based acid generators, and disulfone-based acid generators.

Examples of the onium salt-based acid generators include a compound represented by General Formula (b-1) (hereinafter, also referred to as “component (b-1)”), a compound represented by General Formula (b-2) (hereinafter, also referred to as “component (b-2)”), and a compound represented by General Formula (b-3) (hereinafter, also referred to as “component (b-3)”).

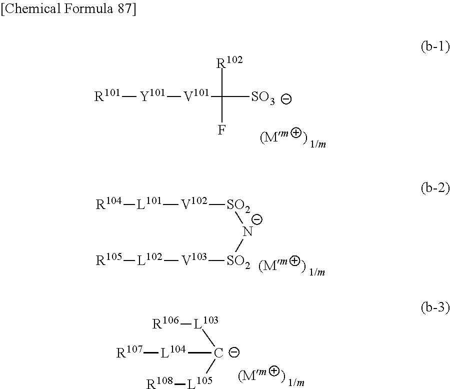

[In the formulae, R¹⁰¹ and R¹⁰⁴ to R¹⁰⁸ each independently represents a cyclic group which may have a substituent, a chain-like alkyl group which may have a substituent, or a chain-like alkenyl group which may have a substituent. R¹⁰⁴ and R¹⁰⁵ may be bonded to each other to form a ring structure. R¹⁰² represents a fluorinated alkyl group having 1 to 5 carbon atoms or a fluorine atom. Y¹⁰¹ represents a divalent linking group having an oxygen atom or a single bond. V¹⁰¹ to V¹⁰³ each independently represents a single bond, an alkylene group, or a fluorinated alkylene group. Here, Y¹⁰¹ and V¹⁰¹ do not represent a single bond at the same time. L¹⁰¹ and L¹⁰² each independently represents a single bond or an oxygen atom. L¹⁰³ to L¹⁰⁵ each independently represents a single bond, —CO—, or —SO₂—. m represents an integer of 1 or greater, and M′^{m+} represents an m-valent onium cation.]

### {Anion Moiety}

Anions in Component (b-1)

In Formula (b-1), R¹⁰¹ represents a cyclic group which may have a substituent, a chain-like alkyl group which may have a substituent, or a chain-like alkenyl group which may have a substituent.

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group that has no aromaticity. In addition, it is preferable that the aliphatic hydrocarbon group be saturated.

The aromatic hydrocarbon group as R¹⁰¹ is a hydrocarbon group having an aromatic ring. The aromatic hydrocarbon group preferably has 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring contained in the aromatic hydrocarbon group as R¹⁰¹ include benzene, fluorene, naphthalene, anthracene, phenanthrene, biphenyl, or an aromatic heterocyclic ring in which some carbon atoms constituting any of these aromatic rings have been substituted with hetero atoms. Examples of the heteroatom in the aromatic heterocyclic rings include an oxygen atom, a sulfur atom, and a nitrogen atom.

Specific examples of the aromatic hydrocarbon group as R¹⁰¹ include a group obtained by removing one hydrogen atom from the aromatic ring (an aryl group such as a phenyl group or a naphthyl group) and a group in which one hydrogen atom of the aromatic ring is substituted with an alkylene group (for example, a benzyl group, a phenethyl group, or a 1-naphthylmethyl group). The number of carbon atoms in the alkylene group (alkyl chain in the arylalkyl group) is preferably in a range of 1 to 4, more preferably 1 or 2 carbon atoms, and particularly preferably 1.

Examples of the cyclic aliphatic hydrocarbon group as R¹⁰¹ include an aliphatic hydrocarbon group having a ring in the structure thereof.

Examples of the aliphatic hydrocarbon group having a ring in the structure thereof include an alicyclic hydrocarbon group (group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group preferably has 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane preferably has 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring polycyclic skeleton such as adamantane, norbornane, isobornane, tricyclo[5.2.1.0^{2,6}]decane, or tetracyclododecane; and a polycycloalkane having a condensed ring polycyclic skeleton such as a cyclic group having a steroid skeleton are preferable as the polycycloalkane.

Among these examples, as the cyclic aliphatic hydrocarbon group as R¹⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is still more preferable, and an adamantyl group is particularly preferable.

The linear aliphatic hydrocarbon group which may be bonded to the alicyclic hydrocarbon group preferably has 1 to 10 carbon atoms, more preferably 1 to 6 carbon atoms, still more preferably 1 to 4 carbon atoms, and most preferably 1 to 3 carbon atoms. As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group which may be bonded to the alicyclic hydrocarbon group preferably has 2 to 10 carbon atoms, more preferably 3 to 6 carbon atoms, still more preferably 3 or 4 carbon atoms, and most preferably 3 carbon atoms. As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH—, and —C(CH₂CH₃)₂—; alkylethylene groups such as — CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

Further, the cyclic hydrocarbon group as R¹⁰¹ may have a hetero atom such as a heterocyclic ring. Specific examples thereof include lactone-containing cyclic groups each represented by General Formulae (a2-r-1) to (a2-r-7), —SO₂-containing cyclic groups each represented by General Formulae (b5-r-1) to (b5-r-4), and other heterocyclic groups each represented by Chemical Formulae (r-hr-1) to (r-hr-16).

Examples of the substituent for the cyclic group as R¹⁰¹ include an alkyl group, an alkoxy group, a halogen atom, a halogenated alkyl group, a hydroxyl group, a carbonyl group, and a nitro group.

An alkyl group having 1 to 5 carbon atoms is preferable as the alkyl group serving as a substituent.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

A fluorine atom, a bromine atom, or an iodine atom is preferable as the halogen atom serving as a substituent.

An example of the above-described halogenated alkyl group as the substituent includes a group in which some or all hydrogen atoms in an alkyl group having 1 to 5 carbon atoms such as a methyl group, an ethyl group, a propyl group, an n-butyl group, or a tert-butyl group have been substituted with the above-described halogen atoms.

The carbonyl group as the substituent is a group that substitutes a methylene group (—CH₂—) constituting the cyclic hydrocarbon group.

The cyclic hydrocarbon group as R¹⁰¹ may be a condensed cyclic group having a condensed ring in which an aliphatic hydrocarbon ring and an aromatic ring are condensed. Examples of the condensed ring include those obtained by condensing one or more aromatic rings with a polycycloalkane having a crosslinked ring-based polycyclic skeleton. Specific examples of the crosslinked ring-based polycycloalkane include a bicycloalkane such as bicyclo[2.2.1]heptane (norbornane) and bicyclo[2.2.2]octane. As the condensed cyclic group, a group having a condensed ring in which two or three aromatic rings are condensed with a bicycloalkane is preferable, and a group having a condensed ring in which two or three aromatic rings are condensed with bicyclo[2.2.2]octane is more preferable. Specific examples of the condensed cyclic group as R¹⁰¹ include groups represented by Formulae (r-br-1) and (r-br-2). In the formulae, * represents a bonding site with respect to Y¹⁰¹ in Formula (b-1).

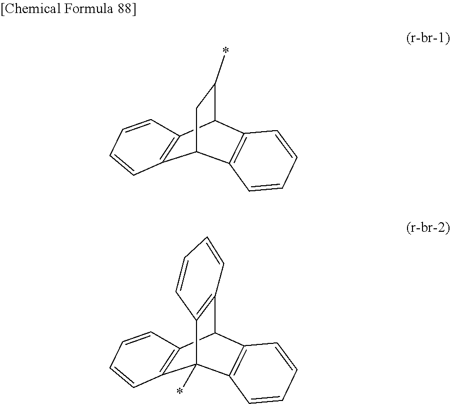 [SMILES: *C1CC2c3ccccc3C1c1ccccc12 ; *C12c3ccccc3C(c3ccccc31)c1ccccc12]

Examples of the substituent that the condensed cyclic group as R¹⁰¹ may have include an alkyl group, an alkoxy group, a halogen atom, a halogenated alkyl group, a hydroxyl group, a carbonyl group, a nitro group, an aromatic hydrocarbon group, and an alicyclic hydrocarbon group.

Examples of the alkyl group, the alkoxy group, the halogen atom, and the halogenated alkyl group as the substituent of the condensed cyclic group include those exemplified as the substituent of the cyclic group as R¹⁰¹.

Examples of the aromatic hydrocarbon group as the substituent of the condensed cyclic group include a group in which one hydrogen atom has been removed from the aromatic ring (an aryl group such as a phenyl group or a naphthyl group), a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group), and a heterocyclic group represented by any of Formulae (r-hr-1) to (r-hr-6).

Examples of the alicyclic hydrocarbon group as the substituent of the condensed cyclic group include a group in which one hydrogen atom has been removed from a monocycloalkane such as cyclopentane or cyclohexane; a group in which one hydrogen atom has been removed from a polycycloalkane such as adamantane, norbornane, isobornane, tricyclo[5.2.1.0^{2,6}]decane, or tetracyclododecane; a lactone-containing cyclic group represented by any of General Formulae (a2-r-1) to (a2-r-7); a —SO₂-containing cyclic group represented by any of General Formulae (b5-r-1) to (b5-r-4); and a heterocyclic group represented by any of Formulae (r-hr-7) to (r-hr-16).

Chain-Like Alkyl Group which May have Substituent:

The chain-like alkyl group as R¹⁰¹ may be linear or branched.

The number of carbon atoms in the linear alkyl group is preferably in a range of 1 to 20, more preferably in a range of 1 to 15, and most preferably in a range of 1 to 10.

The branched alkyl group preferably has 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-Like Alkenyl Group which May have Substituent:

The chain-like alkenyl group as R¹⁰¹ may be linear or branched, and the number of carbon atoms thereof is preferably 2 to 10, more preferably 2 to 5, still more preferably 2 to 4, and particularly preferably 3. Examples of the linear alkenyl group include a vinyl group, a propenyl group (allyl group), and a butenyl group. Examples of the branched alkenyl group include a 1-methylvinyl group, a 2-methylvinyl group, a 1-methylpropenyl group, and a 2-methylpropenyl group.

Among the examples, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R¹⁰¹ include an alkoxy group, a halogen atom, a halogenated alkyl group, a hydroxyl group, a carbonyl group, a nitro group, an amino group, and a cyclic group as R¹⁰¹.

In Formula (b-1), Y¹⁰¹ represents a single bond or a divalent linking group having an oxygen atom.

In a case where Y¹⁰¹ represents a divalent linking group having an oxygen atom, Y¹⁰¹ may have an atom other than the oxygen atom. Examples of atoms other than an oxygen atom include a carbon atom, a hydrogen atom, a sulfur atom, and a nitrogen atom.

Examples of the divalent linking group having an oxygen atom include linking groups each represented by General Formulae (y-a1-1) to (y-a1-7). Further, in General Formulae (y-a1-1) to (y-a1-7). V′¹⁰¹ in General Formulae (y-a1-1) to (y-a1-7) is bonded to R¹⁰¹ in Formula (b-1).

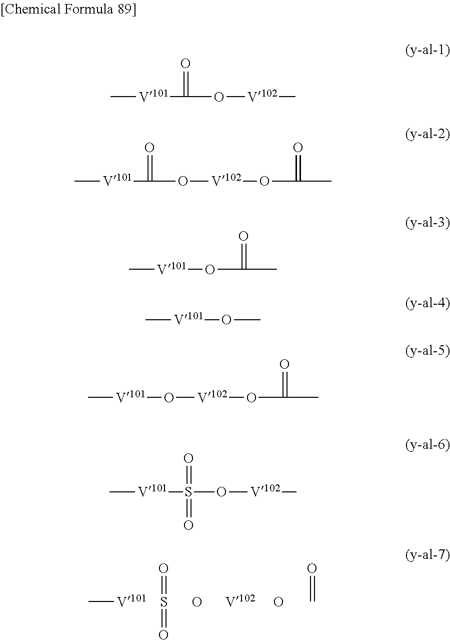

[In the formulae, V′¹⁰¹ represents a single bond or an alkylene group having 1 to 5 carbon atoms, and V′¹⁰² represents a divalent saturated hydrocarbon group having 1 to 30 carbon atoms.]

As the divalent saturated hydrocarbon group as V′¹⁰², an alkylene group having 1 to 30 carbon atoms is preferable, an alkylene group having 1 to 10 carbon atoms is more preferable, and an alkylene group having 1 to 5 carbon atoms is still more preferable.

The alkylene group as V′¹⁰¹ and V′¹⁰² may be a linear alkylene group or a branched alkylene group, and a linear alkylene group is preferable.

Specific examples of the alkylene group as V′¹⁰¹ and V′¹⁰² include a methylene group [—CH₂—]; an alkylmethylene group such as —CH(CH₃)—, —CH(CH₂CH₃)—, —C(CH₃)₂—, —C(CH₃)(CH₂CH₃)—, —C(CH₃)(CH₂CH₂CH₃)—, or —C(CH₂CH₃)₂—; an ethylene group [—CH₂CH₂—]; an alkylethylene group such as —CH(CH₃)CH₂—, —CH(CH₃)CH(CH₃)—, —C(CH₃)₂CH₂—, or —CH(CH₂CH₃)CH₂—; a trimethylene group (n-propylene group) [—CH₂CH₂CH₂—]; an alkyltrimethylene group such as —CH(CH₃)CH₂CH₂— or —CH₂CH(CH₃)CH₂—; a tetramethylene group [—CH₂CH₂CH₂CH₂—]; an alkyltetramethylene group such as —CH(CH₃)CH₂CH₂CH₂— or —CH₂CH(CH₃)CH₂CH₂—; and a pentamethylene group [—CH₂CH₂CH₂CH₂CH₂—].

Further, part of a methylene group in the alkylene group as V′¹⁰¹ and V′¹⁰² may be substituted with a divalent aliphatic cyclic group having 5 to 10 carbon atoms. As the aliphatic cyclic group, a divalent group in which one hydrogen atom has been further removed from the cyclic aliphatic hydrocarbon group (a monocyclic aliphatic hydrocarbon group or a polycyclic aliphatic hydrocarbon group) as Ra′³ in Formula (a1-r-1) is preferable, and a cyclohexylene group, a 1,5-adamantylene group, or a 2,6-adamantylene group is more preferable.

In Formula (b-1), V¹⁰¹ represents a single bond, an alkylene group, or a fluorinated alkylene group. Among the examples, it is preferable that V¹⁰¹ represent a single bond or a linear fluorinated alkylene group having 1 to 4 carbon atoms.

In Formula (b-1), R¹⁰² represents a fluorine atom or a fluorinated alkyl group having 1 to 5 carbon atoms. R¹⁰² preferably represents a fluorine atom or a perfluoroalkyl group having 1 to 5 carbon atoms and more preferably a fluorine atom.

In a case where Y¹⁰¹ represents a single bond, specific examples of the anion moiety represented by Formula (b-1) include a fluorinated alkylsulfonate anion such as a trifluoromethanesulfonate anion or a perfluorobutanesulfonate anion. Further, in a case where Y¹⁰¹ represents a divalent linking group having an oxygen atom, specific examples thereof include an anion represented by any of Formulae (an-1) to (an-3).

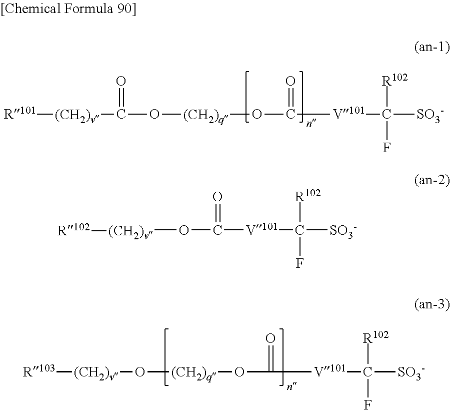

[In the formulae, R″¹⁰¹ represents an aromatic cyclic group which may have a substituent, a monovalent heterocyclic group represented by any of Chemical Formulae (r-hr-1) to (r-hr-6), a condensed cyclic group represented by Formula (r-br-1) or (r-br-2), a chain-like alkyl group which may have a substituent, or an aromatic cyclic group which may have a substituent. R″¹⁰² represents an aliphatic cyclic group which may have a substituent, a condensed cyclic group represented by Formula (r-br-1) or (r-br-2), a lactone-containing cyclic group represented by any of General Formulae (a2-r-1) and (a2-r-3) to (a2-r-7), or a —SO₂-containing cyclic group represented by any of General Formulae (b5-r-1) to (b5-r-4). R″¹⁰³ represents an aromatic cyclic group which may have a substituent, an aliphatic cyclic group which may have a substituent, or a chain-like alkenyl group which may have a substituent. V″¹⁰¹ represents a single bond, an alkylene group having 1 to 4 carbon atoms, or a fluorinated alkylene group having 1 to 4 carbon atoms. R¹⁰² represents a fluorine atom or a fluorinated alkyl group having 1 to 5 carbon atoms. Each v″ independently represents an integer of 0 to 3, each q″ independently represents an integer of 0 to 20, and n″ represents 0 or 1.]

As the aliphatic cyclic group as R″¹⁰¹, R″¹⁰², and R″¹⁰³ which may have a substituent, the same groups as those for the cyclic aliphatic hydrocarbon group as R¹⁰¹ in Formula (b-1) are preferable. Examples of the substituent include the same groups as those for the substituent which may substitute the cyclic aliphatic hydrocarbon group as R¹⁰¹ in Formula (b-1).

As the aromatic cyclic group which may have a substituent as R″¹⁰¹ and R″¹⁰³, the same groups as those for the aromatic hydrocarbon group in the cyclic hydrocarbon group as R¹⁰¹ in Formula (b-1) are preferable. Examples of the substituent include the same groups as those for the substituent which may substitute the aromatic hydrocarbon group as R¹⁰¹ in Formula (b-1).

As the chain-like alkyl group as R″¹⁰¹ which may have a substituent, the same groups as those for the chain-like alkyl group as R¹⁰¹ in Formula (b-1) are preferable.

As the chain-like alkenyl group as R″¹⁰³ which may have a substituent, the same groups as those for the chain-like alkenyl group as R¹⁰¹ in Formula (b-1) are preferable.

Anions in Component (b-2)

In Formula (b-2), R¹⁰⁴ and R¹⁰⁵ each independently represents a cyclic group which may have a substituent, a chain-like alkyl group which may have a substituent, or a chain-like alkenyl group which may have a substituent, and examples thereof include the same groups as those for R¹⁰¹ in Formula (b-1). Here, R¹⁰⁴ and R¹⁰⁵ may be bonded to each other to form a ring.

R¹⁰⁴ and R¹⁰⁵ preferably represent a chain-like alkyl group which may have a substituent and more preferably a linear or branched alkyl group or a linear or branched fluorinated alkyl group.

The chain-like alkyl group preferably has 1 to 10 carbon atoms, more preferably 1 to 7 carbon atoms, and still more preferably 1 to 3 carbon atoms. It is preferable that the number of carbon atoms in the chain-like alkyl group as R¹⁰⁴ and R¹⁰⁵ decrease within the range of the number of carbon atoms from the viewpoint that the solubility in a solvent for a resist is also satisfactory. Further, in the chain-like alkyl group as R¹⁰⁴ and R¹⁰⁵, it is preferable that the number of hydrogen atoms substituted with fluorine atoms be as large as possible from the viewpoint that the acid strength increases and the transparency to high energy light or electron beams having a wavelength of 250 nm or less is improved. The proportion of fluorine atoms in the chain-like alkyl group, that is, the fluorination ratio is preferably in a range of 70% to 100% and more preferably in a range of 90% to 100%, and it is most preferable that the chain-like alkyl group be a perfluoroalkyl group in which all hydrogen atoms have been substituted with fluorine atoms.

In Formula (b-2), V¹⁰² and V¹⁰³ each independently represents a single bond, an alkylene group, or a fluorinated alkylene group, and examples thereof include the same groups as those for V¹⁰¹ in Formula (b-1).

In Formula (b-2), L¹⁰¹ and L¹⁰² each independently represents a single bond or an oxygen atom.

Anions in Component (b-3)

In Formula (b-3), R¹⁰⁶ to R¹⁰⁸ each independently represents a cyclic group which may have a substituent, a chain-like alkyl group which may have a substituent, or a chain-like alkenyl group which may have a substituent, and examples thereof include the same groups as those for R¹⁰¹ in Formula (b-1).

In Formula (b-3), L¹⁰³ to L¹⁰⁵ each independently represents a single bond, —CO—, or —SO₂—.

Among these, as the anion moiety of the component (B), an anion of the component (b-1) is preferable, and an anion represented by Formula (an-1) is more preferable.

### [Cation Moiety]

In Formulae (b-1), (b-2), and (b-3). M′^{m+} represents an m-valent onium cation. Among them, a sulfonium cation and an iodonium cation are preferable, in represents an integer of 1 or greater.

As the cation moiety of the component (B), a sulfonium cation is preferable, the cations each represented by Formulae (ca-1) to (ca-3) are more preferable, the cation represented by Formula (ca-1) is still more preferable, and the cations each represented by Formulae (ca-1-1) to (ca-1-83) are particularly preferable.

In the resist composition according to the present embodiment, the component (B) may be used alone or in combination of two or more kinds thereof.

Since the resist composition according to the present embodiment has the constitutional unit (a0) in the component (A1), the resist composition may contain no component (B).

The content of the component (B) in the resist composition is preferably in a range of 0 to 30 parts by mass, more preferably in a range of 0 to 20 parts by mass, still more preferably in a range of 0 to 10 parts by mass, and particularly preferably in a range of 0 to 8 parts by mass with respect to 100 parts by mass of the component (A).

It is preferable that the content of the component (B) be set to be in the above-described preferable ranges from the viewpoint that a uniform solution is likely to be obtained in a case where each component of the resist composition is dissolved in an organic solvent, and the storage stability of the resist composition is enhanced.

### <<Base Component (D)>>

The resist composition according to the present embodiment may contain, in addition to the component (A), a base component (component (D)) that traps an acid generated upon light exposure (that is, controls diffusion of an acid). The component (D) acts as a quencher (an acid diffusion control agent) which traps the acid generated in the resist composition upon light exposure.

Examples of the component (D) include a photodecomposable base (D) having acid diffusion controllability (hereinafter, referred to as “component (D1)”) which is lost by the decomposition upon light exposure and a nitrogen-containing organic compound (D2) (hereinafter, referred to as “component (D2)”) which does not correspond to the component (D1). Among these, the photodecomposable base (component (D1)) is preferable from the viewpoint that all the characteristics of high sensitivity, roughness reduction, and suppression of the occurrence of coating defects are likely to be enhanced.

The component (D1) and the component (D2) may be contained in the form of a compound, in the form in which the component (D1) and the component (D2) are incorporated into the component (A1) as the constitutional unit, or in both forms.

### In Regard to Component (D1)

The component (D1) is not particularly limited as long as the component is decomposed upon light exposure and loses acid diffusion controllability, and one or more compounds selected from the group consisting of a compound represented by General Formula (d1-1) (hereinafter, referred to as “component (d1-1)”), a compound represented by General Formula (d1-2) (hereinafter, referred to as “component (d1-2)”), and a compound represented by General Formula (d1-3) (hereinafter, referred to as “component (d1a-3)”) are preferable.

Since the components (d1-1) to (d1-3) are decomposed and lose acid diffusion controllability (basicity), the components (d1-1) to (d1-3) do not act as a quencher at the exposed portion of the resist film, but act as a quencher at the unexposed portion of the resist film.

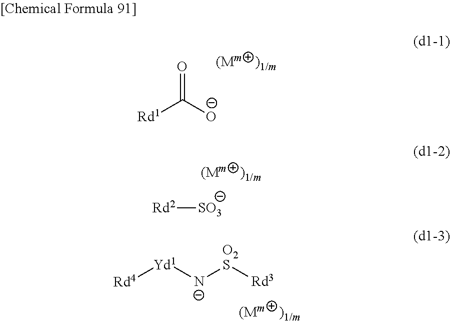

[In the formulae, Rd¹ to Rd⁴ represent a cyclic group which may have a substituent, a chain-like alkyl group which may have a substituent, or a chain-like alkenyl group which may have a substituent. Here, no fluorine atom is bonded to the carbon atom adjacent to the S atom in Rd² of Formula (d1-2). Yd¹ represents a single bond or a divalent linking group. m represents an integer of 1 or greater, and M^{m+}'s each independently represents an m-valent organic cation.]

{Component (d1-1)}

### Anion Moiety

In Formula (d1-1), Rd¹ represents a cyclic group which may have a substituent, a chain-like alkyl group which may have a substituent, or a chain-like alkenyl group which may have a substituent, and examples thereof include the same groups as those for R′²⁰¹.

Among these, it is preferable that Rd¹ represent an aromatic hydrocarbon group which may have a substituent, an aliphatic cyclic group which may have a substituent, or a chain-like alkyl group which may have a substituent. Examples of the substituent that may be included in these groups include a hydroxyl group, an oxo group, an alkyl group, an aryl group, a fluorine atom, a fluorinated alkyl group, a lactone-containing cyclic group represented by any of General Formulae (a2-r-1) to (a2-r-8), an ether bond, an ester bond, and a combination thereof. In a case where an ether bond or an ester bond is included as the substituent, the substituent may be bonded through an alkylene group, and a linking group represented by any of Formulae (y-a1-1) to (y-a1-5) is preferable as the substituent. Further, in a case where the aromatic hydrocarbon group, the aliphatic cyclic group, or the chain-like alkyl group as Rd¹ contain a linking group represented by each of General Formulae (y-a1-1) to (y-a1-7) as a substituent, V′¹⁰¹ in General Formulae (y-a1-1) to (y-a1-7) is bonded to the carbon atom constituting the aromatic hydrocarbon group, the aliphatic cyclic group, or the chain-like alkyl group as Rd¹ in Formula (d1-1), in General Formulae (y-a1-1) to (y-a1-7).

Suitable examples of the aromatic hydrocarbon group include a phenyl group, a naphthyl group, and a polycyclic structure having a bicyclooctane skeleton (a polycyclic structure formed of a bicyclooctane skeleton and a ring structure other than the bicyclooctane skeleton).

As the aliphatic cyclic group, a group in which one or more hydrogen atoms have been removed from a polycycloalkane such as adamantane, norbornane, isobornane, tricyclo[5.2.1.0^{2,6}]decane, or tetracyclododecane is more preferable.

It is preferable that the chain-like alkyl group have 1 to 10 carbon atoms, and specific examples thereof include a linear alkyl group such as a methyl group, an ethyl group, a propyl group, a butyl group, a pentyl group, a hexyl group, a heptyl group, an octyl group, a nonyl group, or a decyl group; and a branched alkyl group such as a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, or a 4-methylpentyl group.

In a case where the chain-like alkyl group is a fluorinated alkyl group having a fluorine atom or a fluorinated alkyl group as a substituent, the fluorinated alkyl group preferably has 1 to 11 carbon atoms, more preferably 1 to 8 carbon atoms, and still more preferably 1 to 4 carbon atoms. The fluorinated alkyl group may have an atom other than a fluorine atom. Examples of the atom other than a fluorine atom include an oxygen atom, a sulfur atom, and a nitrogen atom.

Specific preferred examples of the anion moiety in the component (d1-1) are described below.

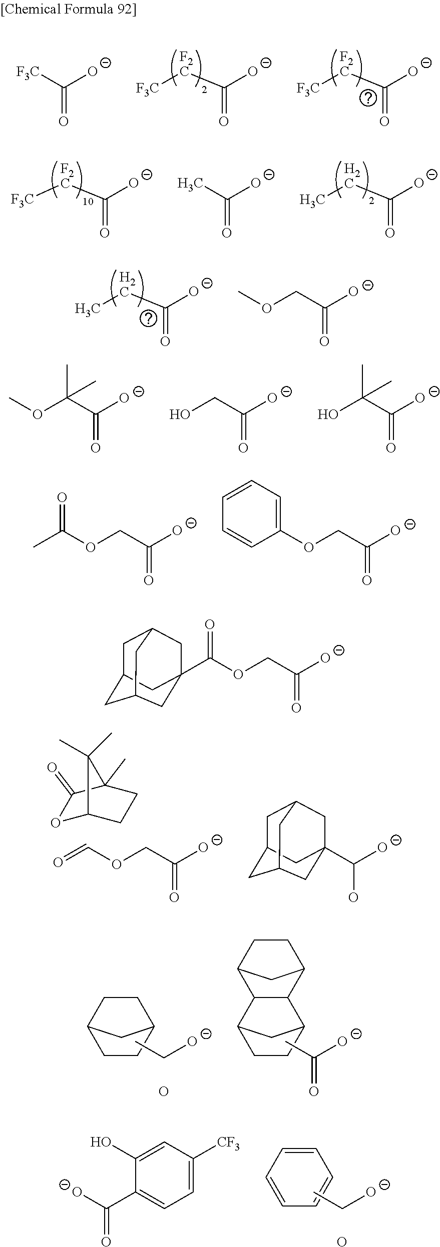

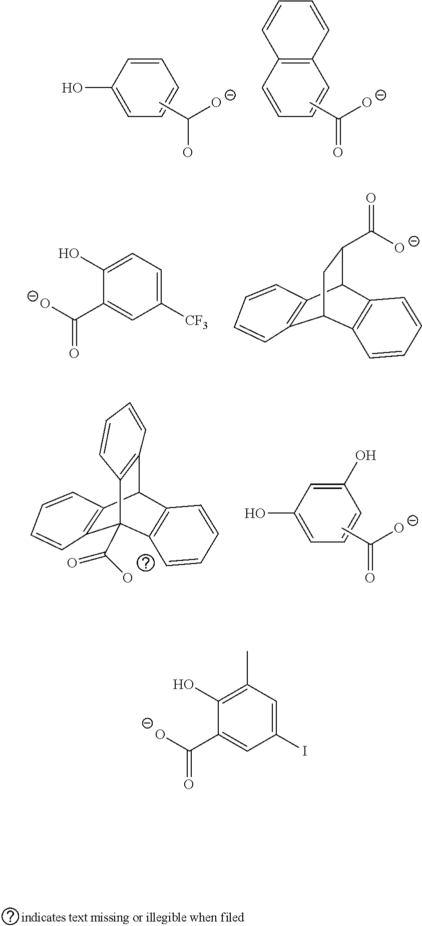

### Cation Moiety

In Formula (d1-1), M^{m+} represents an m-valent organic cation.

Suitable examples of the organic cation as M^{m+} include the same cations as those each represented by General Formulae (ca-1) to (ca-3). Among these, a cation represented by General Formula (ca-1) is more preferable, and a cation represented by any of Formulae (ca-1-1) to (ca-1-84) is still more preferable.

The component (d1-1) may be used alone or in combination of two or more kinds thereof.

[Component (d1-2)]

### Anion Moiety

In Formula (d1-2), Rd² represents a cyclic group which may have a substituent, a chain-like alkyl group which may have a substituent, or a chain-like alkenyl group which may have a substituent, and examples thereof include the same groups as those for R′ ²⁰¹.

Here, no fluorine atom is bonded to the carbon atom adjacent to the S atom in Rd² (the carbon atom is not substituted with fluorine). As a result, the anion of the component (d1-2) becomes an appropriately weak acid anion, thereby improving the quenching ability of the component (D).

Rd² preferably represents a chain-like alkyl group which may have a substituent or an aliphatic cyclic group which may have a substituent and more preferably an aliphatic cyclic group which may have a substituent.

The chain-like alkyl group preferably has 1 to 10 carbon atoms and more preferably 3 to 10 carbon atoms.

As the aliphatic cyclic group, a group in which one or more hydrogen atoms have been removed from adamantane, norbornane, isobornane, tricyclo[5.2.1.0^{2,6}]decane, or tetracyclododecane (a group which may have a substituent); and a group in which one or more hydrogen atoms have been removed from camphor are more preferable.

The hydrocarbon group as Rd² may have a substituent, and examples of the substituent include the same groups as those for the substituent that the hydrocarbon group (such as an aromatic hydrocarbon group, an aliphatic cyclic group, or a chain-like alkyl group) as Rd¹ in Formula (d1-1) may have.

Specific preferred examples of the anion moiety in the component (d1-2) are described below.

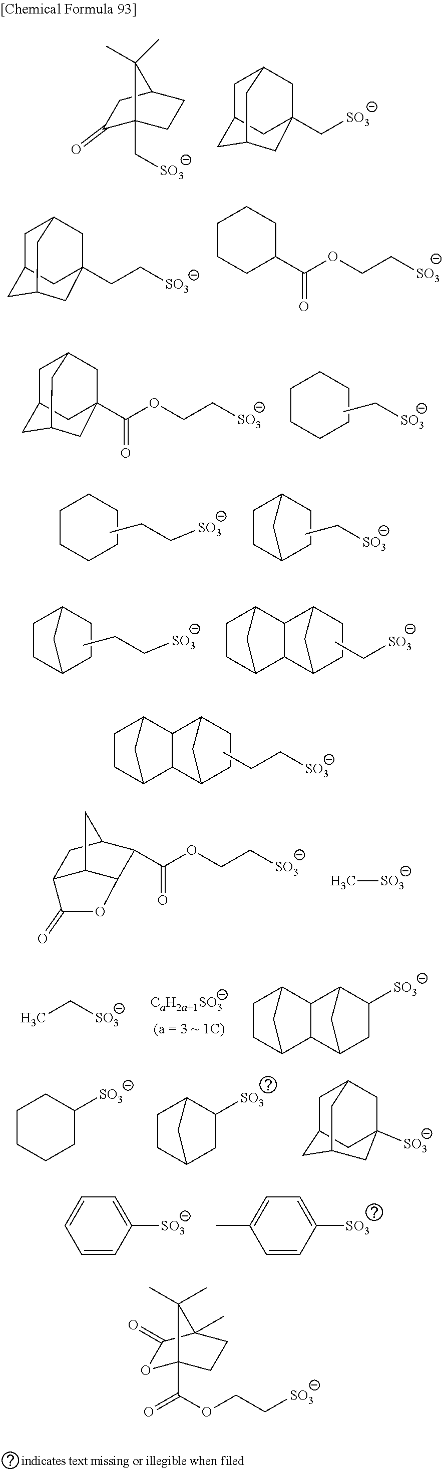

### Cation Moiety

In Formula (d1-2), M^{m+} represents an m-valent organic cation and has the same definition as that for M^{m+} in Formula (d1-1).

The component (d1-2) may be used alone or in combination of two or more kinds thereof.

[Component (d1-3)]

### Anion Moiety

In Formula (d1-3), Rd³ represents a cyclic group which may have a substituent, a chain-like alkyl group which may have a substituent, or a chain-like alkenyl group which may have a substituent, and examples thereof include the same groups as those for R′²⁰¹. Among these, a cyclic group having a fluorine atom, a chain-like alkyl group, or a chain-like alkenyl group is preferable. Among these, a fluorinated alkyl group is preferable, and the same groups as those for the fluorinated alkyl group represented by Rd¹ fare more preferable.

In Formula (d1-3), Rd⁴ represents a cyclic group which may have a substituent, a chain-like alkyl group which may have a substituent, or a chain-like alkenyl group which may have a substituent, and examples thereof include the same groups as those for R′²⁰¹.

Among these, an alkyl group which may have a substituent, an alkoxy group which may have a substituent, an alkenyl group which may have a substituent, or a cyclic group which may have a substituent is preferable.

It is preferable that the alkyl group as Rd⁴ be a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. Some hydrogen atoms in the alkyl group as Rd⁴ may be substituted with a hydroxyl group, a cyano group, or the like.

It is preferable that the alkoxy group as Rd⁴ be an alkoxy group having 1 to 5 carbon atoms, and specific examples of the alkoxy group having 1 to 5 carbon atoms include a methoxy group, an ethoxy group, an n-propoxy group, an iso-propoxy group, an n-butoxy group, and a tert-butoxy group. Among these, a methoxy group and an ethoxy group are preferable.

Examples of the alkenyl group as Rd⁴ include the same groups as those for the alkenyl group as R′²⁰¹. Among these, a vinyl group, a propenyl group (an allyl group), a 1-methylpropenyl group, and a 2-methylpropenyl group are preferable. These groups may further contain an alkyl group having 1 to 5 carbon atoms or a halogenated alkyl group having 1 to 5 carbon atoms as a substituent.

Examples of the cyclic group as Rd⁴ include the same groups as those for the cyclic group as R′²⁰¹. Among these, an alicyclic group in which one or more hydrogen atoms have been removed from a cycloalkane such as cyclopentane, cyclohexane, adamantane, norbornane, isobornane, tricyclo[5.2.1.0^{2,6}]decane, or tetracyclododecane or an aromatic group such as a phenyl group or a naphthyl group is preferable. In a case where Rd⁴ represents an alicyclic group, the resist composition is satisfactorily dissolved in an organic solvent so that the lithography characteristics are enhanced. Further, in a case where Rd⁴ represents an aromatic group, the resist composition has excellent light absorption efficiency in lithography using EUV or the like as an exposure light source, and thus the sensitivity and lithography characteristics are enhanced.

In Formula (d1-3). Yd¹ represents a single bond or a divalent linking group.

The divalent linking group as Yd¹ is not particularly limited, and examples thereof include a divalent hydrocarbon group (an aliphatic hydrocarbon group or an aromatic hydrocarbon group) which may have a substituent and a divalent linking group having a heteroatom. Examples of the divalent linking groups are the same as those for the divalent hydrocarbon group which may have a substituent and the divalent linking group having a hetero atom described in the section of the divalent linking group as Ya²¹ in Formula (a2-1).

It is preferable that Yd¹ represent a carbonyl group, an ester bond, an amide bond, an alkylene group, or a combination thereof. As the alkylene group, a linear or branched alkylene group is more preferable, and a methylene group or an ethylene group is still more preferable.

Specific preferred examples of the anion moiety in the component (d1-3) are described below.

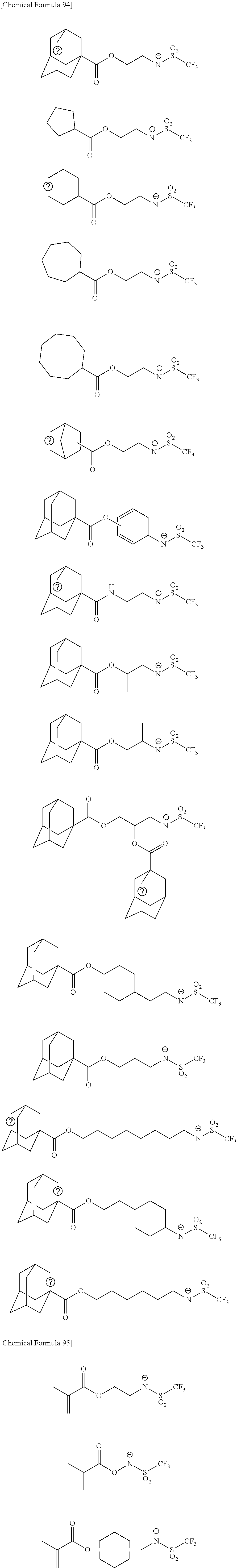

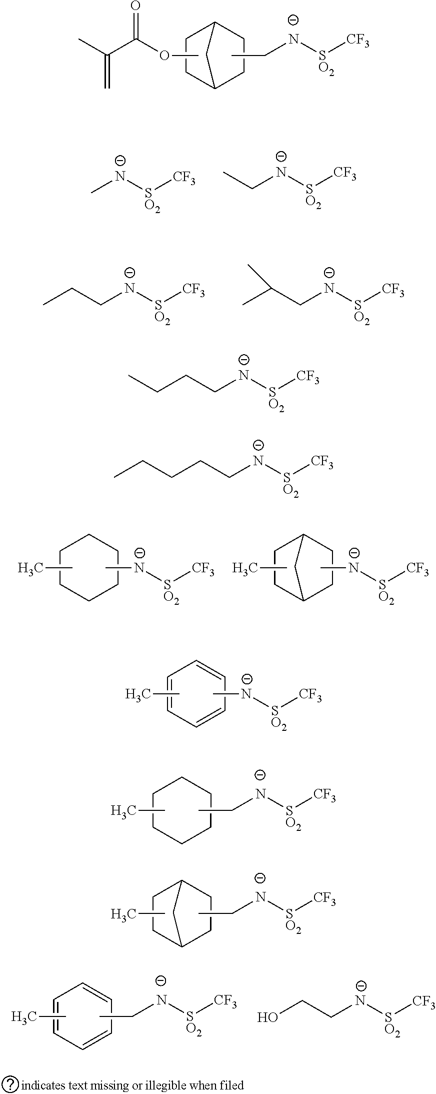

### Cation Moiety

In Formula (d1-3), M^{m+} represents an m-valent organic cation and has the same definition as that for M^{m+} in Formula (d1-1).

The component (d1-3) may be used alone or in combination of two or more kinds thereof.

As the component (D1), only any one of the above-described components (d1-1) to (d1-3) or a combination of two or more kinds thereof may be used.

In a case where the resist composition contains the component (D1), the content of the component (D1) in the resist composition is preferably in a range of 0.5 to 15 parts by mass, more preferably in a range of 1 to 12 parts by mass, and still more preferably in a range of 2 to 10 parts by mass with respect to 100 parts by mass of the component (A).

It is preferable that the component (D1) contain the component (d1-1).

The content of the component (d1-1) in the total component (D1) is preferably 50% by mass or more, preferably 70% by mass or more, and still more preferably 90% by mass or more, and the component (D1) may consist of only a compound for the component (d1-1).

### Method of Producing Component (D1):

The methods of producing the component (d1-1) and the component (d1-2) are not particularly limited, and these components can be produced by known methods.

Further, the method of producing the component (d1-3) is not particularly limited, and the component is produced by the same method as disclosed in United States Patent Application, Publication No. 2012-0149916.

### In Regard to Component (D2)

The component (D) may contain a nitrogen-containing organic compound component (hereinafter, referred to as a “component (D2)”) which does not correspond to the component (D1).

The component (D2) is not particularly limited as long as it acts as an acid diffusion control agent and does not correspond to the component (D1), and any known compound may be used. Among the examples, an aliphatic amine is preferable, and particularly a secondary aliphatic amine and a tertiary aliphatic amine are more preferable.

The aliphatic amine is an amine containing one or more aliphatic groups, and the number of carbon atoms in the aliphatic group is preferably in a range of 1 to 12.

Examples of the aliphatic amine include amines in which at least one hydrogen atom of ammonia NH₃ has been substituted with an alkyl group or hydroxyalkyl group having 12 or less carbon atoms (alkylamines or alkylalcoholamines), and cyclic amines.

Specific examples of the alkylamines and the alkylalcoholamines include monoalkylamines such as n-hexylamine, n-heptylamine, n-octylamine, n-nonylamine, and n-decylamine; dialkylamines such as diethylamine, di-n-propylamine, di-n-heptylamine, di-n-octylamine, and dicyclohexylamine; trialkylamines such as trimethylamine, triethylamine, tri-n-propylamine, tri-n-butylamine, tri-n-pentylamine, tri-n-hexylamine, tri-n-heptylamine, tri-n-octylamine, tri-n-nonylamine, tri-n-decylamine, and tri-n-dodecylamine; and alkylalcoholamines such as diethanolamine, triethanolamine, diisopropanolamine, triisopropanolamine, di-n-octanolamine, and tri-n-octanolamine. Among these, a trialkylamine having 6 to 30 carbon atoms is still more preferable, and tri-n-pentylamine or tri-n-octylamine is particularly preferable.

Examples of the cyclic amine include a heterocyclic compound having a nitrogen atom as a heteroatom. The heterocyclic compound may be a monocyclic compound (aliphatic monocyclic amine) or a polycyclic compound (aliphatic polycyclic amine).

Specific examples of the aliphatic monocyclic amine include piperidine and piperazine.

It is preferable that the aliphatic polycyclic amine have 6 to 10 carbon atoms, and specific examples thereof include 1,5-diazabicyclo[4.3.0]-5-nonene, 1,8-diazabicyclo[5.4.0]-7-undecene, hexamethylenetetramine, and 1,4-diazabicyclo[2.2.2]octane.

Examples of other aliphatic amines include tris(2-methoxymethoxyethyl)amine, tris{2-(2-methoxyethoxy)ethyl}amine, tris{2-(2-methoxyethoxymethoxy)ethyl}amine, tris{2-(1-methoxyethoxy)ethyl}amine, tris{2-(1-ethoxyethoxy)ethyl}amine, tris{2-(1-ethoxypropoxy)ethyl}amine, tris[2-(2-(2-hydroxyethoxy)ethoxy)ethyl]amine, and triethanolamine triacetate, and triethanolamine triacetate and 2,6-di-tert-butylpyridine are preferable.

As the component (D2), an aromatic amine may be used.

Examples of the aromatic amine include 4-dimethylaminopyridine, pyrrole, indole, pyrazole, imidazole, and derivatives thereof, tribenzylamine, 2,6-diisopropylaniline. N-tert-butoxycarbonylpyrrolidine, 2,6-di-tert-butylpyridine, and 2,6-tert-butylpyridine.

The component (D2) may be used alone or in combination of two or more kinds thereof.

In a case where the resist composition contains the component (D2), the content of the component (D2) in the resist composition is typically in a range of 0.01 to 5 parts by mass with respect to 100 parts by mass of the component (A). In a case where the content thereof is set to be in the above-described range, the resist pattern shape, the post-exposure temporal stability, and the like are improved.

<<At Least One Compound (E) Selected from Group Consisting of Organic Carboxylic Acids, Phosphorus Oxo Acids, and Derivatives Thereof>>

For the purpose of preventing any deterioration in sensitivity and improving the resist pattern shape, the post-exposure temporal stability, and the like, the resist composition according to the present embodiment may contain, as an optional component, at least one compound (E) (hereinafter referred to as “component (E)”) selected from the group consisting of an organic carboxylic acid, and a phosphorus oxo acid and a derivative thereof.

Specific examples of the organic carboxylic acid include acetic acid, malonic acid, citric acid, malic acid, succinic acid, benzoic acid, and salicylic acid. Among these, salicylic acid is preferable.

Examples of the phosphorus oxo acid include phosphoric acid, phosphonic acid, and phosphinic acid. Among these, phosphonic acid is particularly preferable.

In the resist composition according to the present embodiment, the component (E) may be used alone or in combination of two or more kinds thereof.

In a case where the resist composition contains the component (E), the content of the component (E) is preferably in a range of 0.01 to 5 parts by mass and more preferably in a range of 0.05 to 3 parts by mass with respect to 100 parts by mass of the component (A). Within the above range, the lithography characteristics are further improved.

### <<Fluorine Additive Component (F)>>

The resist composition according to the present embodiment may further contain a fluorine additive component (hereinafter, referred to as “component (F)”) as a hydrophobic resin. The component (F) is used to impart water repellency to the resist film and used as a resin different from the component (A), whereby the lithography characteristics can be improved.

As the component (F), for example, the fluorine-containing polymer compounds described in Japanese Unexamined Patent Application, First Publication Nos. 2010-002870, 2010-032994, 2010-277043, 2011-13569, and 2011-128226 can be used.

Specific examples of the component (F) include a polymer having a constitutional unit (f1) represented by General Formula (f1-1). As the polymer, a polymer (homopolymer) formed of only the constitutional unit (f1) represented by Formula (f1-1) a copolymer of the constitutional unit (f1) and the constitutional unit (a1); or a copolymer of the constitutional unit (f1), a constitutional unit derived from acrylic acid or methacrylic acid, and the constitutional unit (a1) is preferable, and a copolymer of the constitutional unit (f1) and the constitutional unit (a1) is more preferable. Here, as the constitutional unit (a1) copolymerized with the constitutional unit (f1), a constitutional unit derived from 1-ethyl-1-cyclooctyl (meth)acrylate or a constitutional unit derived from 1-methyl-1-adamantyl (meth)acrylate is preferable, and a constitutional unit derived from 1-ethyl-1-cyclooctyl (meth)acrylate is more preferable.

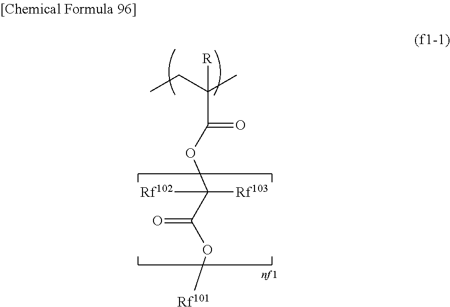

[In the formula, R has the same definition as described above, Rf¹⁰² and Rf¹⁰³ each independently represents a hydrogen atom, a halogen atom, an alkyl group having 1 to 5 carbon atoms, or a halogenated alkyl group having 1 to 5 carbon atoms, and Rf¹⁰² and Rf¹⁰³ may be the same as or different from each other. nf¹ represents an integer of 0 to 5, and Rf¹⁰¹ represents an organic group having a fluorine atom.]

In Formula (f1-1). R bonded to the carbon atom at the α-position has the same definition as described above. It is preferable that R represent a hydrogen atom or a methyl group.

In Formula (f1-1), a fluorine atom is preferable as the halogen atom as Rf¹⁰² and Rf¹⁰³. Examples of the alkyl group having 1 to 5 carbon atoms as Rf¹⁰² and Rf¹⁰³ include the same groups as those for the alkyl group having 1 to 5 carbon atoms as R. Among the examples, a methyl group or an ethyl group is preferable. Specific examples of the halogenated alkyl group having 1 to 5 carbon atoms as Rf¹⁰² and Rf¹⁰³ include groups in which some or all hydrogen atoms of an alkyl group having 1 to 5 carbon atoms have been substituted with halogen atoms. Among these, a fluorine atom is preferable as the halogen atom. Among these, Rf¹⁰² and Rf¹⁰³ preferably represent a hydrogen atom, a fluorine atom, or an alkyl group having 1 to 5 carbon atoms, more preferably a hydrogen atom, a fluorine atom, a methyl group, or an ethyl group, and still more preferably a hydrogen atom.

In Formula (f1-1), nf¹ represents an integer of 0 to 5, preferably an integer of 0 to 3, and more preferably 1 or 2.

In Formula (f1-1), Rf¹⁰¹ represents an organic group having a fluorine atom and preferably a hydrocarbon group having a fluorine atom.

The hydrocarbon group having a fluorine atom may be linear, branched, or cyclic, and the number of carbon atoms thereof is preferably in a range of 1 to 20, more preferably in a range of 1 to 15, and particularly preferably in a range of 1 to 10.

In the hydrocarbon group having a fluorine atom, preferably 25% or greater of the hydrogen atoms in the hydrocarbon group are fluorinated, more preferably 50% or greater thereof are fluorinated, and particularly preferably 60% or greater thereof are fluorinated from the viewpoint of increasing the hydrophobicity of the resist film during immersion exposure.

Among the examples, Rf¹⁰¹ more preferably represents a fluorinated hydrocarbon group having 1 to 6 carbon atoms and particularly preferably a trifluoromethyl group, —CH₂—CF₃, —CH₂—CF₂—CF₃, —CH(CF₃)₂, —CH₂—CH₂—CF₃, or —CH₂—CH₂—CF₂—CF₂—CF₂—CF₃.

The weight-average molecular weight (Mw) (in terms of polystyrene according to gel permeation chromatography) of the component (F) is preferably in a range of 1000 to 50000, more preferably in a range of 5000 to 40000, and most preferably in a range of 10000 to 30000. In a case where the weight-average molecular weight thereof is less than or equal to the upper limits of the above-described ranges, the resist composition exhibits a satisfactory solubility in a solvent for a resist enough to be used as a resist. Meanwhile, in a case where the weight-average molecular weight thereof is greater than or equal to the lower limits of the above-described ranges, water repellency of the resist film is satisfactory.

Further, the dispersity (Mw/Mn) of the component (F) is preferably in a range of 1.0 to 5.0, more preferably in a range of 1.0 to 3.0, and most preferably in a range of 1.0 to 2.5.

In the resist composition according to the present embodiment, the component (F) may be used alone or in combination of two or more kinds thereof.

In a case where the resist composition contains the component (F), the content of the component (F) is preferably in a range of 0.5 to 10 parts by mass and more preferably in a range of 1 to 10 parts by mass with respect to 100 parts by mass of the component (A).

### <<Organic Solvent Component (S)>>

The resist composition according to the present embodiment may be produced by dissolving the resist materials in an organic solvent component (hereinafter, referred to as a “component (S)”).

In the resist composition according to the present embodiment, the component (S) may be used alone or in the form of a mixed solvent of two or more kinds thereof. Among these, propylene glycol monomethyl ether acetate (PGMEA), propylene glycol monomethyl ether (PGME), γ-butyrolactone, ethyl lactate (EL), or cyclohexanone is preferable.

Further, a mixed solvent obtained by mixing PGMEA with a polar solvent is also preferable as the component (S). The blending ratio (mass ratio) may be appropriately determined in consideration of the compatibility or the like between PGMEA and the polar solvent.

A mixed solvent of at least one selected from PGMEA and EL, and γ-butyrolactone is also preferable as the (S) component. In this case, as the mixing ratio, the mass ratio between the former and the latter is preferably in a range of 70:30 to 95:5.

The amount of the component (S) to be used is not particularly limited and is appropriately set to have a concentration which enables coating a substrate or the like depending on the thickness of the coated film. The component (S) is typically used in an amount such that the solid content concentration of the resist composition is set to be in a range of 0.1% to 20% by mass and preferably in a range of 0.2% to 15% by mass.

After the resist material is dissolved in the component (S), impurities and the like may be removed from the resist composition according to the present embodiment using a porous polyimide film, a porous polyamideimide film, or the like. For example, the resist composition may be filtered using a filter formed of a porous polyimide film, a filter formed of a porous polyamideimide film, a filter formed of a porous polyimide film and a porous polyamideimide film, or the like. Examples of the porous polyimide film and the porous polyamideimide film include those described in Japanese Unexamined Patent Application, First Publication No. 2016-155121.

The resist composition according to the present embodiment described above contains the resin component (A1) having the constitutional unit (a0) represented by General Formula (a0-1).

The constitutional unit (a0) has a group that generates an acid upon exposure, and a cyclic ether structure as a structure constituting a main chain. The cyclic ether structure as a structure constituting the main chain has appropriate polarity, and the resin component (A1) whose solubility in a developing solution is improved. In addition, in a case where the acid generator component is bonded to the resin component (A1), the diffusion of the acid in the exposed portion is suppressed. As a result, it is presumed that the sensitivity is favorably maintained and the resolution is improved. Furthermore, since the structure constituting the main chain forms a cyclic ether structure, the constitutional unit (a0) is appropriately bulky. Therefore, it is considered that the constitutional unit (a0) is appropriately dispersed and disposed in the resin component (A1). As a result, it is presumed that the roughness is improved.

### (Method for Forming Resist Pattern)

A method for forming a resist pattern according to the second aspect according to the present invention is a method including a step of forming a resist film on a support using the resist composition according to the first aspect of the present invention described above, a step of exposing the resist film to light, and a step of developing the resist film exposed to light to form a resist pattern.

According to the embodiment of the method for forming a resist pattern, a resist pattern formation method by performing processes as described below is an exemplary example.

First, a support is coated with the resist composition according to the present embodiment using a spinner or the like, and a bake (post-apply bake (PAB)) treatment is performed, for example, under a temperature condition of 80° C. to 150° C. for 40 to 120 seconds and preferably 60 to 90 seconds to form a resist film.

Following the selective exposure carried out on the resist film by, for example, exposure through a mask (mask pattern) having a predetermined pattern formed on the mask using an exposure apparatus such as an electron beam lithography apparatus or an ArF exposure apparatus, or direct irradiation of the resist film for drawing or the like with an electron beam without using a mask pattern, a bake treatment (post-exposure bake (PEB)) is carried out, for example, under a temperature condition in a range of 80° C. to 150° C. for 40 to 120 seconds and preferably 60 to 90 seconds.

Next, the resist film is subjected to a developing treatment. The developing treatment is conducted using an alkali developing solution in a case of an alkali developing process and using a developing solution containing an organic solvent (organic developing solution) in a case of a solvent developing process.

After the developing treatment, it is preferable to conduct a rinse treatment. As the rinse treatment, water rinsing using pure water is preferable in a case of the alkali developing process, and rinsing using a rinse solution containing an organic solvent is preferable in a case of the solvent developing process.

In a case of the solvent developing process, after the developing treatment or the rinse treatment, the developing solution or the rinse solution attached onto the pattern may be removed by treatment using a supercritical fluid.

After the developing treatment or the rinse treatment, drying is conducted. A bake treatment (post bake) may be conducted after the developing treatment in some cases.

The support is not particularly limited and a known support of the related art can be used, and examples thereof include a substrate for an electronic component and a substrate on which a predetermined wiring pattern has been formed. Specific examples thereof include a metal substrate such as a silicon wafer, copper, chromium, iron, or aluminum; and a glass substrate. As the materials of the wiring pattern, for example, copper, aluminum, nickel, or gold can be used.

The wavelength used for light exposure is not particularly limited and light exposure can be conducted using radiation such as an ArF excimer laser, a KrF excimer laser, an F₂ excimer laser, extreme ultraviolet rays (EUV), vacuum ultraviolet rays (VUV), electron beams (EB), X-rays, and soft X-rays.

The resist pattern formation method according to the present embodiment is particularly suitably used for a method in which the resist film is exposed to extreme ultraviolet (EUV) rays or electron beams (EB) in the step of exposing the resist film to light.

The method of exposing the resist film to light may be general exposure (dry exposure) conducted in air or an inert gas such as nitrogen, or liquid immersion lithography.

The liquid immersion lithography is an exposure method in which the region between the resist film and the lens at the lowermost position of the exposure apparatus is filled with a solvent (liquid immersion medium) in advance that has a refractive index greater than the refractive index of air, and the exposure (immersion exposure) is conducted in this state.

As the liquid immersion medium, a solvent having a refractive index greater than the refractive index of air but less than the refractive index of the resist film to be exposed is preferable, and examples thereof include water, a fluorine-based inert liquid, a silicon-based solvent, and a hydrocarbon-based solvent.

As the liquid immersion medium, water is preferably used.

As the alkali developing solution used for the developing treatment in the alkali developing process, a 0.1 to 10 mass % tetramethylammonium hydroxide (TMAH) aqueous solution is an exemplary example.

The organic solvent contained in the organic developing solution used for the developing treatment in the solvent developing process may be any solvent that is capable of dissolving the component (A) (the component (A) before light exposure) and can be appropriately selected from known organic solvents. Specific examples thereof include a polar solvent such as a ketone-based solvent, an ester-based solvent, an alcohol-based solvent, a nitrile-based solvent, an amide-based solvent, and an ether-based solvent, and a hydrocarbon-based solvent.

Examples of the ester-based solvent include methyl acetate, butyl acetate, ethyl acetate, isopropyl acetate, pentyl acetate, isopentyl acetate, amyl acetate, propylene glycol monomethyl ether acetate, ethylene glycol monoethyl ether acetate, diethylene glycol monobutyl ether acetate, diethylene glycol monoethyl ether acetate, ethyl-3-ethoxypropionate, 3-methoxybutyl acetate, 3-methyl-3-methoxybutyl acetate, methyl formate, ethyl formate, butyl formate, propyl formate, ethyl lactate, butyl lactate, propyl lactate, butyl butanoate, methyl 2-hydroxyisobutyrate, isoamyl acetate, isobutyl isobutyrate, and butyl propionate.

Examples of the nitrile-based solvent include acetonitrile, propionitrile, valeronitrile, and butyronitrile.

Known additives can be blended into the organic developing solution as necessary. Examples of the additive include a surfactant. The surfactant is not particularly limited, and for example, an ionic or non-ionic fluorine-based and/or silicon-based surfactant can be used.

The developing treatment can be performed according to a known developing method, and examples thereof include a method of immersing a support in a developing solution for a certain time (a dip method), a method of raising a developing solution on the surface of a support using surface tension and maintaining the state for a certain time (a puddle method), a method of spraying a developing solution on the surface of a support (spray method), and a method of continuously ejecting a developing solution onto a support rotating at a certain rate while scanning a developing solution ejection nozzle at a certain rate (dynamic dispense method).

As the organic solvent contained in the rinse solution used for the rinse treatment after the developing treatment in the solvent developing process, for example, a solvent that is unlikely to dissolve a resist pattern can be appropriately selected from the organic solvents described as the organic solvent used in the organic developing solution and then used. Typically, at least one solvent selected from a hydrocarbon-based solvent, a ketone-based solvent, an ester-based solvent, an alcohol-based solvent, an amide-based solvent, and an ether-based solvent is used.

These organic solvents may be used alone or in combination of two or more kinds thereof. Further, an organic solvent other than the above-described solvents and water may be mixed and used.

The rinse treatment carried out using a rinse solution (washing treatment) can be performed according to a known rinse method. Examples of the method of performing the rinse treatment include a method of continuously ejecting a rinse solution onto a support rotating at a certain rate (rotary coating method), a method of immersing a support in a rinse solution for a certain time (dip method), and a method of spraying a rinse solution on the surface of a support (spray method).

According to the resist pattern formation method according to the present embodiment described above, since the above-described resist composition is used, sensitivity is satisfactorily maintained in a case of forming a resist pattern, and a resist pattern having a good shape in which lithography characteristics such as roughness reduction and resolution improvement of the resist pattern are further enhanced can be formed.

It is preferable that the various materials that are used in the resist composition according to the above-described embodiment and the pattern formation method according to the above-described embodiment (for example, a resist solvent, a developing solution, a rinse solution, a composition for forming an antireflection film, and a composition for forming a top coat) not contain impurities such as a metal, a metal salt containing halogen, an acid, an alkali, and a component having a sulfur atom or phosphorus atom. Here, examples of the impurities containing metal atoms include Na, K, Ca, Fe, Cu, Mn, Mg, Al, Cr, Ni, Zn, Ag, Sn, Pb, Li, and salts thereof. The content of the impurities contained in these materials is preferably 200 ppb or less, more preferably 1 ppb or less, still more preferably 100 parts per trillion (ppt) or less, and particularly preferably 10 ppt or less, where it is most preferable that the impurities be substantially free (less than or equal to the detection limit of the measuring device).

### (Compound)

A compound according to a third aspect of the present invention is a compound represented by General Formula (m0-1) (hereinafter, also referred to as “compound (M0)”).

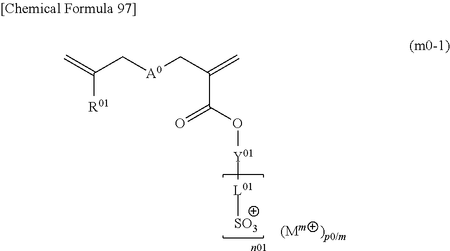

[In the formula, A⁰ represents an oxygen atom or a sulfur atom. Y^{0′} represents an (n01+1)-valent linking group. L⁰¹ represents a single bond or a divalent linking group. R⁰¹ represents a hydrogen atom or a group represented by General Formula (r01-1) or (r01-2). n01 represents an integer of 1 or greater as long as the valence is allowed. m represents an integer of 1 or greater, and M^{m+} represents an m-valent cation. p0=n01 in a case where R⁰¹ is a hydrogen atom or a group represented by General Formula (r0-2), and p0=n01+n02 in a case where R⁰¹ is a group represented by General Formula (r01-1).]

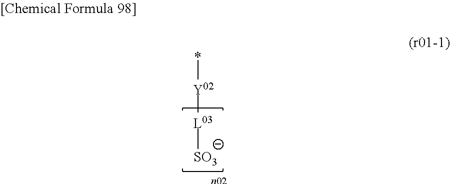

[In the formula, Y⁰² represents an (n02+1)-valent linking group. L⁰² represents a single bond or a divalent linking group. n02 represents an integer of 1 or greater as long as the valence is allowed.]

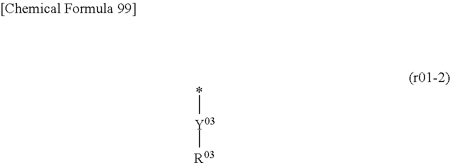

[In the formula. Y⁰³ represents a single bond or a divalent linking group. R⁰³ represents a hydrocarbon group which may have a substituent.]

In Formula (m0-1), A⁰, Y⁰¹, L⁰¹, R⁰¹, n01, M^{m+}, and p0 are each the same as A⁰, Y⁰¹, L⁰¹, R⁰¹, n01, M^{m+}, and p0 in Formula (a0-1).

Formula (r01-1) and Formula (r01-1) are each the same as Formula (a0-1).

Examples of the compound (M0) include a compound represented by General Formula (m01-1), (m02-1), or (m03-1).

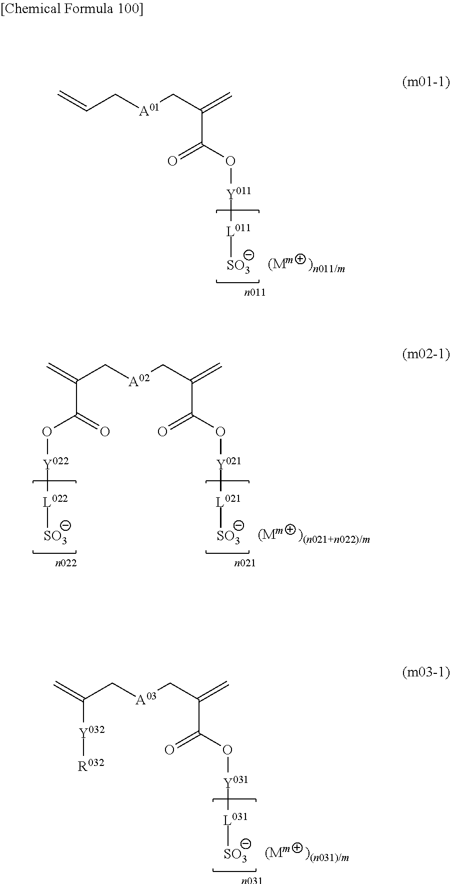

[In the formulae, A⁰¹ to A⁰³ each independently represents an oxygen atom or a sulfur atom. Y⁰¹¹ represents an (n011+1)-valent linking group. Y⁰²¹ represents an (n021+1)-valent linking group. Y⁰²² represents an (n022+1)-valent linking group. Y⁰³¹ represents an (n031+1)-valent linking group. L⁰¹¹, L⁰²¹, L⁰²², L⁰³¹, and Y⁰³² each independently represents a single bond or a divalent linking group. R⁰³² represents a hydrocarbon group which may have a substituent. n011, n021, n022, and n031 each independently represents an integer of 1 or greater as long as the valence is allowed. m represents an integer of 1 or greater, and M^{m+} represents an m-valent cation.

A⁰¹, A⁰², A⁰³, Y⁰¹¹, Y⁰²¹, Y⁰²², Y⁰³¹, L¹¹, L⁰²¹, L⁰²², L⁰³¹, Y⁰³², R⁰³², n011, n021, n022, n031, and M^{m+} in Formulae (m01-1), (m02-1), and (m03-1) are each the same as those in Formulae (a01-1), (a02-1), and (a03-1).

As the compound (M0), a compound represented by General Formula (m01-1-1), (m02-1-1), or (m03-1-1) is preferable.

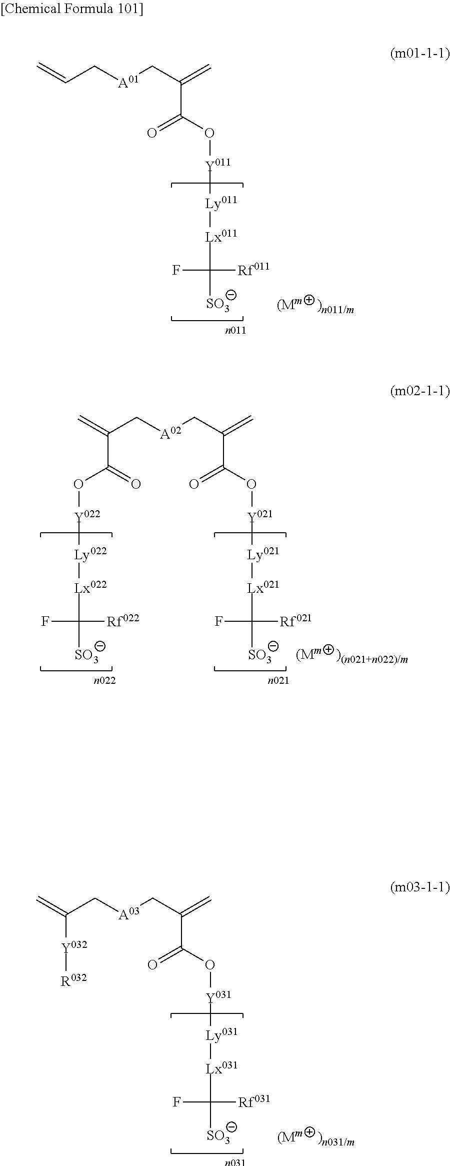

[In the formulae, A⁰¹ to A⁰³, Y⁰¹¹, Y⁰²¹, Y⁰²², Y⁰³¹, Y⁰³², R⁰³², n011, n021, n022, and n031 are each the same as those in Formulae (a01-1) to (a03-1). Ly⁰¹¹, Ly⁰²¹, Ly⁰²², and Ly⁰³¹ each independently represents a single bond or a divalent linking group. Lx⁰¹¹, Lx⁰²¹, L⁰²², and Lx⁰³¹ each independently represents a single bond, an alkylene group, or a fluorinated alkylene group. Rf⁰¹¹, R⁰²¹, Rf⁰²², and Rf⁰³¹ each independently represents a fluorinated alkyl group or a fluorine atom. In represents an integer of 1 or greater, and M^{m+} represents an m-valent cation.

A⁰¹, A⁰², A⁰³, Y⁰¹¹, Y⁰²¹, Y⁰²², Y⁰³¹, Y⁰³¹, R⁰³², n⁰¹¹, n⁰²¹, n⁰²², n⁰³¹, Ly⁰¹¹, Ly⁰²¹, Ly⁰²², Ly⁰³¹, Lx⁰¹¹, Lx⁰²¹, Lx⁰²², Lx⁰³¹, Rf⁰¹¹, Rf⁰²¹, Rf⁰²², Rf⁰³¹, and M^{m+} in Formulae (m01-1-1), (m02-1-1), and (m03-1-1) are each the same as those in Formulae (a01-1-1), (a02-1-1), and (a03-1-1).

Specific examples of the compound (M0) are shown below, but the compound (M0) is not limited thereto. In the following formula, M^{m+} represents an m-valent cation. m represents an integer of 1 or greater.]

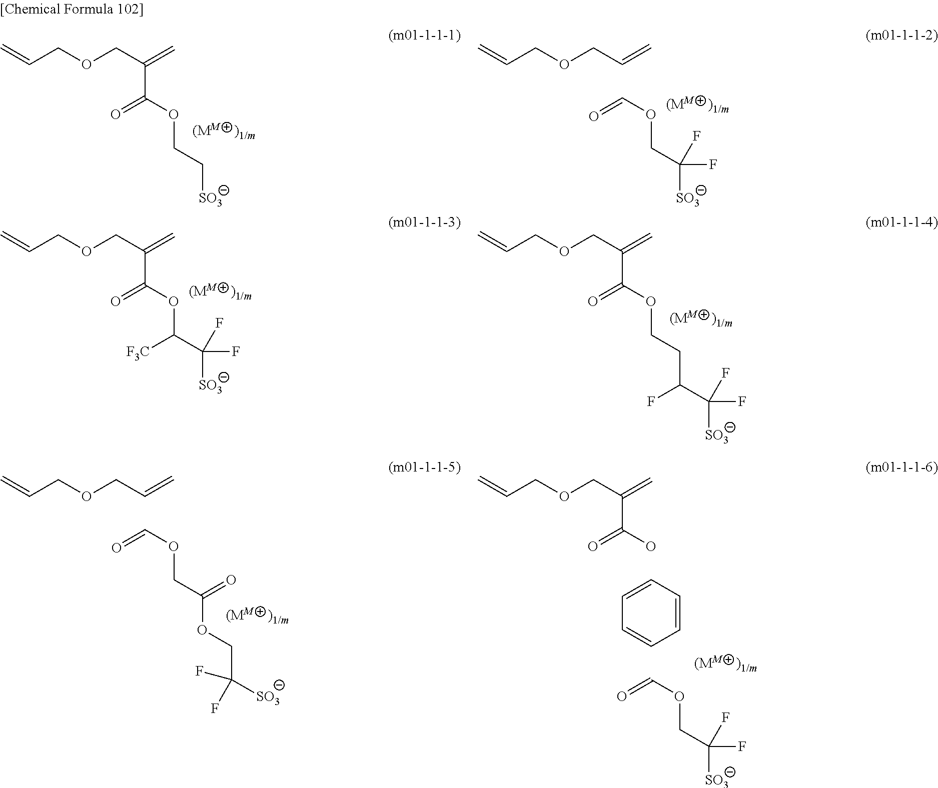

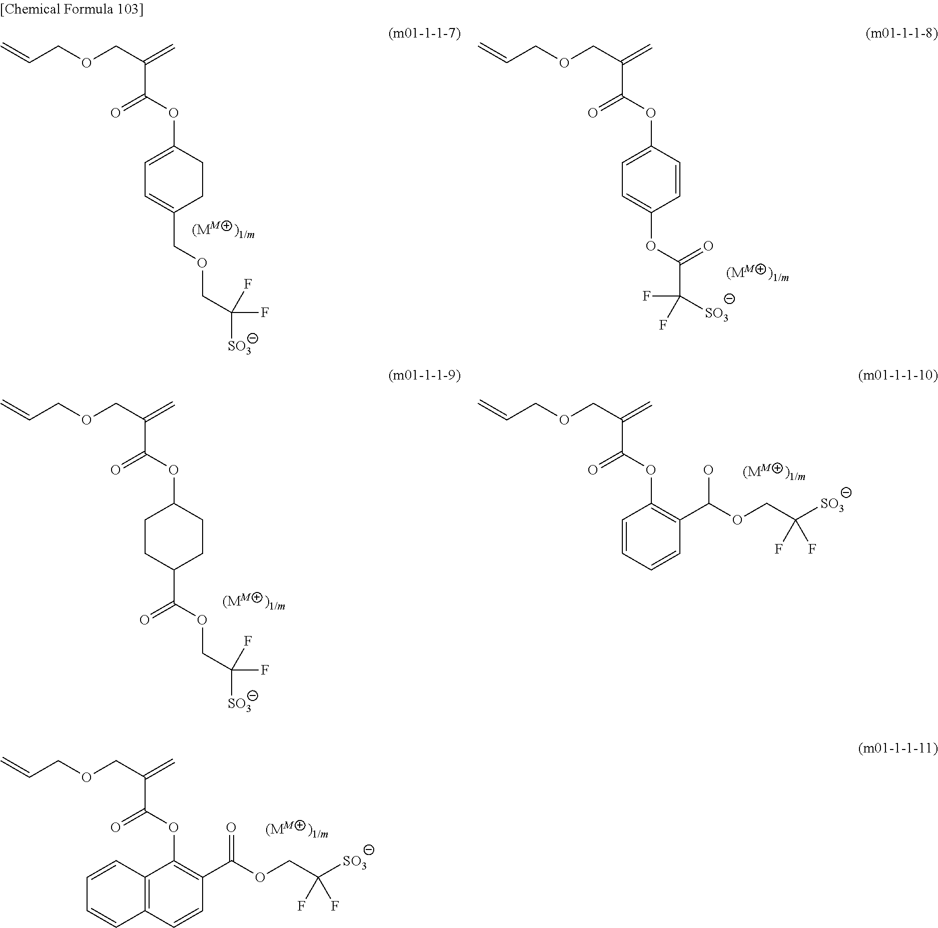

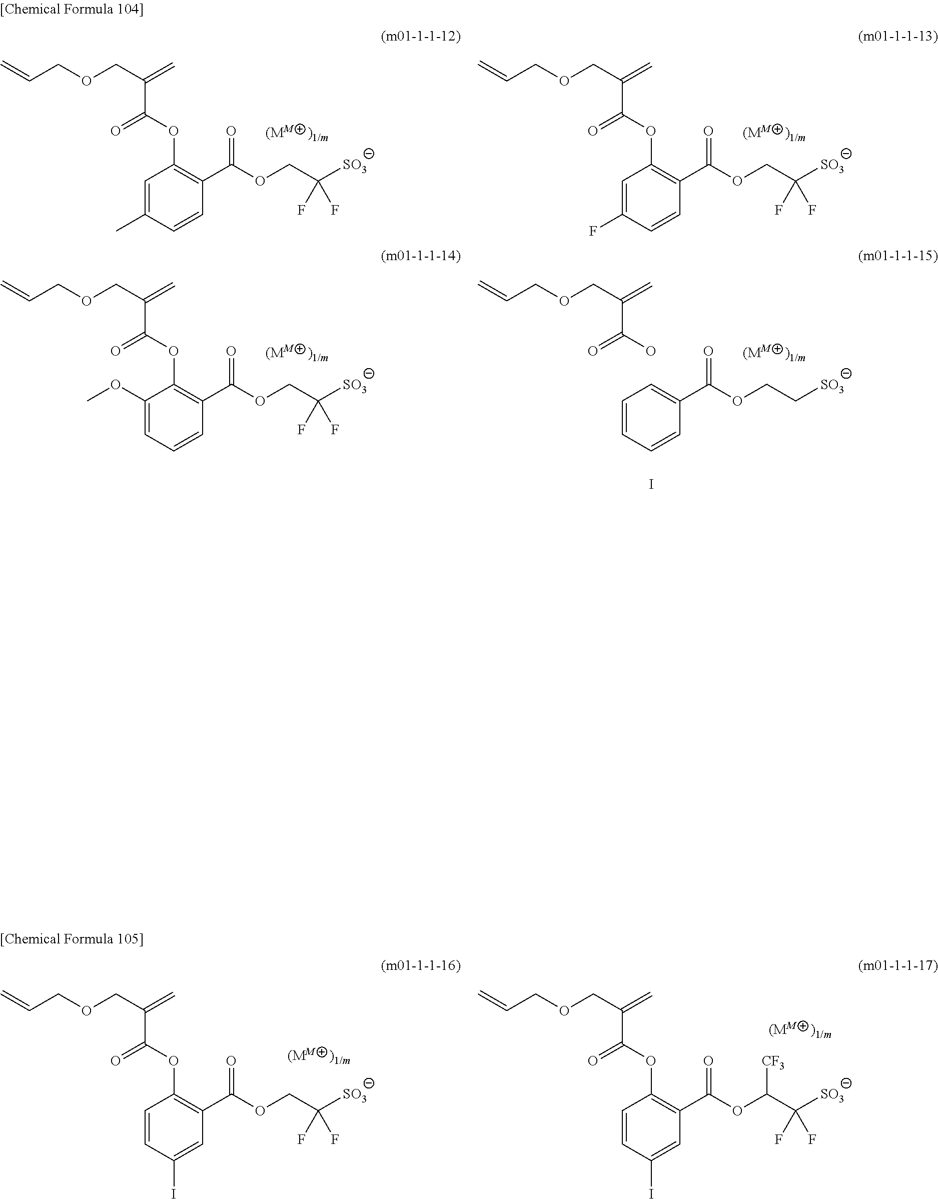

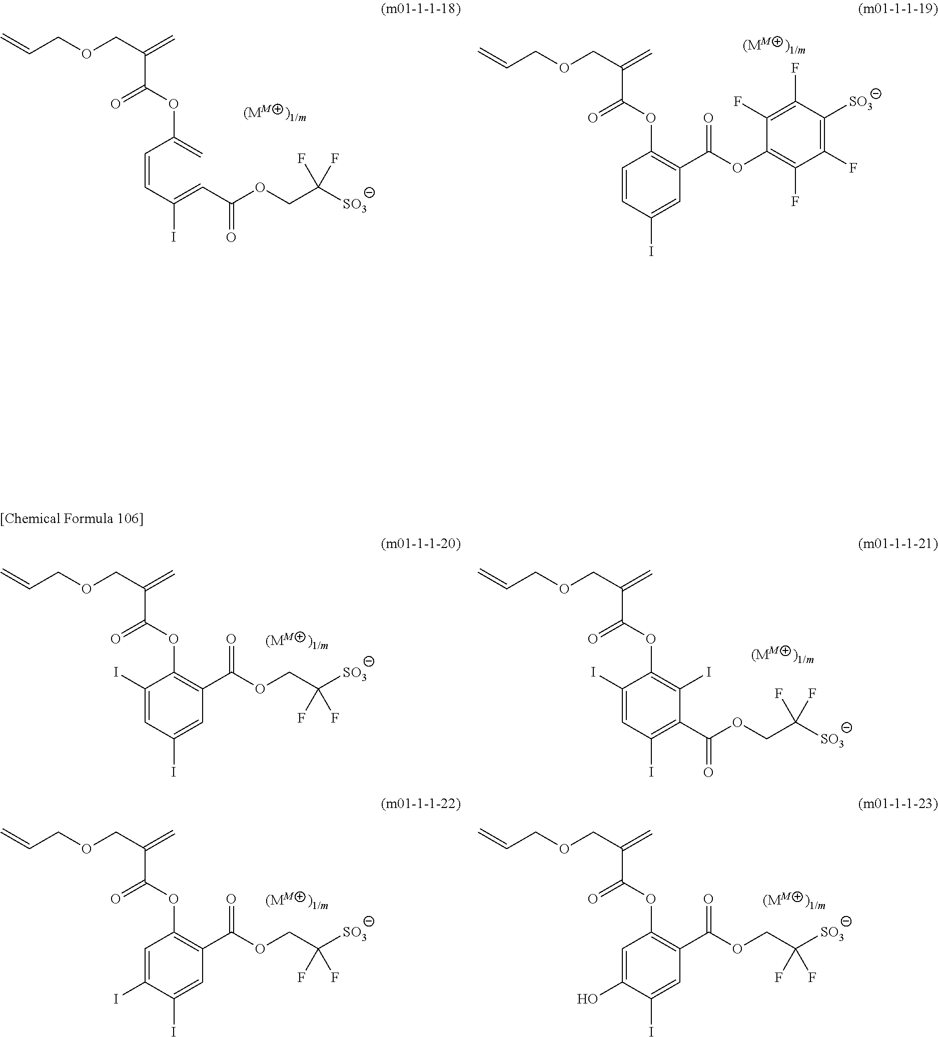

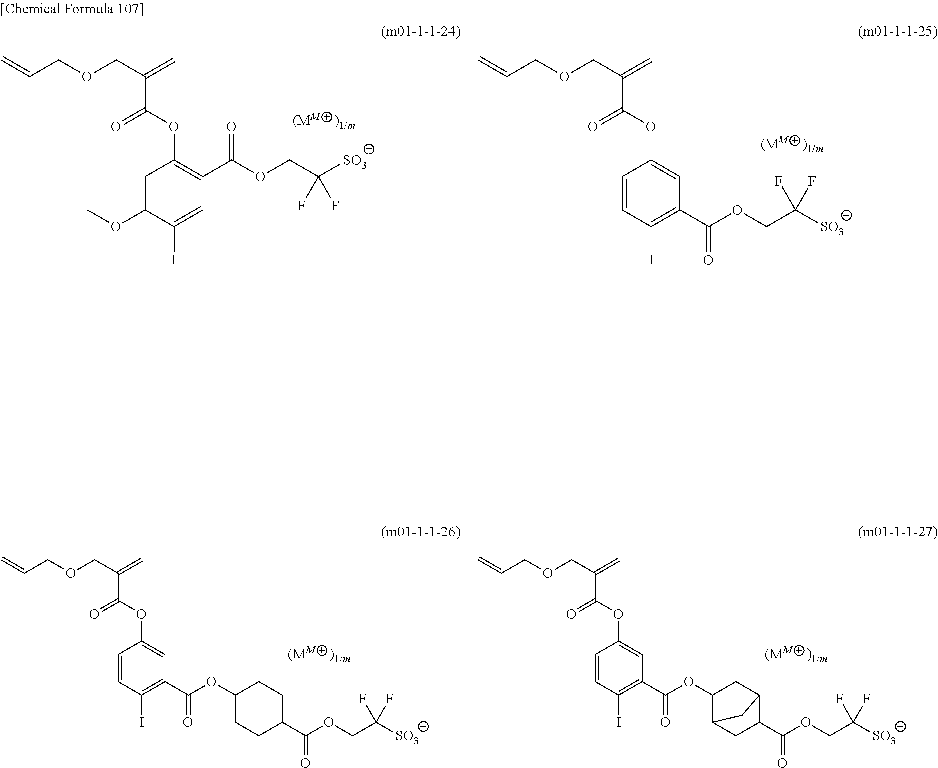

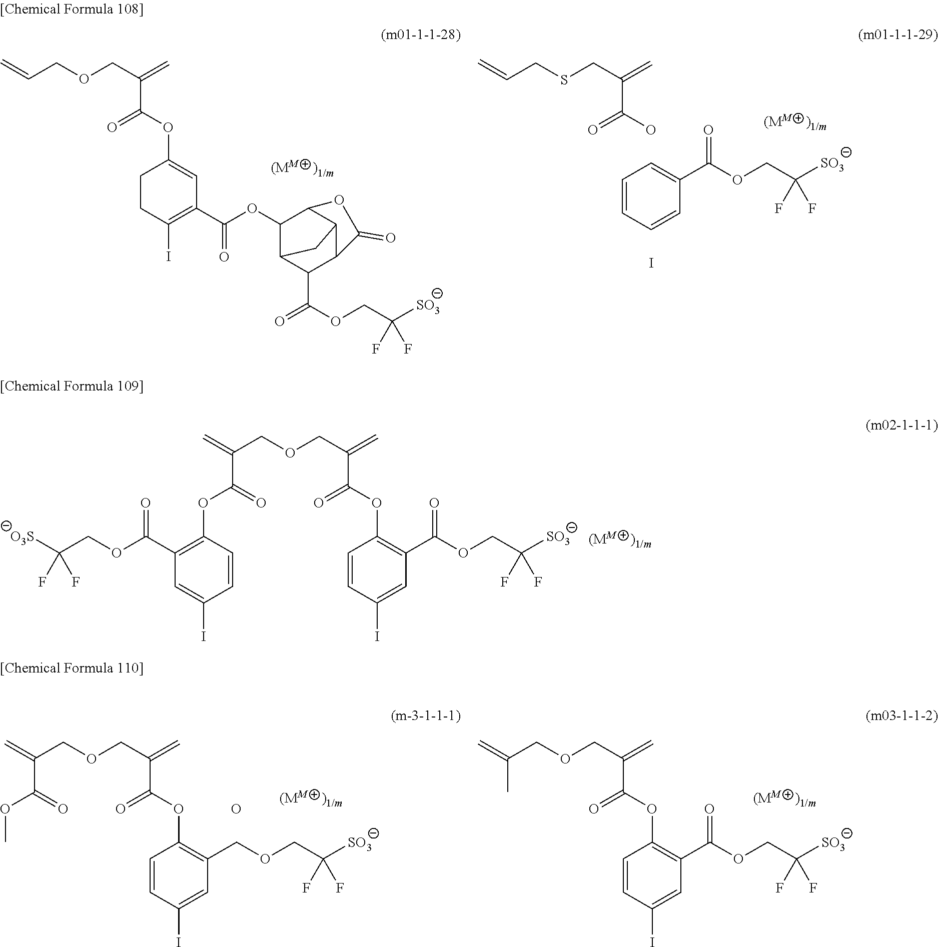

### <Method of Producing Compound (M0a)>

The compound (B0) can be produced by appropriately combining known methods as in <Synthesis example of compound> described later in Examples. The compound (M0) can be produced, for example, by the reactions (1) and (II) described later.

### <<Reaction (1)>>

In the reaction (1), for example, a compound represented by General Formula (m0-pre) is obtained by reacting a compound represented by General Formula (a) and a compound represented by General Formula (b-1). Alternatively, a compound represented by General Formula (m0-pre) is obtained by reacting a compound represented by General Formula (a), a compound represented by General Formula (b-1), and a compound represented by General Formula (b-2).

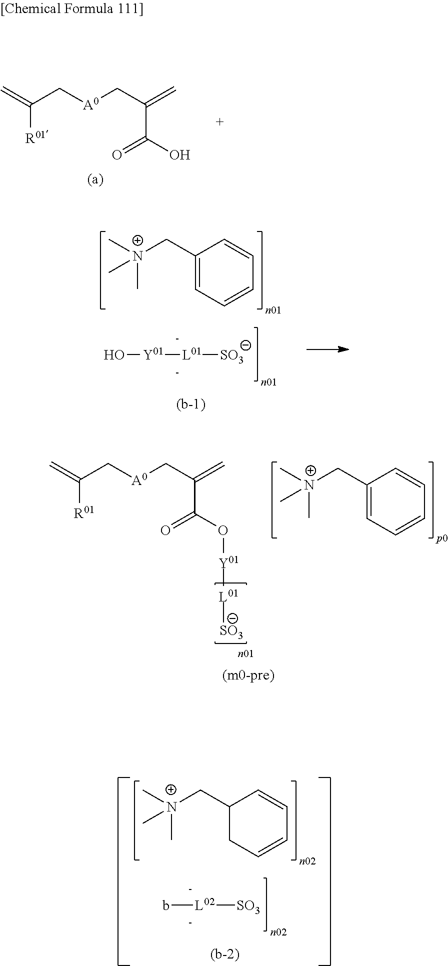

[in the reaction formula, A⁰, Y⁰¹, L⁰¹, R⁰¹, n01, and p0 are each the same as those in Formula (m0-1). R^{01′} represents a hydrogen atom, a group represented by Formula (r01-2), or a group capable of forming Y⁰² in Formula (r01-1) by a condensation reaction with b in General Formula (b-2). b represents an (n02+1)-valent linking group, and represents a group capable of forming Y⁰² in Formula (r01-1) by a condensation reaction with R⁰¹′.]

A⁰, Y⁰¹, L⁰¹, R⁰¹, and n01 in the reaction formulae are each the same as those in Formula (m0-1).

In a case where R⁰¹ represents a hydrogen atom or a group represented by Formula (r01-2), a compound represented by General Formula (m0-pre) is obtained by reacting the compound represented by General Formula (a) and the compound represented by General Formula (b-1).

In a case where R⁰¹ represents a hydrogen atom or a group represented by Formula (r01-2), a compound represented by General Formula (m0-pre) is obtained by reacting the compound represented by General Formula (a), the compound represented by General Formula (b-1), and the compound represented by General Formula (b-2).

### <<Reaction (II)>>

In the reaction (II), a compound (M0) is obtained by performing a salt exchange reaction between the compound represented by General Formula (m0-pre) and the compound represented by General Formula (c).

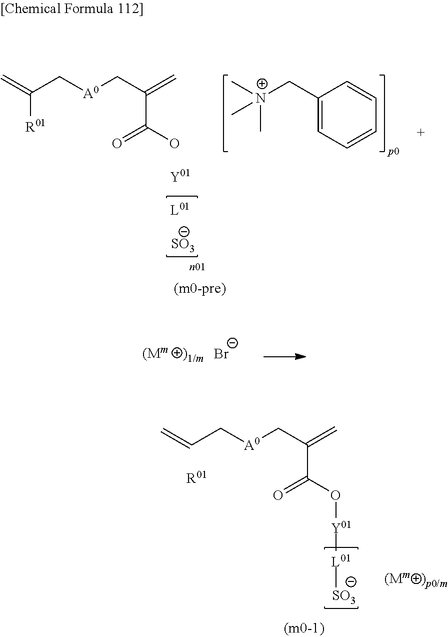

[in the reaction formula, A⁰, Y⁰¹, L⁰¹, R⁰¹, n01, M^{m+}, and p0 are each the same as those in Formula (m0-1)]

The temperature conditions for the reaction (I) are not particularly limited, and are, for example, about −10° C. to 120° C. preferably in a range of 0° C. to 100° C., and more preferably in a range of 10° C. to 70° C.

The reaction time of the reaction (1) is not particularly limited, and is, for example, about 1 to 72 hours and preferably in a range of 1 to 24 hours.

Examples of the reaction solvent used in the reaction (I) include dichloromethane, dichloroethane, chloroform, tetrahydrofuran, N,N-dimethylformamide, acetonitrile, propionitrile, N,N′-dimethylacetamide, and dimethyl sulfoxide.

The condensation reaction in the reaction (1) may be carried out in the presence of a condensing agent.

Specific examples of the condensing agent include N,N′-dicyclohexylcarbodiimide. N,N′-diisopropylcarbodiimide (DIC), 1-ethyl-3-(3-dimethylaminopropyl)carbodiimide hydrochloride, and carbonyldiimidazole (CDI).

A basic catalyst may be used in the reaction (I).

Specific examples of the basic catalyst include tertiary amines such as trimethylamine, triethylamine, and tributylamine, aromatic amines such as pyridine, dimethylaminopyridine (DMAP), and pyrrolidinopyridine, diazabicyclononene (DBN), and diazabicycloundecene (DBU).

The temperature conditions for the reaction (II) are not particularly limited, and are, for example, about 0° C. to 50° C.

The reaction time of the reaction (II) is not particularly limited, and is, for example, about 1 minute to 24 hours.

As the reaction solvent for the reaction (11), for example, a mixed solvent of an organic solvent and water is preferable. Examples of the organic solvent include a ketone-based solvent such as cyclohexanone, methyl ethyl ketone, or diethyl ketone, an ether-based solvent such as diethyl ether, t-butyl methyl ether, or diisopropyl ether, a halogen-based solvent such as tetrahydrofuran, 1,3-dioxolane, dichloromethane (methylene chloride), or 1,2-dichloroethane, an ester-based solvent such as ethyl acetate or propylene glycol monomethyl ether acetate, propionitrile, and a mixed solvent thereof.

After completion of the reaction (11), the compound (M0) in the reaction solution may be isolated and purified.

Known methods in the related art can be used for the isolation and purification, and for example, any one or a combination of two or more of concentration, solvent extraction, distillation, crystallization, recrystallization, chromatography, and the like can be used.

The structure of the compound obtained as described above can be identified by typical organic analysis methods such as ¹H-nuclear magnetic resonance (NMR) spectroscopy, ¹³C-NMR spectroscopy, ¹⁹F-NMR spectroscopy, infrared (IR) absorption spectroscopy, mass spectrometry (MS), elemental analysis, and X-ray crystal diffraction.

The compound according to the present embodiment can be used for producing a polymer compound according to a fourth aspect described below.

### (Polymer Compound)

The polymer compound according to a fourth aspect of the present invention is a polymer compound having a constitutional unit (a0) represented by General Formula (a0-1).

The polymer compound according to the fourth aspect is the same as the component (A1) described above.

The polymer compound according to the fourth aspect is a polymer compound having a constitutional unit derived from the compound (M0) according to the third aspect.

The polymer compound according to the fourth aspect can be used for producing the resist composition according to the first aspect.

### EXAMPLES

Hereinafter, the present invention will be described in more detail based on examples, but the present invention is not limited to these examples.

### Synthesis of Compounds

### Monomer Synthesis Example 1

7.1 g of the compound (a-1), 15.5 g of the compound (b-1), 11.3 g of diisopropylcarbodiimide (DIC), and a catalytic amount of dimethylaminopyridine (DMAP) were dissolved in 200 g of dichloromethane and stirred at room temperature for 6 hours. The insoluble matter in the obtained reaction solution was removed by filtration, and then the solvent was distilled off. The obtained residue was purified by silica gel column chromatography to obtain 15.3 g (yield: 70%) of a compound (M0-1pre).

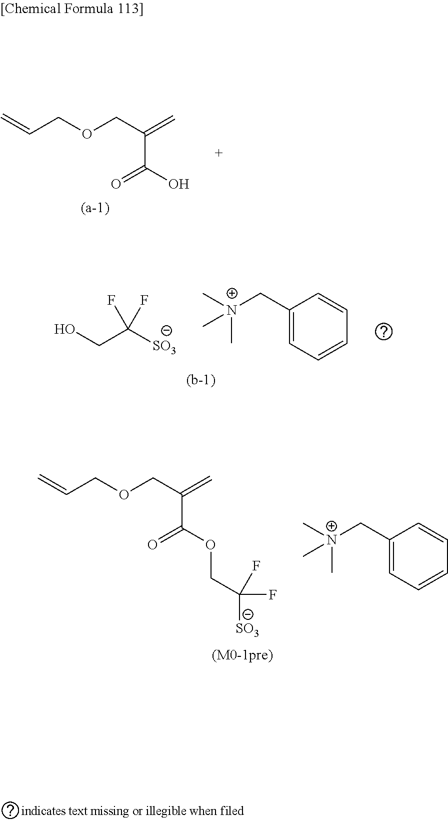

10.9 g of the compound (M0-1pre) and 9.4 g of triphenylsulfonium bromide were dissolved in 100 g of dichloromethane. 100 g of water was added thereto, the mixture was stirred at room temperature for 1 hour, and then the aqueous layer was removed. After repeating washing of the obtained organic layer with water, the solvent was distilled off and dried to obtain 13.0 g (yield: 95%) of a compound (M0-1).

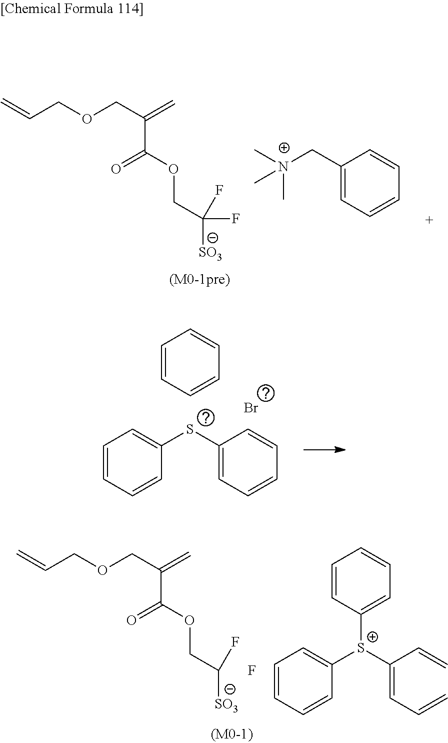

The obtained compound (M0-1) was subjected to NMR measurement, and the structure thereof was identified from the following analysis results.

¹H-NMR (dmso-d6, 400 MHz) δ (ppm)=7.7-7.9 (m, -Ph, 15H), 6.0-6.2 (m, =CH2+-CH═CH2, 2H), 5.7 (s, ═CH2, 1H), 5.1-5.4 (m, —CH═CH2, 2H), 4.6-4.7 (m, —CH2-, 2H), 4.0-4.2 (m, —CH2-, 4H)

### Monomer Synthesis Examples 2 to 11

Compounds (M0-2) to (M0-11) were obtained by the same method as in synthesis example 1, except that the compound (a-1) and triphenylsulfonium bromide were each changed to the corresponding compounds.

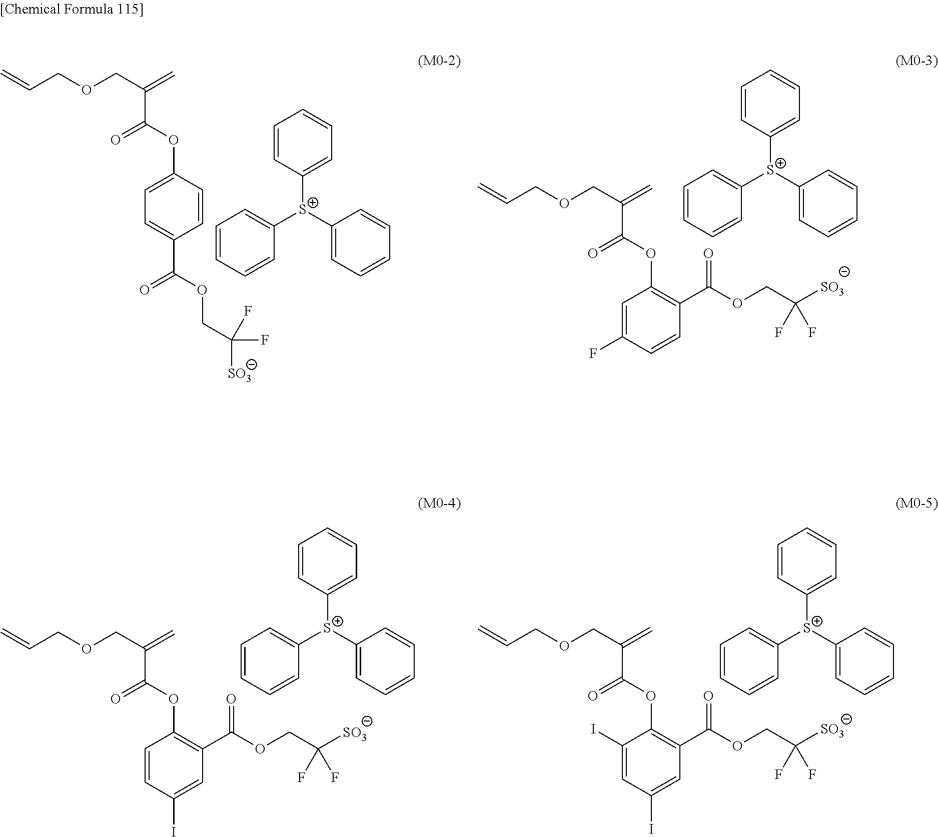

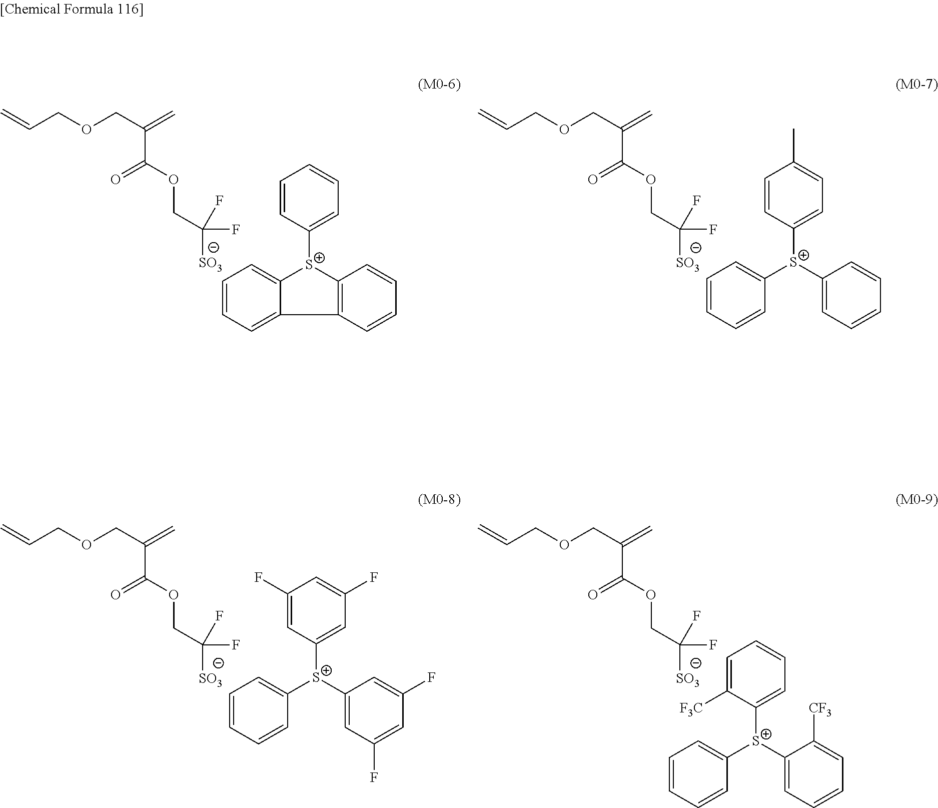

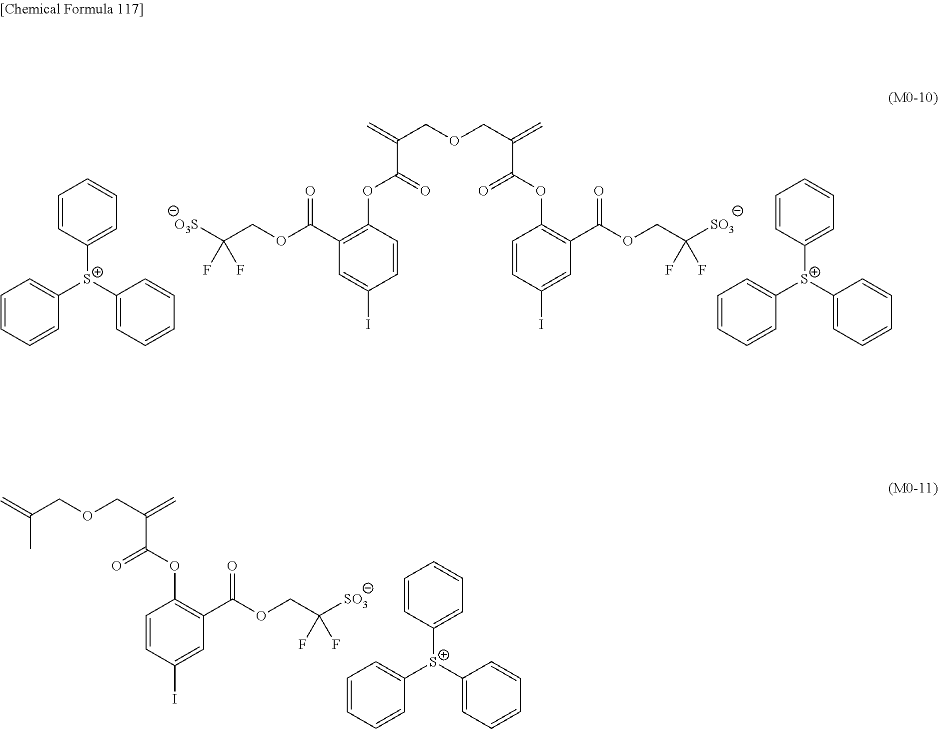

The obtained compound (M0-2) was subjected to NMR measurement, and the structure thereof was identified from the following analysis results.

¹H-NMR (dmso-d6, 400 MHz) δ (ppm)=8.0 (m, —Ar, 2H), 7.7-7.9 (m, -Ph, 15H), 7.2 (m, —Ar, 2H), 6.0-6.2 (m, =CH2+-CH═CH2, 2H), 5.7 (s, ═CH2, 1H), 5.1-5.4 (m, —CH═CH2, 2H), 4.6-4.7 (m, —CH2-, 2H), 4.0-4.2 (m, —CH2-, 4H)

The obtained compound (M0-3) was subjected to NMR measurement, and the structure thereof was identified from the following analysis results.

¹H-NMR (dmso-d6, 400 MHz) δ (ppm)=8.1 (m, —Ar, 1H), 7.7-7.9 (m, -Ph, 15H), 7.4 (m, —Ar, 1H), 7.2 (m, —Ar, 1H), 6.0-6.2 (m, =CH2+-CH═CH2, 2H), 5.7 (s. ═CH2, 1H), 5.1-5.4 (m, —CH═CH2, 2H), 4.6-4.7 (m, —CH2-, 2H), 4.0-4.2 (in, —CH2-, 4H)

The obtained compound (M0-4) was subjected to NMR measurement, and the structure thereof was identified from the following analysis results.

¹H-NMR (dmso-d6, 400 MHz) δ (ppm)=8.2 (m, —Ar, 1H), 7.7-7.9 (m, -Ph, 15H), 7.7 (m, —Ar, 1H), 7.0 (m, —Ar, 1H), 6.0-6.2 (m, =CH2+-CH═CH2, 2H), 5.7 (s, ═CH2, 1H), 5.1-5.4 (m, —CH═CH2, 2H), 4.6-4.7 (m, —CH2-, 2H), 4.0-4.2 (m, —CH2-, 4H)

The obtained compound (M0-5) was subjected to NMR measurement, and the structure thereof was identified from the following analysis results.

¹H-NMR (dmso-d6, 400 MHz) δ (ppm)=8.6 (m, —Ar, 1H), 8.3 (m, —Ar, 1H), 7.7-7.9 (m, -Ph, 15H), 6.0-6.2 (m, =CH2+-CH═CH2, 2H), 5.7 (s, ═CH2, 1H), 5.1-5.4 (m, —CH═CH2, 2H), 4.6-4.7 (m, —CH2-, 2H), 4.0-4.2 (m, —CH2-, 4H)

The obtained compound (M0-6) was subjected to NMR measurement, and the structure thereof was identified from the following analysis results.

¹H-NMR (dmso-d6, 400 MHz) δ (ppm)=8.5 (m, —Ar, 2H), 8.4 (in, —Ar, 2H), 8.0 (m, —Ar, 2H), 7.6-7.8 (in, —Ar, 7H), 6.0-6.2 (m, =CH2+-CH═CH2, 2H), 5.7 (s, ═CH2, 1H), 5.1-5.4 (m, —CH═CH2, 2H), 4.6-4.7 (m, —CH2-, 2H), 4.0-4.2 (m, —CH2-, 4H)

The obtained compound (M0-7) was subjected to NMR measurement, and the structure thereof was identified from the following analysis results.

¹H-NMR (dmso-d6, 400 MHz) δ (ppm)=8.2 (m, —Ar, 2H), 7.7-7.9 (m, —Ar, 10H), 7.5 (m, —Ar, 2H), 6.0-6.2 (m, =CH2+-CH═CH2, 2H), 5.7 (s, ═CH2, 1H), 5.1-5.4 (m, —CH═CH2, 2H), 4.6-4.7 (m, —CH2-, 2H), 4.0-4.2 (m, —CH2-, 4H)

The obtained compound (M0-8) was subjected to NMR measurement, and the structure thereof was identified from the following analysis results.

¹H-NMR (dmso-d6, 400 MHz) δ (ppm)=7.7-8.0 (m, —Ar, 11H), 6.0-6.2 (m, =CH2+-CH═CH2, 2H), 5.7 (s, ═CH2, 1H), 5.1-5.4 (m, —CH═CH2, 2H), 4.6-4.7 (m, —CH2-, 2H), 4.0-4.2 (m, —CH2-, 4H)

The obtained compound (M0-9) was subjected to NMR measurement, and the structure thereof was identified from the following analysis results.

¹H-NMR (dmso-d6, 400 MHz) δ (ppm)=8.3 (m, —Ar, 2H), 8.1-8.2 (m, —Ar, 4H), 7.9-8.0 (m, —Ar, 5H), 7.6 (m, —Ar, 2H), 6.0-6.2 (m, ═CH2+-CH═CH2, 2H), 5.7 (s, ═CH2, 1H), 5.1-5.4 (m, —CH═CH2, 2H), 4.6-4.7 (m, —CH2-, 2H), 4.0-4.2 (m, —CH2-, 4H)

The obtained compound (M0-10) was subjected to NMR measurement, and the structure thereof was identified from the following analysis results.

¹H-NMR (dmso-d6, 400 MHz) δ (ppm)=8.2 (m, —Ar, 2H), 7.7-7.9 (m, -Ph, 30H), 7.7 (m, —Ar, 2H), 7.0 (m, —Ar, 2H), 6.2 (s, ═CH2, 2H), 5.8 (s, ═CH2, 2H), 4.6 (m, —CH2-, 4H), 4.1 (s. —CH2-, 4H)

The obtained compound (M0-11) was subjected to NMR measurement, and the structure thereof was identified from the following analysis results.

¹H-NMR (dmso-d6, 400 MHz) δ (ppm)=8.2 (in, —Ar, 1H), 7.7-7.9 (m, -Ph, 15H), 7.7 (in, —Ar, 1H), 7.0 (m, —Ar, 1H), 6.2 (s, ═CH2, 1H), 5.8 (s, ═CH2, 1H), 4.9 (s, ═CH2, 1H), 4.8 (s, ═CH2, 1H), 4.6 (m, —CH2-, 2H), 4.1 (s, —CH2-, 2H), 3.9 (s, —CH2-, 2H), 1.7 (s, —CH3, 3H)

### Synthesis Example of Polymer Compound

### Polymer Synthesis Example 1

2.9 g of the compound (M0-1), 3.0 g of the compound (m10-1pre), 5.9 g of the compound (m1-1), and 0.9 g of dimethyl azobis(isobutyrate) (V-601) as a polymerization initiator were dissolved in 40 g of methyl ethyl ketone (MEK), heated to 80° C. under a nitrogen atmosphere, and stirred for 6 hours. Thereafter, 2.0 g of acetic acid and 60 g of methanol were added to the reaction solution, and the reaction solution was stirred at 30° C. for 18 hours. After completion of the reaction, the obtained reaction solution was mixed with 600 g of heptane to obtain a precipitate. The obtained precipitate was washed. The obtained white solid was filtered and dried under reduced pressure overnight to obtain 5.9 g of a target polymer compound (A1-1).

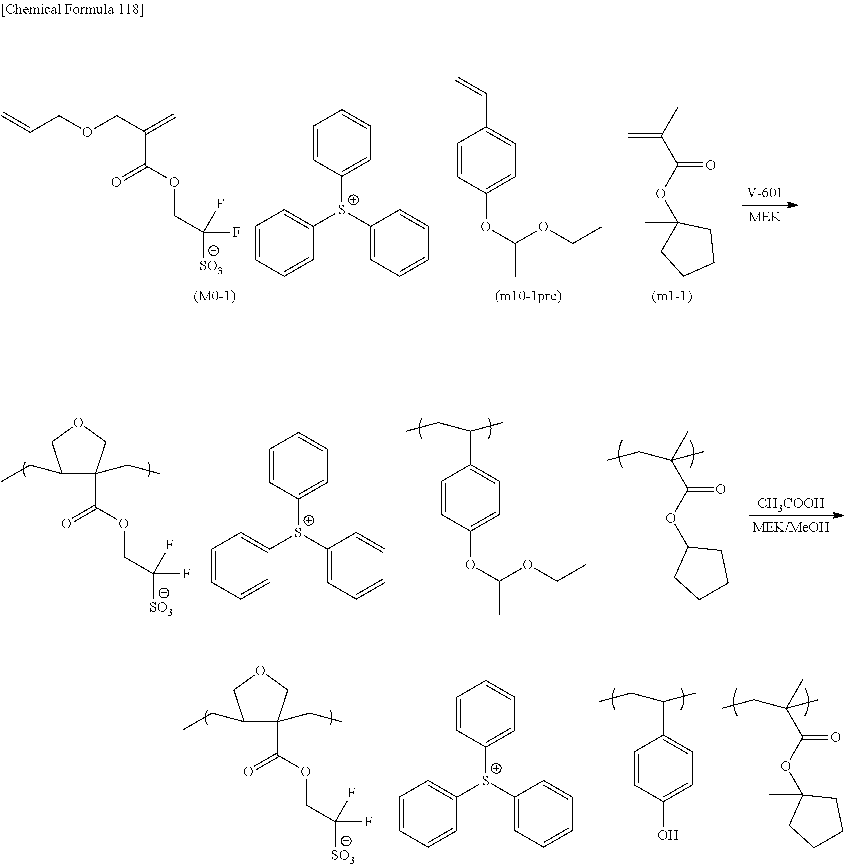

### Polymer Synthesis Examples 2 to 16

Polymer compounds (A1-2) to (A1-16) were obtained in the same manner as in the polymer synthesis example 1, except that the compound used in the polymerization reaction was changed.

The obtained polymer compounds (A1-1) to (A1-16) are shown below. In the following formula, l, m, n, and o represent a compositional ratio (molar ratio) of each constitutional unit.

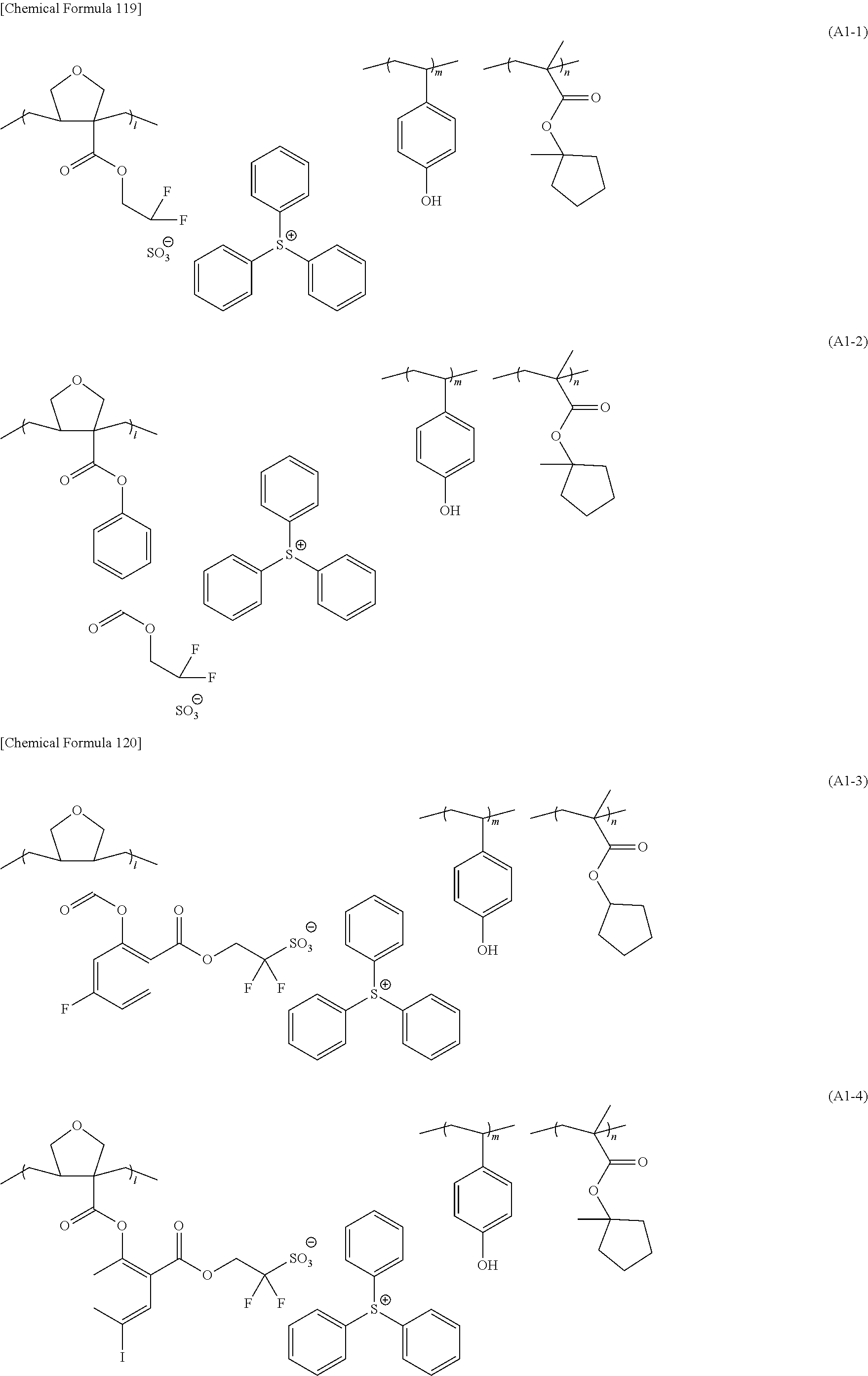

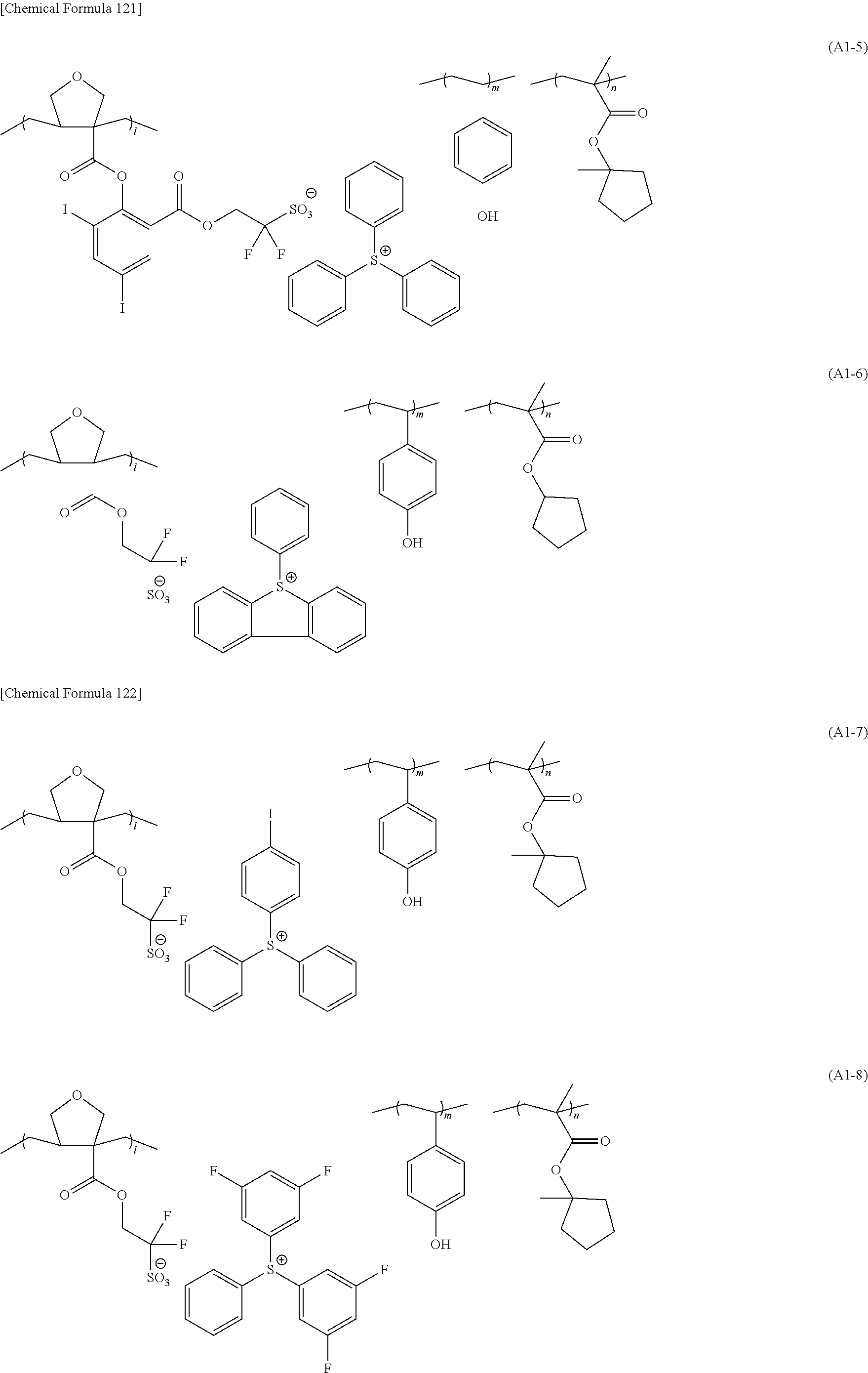

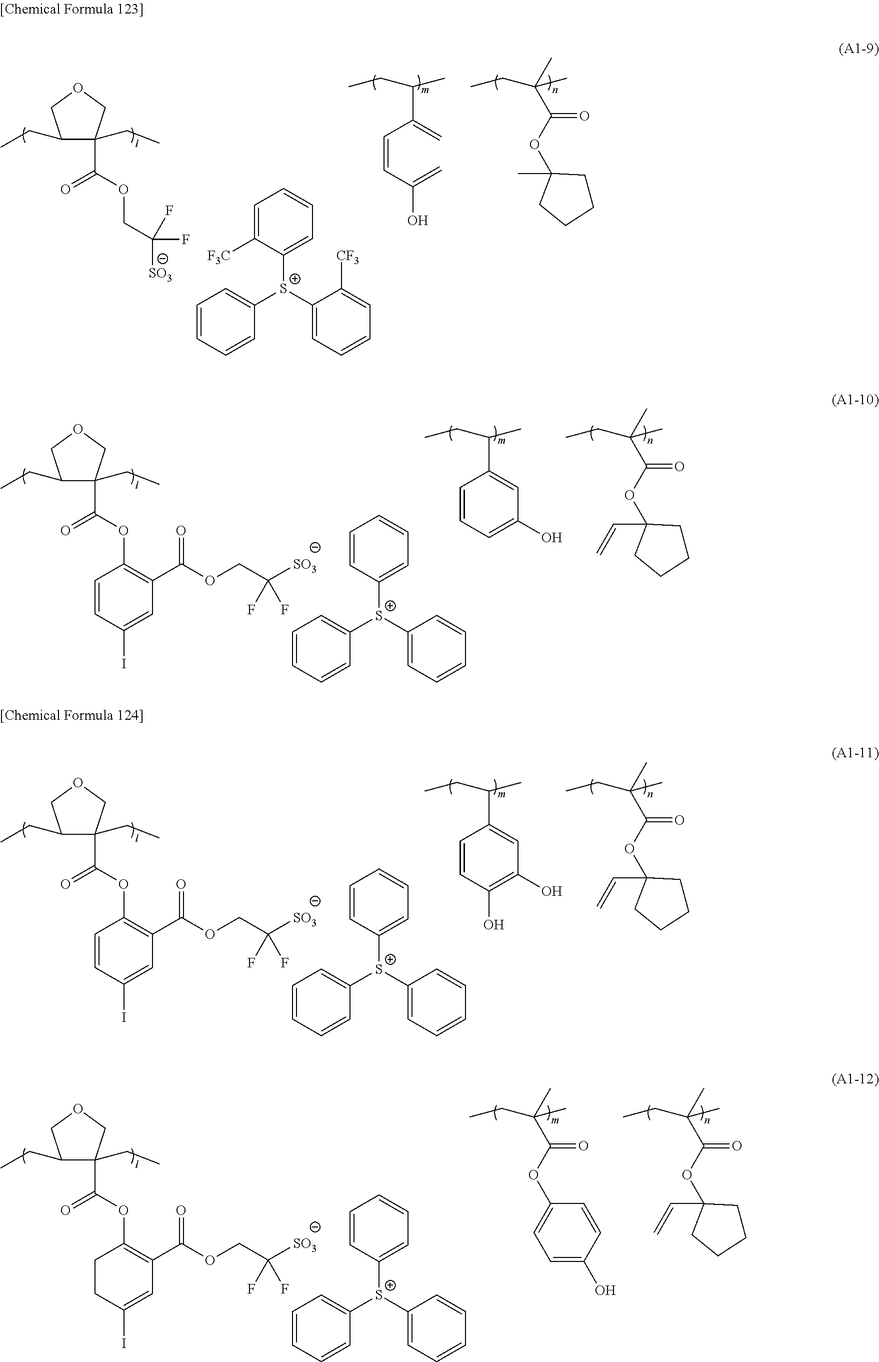

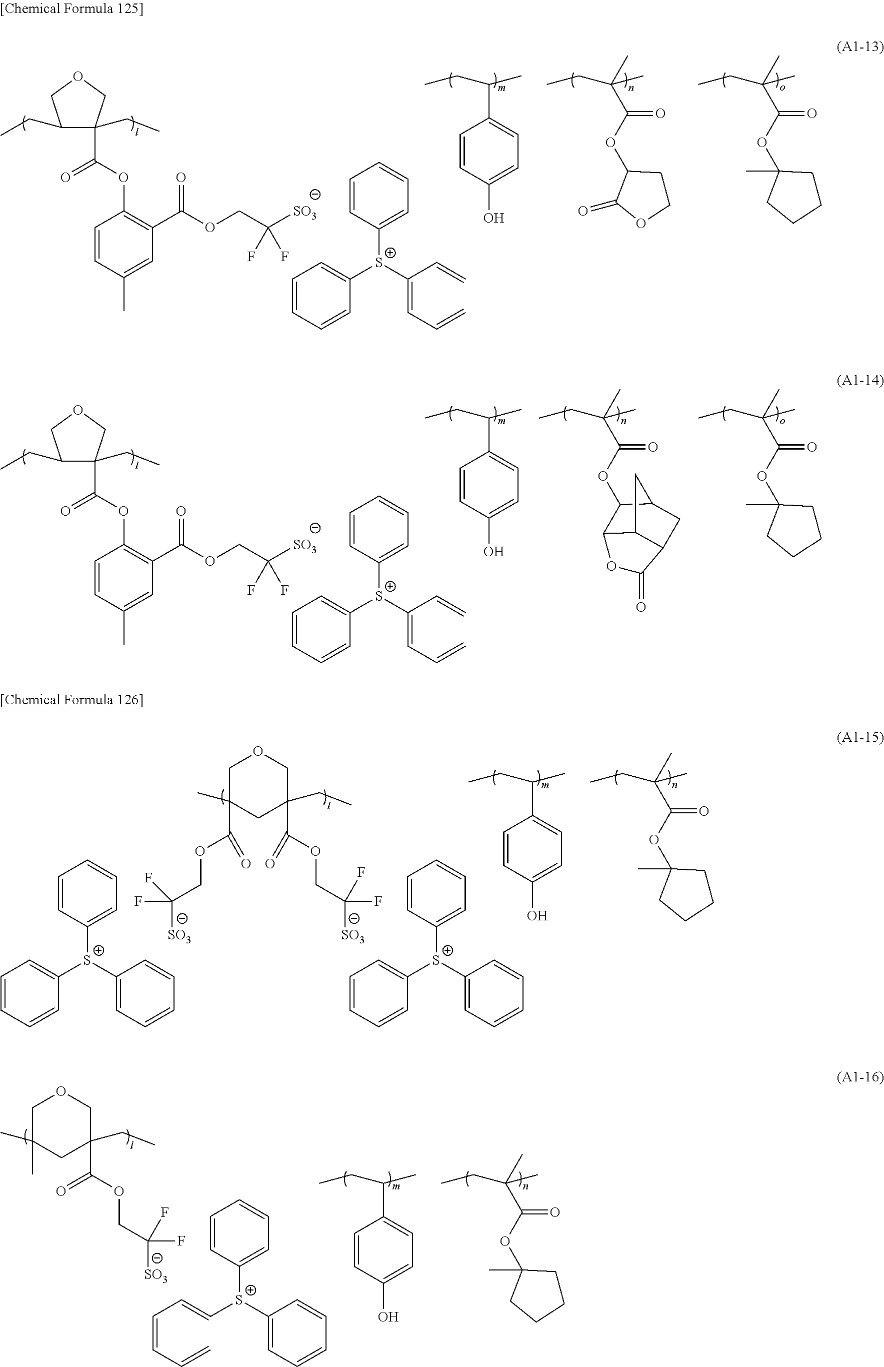

### Polymer Synthesis Example of Comparative Example

Polymer compounds (A2-1) to (A2-3) were obtained in the same manner as in the polymer synthesis example 1, except that the compound used in the polymerization reaction was changed. The obtained polymer compounds (A2-1) to (A2-3) are shown below. In the following formula, 1, m, and n represent a compositional ratio (molar ratio) of each constitutional unit.

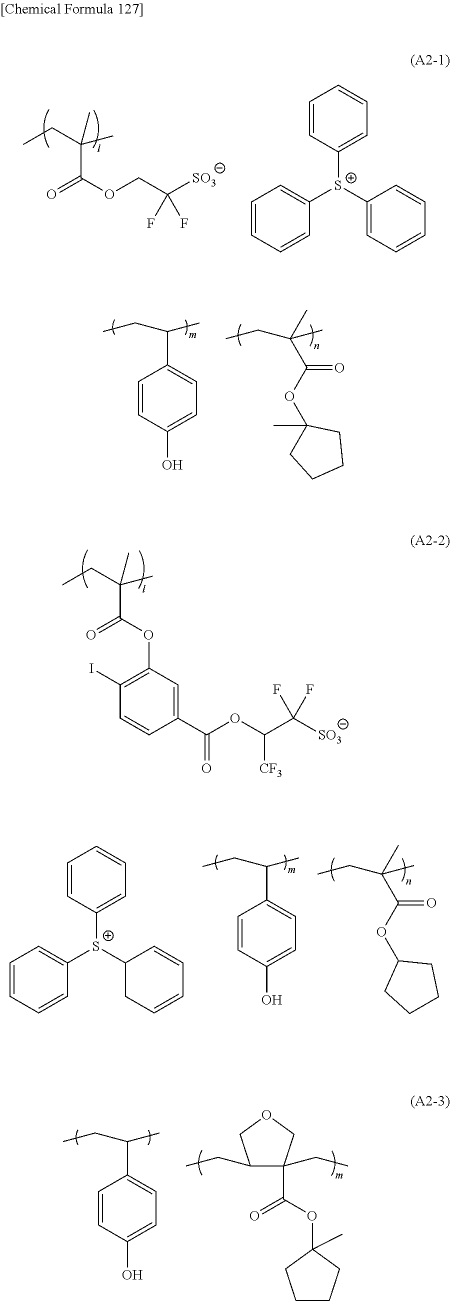

For each of the obtained polymer compounds (A1-1) to (A1-16) and the polymer compounds (A2-1) to (A2-3), a weight-average molecular weight (Mw) and a molecular weight dispersity (Mw/Mn) were determined by GPC measurement (in terms of standard polystyrene). In addition, for the polymer compounds (A1-1) to (A1-16) and the polymer compounds (A2-1) to (A2-3), the copolymerization composition ratio (the proportion (molar ratio) of each constitutional unit in the structural formula) was determined by carbon-13 nuclear magnetic resonance spectroscopy (600 MHz, ¹³C-NMR). The results are listed in Table 1.

TABLE 1

| Polymer | | | |
|---|---|---|---|
| compound | Molar ratio | Mw | Mw/Mn |
| A1-1 | l/m/n = 10/30/60 | 7600 | 1.70 |
| A1-2 | l/m/n = 10/30/60 | 8200 | 1.72 |
| A1-3 | l/m/n = 10/30/60 | 9300 | 1.67 |
| A1-4 | l/m/n = 10/30/60 | 8500 | 1.66 |
| A1-5 | l/m/n = 10/30/60 | 7200 | 1.71 |
| A1-6 | l/m/n = 10/30/60 | 6700 | 1.73 |
| A1-7 | l/m/n = 10/30/60 | 6500 | 1.67 |
| A1-8 | l/m/n = 10/30/60 | 8300 | 1.72 |
| A1-9 | l/m/n = 10/30/60 | 9200 | 1.61 |
| A1-10 | l/m/n = 10/30/60 | 8500 | 1.67 |
| A1-11 | l/m/n = 15/20/65 | 8700 | 1.68 |
| A1-12 | l/m/n = 15/30/55 | 7200 | 1.72 |
| A1-13 | l/m/n/o = 10/30/10/50 | 7600 | 1.66 |
| A1-14 | l/m/n/o = 10/30/10/50 | 9100 | 1.71 |
| A1-15 | l/m/n = 5/30/65 | 8300 | 1.68 |
| A1-16 | l/m/n = 10/30/60 | 7200 | 1.67 |
| A2-1 | l/m/n = 10/30/60 | 8700 | 1.71 |
| A2-2 | l/m/n = 10/30/60 | 9700 | 1.76 |
| A2-3 | l/m = 35/65 | 7500 | 1.62 |

### Preparation of Resist Composition

### Examples 1 to 20 and Comparative Examples 1 to 3

Each of the components shown in Tables 2 and 3 was mixed and dissolved to prepare a resist composition of each Example.

TABLE 2

| | Component | Component | Component | Component |
|---|---|---|---|---|
| | (A) | (B) | (D) | (S) |
| Example 1 | (A1)-1 | — | (D1)-1 | (S)-1 |
| | [100] | | [6.0] | [6400] |
| Example 2 | (A1)-2 | — | (D1)-1 | (S)-1 |
| | [100] | | [6.0] | [6400] |
| Example 3 | (A1)-3 | — | (D1)-1 | (S)-1 |
| | [100] | | [6.0] | [6400] |
| Example 4 | (A1)-4 | — | (D1)-1 | (S)-1 |
| | [100] | | [6.0] | [6400] |
| Example 5 | (A1)-5 | — | (D1)-1 | (S)-1 |
| | [100] | | [6.0] | [6400] |
| Example 6 | (A1)-6 | — | (D1)-1 | (S)-1 |
| | [100] | | [6.0] | [6400] |
| Example 7 | (A1)-7 | — | (DI)-1 | (S)-1 |
| | [100] | | [6.0] | [6400] |
| Example 8 | (A1)-8 | — | (DI)-1 | (S)-1 |
| | [100] | | [6.0] | [6400] |
| Example 9 | (A1)-9 | — | (DI)-1 | (S)-1 |
| | [100] | | [6.0] | [6400] |
| Example 10 | (A1)-10 | — | (D1)-1 | (S)-1 |
| | [100] | | [6.0] | [6400] |
| Example 11 | (A1)-11 | — | (D1)-1 | (S)-1 |
| | [100] | | [6.0] | [6400] |
| Example 12 | (A1)-12 | — | (D1)-1 | (S)-1 |
| | [100] | | [6.0] | [6400] |
| Example 13 | (A1)-13 | — | (D1)-1 | (S)-1 |
| | [100] | | [6.0] | [6400] |
| Example 14 | (A1)-14 | — | (DI)-1 | (S)-1 |
| | [100] | | [6.0] | [6400] |
| Example 15 | (A1)-4 | — | (D1)-2 | (S)-1 |
| | [100] | | [7.0] | [6400] |
| Example 16 | (A1)-4 | — | (D1)-3 | (S)-1 |
| | [100] | | [9.8] | [6400] |
| Example 17 | (A1)-4 | (B1)-1 | (D1)-1 | (S)-1 |
| | [100] | [4.0] | [6.5] | [6400] |
| Example 18 | (A1)-4 | (B1)-2 | (D1)-1 | (S)-1 |
| | [100] | [6.2] | [6.5] | [6400] |
| Example 19 | (A1)-15 | — | (D1)-1 | (S)-1 |
| | [100] | | [6.0] | [6400] |
| Example 20 | (A1)-16 | — | (D1)-1 | (S)-1 |
| | [100] | | [6.0] | [6400] |

TABLE 3

| | Component | Component | Component | Component |
|---|---|---|---|---|
| | (A) | (B) | (D) | (S) |
| Comparative | (A2)-1 | — | (D1)-1 | (S)-1 |
| Example 1 | [100] | | [6.0] | [6400] |
| Comparative | (A2)-2 | — | (D1)-1 | (S)-1 |
| Example 2 | [100] | | [6.0] | [6400] |
| Comparative | (A2)-3 | (B1)-1 | (D1)-1 | (S)-1 |
| Example 3 | [100] | [30.6] | [6.0] | [6400] |

In Tables 2 and 3, each abbreviation has the following meaning. The numerical values in the brackets are blending amounts (parts by mass).
(A1)-1 to (A1)-16: high molecular weight compounds (A1-1) to (A1-16) shown above
(A2)-1 to (A2)-3: high molecular weight compounds (A2-1) to (A2-3) shown above
(B1)-1: An acid generator consisting of the following compound (B1-1)
(B1)-2: An acid generator consisting of the following compound (B1-2)

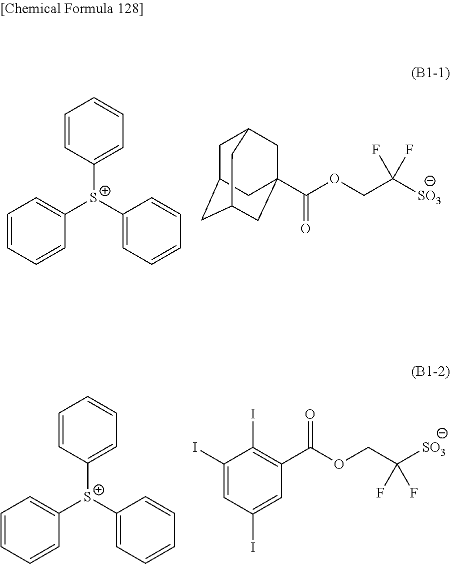

(D1)-1: acid diffusion control agent consisting of compound (D1-1) shown below
(D1)-2: acid diffusion control agent consisting of compound (D1-2) shown below
(D1)-3: acid diffusion control agent consisting of compound (D1-3) shown below

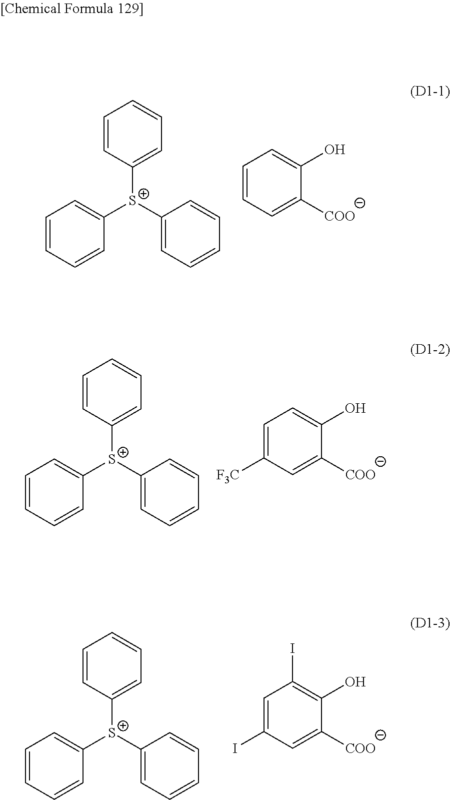

(S)-1: mixed solvent of propylene glycol monomethyl ether acetate/propylene glycol monomethyl ether at mass ratio of 60/40

### <Resist Pattern Formation>

The resist composition of each example was applied onto an 8-inch silicon substrate which had been subjected to a hexamethyldisilazane (HMDS) treatment using a spinner, the coated film was subjected to a pre-bake (PAB) treatment on a hot plate at a temperature of 110° C. for 60 seconds so that the coated film was dried to form a resist film having a film thickness of 60 nm.

Next, the resist film was subjected to drawing (light exposure) to obtain a contact hole pattern (hereinafter, referred to as “CH pattern”) in which holes having a diameter of 26 nm were arranged at equal intervals (pitch: 46 nm) using an electron beam lithography apparatus JEOL-JBX-9300FS (manufactured by JEOL Ltd.) at an acceleration voltage of 100 kV.

Thereafter, a post exposure bake (PEB) treatment was performed thereon at 100° C. for 60 seconds.

Subsequently, alkali development was performed at 23° C. for 60 seconds using a 2.38 mass % tetramethylammonium hydroxide (TMAH) aqueous solution “NMD-3” (trade name, manufactured by TOKYO OHKA KOGYO CO., LTD.).

Thereafter, water rinsing was performed for 15 seconds using pure water.

As a result of the above, a CH pattern in which holes having a diameter of 26 nm were arranged at equal intervals (pitch: 46 nm) was formed.

### [Evaluation of Sensitivity]

According to <Resist pattern formation> described above, an optimum exposure amount Hop (μC/cm²) at which a CH pattern having the target size was formed was determined. The results thereof are listed in the columns of “Fop (μC/cm²)” in Tables 4 and 5.

[Evaluation of in-Plane Uniformity (CDU) of Pattern Dimensions]

The CH pattern formed according to <Resist pattern formation> described above was observed from the upper side of the CH pattern, and the hole diameter (nm) of each of the holes was measured with a scanning electron microscope (CD-SEM, acceleration voltage: 500 V, trade name: CG5000, manufactured by Hitachi High-Tech Corporation). A triple value (3a) of the standard deviation (o) calculated from the measurement results was determined. The lower the value of 3a determined as described above is, the higher the critical dimension (CD) uniformity of the plurality of holes formed in the resist film is. The results are listed in the columns of “CDU (nm)” of Tables 4 and 5.

### [Evaluation of Fine Resolution]

The limiting resolution at the optimum exposure amount (Eop) at which the CH pattern was formed by <Resist pattern formation> described above, specifically, in a case where the exposure amount was gradually decreased from the optimum exposure amount (Eop) to form the CH pattern, the hole diameter (nm) of the pattern to be resolved was determined using a scanning electron microscope (SEM) (accelerating voltage: 500 V, trade name: CG5000, manufactured by Hitachi High-Tech Corporation). The results are listed in the columns of “Resolution (nm)” in Tables 4 and 5.

TABLE 4

| | Eop | CDU | Resolution |
|---|---|---|---|
| | (μC/cm²) | (nm) | (nm) |
| Example 1 | 121 | 5.1 | 21 |
| Example 2 | 118 | 4.9 | 20 |
| Example 3 | 114 | 4.7 | 20 |
| Example 4 | 109 | 4.4 | 19 |
| Example 5 | 105 | 4.3 | 19 |
| Example 6 | 122 | 5.0 | 21 |
| Example 7 | 116 | 4.9 | 21 |
| Example 8 | 114 | 4.7 | 20 |
| Example 9 | 116 | 4.8 | 21 |
| Example 10 | 110 | 4.2 | 19 |
| Example 11 | 107 | 4.3 | 20 |
| Example 12 | 109 | 4.2 | 20 |
| Example 13 | 107 | 4.5 | 20 |
| Example 14 | 109 | 4.5 | 20 |
| Example 15 | 105 | 4.2 | 20 |
| Example 16 | 102 | 4.1 | 19 |
| Example 17 | 105 | 4.7 | 21 |
| Example 18 | 101 | 4.5 | 20 |
| Example 19 | 112 | 4.2 | 19 |
| Example 20 | 108 | 4.5 | 19 |

TABLE 5

| | Eop | CDU | Resolution |
|---|---|---|---|
| | (μC/cm²) | (nm) | (nm) |
| Comparative Example 1 | 123 | 6.4 | 24 |
| Comparative Example 2 | 113 | 5.8 | 24 |
| Comparative Example 3 | 108 | 6.0 | 26 |

As shown in the results listed in Tables 4 and 5, it was confirmed that the resist compositions of Examples 1 to 20 had good sensitivity, CDU, and resolution. On the other hand, the resist compositions of Comparative Examples 1 to 3 were inferior in CDU and resolution as compared with the resist compositions of Examples 1 to 20.

Hereinbefore, preferred examples of the present invention have been described, but the present invention is not limited to these examples. Additions, omissions, substitutions, and other modifications can be made without departing from the scope of the present invention. The present invention is not limited by the above description, but only by the scope of the appended claims.

What is claimed is:

1. A resist composition which generates an acid upon light exposure and whose solubility in a developing solution is changed by an action of the acid, the resist composition comprising:
a resin component (A1) whose solubility in a developing solution is changed by the action of the acid,
wherein the resin component (A1) has a constitutional unit (a0) represented by General Formula (a0-1), 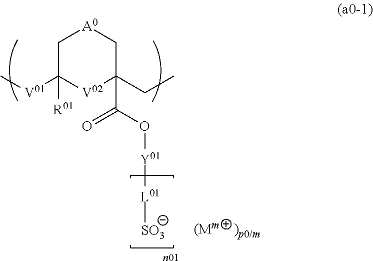
wherein A⁰ represents an oxygen atom or a sulfur atom, Y⁰¹ represents an (n01+1)-valent linking group, L⁰¹ represents a single bond or a divalent linking group, R⁰¹ represents a hydrogen atom or a group represented by General Formula (r01-1) or (r01-2), V⁰¹ and V⁰² each represents a single bond or a methylene group, where V⁰² represents a methylene group in a case where V⁰¹ represents a single bond and V⁰² represents a single bond in a case where V⁰¹ represents a methylene group, n01 represents an integer of 1 or greater as long as a valence is allowed, m represents an integer of 1 or greater and M^{m+} represents an m-valent cation, and p0=n01 in a case where R⁰¹ represents a hydrogen atom or a group represented by General Formula (r01-2), and p0=n01+n02 in a case where R⁰¹ represents a group represented by General Formula (r01-1), 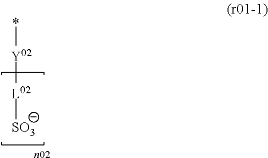
wherein Y⁰² represents an (n02+1)-valent linking group, L⁰² represents a single bond or a divalent linking group, and n02 represents an integer of 1 or greater as long as a valence is allowed, 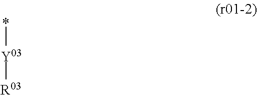
wherein Y⁰³ represents a single bond or a divalent linking group, and R⁰³ represents a hydrocarbon group which may have a substituent.

2. The resist composition according to claim 1, wherein the constitutional unit (a0) is a constitutional unit represented by General Formula (a01-1), (a02-1), or (a03-1), 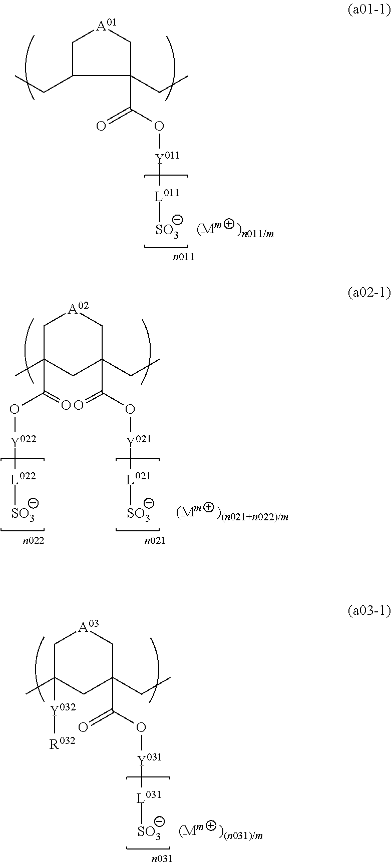
wherein A⁰¹ to A⁰³ each independently represents an oxygen atom or a sulfur atom, Y⁰¹¹ represents an (n011+1)-valent linking group, Y⁰²¹ represents an (n021+1)-valent linking group, Y⁰²² represents an (n022+1)-valent linking group, Y⁰³¹ represents an (n031+1)-valent linking group, L⁰¹¹, L⁰²¹, L⁰²², L⁰³¹, and Y⁰³² each independently represents a single bond or a divalent linking group, R⁰³² represents a hydrocarbon group which may have a substituent, n011, n021, n022, and n031 each independently represents an integer of 1 or greater as long as a valence is allowed, and m represents an integer of 1 or greater and M^{m+} represents an m-valent cation.

3. A resist pattern formation method, comprising:
forming a resist film on a support using the resist composition according to claim 1;
exposing the resist film to light; and
developing the resist film exposed to light to form a resist pattern.

4. A compound represented by General Formula (m0-1), 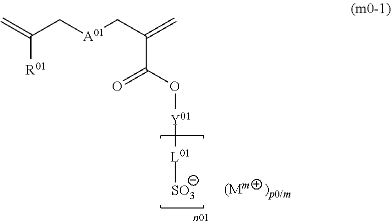
wherein A⁰ represents an oxygen atom or a sulfur atom, Y⁰¹ represents an (n01+1)-valent linking group, L⁰¹ represents a single bond or a divalent linking group, R⁰¹ represents a hydrogen atom or a group represented by General Formula (r01-1) or (r01-2), n01 represents an integer of 1 or greater as long as a valence is allowed, m represents an integer of 1 or greater and M^{m+} represents an m-valent cation, and p0=n01 in a case where R⁰¹ represents a hydrogen atom or a group represented by General Formula (r01-2), and p0=n01+n02 in a case where R⁰¹ represents a group represented by General Formula (r01-1), 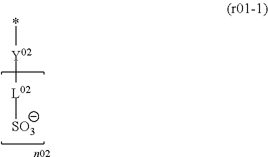
wherein Y⁰² represents an (n02+1)-valent linking group, L⁰² represents a single bond or a divalent linking group, and n02 represents an integer of 1 or greater as long as a valence is allowed, 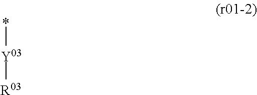
wherein Y⁰³ represents a single bond or a divalent linking group, and R⁰³ represents a hydrocarbon group which may have a substituent.

5. A polymer compound comprising:
a constitutional unit (a0) represented by General Formula (a0-1), 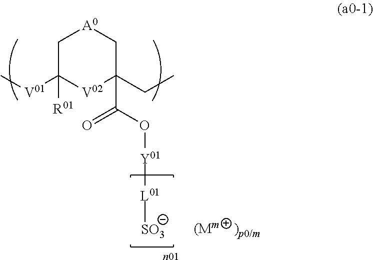
wherein A⁰ represents an oxygen atom or a sulfur atom, Y⁰¹ represents an (n01+1)-valent linking group, L⁰¹ represents a single bond or a divalent linking group, R⁰¹ represents a hydrogen atom or a group represented by General Formula (r01-1) or (r01-2), V⁰¹ and V⁰² each represents a single bond or a methylene group, where V⁰² represents a methylene group in a case where V⁰¹ represents a single bond and V⁰² represents a single bond in a case where V⁰¹ represents a methylene group, n01 represents an integer of 1 or greater as long as a valence is allowed, m represents an integer of 1 or greater and M^{m+} represents an m-valent cation, and p0=n01 in a case where R⁰¹ represents a hydrogen atom or a group represented by General Formula (r01-2), and p0=n01+n02 in a case where R⁰¹ represents a group represented by General Formula (r01-1), 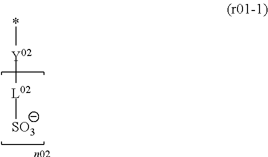
wherein Y⁰² represents an (n02+1)-valent linking group, L⁰² represents a single bond or a divalent linking group, and n02 represents an integer of 1 or greater as long as a valence is allowed, 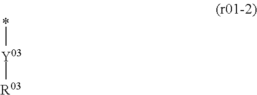
wherein Y⁰³ represents a single bond or a divalent linking group, and R⁰³ represents a hydrocarbon group which may have a substituent.
